SEQUENCE LISTING (52 sequences)

SEQ ID NO: 1 - protein, 102 aa, Homo sapiens
  ref <308>: DATABASE ACCESSION NUMBER: Q04984
  ref <309>: DATABASE ENTRY DATE: 2003-09-15
  ref <313>: RELEVANT RESIDUES: (1)..(102)
  ref <308>: DATABASE ACCESSION NUMBER: AAH23518
  ref <309>: DATABASE ENTRY DATE: 2004-06-29
  ref <313>: RELEVANT RESIDUES: (1)..(102)
MAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 2 - DNA, 538 bp, Homo sapiens
  ref <308>: DATABASE ACCESSION NUMBER: NM_002157
  ref <309>: DATABASE ENTRY DATE: 2004-12-18
  ref <313>: RELEVANT RESIDUES: (1)..(538)
gctacactagagcagagtacgagtctgaggcggagggagtaatggcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactgaaataagtcactattgaaatggcatcaacatgatgctgcccattccactgaagttctgaaatctttcgtcatgtaaataatttccatatttctcttttataataaactaatgataactaatgacatccagtgtctccaaaattgtttccttgtactgatataaacacttccaaataaaaatatgtaaat

SEQ ID NO: 3 - protein, 93 aa, Homo sapiens
  ref <308>: DATABASE ACCESSION NUMBER: P05109
  ref <309>: DATABASE ENTRY DATE: 2004-10-25
  ref <313>: RELEVANT RESIDUES: (1)..(93)
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 4 - DNA, 4205 bp, Homo sapiens
  ref <308>: DATABASE ACCESSION NUMBER: A12027
  ref <309>: DATABASE ENTRY DATE: 1994-11-30
  ref <313>: RELEVANT RESIDUES: (1)..(4205)
cttgggttgcttccaccttttggctcttgtaaataatgctgctatgaacatgaatgtacaaacatctgtttgaatccctgcattcaattcttttgcatatatacccaggagcagaatgatggatcatatggtaattctgtgtttatttatttgaggaacaaacttgccgttttccataacagctgcactattttacattcccactaacagtgcattaggcttccaattctctatgccctcaccaacacttgttttctgggttttaaaagaagtagtagtcatccttgtaggtgtcaggtggtatctcattgtcgttttgcttcatgttttcctaaagattagtaattttcatatgcttattgaccatttgtatatcttcttcggagaagtgtctatttgagtctttccccaattttgattggtttgtttgttttttgttgttgagttgtagggattcttttatattctggatattaatcccttatcagatatttgttttacaaatattttctttgtaacaacagaaacacaccacagtcttcaaggttggaagccagttaatctgagtagcattttgttagtggtggggagaggatttgttcctcctgaaatcctggggaattggccacctcctcttctcctcttaggcatgaagcgcgtctggcttctccaaagaactcttcccctccactacctcagagttagcttcctctcttcagccagtgatcctggggtcccagacacaataattaaccaagagagggtgaaaggctccctgctgtgtttatgcaatggctcaggcccttgtgaagtgccgagggaccccaagcagcctccatctcccagggcatggtccatccccagctttcacagaacaggaaagctgtggaggagtgtgggcagcagggtaggaatggatatagcccttggcaacaacacatttccccacaaagcacccacccaaaagaacaacaacgatagttttagtttttagtaatgagaacaatagttctcatgactaaaagccatcagccaggacactgttctcaacccttttgcggtctttggaccctttgaaactctgacagaagccatggaggaatgttctcactgagtgcatgcactcaaaatgatgcattcaacttcaattcagtttcagggatgtatggcctgaccaccaatgcaggggattagcaatcgcaatagtggagagggcatgggagtgggaatctggctggatcaagcaagtggatgccagcagcccagaaaaagagcccccctacctgctttttccttcctgggcactattgcccagcaaatgccttcctctttccgcttctcctacctccccacccaaaattttcattctgcacagtgattgccacattcactggttgagaaacagagactgtagcaactctggcagggagaagctgtctctgatggcctgaagctgtgggcagctggccaagcctaaccgctataaaaaggagctgcctctcagccctgcatgtctcttgtcagctgtctttcagaagacctggtaagtgggactgtctgggttggccccgcactttgggcttctcttggggagggtcagggaagtggagcagccttcctgagagaggagagagaaagctcagggaggtctggagcaaagatactcctggaggtggggagtgaggcagggataaggaaggagagtatcctccagcaccttccagtgggtaagggcacattgtctcctaggctggacttttcttgagcagagggtggggtggtaaggaaagtctacgggcccccgtgtgtgtgcacatgtctctgtgtgaatggacccttccccttcccacacgtgtatccctatcatcccacccttcccaccagaggccatagccatctgctggtttggttatttgagagtgcaggccaggacaaggccatcgcttggggcatgaatcctctgcgtactgccctggccagatgcaaattccctgccatgggattccccagaaggttctgtttttcaggtggggcaagttccgtgggcatcatgttgaccgagctggagaaagccttgaactctatcatcgacgtctaccacaagtactccctgataaaggggaatttccatgccgtctacagggatgacctgaagaaattgctagagaccgagtgtcctcagtatatcagggtgaggaggggctgggtgtggcgggggctctctgcctggtcctggggctgccctgggccagcggtcctccctgccacccttcatagatgctatgcctcggctctctctgagatctttaaactctggcttcttcctcctcaatcttgacagaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtagctgagttactgggcccagaggctgggcccctggacatgtacctgcagaataataaagtcatcaatacctcatgcctctctcttatgcttttgtggaatgaggttcctcggtgtggagggagggttggaaaacccaaaggaagaaaaagaaatctatgttatcccaccctacctctcacaagcctttcctgctttacccctcacctggcctctgccccacattccttcagcccctcatttcgagcattggatttgaggcttaaggattcaaaaagtcgtcatgaatatagctgatgattttatagtggttctgaaatgggtcggggatttgggaacagggtggtagtataagaacaactgatactgttctctaagctaaatcttagcttccagctacctgtcttagatgtggctcttgggaaccttagagtgatagctacatagaagtgtgtgggtgtgtgtgtgtgtgtctgtgtgtgtgtgtgtgagagagagacagacagaaagagagcaagagagggaaggggggagaggctgattgtgtgtgtggtgtgatgtaggtggacaatgttcagagtcctccattaacaggataatcctcacacctgtccacatacctgtagtttgtccttggggattttgaaaatttttcctccctctccactcccaaactcccaactcaattaaatgataaaggaataggcaaataggaaaataaattagtaaaacttaagtcaaagaataggttattcatacgctgcctatgggattctatgctttgtgatcagaaaattatctaaaaaatacttcccaagggctggtacaagggaggccagaagacgagtggttcttctctgaggtggacattaaaaaaagaagaaaatgaaggggaaccttttgacaagaatgtcaccccaaactggattttcatgctgtggtgtggggaattttctgttgtcctcacttaggtgctggggcagtggtgttagtgatgggtaaaaaggtaggaagctgtcacagaatcactaaaccagggttcttaacttgtctgtctatacatctctgaaattgggttgaagttgtgtgcatcattttgagtgacgcactgagaacattcctccacggcttccatcgagagtctcgaaaaggcccaacacctcaaaaaggttaagaacacttgtcctgcttactggtttttagtaacaaatggcagagtatttctctctgtctctctctctttttttttttttttttttgagacacagggtcttgtctgtcacgtggactagagtacaatgggcatgatcatgggctcactgtagcctcgaacacctgggctcaagtaatcctcccacctcagcctctttagtagctgggactacagcatgagccactgcccttggctaatttttaaattatttttttgtagagatggaaacttgctatgttgcccaggctagtctcaaactcctggactcaagcgatcctcctaccttggcctcccaaagtgctgagattacagtgtgatccacaccacacctggccaaagattggagtatttttattgctattgttgtgctgggtgggtgggtgggtgtatgctttgtggggacgtgtgttgttgccaagggctaaatcagttcctaccctgctgcccacagtcctccacagctttcctgctctgtgaagctaaggatacaccccgatgataagctgtcaacata

SEQ ID NO: 5 - DNA, 428 bp, Homo sapiens
  ref <308>: DATABASE ACCESSION NUMBER: NM_002964
  ref <309>: DATABASE ENTRY DATE: 2004-10-26
  ref <313>: RELEVANT RESIDUES: (1)..(428)
atgtctcttgtcagctgtctttcagaagacctggtggggcaagtccgtgggcatcatgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtagctgagttactgggcccagaggctgggcccctggacatgtacctgcagaataataaagtcatcaatacctcaaaaaaaaaaaaaaaaaaaaa

SEQ ID NO: 6 - protein, 114 aa, Homo sapiens
  ref <308>: DATABASE ACCESSION NUMBER: P06702
  ref <309>: DATABASE ENTRY DATE: 1993-09-12
  ref <313>: RELEVANT RESIDUES: (1)..(114)
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 7 - DNA, 462 bp, Homo sapiens
  ref <308>: DATABASE ACCESSION NUMBER: X06233
  ref <309>: DATABASE ENTRY DATE: 1993-09-12
  ref <313>: RELEVANT RESIDUES: (1)..(462)
aaaacactctgtgtggctcctcggctttgacagagtgcaagacgatgacttgcaaaatgtcgcagctggaacgcaacatagagaccatcatcaacaccttccaccaatactctgtgaagctggggcacccagacaccctgaaccagggggaattcaaagagctggtgcgaaaagatctgcaaaattttctcaagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccctaagaccacagtggccaagatcacagtggccacggccacggccacagtcatggtggccacggccacagccacccat

SEQ ID NO: 8 - DNA, 4439 bp, Homo sapiens
  ref <308>: DATABASE ACCESSION NUMBER: M21064
  ref <309>: DATABASE ENTRY DATE: 1993-04-27
  ref <313>: RELEVANT RESIDUES: (1)..(4439)
atcactgtggagtaggggaagggcactcctggggtggcaaggtgggaggtgggccctgtgttcccacagtgggcagggaggtagtgaaagggaagctggccggacaggaagggccattccaagagggctttgtgcgcagggctaagccaagctttctccataggcaatggggagcaactggaggttcgtagcaggagaaggacacatcaagcccaccaggaggctaagtaaaaacagttgtctcccaagttataagttcctggaacccttgctgggagcaggatttagaaaaatgatgctgagagatgctagaaacatattcgccctgaggctctctcactcagactgcaagaggaaggtatcatcagaattgcccttaaccaggaaccagaatagctgggtccccttcctgccaagtcagcaaccagctatgtgaccttgctcaggtccatctccgggtgtcagtttcttcatctacaatgcaagagggttgcccacctctgagaacccttctaaccccaaatctcaccctatgaatctaagaacacaacccctcgccatcctaagtatcacagagccaggcaagcatgggtgagagctcagaccatccttgttggactaaaaggaaggggcagactgccatggggggcagccgagagggtcaggcccccataggtcctcagcctgcttcaacctcaaaggggatggggggctgagtggtgccagaggagcagcaggctcgctcggggagagtagggccttaggatagaagggaaatgaactaaacaaccagcttcctgcaaaccagtttcaggccagggctgggaatttcacaaaaaagcagaaggcgctctgtgaacatttcctgccccgccccagcccccttcctggcagcattagcacactgctcacctgtgaagcaatcttccggagacagggccaaagggcaagtgccccagtcaggagctgcctataaatgccgagcctgcacagctctggcaaacactctgtgtggctcctcggctttggtaagtgagctgccagcttccccaggcagaagcctgcctgccgattccttctttccttccctgacccaacttccttccaaatcctcctcctagaagccctccttggttggccctgcctactttaaagcttctttcacattttcttaggtcatgttcccctggggcctcctgccctcaaatgctttgctttttggcactctgtagatattctaaaaaatcattttgtacatgtgtgtgacaggccatctcccagttaagttgcagcctgtgctttctttttattttgcacttcccccactatttctgtgagtgcttagtaggaagtgtcaaagaagcttgacagcattttcttctaagtgtcccaactcttggttttccattacacagacagagtgcaagacgatgacttgcaaaatgtcgcagctggaacgcaacatagagaccatcatcaacaccttccaccaatactctgtgaagctggggcacccagacaccctgaaccagggggaattcaaagagctggtgcgaaaagatctgcaaaattttctcaaggtagggctggactctggcaggtctgacccagcctcaccgcagtttgggttgacaagggaggatgggagtatgggctacagcaatcaaggggaagatttgagctcctggagcccagccccaagacgcagcgagtgtcctgttatacagggcaggtgctcacagttacacaggacgacagggtcaagaaattgctcaattgaacacctgctatttgtcgggccctgttctgggcagagggatgtagtggtaaatgggagcccactattccatgaggagacacacagtaaagttgttggccaataaagagcacagataaagccaaatgccaataagtgcctggaagaaaatgagatagagtgcgctgtgggcaatggggctgggtggggtggaggtgaccagttagggtacatgagaagggcctctttgaggaggtaacatttgagctgagccccgaatgttggggagggaagcccctgaggatgacacttggcacaaagctgaggagaccctaagcctcagggcgaacttggggtggaagacttgggggcttttctaatcctaagggtctgcggtggaaaatgaatgcataaagagcacatggagagcacctgcacagcactcagggaactgggaggtttttcccccgctccaaaaatgattaggcagttctaagaaaaaggctgagcacttccaacagcctttttgttttcttttcaaatttggggaaagtcgggaaacagaggcctgcattaagaagggtggaacacatgggtctcagtctcagttccagtcccggagccagacatcctggggtaggtccccagccctcccagtgcccctccctccgccttggtaaggtggagaattgcagccttcagagttaggggccctgacagctctccataggtggaggcctcaggcaggcaggatgctgggtggggtaggcaagaaagggcccagcagagaggccgcatcggaaaactatcctccatgtgaccccctatgcccgcttcaccccccacctgacatcccccaccagaagcaaagcgatgctgtgggaaaggaagcagagcctcatggatgggctgcacaggagagtgctcgcattggctgggtaccccacaggttctgggaggggacttagcgaggtgactcagtgcctcggcctcccaaagtgctgggattacaagcatgagccaccctgtccgaccatctccccttttatactttatcacacccttgaggtcagcggagcacatactctgctctctgaccctccatctcccctgcccacacctaggtttttctagtgtttccccgttgtattggttgaaataagtttcactaattggtaacctccagagggaagggaagggagggcaggggaaggagtgaagtgcagaggggtagcagagtggaactggcctctaagtcagatctgaatttgcatgccctcaatagtcaagcctgtgaaaactaatgaccctctctaggactggtttcaagtcttcctccaggaagataccattcctagctgttaaagttgttataaggaccaaatgaggtgacatttccaggcttactcatgccatgaccagggcaagaccctggaactcagcttcctcttctataaatagagaatcagcacccaagtcacagggtcatggagggaataaactggagagcgtttggtatgtgctcagtgtctgctccattgtgcgcactcagcctatggtcatttttaatttttaaatccagccccagggtcgaggcttccttgtacatttgccagctggtcatttactgtgctcccagtccccacctctggccacacccagctctcacagccttctctccccacccgcagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccctaagaccacagtggccaagatcacagtggccacggccacggccacagtcatggtggccacggccacaggccactaatcaggaggccaggccaccctgcctctacccaaccagggccccggggctgttatgtcaaactgtcttggctgtggggctaggggctggggcaaataagtctcttcctccaagtcagtgctctgtgtgcttcttccacctcttctccaaccctgccttcccagggctctggcatttagacagccctgtccttatctgtgactcagccccctcattcagtattaacaaaatgagaagcagcaaaacatgggtctgtgctgggccccttggctcacctccctgaccatgtcctcacctctgacttcaggccccactgttcagatcccaggctccctgccccatctcagacaccctgtccagcctgtccagcctgacaaatggcccttgtcactgtacactgtagaaagcaaaaaggcatatctctaccccttgatatgcctgctacctcaccaaccagccccaagcctgtcttcacccatcactgtctacacagccctctctctctcctaacagaattctattcctctgaaagtcttcagaaactggacctagatagtgccatgtctggggaggaatatggcaccaggcagtggaaacaaggacagatcggtgtgttatctcacatttgatcagagagcatgatctctcttaacagacctgccaccctaatcaacgggagtgctcacacaagtgggagtctgagagcttagccctatgcccaccctgg

SEQ ID NO: 9 - protein, 764 aa, Homo sapiens
  ref <308>: DATABASE ACCESSION NUMBER: P01833
  ref <309>: DATABASE ENTRY DATE: 2004-06-15
  ref <313>: RELEVANT RESIDUES: (1)..(764)
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 10 - DNA, 4266 bp, Homo sapiens
  ref <308>: DATABASE ACCESSION NUMBER: NM_002644
  ref <309>: DATABASE ENTRY DATE: 2004-10-27
  ref <313>: RELEVANT RESIDUES: (1)..(4266)
agagtttcagttttggcagcagcgtccagtgccctgccagtagctcctagagaggcaggggttaccaactggccagcaggctgtgtccctgaagtcagatcaacgggagagaaggaagtggctaaaacattgcacaggagaagtcggcctgagtggtgcggcgctcgggacccaccagcaatgctgctcttcgtgctcacctgcctgctggcggtcttcccagccatctccacgaagagtcccatatttggtcccgaggaggtgaatagtgtggaaggtaactcagtgtccatcacgtgctactacccacccacctctgtcaaccggcacacccggaagtactggtgccggcagggagctagaggtggctgcataaccctcatctcctcggagggctacgtctccagcaaatatgcaggcagggctaacctcaccaacttcccggagaacggcacatttgtggtgaacattgcccagctgagccaggatgactccgggcgctacaagtgtggcctgggcatcaatagccgaggcctgtcctttgatgtcagcctggaggtcagccagggtcctgggctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtaaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaggctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggtaccagggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctgaggaacaaggtggaagctccagagcgctggtctccaccctggtgcccctgggcctggtgctggcagtgggagccgtggctgtgggggtggccagagcccggcacaggaagaacgtcgaccgagtttcaatcagaagctacaggacagacattagcatgtcagacttcgagaactccagggaatttggagccaatgacaacatgggagcctcttcgatcactcaggagacatccctcggaggaaaagaagagtttgttgccaccactgagagcaccacagagaccaaagaacccaagaaggcaaaaaggtcatccaaggaggaagccgagatggcctacaaagacttcctgctccagtccagcaccgtggccgccgaggcccaggacggcccccaggaagcctagacggtgtcgccgcctgctccctgcacccatgacaatcaccttcagaatcatgtcgatcctggggccctcagctcctggggaccccactccctgctctaacacctgcctaggtttttcctactgtcctcagaggcgtgctggtcccctcctcagtgacatcaaagcctggcctaattgttcctattggggatgagggtggcatgaggaggtcccacttgcaacttctttctgttgagagaacctcaggtacggagaagaatagaggtcctcatgggtcccttgaaggaagagggaccagggtgggagagctgattgcagaaaggagagacgtgcagcgcccctctgcacccttatcatgggatgtcaacagaatttttccctccactccatccctccctcccgtccttcccctcttcttctttccttccatcaaaagatgtatttgaattcatactagaattcaggtgctttgctagatgctgtgacaggtatgccaccaacactgctcacagcctttctgaggacaccagtgaaagaagccacagctcttcttggcgtatttatactcactgagtcttaacttttcaccaggggtgctcacctctgcccctattgggagaggtcataaaatgtctcgagtcctaaggccttaggggtcatgtatgatgagcatacacacaggtaattataaacccacattcttaccatttcacacataagaaaattgaggtttggaagagtgaagcgtttttctttttcttttttttttttgagacggagtctctcactgtcgcccaggctggagtgcagtggcgcaatctcggctcactgcaacctccgcctcccaggttgacaccattctcctgcctcaccctcccaagtagctgggactacaggcgcctgccagcacgcctggctaattttttgtatttttagtagagacagggtttcaccgtgttagccaggatggtctcgatctcctgacctcgtgatccgcctgcctctgcctcccaaagtgctgggattacaggcgtgagccaccgcgtccggcctctttttttcttttcttttttttgagacaaagtctcactgtgtcacccagactggaatgcagtgacacaatctcggctcactgaaacctctgccttccaggttcaagctattctcatgcctcagcctctcaagtagctgggactacagatgtgggccaccatgtctggctaattttttttttttttttttttttttgtagagacagggtttcgccatgttgacgagactggtctcgaactcctggcctcaagtgatctgccgcctcagcttctcaaagtactgggattatataggcatgagccactgagcctggccctgaagcgtttttctcaaaggccctcagtgagataaattagatttggcatctcctgtcctgggccagggatctctctacaagagcccctgcccctctgttggaggcacagttttagaataaggaggaggagggagaagagaaaatgtaaaggagggagatctttcccaggccgcaccatttctgtcactcacatggacccaagataaaagaatggccaaaccctcacaacccctgatgtttgaagagttccaagttgaagggaaacaaagaagtgtttgatggtgccagagaggggctgctctccagaaagctaaaatttaatttcttttttcctctgagttctgtacttcaaccagcctacaagctggcacttgctaacaaatcagaaatatgacaattaatgattaaagactgtgattgcc

SEQ ID NO: 11 - protein, 187 aa, Homo sapiens
  ref <308>: DATABASE ACCESSION NUMBER: P30086
  ref <309>: DATABASE ENTRY DATE: 2004-10-25
  ref <313>: RELEVANT RESIDUES: (1)..(187)
MPVDLSKWSGPLSLQEVDEQPQHPLHVTYAGAAVDELGKVLTPTQVKNRPTSISWDGLDSGKLYTLVLTDPDAPSRKDPKYREWHHFLVVNMKGNDISSGTVLSDYVGSGPPKGTGLHRYVWLVYEQDRPLKCDEPILSNRSGDHRGKFKVASFRKKYELRAPVAGTCYQAEWDDYVPKLYEQLSGK

SEQ ID NO: 12 - DNA, 1507 bp, Homo sapiens
  ref <308>: DATABASE ACCESSION NUMBER: NM_002567
  ref <309>: DATABASE ENTRY DATE: 2004-10-27
  ref <313>: RELEVANT RESIDUES: (1)..(1507)
tgggcggcggctgaggcgcgtgctctcgcgtggtcgctgggtctgcgtcttcccgagccagtgtgctgagctctccgcgtcgcctctgtcgcccgcgcctggcctaccgcggcactcccggctgcacgctctgcttggcctcgccatgccggtggacctcagcaagtggtccgggcccttgagcctgcaagaagtggacgagcagccgcagcacccgctgcatgtcacctacgccggggcggcggtggacgagctgggcaaagtgctgacgcccacccaggttaagaatagacccaccagcatttcgtgggatggtcttgattcagggaagctctacaccttggtcctgacagacccggatgctcccagcaggaaggatcccaaatacagagaatggcatcatttcctggtggtcaacatgaagggcaatgacatcagcagtggcacagtcctctccgattatgtgggctcggggcctcccaagggcacaggcctccaccgctatgtctggctggtttacgagcaggacaggccgctaaagtgtgacgagcccatcctcagcaaccgatctggagaccaccgtggcaaattcaaggtggcgtccttccgtaaaaagtatgagctcagggccccggtggctggcacgtgttaccaggccgagtgggatgactatgtgcccaaactgtacgagcagctgtctgggaagtagggggttagcttggggacctgaactgtcctggaggccccaagccatgttccccagttcagtgttgcatgtataatagatttctcctcttcctgccccccttggcatgggtgagacctgaccagtcagatggtagttgagggtgacttttcctgctgcctggcctttataattttactcactcactctgatttatgttttgatcaaatttgaacttcattttggggggtattttggtactgtgatggggtcatcaaattattaatctgaaaatagcaacccagaatgtaaaaaagaaaaaactggggggaaaaagaccaggtctacagtgatagagcaaagcatcaaagaatctttaagggaggtttaaaaaaaaaaaaaaaaaaaaagattggttgcctctgcctttgtgatcctgagtccagaatggtacacaatgtgattttatggtgatgtcactcacctagacaaccagaggctggcattgaggctaacctccaacacagtgcatctcagatgcctcagtaggcatcagtatgtcactctggtccctttaaagagcaatcctggaagaagcaggagggagggtggctttgctgttgttgggacatggcaatctagaccggtagcagcgctcgctgacagcttgggaggaaacctgagatctgtgttttttaaattgatcgttcttcatgggggtaagaaaagctggtctggagttgctgaatgttgcattaattgtgctgtttgcttgtagttgaataaaaatagaaacctgaatgaagaaaaaaaaaaaaaaaa

SEQ ID NO: 13 - protein, 249 aa, Homo sapiens
  ref <308>: DATABASE ACCESSION NUMBER: P39687
  ref <309>: DATABASE ENTRY DATE: 2004-06-15
  ref <313>: RELEVANT RESIDUES: (1)..(249)
MEMGRRIHLELRNRTPSDVKELVLDNSRSNEGKLEGLTDEFEELEFLSTINVGLTSIANLPKLNKLKKLELSDNRVSGGLEVLAEKCPNLTHLNLSGNKIKDLSTIEPLKKLENLKSLDLFNCEVTNLNDYRENVFKLLPQLTYLDGYDRDDKEAPDSDAEGYVEGLDDEEEDEDEEEYDEDAQVVEDEEDEDEEEEGEEEDVSGEEEEDEEGYNDGEVDDEEDEEELGEEERGQKRKREPEDEGEDDD

SEQ ID NO: 14 - DNA, 1136 bp, Homo sapiens
  ref <308>: DATABASE ACCESSION NUMBER: NM_006305
  ref <309>: DATABASE ENTRY DATE: 2004-10-27
  ref <313>: RELEVANT RESIDUES: (1)..(1136)
cgggtgctgggggctcgagaaccgagcggagctggttgagccttcaaagtcctaaaacgcgcggccgtgggttcggggtttattgattgaattccgccggcgcgggagcctctgcagagagagagcgcgagagatggagatgggcagacggattcatttagagctgcggaacaggacgccctctgatgtgaaagaacttgtcctggacaacagtcggtcgaatgaaggcaaactcgaaggcctcacagatgaatttgaagaactggaattcttaagtacaatcaacgtaggcctcacctcaatcgcaaacttaccaaagttaaacaaacttaagaagcttgaactaagcgataacagagtctcagggggcctggaagtattggcagaaaagtgtccgaacctcacgcatctaaatttaagtggcaacaaaattaaagacctcagcacaatagagccactgaaaaagttagaaaacctcaagagcttagaccttttcaattgcgaggtaaccaacctgaacgactaccgagaaaatgtgttcaagctcctcccgcaactcacatatctcgacggctatgaccgggacgacaaggaggcccctgactcggatgctgagggctacgtggagggcctggatgatgaggaggaggatgaggatgaggaggagtatgatgaagatgctcaggtagtggaagacgaggaggacgaggatgaggaggaggaaggtgaagaggaggacgtgagtggagaggaggaggaggatgaagaaggttataacgatggagaggtagatgacgaggaagatgaagaagagcttggtgaagaagaaaggggtcagaagcgaaaacgagaacctgaagatgagggagaagatgatgactaagtggaataacctattttgaaaaattcctattgtgatttgactgtttttacccatatcccctctcccccccccctccaatcctgccccctgaaacttatttttttctgattgtaacgttgctgtgggaacgagaggggaagagtgtactgggggttgcggggggagggatggcgggtgggggtggaataaaatactatttttactgccactctttaaaaaaaaaaaaaaaaaaaaaaaaaaaaaaaaaaaaaaa

SEQ ID NO: 15 - protein, 643 aa, Homo sapiens
  ref <308>: DATABASE ACCESSION NUMBER: P17066
  ref <309>: DATABASE ENTRY DATE: 2003-09-15
  ref <313>: RELEVANT RESIDUES: (1)..(643)
MQAPRELAVGIDLGTTYSCVGVFQQGRVEILANDQGNRTTPSYVAFTDTERLVGDAAKSQAALNPHNTVFDAKRLIGRKFADTTVQSDMKHWPFRVVSEGGKPKVRVCYRGEDKTFYPEEISSMVLSKMKETAEAYLGQPVKHAVITVPAYFNDSQRQATKDAGAIAGLNVLRIINEPTAAAIAYGLDRRGAGERNVLIFDLGGGTFDVSVLSIDAGVFEVKATAGDTHLGGEDFDNRLVNHFMEEFRRKHGKDLSGNKRALRRLRTACERAKRTLSSSTQATLEIDSLFEGVDFYTSITRARFEELCSDLFRSTLEPVEKALRDAKLDKAQIHDVVLVGGSTRIPKVQKLLQDFFNGKELNKSINPDEAVAYGAAVQAAVLMGDKCEKVQDLLLLDVAPLSLGLETAGGVMTTLIQRNATIPTKQTQTFTTYSDNQPGVFIQVYEGERAMTKDNNLLGRFELSGIPPAPRGVPQIEVTFDIDANGILSVTATDRSTGKANKITITNDKGRLSKEEVERMVHEAEQYKAEDEAQRDRVAAKNSLEAHVFHVKGSLQEESLRDKIPEEDRRKMQDKCREVLAWLEHNQLAEKEEYEHQKRELEQICRPIFSRLYGGPGVPGGSSCGTQARQGDPSTGPIIEEVD

SEQ ID NO: 16 - DNA, 2664 bp, Homo sapiens
  ref <308>: DATABASE ACCESSION NUMBER: NM_002155
  ref <309>: DATABASE ENTRY DATE: 2004-10-28
  ref <313>: RELEVANT RESIDUES: (1)..(2664)
agagccagcccggaggagctagaaccttccccgcatttctttcagcagcctgagtcagaggcgggctggcctggcgtagccgcccagcctcgcggctcatgccccgatctgcccgaaccttctcccggggtcagcgccgcgccgcgccacccggctgagtcagcccgggcgggcgagaggctctcaactgggcgggaaggtgcgggaaggtgcggaaaggttcgcgaaagttcgcggcggcgggggtcgggtgaggcgcaaaaggataaaaagcccgtggaagcggagctgagcagatccgagccgggctggctgcagagaaaccgcagggagagcctcactgctgagcgcccctcgacggcggagcggcagcagcctccgtggcctccagcatccgacaagaagcttcagccatgcaggccccacgggagctcgcggtgggcatcgacctgggcaccacctactcgtgcgtgggcgtgtttcagcagggccgcgtggagatcctggccaacgaccagggcaaccgcaccacgcccagctacgtggccttcaccgacaccgagcggctggtcggggacgcggccaagagccaggcggccctgaacccccacaacaccgtgttcgatgccaagcggctgatcgggcgcaagttcgcggacaccacggtgcagtcggacatgaagcactggcccttccgggtggtgagcgagggcggcaagcccaaggtgcgcgtatgctaccgcggggaggacaagacgttctaccccgaggagatctcgtccatggtgctgagcaagatgaaggagacggccgaggcgtacctgggccagcccgtgaagcacgcagtgatcaccgtgcccgcctatttcaatgactcgcagcgccaggccaccaaggacgcgggggccatcgcggggctcaacgtgttgcggatcatcaatgagcccacggcagctgccatcgcctatgggctggaccggcggggcgcgggagagcgcaacgtgctcatttttgacctgggtgggggcaccttcgatgtgtcggttctctccattgacgctggtgtctttgaggtgaaagccactgctggagatacccacctgggaggagaggacttcgacaaccggctcgtgaaccacttcatggaagaattccggcggaagcatgggaaggacctgagcgggaacaagcgtgccctgcgcaggctgcgcacagcctgtgagcgcgccaagcgcaccctgtcctccagcacccaggccaccctggagatagactccctgttcgagggcgtggacttctacacgtccatcactcgtgcccgctttgaggaactgtgctcagacctcttccgcagcaccctggagccggtggagaaggccctgcgggatgccaagctggacaaggcccagattcatgacgtcgtcctggtggggggctccactcgcatccccaaggtgcagaagttgctgcaggacttcttcaacggcaaggagctgaacaagagcatcaaccctgatgaggctgtggcctatggggctgctgtgcaggcggccgtgttgatgggggacaaatgtgagaaagtgcaggatctcctgctgctggatgtggctcccctgtctctggggctggagacagcaggtggggtgatgaccacgctgatccagaggaacgccactatccccaccaagcagacccagactttcaccacctactcggacaaccagcctggggtcttcatccaggtgtatgagggtgagagggccatgaccaaggacaacaacctgctggggcgttttgaactcagtggcatccctcctgccccacgtggagtcccccagatagaggtgacctttgacattgatgctaatggcatcctgagcgtgacagccactgacaggagcacaggtaaggctaacaagatcaccatcaccaatgacaagggccggctgagcaaggaggaggtggagaggatggttcatgaagccgagcagtacaaggctgaggatgaggcccagagggacagagtggctgccaaaaactcgctggaggcccatgtcttccatgtgaaaggttctttgcaagaggaaagccttagggacaagattcccgaagaggacaggcgcaaaatgcaagacaagtgtcgggaagtccttgcctggctggagcacaaccagctggcagagaaggaggagtatgagcatcagaagagggagctggagcaaatctgtcgccccatcttctccaggctctatggggggcctggtgtccctgggggcagcagttgtggcactcaagcccgccagggggaccccagcaccggccccatcattgaggaggttgattgaatggcccttcgtgataagtcagctgtgactgtcagggctatgctatgggccttctagactgtcttctatgatcctgcccttcagagatgaactttccctccaaagctagaactttcttcccaggataactgaagtcttttgactttttggggggagggcggttcatcctcttctgcttcaaataaaaagtcattaatttattaaaacttgtgtggcactttaacattgctttcacctatattttgtgtactttgttacttgcatgtatgaattttgttatgtaaaatatagttatagacctaaataaaaaaaaaaaaaa

SEQ ID NO: 17 - DNA, 2492 bp, Homo sapiens
  ref <308>: DATABASE ACCESSION NUMBER: X51757
  ref <309>: DATABASE ENTRY DATE: 1998-11-13
  ref <313>: RELEVANT RESIDUES: (1)..(2492)
cccgggcgggcgagaggctctcaactgggcgggaaggtgcgggaaggtgcggaaaggttcgcgaaagttcgcggcggcgggggtcgggtgaggcgcaaaaggataaaaagcccgtggaagcggagctgagcagatccgagccgggctggctgcagagacaccgcagggagagcctcactgctgagcgcccctcgacggcggacgggcagcagcctccgtggcctccagcatccgacaagaagcttcagccatgcaggccccacgggagctcgcggtgggcatcgacctgggcaccacctactcgtgcgtgggcgtgtttcagcagggccgcgtggagatcctggccaacgaccagggcaaccgcaccacgcccagctacgtggccttcaccgacaccgagcggctggtcggggacgcggccaagagccaggcggccctgaacccccacaacaccgtgttcgatgccaagcggctgatcgggcgcaagttcgcggacaccacggtgcagtcggacatgaagcactggcccttccgggtggtgagcgagggcggcaagcccaaggtgccggtatcgtaccgcggggaggacaagacgttctaccccgaggagatctcgtccatggtgctgagcaagatgaaggagacggccgaggcgtacctgggccagcccgtgaagcacgcagtgatcaccgtgcccgcctatttcaatgactcgcagcgccaggccaccaaggacgcgggggccatcgcggggctcaacgtgttgcggatcatcaatgagcccacggcagctgccatcgcctatgggctggaccggcggggcgcgggagagcgcaacgtgctcatttttgacctgggtgggggcaccttcgatgtgtcggttctctccattgacgctggtgtctttgaggtgaaagccactgctggagatacccacctgggaggagaggacttcgacaaccggctcgtgaaccacttcatggaagaattccggcggaagcatgggaaggacctgagcgggaacaagcgtgccctcggcaggctgcgcacagcctgtgagcgcgccaagcgcaccctgtcctccagcacccaggccaccctggagatagactccctgttcgagggcgtggacttctacacgtccatcactcgtgcccgctttgaggaactgtgctcagacctcttccgcagcaccctggagccggtggagaaggccctgcgggatgccaagctggacaaggcccagattcatgacgtcgtcctggtggggggctccactcgcatccccaaggtgcagaagttgctgcaggacttcttcaacggcaaggagctgaacaagagcatcaaccctgatgaggctgtggcctatggggctgctgtgcaggcggccgtgttgatgggggacaaatgtgagaaagtgcaggatctcctgctgctggatgtggctcccctgtctctggggctggagacagcaggtggggtgatgaccacgctgatccagaggaacgccactatccccaccaagcagacccagactttcaccacctactcggacaaccagcctggggtcttcatccaggtgtatgagggtgagagggccatgaccaaggacaacaacctgctggggcgttttgaactcagtggcatccctcctgccccacgtggagtcccccagatagaggtgacctttgacattgatgctaatggcatcctgagcgtgacagccactgacaggagcacaggtaaggctaacaagatcaccatcaccaatgacaagggccggctgagcaaggaggaggtggagaggatggttcatgaagccgagcagtacaaggctgaggatgaggcccagagggacagagtggctgccaaaaactcgctggaggcccatgtcttccatgtgaaaggttctttgcaagaggaaagccttagggacaagattcccgaagaggacaggcgcaaaatgcaagacaagtgtcgggaagtccttgcctggctggagcacaaccagctggcagagaaggaggagtatgagcatcagaagagggagctggagcaaatctgtcgccccatcttctccaggctctatggggggcctggtgtccctgggggcagcagttgtggcactcaagcccgccagggggaccccagcaccggccccatcattgaggaggttgattgaatggcccttcgtgataagtcagctgtgactgtcagggctatgctatgggccttctagactgtcttctatgatcctgcccttcagagatgaactttccctccaaagctagaactttcttcccaggataactgaagtcttttgactttttgcggggagggcggttcatcctcttctgcttcaaataaaaagtcattaatttattaaaacttgtgtggcactttaacattgctttcacctatattttgtgtactttgttacttgcatgtatgaattttgttatgtaaaatatagttatagacctaaataagct

SEQ ID NO: 18 - protein, 115 aa, Homo sapiens
  ref <308>: DATABASE ACCESSION NUMBER: P14174
  ref <309>: DATABASE ENTRY DATE: 2005-01-25
  ref <313>: RELEVANT RESIDUES: (1)..(115)
MPMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFA

SEQ ID NO: 19 - DNA, 561 bp, Homo sapiens
  ref <308>: DATABASE ACCESSION NUMBER: NM_002415
  ref <309>: DATABASE ENTRY DATE: 2004-10-26
  ref <313>: RELEVANT RESIDUES: (1)..(561)
accacagtggtgtccgagaagtcaggcacgtagctcagcggcggccgcggcgcgtgcgtctgtgcctctgcgcgggtctcctggtccttctgccatcatgccgatgttcatcgtaaacaccaacgtgccccgcgcctccgtgccggacgggttcctctccgagctcacccagcagctggcgcaggccaccggcaagcccccccagtacatcgcggtgcacgtggtcccggaccagctcatggccttcggcggctccagcgagccgtgcgcgctctgcagcctgcacagcatcggcaagatcggcggcgcgcagaaccgctcctacagcaagctgctgtgcggcctgctggccgagcgcctgcgcatcagcccggacagggtctacatcaactattacgacatgaacgcggccaatgtgggctggaacaactccaccttcgcctaagagccgcagggacccacgctgtctgcgctggctccacccgggaacccgccgcacgctgtgttctaggcccgcccaccccaaccttctggtggggagaaataaacggtttagagact

SEQ ID NO: 20 - DNA, 2167 bp, Homo sapiens
  ref <308>: DATABASE ACCESSION NUMBER: HUMMIF
  ref <309>: DATABASE ENTRY DATE: 1994-09-29
  ref <313>: RELEVANT RESIDUES: (1)..(2167)
ctgcaggaaccaatacccataggctatttgtataaatgggccatggggcctcccagctggaggctggctggtgccacgagggtcccacaggcatgggtgtccttcctatatcacatggccttcactgagactggtatatggattgcacctatcagagaccaaggacaggacctccctggaaatctctgaggacctggcctgtgatccagttgctgccttgtcctcttcctgctatgtcatggcttatcttctttcacccattcattcattcattcattcattcagcagtattagtcaatgtctcttgatatgcctggcacctgctagatggtccccgagtttaccattagtggaaaagacatttaagaaattcaccaagggctctatgagaggccatacacggtggacctgactagggtgtggcttccctgaggagctgaagttgcccagaggcccagagaaggggagctgagcacgtttgaaccactgaacctgctctggacctcgcctccttccttcggtgcctcccagcatcctatcctctttaaagagcaggggttcagggaagttccctggatggtgattcgcaggggcagctcccctctcacctgccgcatgactaccccgccccatctcaaacacacaagctcacgcatgcgggactggagcccttgaggacatgtggcccaaagacaggaggtacaggggctcagtgcgtgcagtggaatgaactgggcttcatctctggaagggtaaggggccatcttccgggttcaccgccgcatccccacccccggcacagcgcctcctggcgactaacatcggtgacttagtgaaaggactaagaaagacccgaggcgaggccggaacaggccgatttctagccgccaagtggagaacaggttggagcggtgcgccgggcttagcggcggttgctggaggaacgggcggagtcgcccagggtcctgccctgcgggggtcgagccgaggcaggcggtgacttccccactcggggcggagccgcagcctcgcgggggcggggcctggcgccggcggtggcgtcacaaaaggcgggaccacagtggtgtccgagaagtcaggcacgtagctcagcggcggccgcggcgcgtgcgtctgtgcctctgcgcgggtctcctggtccttctgccatcatgccgatgttcatcgtaaacaccaacgtgccccgcgcctccgtgccggacgggttcctctccgagctcacccagcagctggcgcaggccaccggcaagcccccccaggtttgccgggaggggacaggaagaggggggtgcccaccggacgaggggttccgcgctgggagctggggaggcgactcctgaacggagctggggggcggggcggggggaggacggtggctcgggcccgaagtggacgttcggggcccgacgaggtcgctggggcgggctgaccgcgccctttcctcgcagtacatcgcggtgcacgtggtcccggaccagctcatggccttcggcggctccagcgagccgtgcgcgctctgcagcctgcacagcatcggcaagatcggcggcgcgcagaaccgctcctacagcaagctgctgtgcggcctgctggccgagcgcctgcgcatcagcccggacaggtacgcggagtcgcggaggggcgggggaggggcggcggcgcgcggccaggcccgggactgagccacccgctgagtccggcctcctccccccgcagggtctacatcaactattacgacatgaacgcggccaatgtgggctggaacaactccaccttcgcctaagagccgcagggacccacgctgtctgcgctggctccacccgggaacccgccgcacgctgtgttctaggcccgcccaccccaaccttctggtggggagaaataaacggtttagagactaggagtgcctcggggttccttggcttgcgggaggaattggtgcagagccgggacattggggagcgaggtcgggaaacggtgttgggggcgggggtcagggccgggttgctctcctcgaacctgctgttcgggagcccttttgtccagcctgtccctcctacgctcctaacagaggagccccagtgtctttccattctatggcgtacgaagggatgaggagaagttggcactctgccctgggctgcag

SEQ ID NO: 21 - protein, 105 aa, Homo sapiens
  ref <308>: DATABASE ACCESSION NUMBER: P31949
  ref <309>: DATABASE ENTRY DATE: 2004-10-25
  ref <313>: RELEVANT RESIDUES: (1)..(105)
MAKISSPTETERCIESLIAVFQKYAGKDGYNYTLSKTEFLSFMNTELAAFTKNQKDPGVLDRMMKKLDTNSDGQLDFSEFLNLIGGLAMACHDSFLKAVPSQKRT

SEQ ID NO: 22 - DNA, 595 bp, Homo sapiens
  ref <308>: DATABASE ACCESSION NUMBER: NM_005620
  ref <309>: DATABASE ENTRY DATE: 2004-10-26
  ref <313>: RELEVANT RESIDUES: (1)..(595)
gggcaaggctgggccgggaagggcgtgggttgaggagaggctccagacccgcacgccgcgcgcacagagctctcagcgccgctcccagccacagcctcccgcgcctcgctcagctccaacatggcaaaaatctccagccctacagagactgagcggtgcatcgagtccctgattgctgtcttccagaagtatgctggaaaggatggttataactacactctctccaagacagagttcctaagcttcatgaatacagaactagctgccttcacaaagaaccagaaggaccctggtgtccttgaccgcatgatgaagaaactggacaccaacagtgatggtcagctagatttctcagaatttcttaatctgattggtggcctagctatggcttgccatgactccttcctcaaggctgtcccttcccagaagcggacctgaggaccccttggccctggccttcaaacccaccccctttccttccagcctttctgtcatcatctccacagcccacccatcccctgagcacactaaccacctcatgcaggccccacctgccaatagtaataaagcaatgtcacttttttaaaacatgaaa

SEQ ID NO: 23 - protein, 249 aa, Homo sapiens
  ref <308>: DATABASE ACCESSION NUMBER: P00938
  ref <309>: DATABASE ENTRY DATE: 2003-09-15
  ref <313>: RELEVANT RESIDUES: (1)..(249)
MAPSRKFFVGGNWKMNGRKQSLGELIGTLNAAKVPADTEVVCAPPTAYIDFARQKLDPKIAVAAQNCYKVTNGAFTGEISPGMIKDCGATWVVLGHSERRHVFGESDELIGQKVAHALAEGLGVIACIGEKLDEREAGITEKVVFEQTKVIADNVKDWSKVVLAYEPVWAIGTGKTATPQQAQEVHEKLRGWLKSNVSDAVAQSTRIIYGGSVTGATCKELASQPDVDGFLVGGASLKPEFVDIINAKQ

SEQ ID NO: 24 - DNA, 1254 bp, Homo sapiens
  ref <308>: DATABASE ACCESSION NUMBER: NM_000365
  ref <309>: DATABASE ENTRY DATE: 2004-12-18
  ref <313>: RELEVANT RESIDUES: (1)..(1254)
ccttcagcgcctcggctccagcgccatggcgccctccaggaagttcttcgttgggggaaactggaagatgaacgggcggaagcagagtctgggggagctcatcggcactctgaacgcggccaaggtgccggccgacaccgaggtggtttgtgctccccctactgcctatatcgacttcgcccggcagaagctagatcccaagattgctgtggctgcgcagaactgctacaaagtgactaatggggcttttactggggagatcagccctggcatgatcaaagactgcggagccacgtgggtggtcctggggcactcagagagaaggcatgtctttggggagtcagatgagctgattgggcagaaagtggcccatgctctggcagagggactcggagtaatcgcctgcattggggagaagctagatgaaagggaagctggcatcactgagaaggttgttttcgagcagacaaaggtcatcgcagataacgtgaaggactggagcaaggtcgtcctggcctatgagcctgtgtgggccattggtactggcaagactgcaacaccccaacaggcccaggaagtacacgagaagctccgaggatggctgaagtccaacgtctctgatgcggtggctcagagcacccgtatcatttatggaggctctgtgactggggcaacctgcaaggagctggccagccagcctgatgtggatggcttccttgtgggtggtgcttccctcaagcccgaattcgtggacatcatcaatgccaaacaatgagccccatccatcttccctacccttcctgccaagccagggactaagcagcccagaagcccagtaactgccctttccctgcatatgcttctgatggtgtcatctgctccttcctgtggcctcatccaaactgtatcttcctttactgtttatatcttcaccctgtaatggttgggaccaggccaatcccttctccacttactataatggttggaactaaacgtcaccaaggtggcttctccttggctgagagatggaaggcgtggtgggatttgctcctgggttccctaggccctagtgagggcagaagagaaaccatcctctcccttcttacaccgtgaggccaagatcccctcagaaggcaggagtgctgccctctcccatggtgcccgtgcctctgtgctgtgtatgtgaaccacccatgtgagggaataaacctggcactaggaaaaaaaaaaaaaaaaaaaaaaaaaaaaaaaaaa

SEQ ID NO: 25 - DNA, 5005 bp, Homo sapiens
  ref <308>: DATABASE ACCESSION NUMBER: X69723
  ref <309>: DATABASE ENTRY DATE: 1997-03-24
  ref <313>: RELEVANT RESIDUES: (1)..(5005)
ctgcagttcctgccaggccttgccagccggggcgagggttgggatgatcctggcggcctatgcctgtgtgggctgcccctcccgctgtgaaccctgcatttgtcccgcaagttttcactcaggtagactccctgggtacaagggtgcctgctcagcagtcgggcatgagctgctccgatgggcgaaggaggttgtctattccacagttggagaggggccctctctgccccagtgggcgatctgggctacggccaagttgccaccagctagttccgcttgaaaaccacttctggccccgtgggggactcaagtcgccaagcgagggttcccctgagcgccggagctcacaggtctcgccttgtcccgaaagccccgcaatcgaggcggaggcgaccgagcccccgactctcctagaacgttgccacaagaagggggaacgtcggaacagtgcatcatcgggcggcggccggggcggcggcaggagggcgggcggggggcagggctccgggggactgggcgggccatggcggaggacggcgaggaggcggagttccacttcgcggcgctctatataagtgggcagtggccgcgactgcgcgcagacactgaccttcagcgcctcggctccagcgccatggcgccctccaggaagttcttcgttgggggaaactggaagatgaacgggcggaagcagagtctgggggagctcatcggcactctgaacgcggccaaggtgccggccgacaccggtaagccctcgccgaggaggggtctggccgggccggggccgggccggggcaggagtggcagcgcctctcccgaggcccgaggtccgggccggtatccgcgcggacctgatgcagggctgtgggacgagggccgctggggtccgggcaggggcctcgcagccgcagccccgtcggtgcgtcgagggggcagggcggagcacatgatgccccttggactacggggcaggtaaggacgttttgggtctcctggaggaaggcggccccggggcgcgcactggctgtgcccgccaggcgacggggttaggagccgagcccgaggctctgcgggagaccgggggaggctgggccgcgtgggcttccgctccctgccctggcctccgcgtgcgcgccgccgcacgtagccccagactcctccccctcctcgccggcgtcgtcccgcgccgagctgctgctgccctgagcccccagatctgaaccccttcccttcggcaacctgagcgactcccgccttccacggaagggaccgagcccgtgccaaacaggctgagcgatttgggagtgaggagccatcctaccgctttccccaacctggaaacagcaaagcgcaaggcctctgagtcagttaggtctctgccacccacgggcaaaggatgctctcctccatcctccttcctccctccaccgaaatcggagagccgcgggcctgatccaaagaggcatccccttctcgttcattccccagaggcctcaatacaaaccccaggagttggcccctctccttttgctacaaatccttgccttgcaaaggggaggtgaggatgggctattttagaagggaagcagggttgctccctggagaatgctgagtctgtgaggtgcctatgccgagaatagctcgaggaaattggagccccagctgttaaaagagcagagggcagggtgagggccgtggctctcaggggtatctggaaggctcttcgagttgagtgcagacccagccttgggctggaaaatggacaaaggtcatcttgctggggtgaaaagggggagagcagaaccaagaagaagagggtgagggctggggggctccagggcactggttaggaattgtggggaatgaaggctttctttagtctcatccccctgtggtaccatcttgtcctcagaggtggtttgtgctccccctactgcctatatcgacttcgcccggcagaagctagatcccaagattgctgtggctgcgcagaactgctacaaagtgactaatggggcttttactggggagatcaggtgagatcgaggtggagaggggtgtgtgggacccttccctcactttcctcgttgaggggaaagccacagggtgggctccctgctgaaccttggcttcatctcttcctttagccctggcatgatcaaagactgcggagccacgtgggtggtcctggggcactcagagagaaggcatgtctttggggagtcagatgaggttagtagccaagagagaagataagggatgtctttttccaagaaggatgtctcaccaagtctgtttctcaacagctgattgggcagaaagtggcccatgctctggcagagggactcggagtaatcgcctgcattggggagaagctagatgaaagggaagctggcatcactgagaaggttgttttcgagcagacaaaggtcatcgcaggtatctctggagaaagggacctttgagcctatccagggccacagagactcagagggtagggtcaggccctggagcctgtcttggtccccatgctgatccagaaaaggaaaaaggggagggggagtgacaatctttgcttggggcctatgacttctccagccccaaggtagatgccacctggaaatcccccaatgtccactagggggcagtaggccaccgttcttcgtactccggagaacctggctggagagctctttcttgttcacccttccctccatctgtatctctgccctgcagataacgtgaaggactggagcaaggtcgtcctggcctatgagcctgtgtgggccattggtactggcaagactgcaacaccccaacaggtaaccgggcccaggagccctgccctcatcccagcctgcctcaataggtttggacagacacagcccacatggagcaaccccttatttcaaagacacagagaccttgaacccagagacagtgacttgtccaagggcatccagtccagggcctggcttggatcagagccctggtactctgactcagtcagaaaccacactaagtgtccactggtgccagtgatttttcctcttagagaggcagaaaaggtcttacttaggccagcttcttgttctaggcccaggaagtacacgagaagctccgaggatggctgaagtccaacgtctctgatgcggtggctcagagcacccgtatcatttatggaggtgagtggctttggttcccggctgaggtggagtgggctgaggactagactgagccctcggacatggaggtggggatggggcagactcatcccattcttgaccaagcccttgttctgctcccttcccaggctctgtgactggggcaacctgcaaggagctggccagccagcctgatgtggatggcttccttgtgggtggtgcttccctcaagcccgaattcgtggacatcatcaatgccaaacaatgagccccatccatcttccctacccttcctgccaagccagggactaagcagcccagaagcccagtaactgccctttccctgcatatgcttctgatggtgtcatctgctccttcctgtggcctcatccaaactgtatcttcctttactgtttatatcttcaccctgtaatggttgggaccaggccaatcccttctccacttactataatggttggaactaaacgtcaccaaggtggcttctccttggctgagagatggaaggcgtggtgggatttgctcctgggttccctaggccctagtgagggcagaagagaaaccatcctctcccttcttacaccgtgaggccaagatcccctcagaaggcaggagtgctgccctctcccatggtgcccgtgcctctgtgctgtgtatgtgaaccacccatgtgagggaataaacctggcactaggtcttgtggtttgtctgccttcactggacttgcccagataatcttcctttttgaggcagctatataaatgatcatttgtgcaagaaaaaaaaaaaaacaagaacaggtttctataacaacatctcttactatttttacttgaaaaaatgttttgcgtagcagactgtcatagccttgaacgccggctccctttcttcctccctccaagtggctctggggctgttgatttccgcagagcttgggttggggtagggctcagcctcaccagctttcagcagctggtctaggccagcagtgcctccccacctccccaagggagggtggtggcaagacctcagcacagtctgtggtatcacaggctcactggtagagcagtagcgcttcatgcagggggcaagggcagggcagacacctggccgagcggtatccccaggttgtggcgcacacacaggcggctcaggtgcagaagggagtgtggctccgctgggagagagaaggaggggaatgtaagtatgggtgcagccaccagccagatgtcctcaaactacggggtcctcatcagatgcctttctgctttcctgcttcgagtgtgcccacctggctgaaaggggaatttgagatacccggaagttctgcctcccagataagatttcacacatccctagtcagagctgggggtgaagagctggctaaggccctctaaacaacaggccaaggtggctctgacagtggtggagctggcccaggctttgactccagaggcttgggagctggggctgaggtgaggagggatggccctccactctacagcccaacacaactgcagagagcagctccaagccctggacccagtcagttcctggggaggctcctcccctgctgccccaccctaaggcctgcctcctccactgctctcctcctccctggtgcccagggccccagtgtctccatcctgaggtgtggctgaggaaggaagtaggtatgtggcacagagacaggttagagcccagggaatccggtatacagcctgggtacctcgtctgcccatccttcttttggacctgtacatcaaacccagtacctaaccgtttgcacctcttgcctaggggtgattactcctgaattc

SEQ ID NO: 26 - protein, 938 aa, Homo sapiens
  MISC_FEATURE: alfa-1-antitrypsin precursor
  ref <308>: DATABASE ACCESSION NUMBER: Q05586
  ref <309>: DATABASE ENTRY DATE: 2005-01-25
  ref <313>: RELEVANT RESIDUES: (1)..(938)
MSTMRLLTLALLFSCSVARAACDPKIVNIGAVLSTRKHEQMFREAVNQANKRHGSWKIQLNATSVTHKPNAIQMALSVCEDLISSQVYAILVSHPPTPNDHFTPTPVSYTAGFYRIPVLGLTTRMSIYSDKSIHLSFLRTVPPYSHQSSVWFEMMRVYSWNHIILLVSDDHEGRAAQKRLETLLEERESKAEKVLQFDPGTKNVTALLMEAKELEARVIILSASEDDAATVYRAAAMLNMTGSGYVWLVGEREISGNALRYAPDGILGLQLINGKNESAHISDAVGVVAQAVHELLEKENITDPPRGCVGNTNIWKTGPLFKRVLMSSKYADGVTGRVEFNEDGDRKFANYSIMNLQNRKLVQVGIYNGTHVIPNDRKIIWPGGETEKPRGYQMSTRLKIVTIHQEPFVYVKPTLSDGTCKEEFTVNGDPVKKVICTGPNDTSPGSPRHTVPQCCYGFCIDLLIKLARTMNFTYEVHLVADGKFGTQERVNNSNKKEWNGMMGELLSGQADMIVAPLTINNERAQYIEFSKPFKYQGLTILVKKEIPRSTLDSFMQPFQSTLWLLVGLSVHVVAVMLYLLDRFSPFGRFKVNSEEEEEDALTLSSAMWFSWGVLLNSGIGEGAPRSFSARILGMVWAGFAMIIVASYTANLAAFLVLDRPEERITGINDPRLRNPSDKFIYATVKQSSVDIYFRRQVELSTMYRHMEKHNYESAAEAIQAVRDNKLHAFIWDSAVLEFEASQKCDLVTTGELFFRSGFGIGMRKDSPWKQNVSLSILKSHENGFMEDLDKTWVRYQECDSRSNAPATLTFENMAGVFMLVAGGIVAGIFLIFIEIAYKRHKDARRKQMQLAFAAVNVWRKNLQDRKSGRAEPDPKKKATFRAITSTLASSFKRRRSSKDTSTGGGRGALQNQKDTVLPRRAIEREEGQLQLCSRHRES

SEQ ID NO: 27 - DNA, 3109 bp, Homo sapiens
  ref <308>: DATABASE ACCESSION NUMBER: D13515
  ref <309>: DATABASE ENTRY DATE: 1999-02-20
  ref <313>: RELEVANT RESIDUES: (1)..(3109)
gcttcagcgccccttccctcggccgacgtcccgggaccgccgctccgggggagacgtggcgtccgcagcccgcggggccgggcgagcgcaggacggcccggaagccccgcgggggatgcgccgagggccccgcgttcgcgccgcgcagagccaggcccgcggcccgagcccatgagcaccatgcgcctgctgacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccagacggcatcctcgggctgcagctcatcaacggcaagaacgagtcggcccacatcagcgacgccgtgggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggcggagagacagagaagcctcgagggtaccagatgtccaccagactgaagattgtgacgatccaccaggagcccttcgtgtacgtcaagcccacgctgagtgatgggacatgcaaggaggagttcacagtcaacggcgacccagtcaagaaggtgatctgcaccgggcccaacgacacgtcgccgggcagcccccgccacacggtgcctcagtgttgctacggcttttgcatcgacctgctcatcaagctggcacggaccatgaacttcacctacgaggtgcacctggtggcagatggcaagttcggcacacaggagcgggtgaacaacagcaacaagaaggagtggaatgggatgatgggcgagctgctcagcgggcaggcagacatgatcgtggcgccgctaaccataaacaacgagcgcgcgcagtacatcgagttttccaagcccttcaagtaccagggcctgactattctggtcaagaaggagattccccggagcacgctggactcgttcatgcagccgttccagagcacactgtggctgctggtggggctgtcggtgcacgtggtggccgtgatgctgtacctgctggaccgcttcagccccttcggccggttcaaggtgaacagcgaggaggaggaggaggacgcactgaccctgtcctcggccatgtggttctcctggggcgtcctgctcaactccggcatcggggaaggcgcccccagaagcttctcagcgcgcatcctgggcatggtgtgggccggctttgccatgatcatcgtggcctcctacaccgccaacctggcggccttcctggtgctggaccggccggaggagcgcatcacgggcatcaacgaccctcggctgaggaacccctcggacaagtttatctacgccacggtgaagcagagctccgtggatatctacttccggcgccaggtggagctgagcaccatgtaccggcatatggagaagcacaactacgagagtgcggcggaggccatccaggccgtgagagacaacaagctgcatgccttcatctgggactcggcggtgctggagttcgaggcctcgcagaagtgcgacctggtgacgactggagagctgtttttccgctcgggcttcggcataggcatgcgcaaagacagcccctggaagcagaacgtctccctgtccatcctcaagtcccacgagaatggcttcatggaagacctggacaagacgtgggttcggtatcaggaatgtgactcgcgcagcaacgcccctgcgacccttacttttgagaacatggccggggtcttcatgctggtagctgggggcatcgtggccgggatcttcctgattttcatcgagattgcctacaagcggcacaaggatgctcgccggaagcagatgcagctggcctttgccgccgttaacgtgtggcggaagaacctgcaggatagaaagagtggtagagcagagcctgaccctaaaaagaaagccacatttagggctatcacctccaccctggcttccagcttcaagaggcgtaggtcctccaaagacacgagcaccgggggtggacgcggcgctttgcaaaaccaaaaagacacagtgctgccgcgacgcgctattgagagggaggagggccagctgcagctgtgttcccgtcatagggagagctgagactccccgcccgccctcctctgccccctcccccgcagacagacagacagacggacgggacagcggcccggcccacgcagagccccggagcaccacggggtcgggggaggagcacccccag

SEQ ID NO: 28 - protein, 15 aa, Homo sapiens
LLTLLALLFSCSVAR

SEQ ID NO: 29 - protein, 12 aa, Homo sapiens
ITMLCTGSRTLK

SEQ ID NO: 30 - protein, 418 aa, Homo sapiens
  ref <308>: DATABASE ACCESSION NUMBER: ITHU
  ref <309>: DATABASE ENTRY DATE: 2000-09-15
  ref <313>: RELEVANT RESIDUES: (1)..(418)
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 31 - DNA, 1607 bp, Homo sapiens
  ref <308>: DATABASE ACCESSION NUMBER: NM_000295
  ref <309>: DATABASE ENTRY DATE: 2004-12-20
  ref <313>: RELEVANT RESIDUES: (1)..(1607)
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcaaaa

SEQ ID NO: 32 - DNA, 12222 bp, Homo sapiens
  ref <308>: DATABASE ACCESSION NUMBER: K02212
  ref <309>: DATABASE ENTRY DATE: 1995-08-08
  ref <313>: RELEVANT RESIDUES: (1)..(12222)
gaattccaggttggaggggcggcaacctcctgccagccttcaggccactctcctgtgcctgccagaagagacagagcttgaggagagcttgaggagagcaggaaaggtggaacattgctgctgctgctcactcagttccacaggtgggaggaacagcagggcttagagtgggggtcattgtgcagatgggaaaacaaaggcccagagaggggaagaaatgcctaggagctaccgagggcaggcgacctcaaccacagcccagtgctggagctgtgagtggatgtagagcagcggaatatccattcagccagctcaggggaaggacaggggccctgaagccaggggatggagctgcagggaagggagctcagagagaaggggaggggagtctgagctcagtttcccgctgcctgaaaggagggtggtacctactcccttcacagggtaactgaatgagagactgcctggaggaaagctcttcaagtgtggcccaccccaccccagtgacaccagcccctgacacgggggagggagggcagcatcaggaggggctttctgggcacacccagtacccgtctctgagctttccttgaactgttgcattttaatcctcacagcagctcaacaaggtacataccgtcaccatccccattttacagatagggaaattgaggctcggagcggttaaacaactcacctgaggcctcacagccagtaagtgggttccctggtctgaatgtgtgtgctggaggatcctgtgggtcactcgcctggtagagccccaaggtggaggcataaatgggactggtgaatgacagaaggggcaaaaatgcactcatccattcactctgcaagtatctacggcacgtacgccagctcccaagcaggtttgcgggttgcacagcggagcgatgcaatctgatttaggcttttaaaggattgcaatcaagtgggacccactagcctcaaccctgtacctcccctcccctccacccccagcagtctccaaaggcctccaacaaccccagagtgggggccatgtatccaaagaaactccaagctgtatacggatcacactggttttccaggagcaaaaacagaaacagcctgaggctggtcaaaattgaacctcctcctgctctgagcagcctagggggcagactaagcagagggctgtgcagacccacataaagagcctactgtgtgccaggcacttcacccgaggcacttcacaagcatgcttgggaatgaaacttccaactctttgggatgcaggtgaaacagttcctggttcagagaggtgaagcggcctgcctgaggcagcacagctcttctttacagatgtgcttccccacctctaccctgtctcacggccccccatgccagcctgacggttgtgtctgcctcagtcatgctccatttttccatcgggaccatcaagagggtgtttgtgtctaaggctgactgggtaactttggatgagcggtctctccgctccgagcctgtttcctcatctgtcaaacgggctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttccgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggttcagagaggggtggggaggaacgccggctcacattctccatcccctccagatatgaccaggaacagacctgtgccagcctcagccttacatcaaaatgggcctccccatgcaccgtggacctctgggccctcctgtcccagtggaggacaggaagctgtgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctgaacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagcgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgtgtttgcttagcatagggttaggcagctggcaggcagtaaacagttggataatttaatggaaaatttgccaaactcagatgctgttcactgctgagcaggagccccttcctgctgaaatggtcctggggagtgcagcaggctctccgggaagaaatctaccatctctcgggcaggagctcaacctgtgtgcaggtacagggagggcttcctcacctggtgcccactcatgcattacgtcagttattcctcatccctgtccaaaggattcttttctccattgtacagctatgaagctagtgctcaaagaagtgaagtcatttaccccaggccccctgccagtaagtgacagggcctggtcacacttgggtttatttattgcccagttcaacaggttgtttgaccataggcgagattctcttccctgcaccctgccgggttgctcttggtcccttattttatgctcctgggtagaaatggtgcgagattaggcagggagtggacgcttccctgtccctggccccgcaaagagtgctcccacctgccccgatcccagaaatgtcaccatgaagccttcattcttttggtttaaagcttggcctcagtgtccgtacaccatggggtccttggccagatggcgactttctcctctccagtcgccctcccaggcactagcttttaggagtgcagggtgctgcctctgatagaagggccaggagagagcaggttttggagacctgatgttataaggaacagcttgggaggcataatgaacccaacatgatgcttgagaccaatgtcacagcccaattctgacattcatcatctgagatctgaggacacagctgtctcagttcatgatctgagtgctgggaaagccaagacttgttccagctttgtcactgacttgctgtatagcctcaacaaggccctgaccctctctgggcttcaaactcttcactgtgaaaggaggaaaccagagtaggtgatgtgacaccaggaaagatggatgggtgtgggggaatgtgctcctcccagctgtcaccccctcgccaccctccctgcaccagcctctccacctcctttgagcccagaattcccctgtctaggagggcacctgtctcgtgcctagccatgggaattctccatctgttttgctacattgaacccagatgccattctaaccaagaatcctggctgggtgcaggggctctcgcctgtaaccccagcactttgggaggccaaggcaggcggatcaagaggtcaggagttcaagacctgcctggccaacacggtgaaacctcagctctactaaaaatacaaaaattagccaggcgtggtggcacacgcctgtaatcccagctatttgggaagctgagacagaagaatttcttgaacccgggaggtggaggtttcagtgagccgagatcacgccactgcactccaccctggcggataaagcgagactctgtctcaaaaaaaacccaaaaacctatgttagtgtacagagggccccagtgaagtcttctcccagccccactttgcacaactggggagagtgaggccccaggaccagaggattcttgctaaaggccaagtggatagtgatggccctgccaggctagaagccacaacctctggccctgaggccactcagcatatttagtgtccccaccctgcagaggcccaactccctcctgaccactgagccctgtaatgatgggggaatttccataagccatgaaggactgcacaaagttcagttgggagtgaaagagaaattaaagggagatggaaatatacagcactaattttagcaccgtcttcagttctaacaacactagctagctgaagaaaatacaaacatgtattatgtaatgtgtggtctgttccatttggattacttagaggcacgagggccaaggagaaaggtggtggagagaaaccagctttgcacttcatttgttgctttattggaaggaaacttttaaaagtccaagggggttgaagaatctcaatatttgttatttccagctttttttctccagtttttcatttcccaaattcaaggacacctttttctttgtattttgttaagatgatggttttggttttgtgactagtagttaacaatgtggctgccgggcatattctcctcagctaggacctcagttttcccatctgtgaagacggcaggttctacctagggggctgcaggcaggtggtccgaagcctgggcatatctggagtagaaggatcactgtggggcagggcaggttctgtgttgctgtggatgacgttgactttgaccattgctcggcagagcctgctctcgctggttcagccacaggccccaccactccctattgtctcagccccgggtatgaaacatgtattcctcactggcctatcacctgaagcctttgaatttgcaacacctgccaacccctccctcaaaagagttgccctctctagatccttttgatgtaaggtttggtgttgagacttatttcactaaattctcatacataaacatcactttatgtatgaggcaaaatgaggaccagggagatgaatgacttgtcctggctcatacacctggaaagtgacagagtcagattagatcctaggtctatctgaagttaaaagaggtgtcttttcacttcccacctcctccatctactttaaagcagcacaaacccctgctttcaaggagagatgagcgtctctaaagcccctgacagcaagagcccagaactgggacaccattagtgacccagacggcaggtaagctgactgcaggagcatcagcctattcttgtgtctgggaccacagagcattgtggggacagccccgtctcttgggaaaaaaaccctaagggctgaggatccttgtgagtgttgggtgggaacagctcccaggaggtttaatcacagcccctccatgctctctagctgttgccattgtgcaagatgcatttcccttctgtgcagcagtttccctggccactaaatagtgggattagatagaagccctccaagggctccagcttgacatgattcttgattctgatctgacccgattctgataatcgtgggcaggcccattcctcttcttgtgcctcattttcttcttttgtaaaacaatggctgtaccatttgcatcttagggtcattgcagatgaaagtgttgctgtccagagcctgggtgcaggacctagatgtaggattctggttctgctacttcctcagtgacattgaatagctgacctaatctctctggctttggtttcttcatctgtaaaagaaggatattagcattagcacctcacgggattgttacaagaaagcaatgaattaacacatgtgagcacggagaacagtgcttggcatatggtaagcactacgtacattttgctattcttctgattctttcagtgttactgatgtcggcaagtacttggcacaggctggtttaataatccctaggcactttcacgtggtgtcaatccctgatcactgggagtcatcatgtgccttgactcgggcctggcccccccatctctgtcttgcaggacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggtaaggttgctcaaccagcctgagctgtttcccatagaaacaagcaaaaatatttctcaaaccatcagttcttgaactctccttggcaatgcattatgggccatagcaatgcttttcagcgtggattcttcagttttctacacacaaacactaaaatgttttccatcattgagtaatttgaggaaataatagattaaactgtcaaaactactgacgctctgcagaacttttcagagcctttaatgtccttgtgtatactgtatatgtagaatatataatgcttagaactatagaacaaattgtaatacactgcataaagggatagtttcatggaacatactttacacgactctagtgtcccagaatcagtatcagttttgcaatctgaaagacctgggttcaaatcctgcctctaacacaattagcttttgacaaaaacaatgcattctacctctttgaggtgctaatttctcatcttagcatggacaaaataccattcttgctgtcaggtttttttaggattaaacaaatgacaaagactgtggggatggtgtgtggcatacagcaggtgatggactcttctgtatctcaggctgccttcctgcccctgaggggttaaaatgccagggtcctgggggccccagggcattctaagccagctcccactgtcccaggaaaacagcataggggaggggaggtgggaggcaaggccaggggctgcttcctccactctgaggctcccttgctcttgaggcaaaggagggcagtggaggcaagccaggctgcagtcagcacagctaaagtcctggctctgctgtggccttagtgggggcccaggtccctctccagccccagtctcctccttctgtccaatgagaaagctgggatcaggggtccctgaggcccctgtccactctgcatgcctcgatggtgaagctctgttggtatggcagaggggaggctgctcaggcatctgcatttcccctgccaatctagaggatgaggaaagctctcaggaatagtaagcagaatgtttgccctggatgaataactgagctgccaattaacaaggggcagggagccttagacagaaggtaccaaatatgcctgatgctccaacattttatttgtaatatccaagacaccctcaaataaacatatgattccaataaaaatgcacagccacgatggcatctcttagcctgacatcgccacgatgtagaaattctgcatcttcctctagttttgaattatccccacacaatctttttcggcagcttggatggtcagtttcagcaccttttacagatgatgaagctgagcctcgagggatgtgtgtcgtcaagggggctcagggcttctcagggaggggactcatggtttcttattctgctacactcttccaaaccttcactcacccctggtgatgcccaccttcccctctctccaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggtaaatgaactcacccacgatatcatcaccaagttcctggaaaatgaagacagaaggtgattccccaacctgagggtgaccaagaagctgcccacacctcttagccatgttgggactgaggcccatcaggactggccagagggctgaggagggtgaaccccacatccctgggtcactgctactctgtataaacttggcttccagaatgaggccaccactgagttcaggcagcgccgtccatgctccatgaggagaacagtacccagggtgaggaggtaaaggtctcgtccctgggaacttcccactccagtgtggacactgtcccttcccaatatccagtgcccaaggcagggacagcagcaccaccacacgttctggcagaaccaaaaaggaacagatgggcttcctggcaaaggcagcagtggagtgtggagttcaagggtagaatgtccctggggggacgggggaagagcctgtgtggcaaggcccagaaaagcaaggttcggaattggaacagccaggccatgttcgcagaaggcttgcgtttctctgtcactttatcggtgctgttagattgggtgtcctgtagtaagtgatacttaaacatgagccacacattagtgtatgtgtgtgcattcgtgattatgcccatgccctgctgatctagttcgttttgtacactgtaaaaccaagatgaaaatacaaaaggtgtcgggttcataataggaatcgaggctggaatttctctgttccatgccagcacctcctgaggtctctgctccaggggttgagaaagaacaaagaggctgagagggtaacggatcagagagcccagagccagctgccgctcacaccagaccctgctcagggtggcattgtctccccatggaaaaccagagaggagcactcagcctggtgtggtcactcttctcttatccactaaacggttgtcactgggcactgccaccagccccgtgtttctctgggtgtagggccctggggatgttacaggctgggggccaggtgacccaacactacagggcaagatgagacaggcttccaggacacctagaatatcagaggaggtggcatttcaagcttttgtgattcattcgatgttaacattctttgactcaatgtagaagagctaaaagtagaacaaaccaaagccgagttcccatcttagtgtgggtggaggacacaggagtaagtggcagaaataatcagaaaagaaaacacttgcactgtggtgggtcccagaagaacaagaggaatgctgtgccatgccttgaatttcttttctgcacgacaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggtgagatcaccctgacgaccttgttgcaccatggtatctgtagggaagaatgtgtgggggctgcagcactgtcctgaggctgaggaaggggccgagggaaacaaatgaagacccaggctgagctcctgaagatgcccgtgattcactgacacgggacggtgggcaaacagcaaagccaggcaggggctgctgtgcagctggcactttcggggcctcccttgaggttgtgtcactgaccctgaatttcaactttgcccaagaccttctagacattgggccttgatttatccatactgacacagaaaggtttgggctaagttgtttcaaaggaatttctgactccttcgatctgtgagatttggtgtctgaattaatgaatgatttcagctaaagtgacacttattttggaaaactaaaggcgaccaatgaacaacctgcagttccatgaatggctgcattatcttggggtctgggcactgtgaaggtcactgccagggtccgtgtcctcaaggagcttcaagccgtgtactagaaaggagagagccctggaggcagacgtggagtgacgatgctcttccctgttctgagttgtgggtgcacctgagcagggggagaggcgcttgtcaggaagatggacagaggggagccagccccatcagccaaagccttgaggaggagcaaggcctatgtgacagggagggagaggatgtgcagggccagggccgtccagggggagtgagcgcttcctgggaggtgtccacgtgagccttgctcgaggcctgggatcagccttacaacgtgtctctgcttctctcccctccaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgatctgtaaatccctgggatgttttctctg

SEQ ID NO: 33 - protein, 381 aa, Homo sapiens
  ref <308>: DATABASE ACCESSION NUMBER: P12277
  ref <309>: DATABASE ENTRY DATE: 2005-01-25
  ref <313>: RELEVANT RESIDUES: (1)..(381)
MPFSNSHNALKLRFPAEDEFPDLSAHNNHMAKVLTPELYAELRAKSTPSGFTLDDVIQTGVDNPGHPYIMTVGCVAGDEESYEVFKDLFDPIIEDRHGGYKPSDEHKTDLNPDNLQGGDDLDPNYVLSSRVRTGRSIRGFCLPPHCSRGERRAIEKLAVEALSSLDGDLAGRYYALKSMTEAEQQQLIDDHFLFDKPVSPLLLASGMARDWPDARGIWHNDNKTFLVWVNEEDHLRVISMQKGGNMKEVFTRFCTGLTQIETLFKSKDYEFMWNPHLGYILTCPSNLGTGLRAGVHIKLPNLGKHEKFSEVLKRLRLQKRGTGGVDTAAVGGVFDVSNADRLGFSEVELVQMVVDGVKLLIEMEQRLEQGQAIDDLMPAQK

SEQ ID NO: 34 - DNA, 1431 bp, Homo sapiens
  ref <308>: DATABASE ACCESSION NUMBER: NM_001823
  ref <309>: DATABASE ENTRY DATE: 2004-10-27
  ref <313>: RELEVANT RESIDUES: (1)..(1431)
gctgttcgcctgcgtcgctccgggagctgccgacggacggagcgcccccgcccccgcccggccgcccgcccgccgccgccatgcccttctccaacagccacaacgcactgaagctgcgcttcccggccgaggacgagttccccgacctgagcgcccacaacaaccacatggccaaggtgctgacccccgagctgtacgcggagctgcgcgccaagagcacgccgagcggcttcacgctggacgacgtcatccagacaggcgtggacaacccgggccacccgtacatcatgaccgtgggctgcgtggcgggcgacgaggagtcctacgaagtgttcaaggatctcttcgaccccatcatcgaggaccggcacggcggctacaagcccagcgatgagcacaagaccgacctcaaccccgacaacctgcagggcggcgacgacctggaccccaactacgtgctgagctcgcgggtgcgcacgggccgcagcatccgtggcttctgcctccccccgcactgcagccgcggggagcgccgcgccatcgagaagctcgcggtggaagccctgtccagcctggacggcgacctggcgggccgatactacgcgctcaagagcatgacggaggcggagcagcagcagctcatcgacgaccacttcctcttcgacaagcccgtgtcgcccctgctgctggcctcgggcatggcccgcgactggcccgacgcccgcggtatctggcacaatgacaataagaccttcctggtgtgggtcaacgaggaggaccacctgcgggtcatctccatgcagaaggggggcaacatgaaggaggtgttcacccgcttctgcaccggcctcacccagattgaaactctcttcaagtctaaggactatgagttcatgtggaaccctcacctgggctacatcctcacctgcccatccaacctgggcaccgggctgcgggcaggtgtgcatatcaagctgcccaacctgggcaagcatgagaagttctcggaggtgcttaagcggctgcgacttcagaagcgaggcacaggcggtgtggacacggctgcggtgggcggggtcttcgacgtctccaacgctgaccgcctgggcttctcagaggtggagctggtgcagatggtggtggacggagtgaagctgctcatcgagatggagcagcggctggagcagggccaggccatcgacgacctcatgcctgcccagaaatgaagcccggcccacacccgacaccagccctgctgcttcctaacttattgcctgggcagtgcccaccatgcacccctgatgttcgccgtctggcgagcccttagccttgctgtagagacttccgtcacccttggtagagtttatttttttgatggctaagatactgctgatgctgaaataaactagggttttggcctgcctgcgtctg

SEQ ID NO: 35 - DNA, 4200 bp, Homo sapiens
  ref <308>: DATABASE ACCESSION NUMBER: X15334
  ref <309>: DATABASE ENTRY DATE: 1993-04-24
  ref <313>: RELEVANT RESIDUES: (1)..(4200)
gatcagtttttttttttaatcgcacttatgcttattgtttattagcgtttcctcccatctttgcctgaagtctccggggactgcctttgggggtcgggtaaacttgtcccctgcgaagagggcccagggttggggtctggaaactccgaggctgcacttgccagcggcctcttaaggccacagcgtccccgtggtttctggctcgcagccccccgagacccaggacttgtccaaggtcagggcaccgcgggtgcccccgggctgggccgcagcagactgcgcttcccgcgcgccttcgctttgcaccaggatcgcccaggaaatgcctgcgggcaccttgaggaaggtcggcggctccgggccagctcgcactggccggggtggggcgggggccgtacctgctgcggaagccccgaaagctttcgcccggcccctcgccgccgccgcgggggctggctggactaggcgggcaggctcgaggatgcggatgaacccaagcgtcctcgagtgcccggaggctctccgcctcagtttcccgcccagaggcaagggcgtgcgaggggatccagatatccaaggacctgaggtttcggcctcgaggtcttgggcgggggactgggcaggctgcgcggggtcccagcgaggggacagctcgggtgggcggccagggtgttgggggctgcgggcggcggacaaagcggcggcaccaccccgcggcgcgggccaatggaatgaatgggctataaatagccgccaatgggcggcccgcgttgtgccccttaagagccgcgggagcgcggagcggccgctgttcgcctgcgtcgctccgggagctgccgacggacggagcgcccccgcccccgcccggccgcccggtgagtgggcccgggggccgggggcgtccgcgcccgggctaggggcgctgcgagcaaagggggcgcgtcgcctggagcgcgcgccggaccggccgggggtccccggcgatgatggcgctccccgcgcgcgctgcggaccccgctgaccttggccgcgtcccggggggcgccggggggcccggcggcgggggcctgagtggtacgcgggagcccgggaaccccggcgtgccggtcccctctgaccccgcgtctccccgcagcccgccgccgccatgcccttctccaacagccacaacgcactgaagctgcgcttcccggccgaggacgagttccccgacctgagcgcccacaacaaccacatggccaaggtgctgacccccgagctgtacgcggagctgcgcgccaagagcacgccgagcggcttcacgctggacgacgtcatccagacaggcgtggacaacccgggtacgcgacccctcggggccggggtcccggccccccctccccccgcgcagccgcagggtcctcagcagcgcgctcgggcccggcagtgacgtcactgtccccgtcccgcgccccctcccccaggccacccgtacatcatgaccgtgggctgcgtggcgggcgacgaggagtcctacgaagtgttcaaggatctcttcgaccccatcatcgaggaccggcacggcggctacaagcccagcgatgagcacaagaccgacctcaaccccgacaacctgcaggtgcggggctgcgggcgggccgggcgggcggggccggggtcttcgggcgctcactcccgtctcgcctcccagggcggcgacgacctggaccccaactacgtgctgagctcgcgggtgcgcacgggccgcagcatccgtggcttctgcctccccccgcactgcagccgcggggagcgccgagccatcgagaagctcgcggtggaaggtaggggccgggcgggccgaggggcggcggcggccgcgtccccctcccggcgcggtccccgcccgcttttgtttacgtcgcccgggagcggcagccgccgtcgcgctcttatctgcgcgcgcccgggttcagtttcccggacccaccgagggacggaggcccagcccccgcgcccacagcggcctggggcccagggagggcgggtcctggcgcggggtcaccgcctgggaccgtcgcccgggccgtgaggactggacgcccgcagatccgggcgggtggggccctctgacgtcccccgaggtggggcacgggggcgggcgggtccgcgctgcgggctggaggggcgggcgcgggagcccagcgtcctgagcgcacccctcgcagccctgtccagcctggacggcgacctggcgggccgatactacgcgctcaagagcatgacggaggcggagcagcagcagctcatcgacgaccacttcctcttcgacaagcccgtgtcgcccctgctgctggcctcgggcatggcccgcgactggcccgacgcccgcggtatctggtgcgtgtccctctgcgccctctcgcggcgtcctccctccccgctacctccgctttccctctcgcccccctcgcgggggtggggcccctcgcggcgaggaggaggaggaggaggagggaggggccggccgcgctccgggtctgggttccgtgccgcgcctcctcctgcgccggtgaccttggccgagcaggtgcgttaagggactgggccccggcccgtgggggctcaggactcagcaacacctccccaccccgagacgtgaggtgggggcggggctctctggcgcctctccccgacggccctgggagctggagctctttgttttcttttctcactcctccgccgctgggattctaccaggggctggtgacgccaaagcttctccaggggcagggctcctacccccactgtggggggcgggtcgggctgtcctggcggtccctggccccgccccacctcgggccacagcgcatgatggcagctggggttctcctgctgtgaggcgtcccggttcccccgcccgccccgtgttggcgggtggagtcttggcagcagcctccactcctgggcatggcagggagcagcacctcagggacttgggaagttcctttggtctgggggcggcctggggcttttttctgggtatgccctgagaccagccctcccgcaggcacaatgacaataagaccttcctggtgtgggtcaacgaggaggaccacctgcgggtcatctccatgcagaaggggggcaacatgaaggaggtgttcacccgcttctgcaccggcctcacccaggtgccagggacggggcaggcccagaccccagggccccagcagggatgtgggtgccccagcatcagtccccccgggggatttccggcactggggagtctcagggcctgtaggggtttcaggcaggccttctccctcataccctcttctccgtctgcagattgaaactctcttcaagtctaaggactatgagttcatgtggaaccctcacctgggctacatcctcacctgcccatccaacctgggcaccgggctgcgggcaggtgtgcatatcaagctgcccaacctgggcaagcatgagaagttctcggaggtgcttaagcggctgcgacttcagaagcgaggcacaggtgagcagggcaggtgctgcggcttcccgtggcctttgggcagccctgtttcctccgccctgacttgctgtctccccaggcggtgtggacacggctgcggtgggcggggtcttcgacgtctccaacgctgaccgcctgggcttctcagaggtggagctggtgcagatggtggtggacggagtgaagctgctcatcgagatggaacagcggctggagcagggccaggccatcgacgacctcatgcctgcccagaaatgaagcccggcccacacccgacaccagccctgctgcttcctaacttattgcctgggcagtgcccaccatgcacccctgatgttcgccgtctggcgagcccttagccttgctgtagagacttccgtcacccttggtagagtttatttttttgatggctaagatactgctgatgctgaaataaactagggttttggcctgcctgcgtctgagtggtgcctctcctttcccaggggggagggggaagggcagcagccaggccccaggagtcttgagtcctgggcctgctgtgggcctcgccttctgtgagatgggacaagagccaggaggtggccactctgttctgcctgccctacctagtccatgggccccttccctcgtgtctatcgggctgtgcaggcaggaacatgggagagagcgagggagga

SEQ ID NO: 36 - protein, 531 aa, Homo sapiens
  ref <308>: DATABASE ACCESSION NUMBER: P14618
  ref <309>: DATABASE ENTRY DATE: 2004-10-25
  ref <313>: RELEVANT RESIDUES: (1)..(531)
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 37 - DNA, 10368 bp, Homo sapiens
  ref <308>: DATABASE ACCESSION NUMBER: X56494
  ref <309>: DATABASE ENTRY DATE: 1998-11-13
  ref <313>: RELEVANT RESIDUES: (1)..(10368)
ggtcttcacattttgaatgcgcaacattgtatctgtgaatgaaggcaagagttaacagctgtttaattgataactgctcgcatcattagttgctggctaacaactgggaaatcagaaaatgtcttgtagaaaaatgtaagaaaagttccaacaatactgacttaaacacgagcaaaggtgaaaacagaaatgctgactcctgcataggttatcggccctaatgttctgacttgatatttccagatgcccagctctgcgctaatatcaacaccgtctatttactttctactctgaggcattcgctctgcaggattccagaccctactaaattattcacatggccccaaccggtccttccttgttccgcggtcctaacacaatgaatggtcctaagaggaaaacggcctcggctcccgctccaggcccacttcgcagtccctagttctccctactgccgctccagtgccagagcccctccgaaggcggccaggacctccaaccacgcacaagtctgcagctctccccaactttccgttcagctcagtctccgagggtgcgccagagcagacacccggaggagtggggagtggcagggcggggccgggagaatgctgccccggaacccataaattcggccctgcccaggtaggccgggacagctggggtggcctgggccgagagccaagaaaagagaccccatctggacgcccaacttggcggcaacaggtggccggcgcccgggggtctgggaggaaagtcgctccgggcgggccccgttgccccgccgcgtccccattggtcatcaggtttcttaaaatgtgactctgaatctgtgtccttccgccgcagaatttagtcccaccgaaagggcaacctgcccgcgcgttccgccaccgccgccgcgcttcctcctgaaggtgactcgagcccgcggggacgcagggggcggggcccgggtcgcccggagccgggattgggcagagggcggggcggcggagggattgcggcggcccgcagcgggataaccttgaggctgaggcagtggctccttgcacagcagctgcacgcgccgtggctccggatctcttcgtctttgcagcgtagcccgagtcggtcagcagccggaggtgagcggtgcaggcagtacgccatcagtccccaccaagggccagtcgcccggctagtgcggaatcccggcgcgccggccggccccgggcacgcaggcagggcggcgcaggatccctgtgctaaatggtatattaaccacttctcagtcttaccactctctttcaatttgtctcgacccaggacctcagcagccatgtcgaagccccatagtgaagccgggactgccttcattcagacccagcagctgcacgcagccatggctgacacattcctggagcacatgtgccgcctggacattgattcaccacccatcacagcccggaacactggcatcatctgtaccattggtgagtgggtgtcccccttcccccaaaaagggcttcatgggcagtgacctttctctcctgaaaagagctccatgcactttttaaagacttttgagctatttgggagaggaaaaattttcagggaaaaaaattctttaaacttaaagcaaacttaaatgtttttccttggttgaataattaatacttgtggctttaaaacttttcctaataggcccagcttcccgatcagtggagacgttgaaggagatgattaagtctggaatgaatgtggctcgtctgaacttctctcatggaactcatgaggtgagctgtggctggaccctatcctggcaggggaattggagctggattctagtgtgggagcacgcttgtcatcttccttcttttcccccagtaccatgcggagaccatcaagaatgtgcgcacagccacggaaagctttgcttctgaccccatcctctaccggcccgttgctgtggctctagacactaaaggacctgagatccgaactgggctcatcaagggcgtgagtattctgcggagagcgaggggaaggctcagtaggcaatatgccccagagacatgattccttccgaggtgatgctgctactggtgtctccagtttggactcttccttactctcttgtccctagagcggcactgcagaggtggagctgaagaagggagccactctcaaaatcacgctggataacgcctacatggaaaagtgtgacgagaacatcctgtggctggactacaagaacatctgcaaggtggtggaagtgggcagcaagatctacgtggatgatgggcttatttctctccaggtgaagcagaaaggtacgtatgggagctggagtccagttgtctaaaacagtcttttgtctctaaacttctcgtctctgcctccccaacttacccttttttatacaggtgccgacttcctggtgacggaggtggaaaatggtggctccttgggcagcaagaagggtgtgaaccttcctggggctgctgtggacttgcctgctgtgtcggagaaggacatccaggatctgaagtttggggtcgagcaggatgttgatatggtgtttgcgtcattcatccgcaaggcatctgatgtccatgaagttaggaaggtcctgggagagaagggaaagaacatcaagattatcagcaaaatcgagaatcatgagggggttcggaggcaagtccccgttgtccctggtctactgccatacttgtggcctctgttctatataacctctctcccccccactttgtccatcaggtttgatgaaatcctggaggccagtgatgggatcatggtggctcgtggtgatctaggcattgagattcctgcagagaaggtcttccttgctcagaagatgatgattggacggtgcaaccgagctgggaagcctgtcatctgtgctactcaggcatgtgcccacccttccccacattctcatgtgcacactcgcatgtttgtatgggaaagctctggaggctgtctgatctcttcccatggaattgtcgcaacgtaacacacagataatccccttcccccatgtacctacacaaagccatactctgtgtacctactcactatccagaggatcagcttgctgtcatttgtctctgaagacagctcaagctacatctcactaatgctctgtcccctcccagatgctggagagcatgatcaagaagcccccgcccactcgggctgaaggcagtgatgtggccaatgcagtcctggatggagccgactgcatcatgctgtctggagaaacagccaaaggggactatcctctggaggctgtgcgcatgcagcacctggtgagttctgggcctgccccatcccccagggcttcggactgggcctgggatggatgcaagctctggtgcagagctttttaggtttctccatcctcttatgcacagcctttcattatcctccaagttacagcagcaagagggtgggggtggaagtggaggtggctttttttttttctcctgttcctgcattcctgcccacacccccacccctctcatttccttctgctctggaggcacctccttcattggacaccacacagtttatttcacttctgacttcaaggttgtgaattcttcccatggcttaagtcctgggatacttctgcagtgaaaggaggtcttgtacctcttcctcagagtcagaagttctgagtacctttgccctattctgaaaagggctaggggctcctgctcccagctgccctcttcctttggcttccaattcagttccctctgccccgcatcctgcagacaggcgctcccgcagggggcccttgtggacctgcactggagtctgttgccttcactgagctgcctgtgctggccttgcatggtgcctgtagggggatttgctttgctgtgccattggggtacagctgctgctcttactctagaccaaaaagtcgggttgagtgactggtggcagggccaagatagagacagcggggagggtggctgaccctggcggccctggactgagcgtctggaggagtcgtggaggctctttcccttctttctcctctgagagctcgttcttcaggctcttccagcttgtcatgtcgagtgcctggccactgctcagggttggaggctcagtccctttgccctgtctgttccagctctggagctaactcagggatccctgatcagggttacgtaggtttggtaaaatgagtgctggaaattaactttctcccagtagtcttaggtctagctcagtgaacttaaactttatccagatatggtttttccttcagcctttctattccctttctagccagtgaaagacccgctgccctttgacctcagccccctccaagcccccaagtttaaaacgccaccccctgccaccagaaaaaacagaaaaaaaaaaaaaaaaaaaaactaaaacacccatctggtctgggcatcttccttcctttttcactatgtatcctgttactgggcttaaacagctttcagagaagagatgtcatttctattaaatgctctttcagtagcgaactgagttcacacttgactaaggatattttccggactgtctgtcatcagcatccttagtgggtttccccatatttaaattggtagaggccagggatggtggctcacacctgtaatctcagtactttgggaggccaaggtaggtggattgcttgagctcagaagaccagcctgggcaacctggtgaaaccctgtctctactaaaaattcaagttagctagctgggcatggtgatgcacttctgtagtcccagctacttggagagggggtggtgctggggcagcaggatcgcttaaacccaggaggttaaggttgcagtcagccaagatggtaccagcctaggtgacaaagtgacaccctgtctcaaaaaagaaaccaaacaaacataaaaaaaaaaacaaaaaaatcggtagagagtgatttctctcccaggcccacttaatgtagactgggcctggctgacacctcaccattcgtgtgatgtgattgctgttctgatgcttagatactcttggcgcagtctcacaattgccaccatggtaggaaggtgtccaggagacggtgcaccttgaaccagtcaccactaaagtggctgcctttctgggtctctccacacatcccctctctctaatttccctacttaatcgtgtgacttcatggtctcaaaggaggaacagaggctgatcttgacttagatatactgaaccatgaaatcactgcatagaatgtggggacttgaatgtgtctttgggcaagtcatttaacctcttaagacctcatctgtaaaatggattagatatgtttaattatagccttagcattaaatattcattgctgttattattaagtgtctgataagtctctgtgtacatggatgtaatcttcctaactcccattacctccatttatagatgagggttatatggccaataaagcctgggtttgaatctaggtctactgcctccaaagccagtcttctctcctgcaacatcatgctctgtctagcaggagatgagaacaggtctccatttggagcctgtcagtggggtcagagactaagattcaggctcagggtctaaattccgtatcctttcttccataccctggtgtttcctatgaacagatagatactttagggctgcaaggtttggattgcatggcactgctcagaagataagttacaggtctgggctaggctgtagctgcccctccaggtggctagacctttcctttctgtgtcaccagttaacactggccaacagttccttccattaactgttcactgctttctcctgtgtctaactgatgcagtttatgacccataactaagagcagtaccaggtatggctctgtttcctgttcatgtcccctgtcctctgggctgcatgcattccgttcttacagaaagaatacctttaacctagtacatcctgccacacatctgcttctactgtgaaattgatgagggggtattaccgattcttccctctcccatcatttactgagatgctggtgattgcattataatcctcttaagcttacattgtctttctgattcttggtcttatctgagcaagtgatctataaataactcagtggctttctcatgactgttttaattattagattttaatcaagtgtcttattaaatatatctgcatgcttccacaggcatctgtctcttcacatggctgttcagtgtgcctctcacaacttagcccaaactcagttgagctgccttgctttggctttgacccagctttccagcgctgctcaatctgttgccatggcaggccattggaaaggctcagttcatccccgtgcctgaagccaagtgagcgctcactccatgcatgcatggaggctgggcaggagcctgcctaatcaaccagccatgtgaggagggagggcctgttccttcctgtaagctatgtcatgaggcagcgtggtcaagtcctctgccagggagtggcctggcccagcctgggcatgttttcatgccagggtgctagagcctactgccagattgtctccctccacccccaatgaaaaaatccttccagaagggaagagccaatttcccctgtattggaggggaagtggcagcacctcctgaagcagttggactttcatcaccctacctctgcatctgcctgaaggacagatttagccaattaacctaaggttaccttcctctctgataaattccccattctgtcttcccatgtgttgtgtctcgtttttttcctcctccttccctcttccttgccccctcttcccctaaaccttacagatagctcgtgaggctgaggcagccatgttccaccgcaagctgtttgaagaacttgtgcgagcctcaagtcactccacagacctcatggaagccatggccatgggcagcgtggaggcttcttataagtgtttagcagcagctttgatagttctgacggagtctggcaggtagggccctaagggcaggtaacactgttaggataaccagcctcttgctgcacctgccccaggagaagagagaaggcccaacctggcatctgggaacagagcctcttctcgtctgtaggaacaccgccagggaggtcatggcagggcagaccaaagggtcctgtggctcagtaggcacagtagatgtcacaggcacttggtgaaggactggtttctgtggagtcttgatcttggctcagctcagaatctccagtgattgggctcctcttggcctttgttcccaggaacatgttcctcaccagctgtccggtgactcttcccctccctctccttttgtgacaaagctctgacaaagctctgtccccctctcgtccctctggacggatgttgctcccctagattgcccgtgaggcagaggctgccatctaccacttgcaattatttgaggaactccgccgcctggcgcccattaccagcgaccccacagaagccaccgccgtgggtgccgtggaggcctccttcaagtgctgcagtggggccataatcgtcctcaccaagtctggcaggtaggaggcggcagcggctccctggaatgccctgctcagtggtacctcaccttgggggtcctgggagcagtccattgaacaatgctcaggtggcactgagccaaggtaagacccctctgcctgccaccttgggcctgcagggaaggattgagcagagccccttcccagggcccaaaggactctaggtagcactcataaggaatgtcagaacatttggatcaaaagcaaatttatgctggagatttattacataacagtgcacaggctgactacaaatggttatttgatattgaaaatttagtcctctaaaattgtaaaagataccacttttgcttattccagttactatgtgctctttaaaaatttcagttgggaaatgaatttatttaaatgctgtttactgtgcctccatttggcacactagtccctgctgtttttgagccctaaagacaaattgggttccagctcaggagaggttgctgtgctatcttggctgacattctgtgggcctggcagccaggctgaggactgtgtggcctatgctgggcctccaacttgggatcccttccttggcccaggacattgagttaatgtccttcactctcctagttagggagtatgctccttgtccctgtccacaggggagcaagggtttcctggaagaggggagcaaacaggcagtgcccatgcactgaggagcagcagatgggcgtgggcagcccagagaaccaggacacaagctctgtgcagatccctcagcagagggctccagcctcccactcttggctgaacagctccaacccgtagggttgacctttcttaaaaggtccagttcttgctgtttggctattttaagctctagtcttctggggtttcactcagctggtcctggcttcagcaattgcttccctctgaaggccttgcatagaggccaagcgtgaagtgcagggacttctctgctgtgatgtggcttaagtttccctgacacctgttgagtgtcctcataacttcccttctggtgcccctccccagctcctgagaccagctgcagctacaagtgtgcagtgtcagtgttcaagaaagtgcctggcagaggggctttagaagggtcccctgccttccaaaggagctttggcaggcagacgtgctcctgcagcaacactcccatttcctgttcttgcctgctgagtagcacctagatttctaagcctcatctagatactcagatttgattctgggcctttatagcccagttgctgggactgtttcaggagctaggggccatgtggggcagggagagggcacaaaagtagagaagcctgatgttgattcccagggggctggtcagctctgctactgctccttgcagatgtcaagagtcaggtgctagtcacgtgctgcttggcttgtcactgtcattggcagcgagaggaatgggtgctggtgacattgggccagggctgcctctctgtgtcagagttcagggtgtaggaggggttctgccaaccatgggctgtgtggggtaagtgggttgaggctgatctttctgggtcaaggtgatcctgagcccttgcctgtggaatgggggtagagggcaatggtaacctagctagcatgctgtgggggatataggatgaggggctgcccgaccctcgggaggggtcctagggagcagatgttgaagaggccagagccctcagtgagctggatgagggggtgagccgtttgaactccctgagggtacttcctggggcctcgtgtaatggtctcttctgtatgtcccccatcccatctcaggtctgctcaccaggtggccagataccgcccacgtgcccccatcattgctgtgacccggaatccccagacagctcgtcaggcccacctgtaccgtggcatcttccctgtgctgtgcaaggacccagtccaggaggcctgggctgaggacgtggacctccgggtgaactttgccatgaatgttggtacgtggctggagcaggggctagagcctagaggagcttggggatgcttgagcattggccaccaacctcccttctcttcctccaggcaaggcccgaggcttcttcaagaagggagatgtggtcattgtgctgaccggatggcgccctggctccggcttcaccaacaccatgcgtgttgttcctgtgccgtgatggaccccagagcccctcctccagcccctgtcccacccccttcccccagcccatccattaggccagcaacgcttgtagaactcactctgggctgtaacgtggcactggtaggttgggacaccagggaagaagatcaacgcctcactgaaacatggctgtgtttgcagcctgctctagtgggacagcccagagcctggctgccccatcatgtggccccacccaatcaagggaagaaggaggaatgctggactggaggcccctggagccagatggcaagagggtgacagcttcctttcctgtgtgtactctgtccagttcctttagaaaaaatggatgcccagaggactcccaaccctggcttggggtcaagaaacagccagcaagagttaggggtccttagggcactgggctgttgttccattgaagccgactctggccctggcccttacttgcttctctagctctctaggcctctccagtttgcacctgtccccaccctccactcagctgtcctgcagcaaacactccaccctccaccttccatttcccccactactgcagcacctccaggcctgttgctatagagcctacctgtatgtaataaacaacagctgaagcacctgtttcctctctttt

SEQ ID NO: 38 - protein, 201 aa, Homo sapiens
  ref <308>: DATABASE ACCESSION NUMBER: Q01995
  ref <309>: DATABASE ENTRY DATE: 2004-06-15
  ref <313>: RELEVANT RESIDUES: (1)..(201)
MANKGPSYGMSREVQSKIEKKYDEELEERLVEWIIVQCGPDVGRPDRGRLGFQVWLKNGVILSKLVNSLYPDGSKPVKVPENPPSMVFKQMEQVAQFLKAAEDYGVIKTDMFQTVDLFEGKDMAAVQRTLMALGSLAVTKNDGHYRGDPNWFMKKAQEHKREFTESQLQEGKHVIGLQMGSNRGASQAGMTGYGRPRQIIS

SEQ ID NO: 39 - DNA, 1822 bp, Homo sapiens
  ref <308>: DATABASE ACCESSION NUMBER: D84342
  ref <309>: DATABASE ENTRY DATE: 2002-12-25
  ref <313>: RELEVANT RESIDUES: (1)..(1822)
ccgggtgaaagcagagtgctccctgaccctctgcccctccctcctccaccctggcctgctttagctttccccagacatggccaacaagggtccttcctatggcatgagccgcgaagtgcagtccaaaatcgagaagaagtatgacgaggagctggaggagcggctggtggagtggatcatagtgcagtgtggccctgatgtgggccgcccagaccgtgggcgcttgggcttccaggtctggctgaagaatggcgtggtgagtggcaccctgggctagggcgctggggggctggggtgtgaccccctgtgagtcctgggccaatccctgaggactgctaagctgcgtcctatgccctatgcctggtagattctgagcaagctggtgaacagcctgtaccctgatggctccaagccggtgaaggtgcccgagaacccaccctccatggtcttcaagcagatggagcaggtggctcagttcctgaaggcggctgaggactatggggtcatcaagactgacatgttccagactgttgacctctttgaaggtagagaggagaatgctgggggaggaggtgggcaggaggacagggtgctgggacagggagagggtatgaccaaatatgccacaactaggggtgtgctcgcccgcacacagcagggatgggatatgccgagaataacacgccacgctcacagggcccactgagaggcctcccttgaattggggacaactcttggccctggtttggccatttttttgtgagagacgggggcaggccctggcttggagtcttgtttatacgttcttgatgttcatctcctctctcctgtcttctcacaggcaaagacatggcagcagtgcagaggaccctgatggctttgggcagcttggcagtgaccaagaatgatgggcactaccgtggagatcccaactggtttatgaagtatgtggcccccagggagcttgagtctccgcatggggtgggaggtggcttgttctaaggagcttgcgggaaggattaggggaagcagatagccaagaaaggataaagtgagggtctgggatggggaataatgggtccttaatactccttgacccctccctttccaccctcctgcgctcagtctccctagcctatgaggcaagctagattagggaaaaaaagtgcaacaggaaggcaatgggattgggctaggacgtaacagagggatcagaaaacgggtggaaaacacacagttctaccaagtctttatcctgcttcctcctcttctaggaaagcgcaggagcataagagggaattcacagagagccagctgcaggagggaaagcatgtcattggccttcagatgggcagcaacagaggggcctcccaggccggcatgacaggctacggacgacctcggcagatcatcagttagagcggagagggctagccctgagcccggccctcccccagctccttggctgcagccatcccgcttagcctgcctcacccacacccgtgtggtaccttcagccctggccaagctttgaggctctgtcactgagcaatggtaactgcacctgggcagctcctccctgtgcccccagcctcagcccaacttcttacccgaaagcatcactgccttggcccctccctcccggctgcccccatcacctctactgtctcctccctgggctaagcaggggagaagcgggctgggggtagcctggatgtgggccaagtccactgtcctccttggcggcaaaagcccattgaagaagaaccagcccagcctgccccctatcttgtcctggaatatttttggggttggaactctc

SEQ ID NO: 40 - protein, 355 aa, Homo sapiens
  ref <308>: DATABASE ACCESSION NUMBER: Q14103
  ref <309>: DATABASE ENTRY DATE: 2004-10-25
  ref <313>: RELEVANT RESIDUES: (1)..(355)
MSEEQFGGDGAAAAATAAVGGSAGEQEGAMVAATQGAAAAAGSGAGTGGGTASGGTEGGSAESEGAKIDASKNEEDEGHSNSSPRHSEAATAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 41 - DNA, 14983 bp, Homo sapiens
  ref <308>: DATABASE ACCESSION NUMBER: AF026126
  ref <309>: DATABASE ENTRY DATE: 1998-06-13
  ref <313>: RELEVANT RESIDUES: (1)..(14983)
tcgcagaggtgcagccacaccccggcctaacgtgttgttccccccgatactggagtggtggggagggtgagtggactccaggaatcctcggaagggcgggggcggaggcagggggcccctctagccgctacttcgaaacagcattccttgttctcgatggtccccgcgcgactgtcttagctcacgacacttccggttccttttaaaggcccccaaggctgtgcaacgcggagcgtgagaggaaggtataaagggtagcgagagggcgggaccgaggaggaaagggaaaaaaaaaaaactagggggataggggtggggggacgcgcgaagggcgcgctctcgcgtcacgtgaccgggacgcgccgttcttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcgcggggattactttgctgctagtttcggttcgcggcaggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaagggtgagtaaggggcatcccaggatagtcaggcccaactagtccccctccccctctttattcccccgccattagcgctggttcccgctctcagcccgttctccgggccccatgcagcctccttgcactggtctccctttttctatgttgggttccccggaccttcatttccttcttcctttgcctgcttatcgcccccctcccccatcacacacgtttccacctttagccgtcaccatgctgctcctcggcccgccctcctttcctccctcgccatgggtctcctcccaccgacttagccgccagattttttcccgcctgtcgtggggtaccttttttctttccatgctgtcccctctttttccctttttctacagtcttgggcataaaaacacagacacaaacagcttctctgtttgtattactaaggtttatttggtgcttctccaccatcctgaaacgatgcgattgtttataagcacatgttttggggaacgcgtaggctgtccacctctgcctctcccttgtcggccttacgccgactgttttcttgggattttgaataagtttcgcctaaggatttactcattttctccaccatacgctatcgcacaatggcatactcatacaggccctacattttgacatgcagaccaaattgggtctggtgaaatgctccgagtttcttgttggtacattggttttgctccgcgggctctggttaagttcttagtcgatcgggcctgcacttgactggagctgttcctatctccggcctagcatctacccttcccccaccccactgagttattctaaccgcgcacccttttcgcgccctcagctattgggttccccaactgttagtacagattgtaccttactttttatgtgcaacctaattttttacaacccccagccccctttttttcccggtgcccaggatcctattttggtgtcttgatatctgtttcccgcccactaagggaggcctgtagtcctcttaagagaaacaaatcactgtattgtgctatgggatactttttttttctttttggtgcaaatttcttagtattaacttgcttcctccaattgaaataacagttgtatatactacctaaatctgcatttagtgtattaaaatgcagcatttgggattgggaacataatacgggagttagcaggagggactaaatcaggtttcttggttgagttttcttagctccgattactgaccgatgatcgtgggttcacgcttccattcctttttgtaatggggaggggtgtgtgtgtgtctgaatttttttttaactgtaaagagaaaggtgttggtggatataacaagatgtcgctttaatctagtattagaaaacacgatttcttttactgagaaagagcccaggatttggagggaaagttgggaggggagaagcactttggaatatagggtttattaagcccagcttaggcagattcttgtcaggctgtttgatgatagtgctggttttgggggagtggtggtgggaagggaagcaaacttttaaaaattaaacagaaccaactcgaattgtatatagcttatttgagaaaggaagatgttagatatttggaaacttaaaacctttaacattttggttttcgtttgctgaattctgttctttggacagaggcaccaaaatgattaattgtataacttcctgtttggggcaggctttctgttagccctgataattcaaatcgcaaagcagctgactttatagttacctactgttgaagtgtaaatgaattaaagtttttaaccctaacagtgtcaaaaactaaaactagaattttgattggttgctgtaccggttgttaattccctagccattcaaactcctccccacgacactctgaagcagcgacggcacagcgggaagaatggtaaatcttttaaattttatttctgtattataaaggtttcagtagtcataatatgtagaggctgtgtattggttagttgcccattgatccccaggaaattctagaccatagtacatacagtaggtttgtacctgggatatataccttttataggcatcatgtgtaatctttgggcaaaattgatgccgttacatggtatttggtctgtggtaatgcatgtagttgcagtttggcaatttaagctaaacaattagtaacgatatttttatatcgagcaagaaaaaatattaccgtaatcttcacatttgatttattttaagatgaaaaatatttttgttggaatttaagtttaatggctaaatatgaagactccttgatataaaatattgtgtttaaaatgtcatgcatgttatactgaaatattctggagatgtctgaagcagttctttaagcagcactgtgtattatggaaaaatgtttgtcttttacctagcatatttgtaaatagacctttaataaaatgtgtgtgtgtgtgtgtgtgtgtgtgtgtgtatatatatataccattaaaaggtgggctaatgtggctttccactacagctcttaccagattttttattttcagtgtgatttggctggaatattaataacagttggttgacatgtattcacaattgcatgacttctctttggaagctggtaggttgagtattgttctttttatccatcctttcccctcccctaaatgtcagtctttctgcttttaggattatatttgaattttcaggggtttatacaccaccactggagagtagaaggccactaacttgcaggcagttaacttatatctttacaagctacattctattttaagagcctatttagggtaacatttaaagatttgcttatcactgtattctcatccactgaaatcagtccagctttaccttggagaaaaagcagatgggaaacaggcaaggggaaaagcaaaaaagaggaaaaaaaaagactggggtcacagtttcgatgaatgttattccctgtatttggtgagtggtgggcactagcagctcaatttcactgctagctaatagtggcaaattaagattaaactataaactatttgacattactccattatgtattttgagtctcatgtaattgtttccagtgatttgaaatgaccataaacttaaatttgaaggtcagtgttttgactcttcgaagtaattacacttgacctgctacctaaaacgatgtaatattacaacttttttgaataatctcaggaaaaatggagaaatgtttatattcatagttaataaacattctagatagtaaccaactgtcatttactgaaaataaaattttacccaggtttattttatttttgagacggagtttagcttttgttgcccaggctggagtgcagtggtgtgatcttggctcagcgcaacctccacctcccaggttcaagcaattctcctgcctcagcctccaaagtagttgggattacaggcatattccaccatgcctggctaattttgtatttttagtagagacagggtttcttcatgttggtcaggctggtctcgaactcccgacctcaggtgatccgcctgcctcggcctccccaagtgctgtaatcacaggcgtgagccactgcgcccggcctagccaggttgattttaaaattacacttaaaaataatgttctcatttttaagggctctaatatttgacttttcttaactttccaattatgcagccctatcctgttccagacgttaaggctttctttttgaatgaaaaactttcagacttttttctctctctttttttttttttttggtgtctgcactccccaccgtatctgtcccttcctctttctcccattttgagggatattataagggcaacagttttatagtctcaatattgagggtaagactggttaatagatagagtatccaaattgaatttattaaacacagttctgtgtgcatttccttctgcaaatctgagaataaagtgattacagtttcatcctaaaatactttaaaaatcagttggttttagaaataggtttttttccttttgcatgtaagaatggacagctatttttaggaaggcatgcacctgttatcccagctactcaggaggctgaggcaggagaattgcttgaacccgggaggtggaggttgcagtgagcctagatcatgccattgcactccagcccgggcgacagagtgagactctgtttaaaaagaaaaaaataagtatttgtaaatgtatatatatatatatatacacacacacacacacatatatatataaaatcattatgccatcagtttgagggagagcatgtatgccaacattgtaaagactaagtgcccatttatccataattaagataagcagacttttccccattgtactataaagttagagtttggggctgggtatggtacttccgcctgtaatcccagcagtttgggaggctggagcgggcggatcacttgaggccaggagttcaagaccagcctggccaacgaggggaaaccccgtctctactaaaaatgcaaaaatcaagtcaggcgtggtggtgcagcctgtaatccctcccacggaggctgaggaatgagaatcactttgaacctgggaggcagaggttgcagtgagctgagattgtgccactgcgctccagcttgggcgacagagtgagactgtctcaaaaactaagcaaccagaaacagaaaaagatggaatggaattaatatgttccatttagtaaagatggaatagaattggtccagtttgtgttcattgtagcatattcacctatttggtctgcaaccatttttttttttttaaagcctaagcataccctgagaacaacaaccttttttgattagaacagaagaaaaagccagaatgttcttgggctgccaaaatattaatacgtttgtctcaaagacgaaccacttaggtgcttgcttagagacttctactttacacagaatttgaaaagtttgacagctggctacttactctgcaaaacggaagggagactttttagaacagcaactggtgatctggcaaaaatgaaaagtagaacttgtgaagaaatgagatggacttggtgtattaatagctaattttagaagaccctgtggatattctaaatcagcataatagtgatgtctgagccatagtgttaaattatgattactgtatttgaagtataagaaggcagaaaagtgtgatgctgttagaaaaaaatctcatttcaaatgattgaaggttaaaaacaattggggaaagattagagagggcaggcggtgctttaagggaggtggaataatacttggctccttattctccaaaagtgctggatagctgaagtttttaatattctggataattgatatttgattattagtttaggagggatagcatcttcagtgaacccttgtagctctaaggtaggcatattaaacaactaccatactaaaggtggggagtacgtttggagagcactttgctgggccagctgatgatatgcttaggtgatatttaagaaaatctgcttcttgtctgaaaaacatttggggcttcagaataataccacatacctcattcttggtgttttaatttatttctttgtaattgttttctgtttcttaataattttaaattaagggtattttcccaatataaacattgctttttggttaaataaaatcaaaactagttctgtctccatatctgaatagaccagacagagaattttcttgcctttcaacagcttaaaaaatgtttttgttagaactgttgtgttcaggctgaaatactttcaaagtttgttagttattattgagaaatttctgtaataatcatggaaagggtaatttaatagttaaatctcaacttaattgaagttatttctgttgtggaacttatggtcatcttagcaagaggtcattgctttatagtcagttctctttctcattaaaaatatagtgtactcacctttacagggtgaatgttggtaaataacttctgtctgtgaaggaagtattctggacctgtaagttaaaaataaggtgtttatagcaaattatgtaaataaagattgtatattagaaggtacacactattcaaatttaaagaaaatgtatattgagaaaataactcaaattcttccatgaaattggcaagaagtaaacatttcaaatacacaaaacattatgggtatttttgttcatttgattataaaatgccaaagttgttttataaatgctacttcaagcctggaaactttgaggaagtcctgaacattaagcatataaatggcccagctctagaatacatgtaagttgaaaagctaacctgaagtgggaagcgcagtatatacctaagacttactctgcactgaaagtttgctttgtcactagaagtaaaacaagactgtggtaggatagtaagatcagtaacacctcagttaatcaggtatcttggaggaagtgaagaaagaccctaattcaagggacagttaattggccttttattccaagaatgggccttagtggcagtatcttaaaagcccacaagatggagatgtttcctaatgaaaggcctttaatttctttatagagctgagttagtgtcacatcccagtccccacccatgacccttccccagttaaaaagaagagaaaatgttgagcaagtctgatttgattcccatggtgacatttttagccattatgtaacaaattctgacagtttacccttaaaattaaaaacctccagtcctgtctttttaaagggtagaaagaaggttaggtataggatagctttttatttatttatttatttatttttgtacaaagggagcctatgtaaagctgccagatctgaactttctggtgttttgctgtaatgttagtaagatttcgccttaaaatattttattttgagtatatacgtttggtcttagagtgtcttggtggatttccgcttaccacccatcactttctgcattttaaaggcttaaatactttattgctggttaactgaggttctactgtaaacgaatcatctaagttaattagtggattgtacttcaatggataattttcactaaattgtttatattgcacattacttttgtcttaaggactcttagcacatcaaaaaaattggctcacaacttaattgtgagatgtagaattttccattttatgtgctaagagttttgttaatgagagaactgttaaaatagaaaaggagcttcagcatagacaccaatgctggttgctgagatcagtggggaactgcatagcattttaacaagtttaatgaacatttggaagagaaatttgaagctaagattctggtttttttgctgttaactttttaattttttaatctaaggaaaacttttatgtacagtatttttactttgggtatatgtttatcttttagcaagttgaaagacttaatttgctgcttgctcactattttgtaattatttgggagcagcagtaataagccagctttttggaataggatgttcctgatgtgtggttatgtaggaagaatgatgttttaatatactgcccagtaaactggtgcagtttggaaaaggtgtgttattgatgtggataatatttaaggcaattttttttaagcattttaatactgctttttgtctttacaggaaaatgtttataggaggccttagctgggacactacaaagaaagatcctaaggaaaacttttatgtacagtatttttactttgggtatatgtttatcttttagcaagttgaaagacttaatttgctgcttgctcactattttgtaattatttgggagcagcagtaataagccagctttttggaataggatgttcctgatgtgtggttatgtaggaagaatgatgttttaatatactgcccagtaaactggtgcagtttggaaaaggtgtgttattgatgtggataatatttaaggcaattttttttaagcattttaatactgctttttgtctttacaggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtagtgtgttacgtgttctgatcagttaataatataaaatattaacatatggatagtttgatacaatgagtttgcctatttgtggttccccattttgatagtataggaaggaagaatagttcttgccccaatacgttttatgaagatagaggtaggttcaggaattatttcctgaataatttgtgttccaggctctgctaaattttgaaattaactttaaagatactatagacttaaagatgcctagttaaaaggatgtgttttagcaattcacagaagtccatattttgaaattttgttaggcaagcaacttttaactgaatcattattttgatcctgggctaaagggaagtagcagttatgtttgtatatagtgctaaagggaagtagcagtcatgtttgtatatattgaaggtaatggttatctagtaattggttaaatttgtgtatgtcctaccattcttacctttagatttaaacagtatatgttagttgatgttacatcaccaccatgacttgacagtttaatcttgagcaagtcaacatatgcttggcattatctgtatttatagttatttttaagtaagttaatagtctctgaacttcagttaagatatatattttttaaatgaaaacttcaatttccttaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtatgttataaatgttttgacccagtttatgtcaaaattagtgtgaatgtgattgtcccattatggactcagagtcacttggcttttcaaagctgttagggtagattatgtgatctgttttggaataaggatattgtaaatacttcattagcaggtctttgaaggttggataatgtgtttttctcattgagcacctactctatgcagagtattgctggggtagagtataccaaagatgaaatagacaaacattcttaaatacagacaattaagaggaagaaatctagattagaaggagacttttgttgaaaataggaaaggaattaagaatagggtgtagtgccttatcagttgaaatgcatgtgtaagtgcaaatttaatgagactaatcattatagactcattagtgaggctggacgcgatggctcatgctgtagtaatcccagcacttgggaggccgaagtgggtggattactggagaccaggagatgaagaccatcctggacaacatagtggacccgtctcaattaaaagtaaaacaaacttagactcattagtgaaataggtagataatagaggtttcttttatgaattaatgaattaatgaaagttgagaaatttgtggccttgggctccagatacgtcccacagacatttattgcttgattgaaacaataagtgcttttttagtgttgaggatttgagatattgcccacaaaactcagtatctagctttttaaaaaatatttgcaagagcacgcaacatgggaactgacgctgccctcctgtacggcagcatctctcaaactgagtagtagcttccatcttggtttgggcatatgctctccagtttttagtagtcctcaccaccctacttcctgttttctctcaaatacattttttttctgtttttcttagatttgactgttttcttcttgttctttgtgggcatttgaatttgtgacccttgagttaggtagtaaatgtcagtgcgtggtaaagcttatttttgtaaatagttgtgaagaccttagatggaatgggtgttctaatttgaagaattccttaaaaggattagaataaatagggagaaacaggagactagaacttcagtgccaaatacatgttttctttgtgtgttttcccccctctaaacttgtgtttcttttaaggtcaataaaatgcatgttagcatattaaaatttgttttttaataccaggtggaatccatagagctccccatggacaacaagaccaataagaggcgtgggttctgctttattacctttaaggaagaagaaccagtgaagaagataatggaaaagaaataccacaatgttggtcttagtaaagtaagttaagcatccatttacttgtagagaaaactagctgttgtaaagagcttaaccatttatctttctctgtaaaggcttaagttctttgcatgctttaaaaacttctcattggttacttaccattgaccaactttttgtggggagcaggatggacacatttgttagtgtttttgtctaggctttcacaaaaatagtttttagaacttgacaagtaaaatgaagtaacatcactagcaagtactcataagtgattactcttaagtactaaatattggtttaattaataaactgactgaggaaagtttcaaattagcctactctatttaaacatgttggctattctggttattagaaacttatttagcaacttttattttcttgagtcagtttaataatgtaatttttctcttttagtgtgaaataaaagtagccatgtcgaaggaacaatatcagcaacagcaacagtggggatctagaggaggatttgcaggaagagctcgtggaagaggtggtggtaagctagagcctaagtttactctatcttaagcttttctgctttttaattatcctgaagtaaagatctttgctgatcttctgactttagtgaacctattaatgtgctgcaggccccagtcaaaactggaaccagggatatagtaactattggaatcaaggctatggcaactatggatataacagccaaggttacggtggttatggaggatatgactacactggttacaacaactactatggatatggtgattatagcagtaagtactatactttttatattaactgctatttgacatttattttgtacaaatttggataggcagaaaggttagtgtagccttgccaagtgcaaatgtcttcaggtttcaaattcctggaaacttgaaactgcagccattttattgcttggttcctcccagcctatatcacacacacattataagggtagggtgtatgtgtggttctatatatgtttgctgggcatttgttttacttggatttaaaaattttaagctcagttcagatcttttaagctcaaggtattctaaatgtcacttcttggaccaaccaataatcttggcatgatgtgtcttaatcctataaaactgaataataccacatgttgccagttaaaactaatagtatcctcgctttaggattattaatgtagaaactcttaaaacagatgttgagcttgatagaaccaaaaaacttgacttttagacatggaaagccctgacttcattgtgcaactaaggtagttgctctccacctgatttgtagcaactgttgagtcgtcaggtaaagggttctactagaagcaatcttacatttttttggaggagagtggttgcattggttgcattgttttaagtggtttttcttttccttccttggttagaccagttcttggagttatatcctttcttaggtgactaggcctgctgcacaataataggttaattaaagtcagaagaaggtcagcaaagatggattgggtgagattggggcccttttcttagaagggcagagatactaagcactgattgtggttgacaatttgttctaaattttaagatattttttgctggtggttgtgaaagggtcagctgtccatcctttgaaacttaaaacttttaaactgtaagggtgaggggattgtctcccattttatacaataagtcaagtaatcagctcattctgaatgcctgccattgtatgcattcactacatatttggtaaattatttgataaatgattgctcagggtgaatttttcacacttgggaattaagctacccttaattttttgagattgtttaaaattaggtactgttctgattattagtatgtaaccactaccgttctggttctaacacttgttttattttagaccagcagagtggttatgggaaggtatccaggcgaggtggtcatcaaaatagctacaaaccatactaaattattccatttgcaacttatccccaacaggtatgttctaaaaatagtttttttttgtcatttacaatagtagtttttataatctatattgttcataaaacaatgcttaatttaagagtttcacagcacccagaagtgcttaccatattataacatagtgactttcaaaagatatgtaacacaggtgctcttaagcttttgcctttttgtcctattattaacaagtcagtaaagttaacaggtaaagtactgctaatgggtacaaattaaggaattgcagcaaaaaagtattgcctactaactctgacattataccttgtttgtaccgccagcgggaacttcattgcaggccctgtgtcgcgctgacttcagattctcacaggcccgctcaatgcggacagggtaacgagatgctccacgctctcgaatgctgccgtttggtatggtctcttccaacatcctgtatcagcattataaaataaaatggatacttcaagctttgccttcacttatttctttgctttttaaaaactatttgtaatgtaattttaatgcattttttacaggcccagtaatggttaaatacgtcagcttactgaataattttaactatttattcttctaaggatacagcttgtctctggattttccagtcttaattttatattttattaatctattttaatgcttgcttttcccatttatagacgttgtagcagtaattgcaagaagttcttgagctgaattcctgttgtgacaacttcctataattacagtagataactttttcttttagtcgtatataacttttctataacttgtgatggacaagagatatgcttatccaataaaataagcttaaatattagatgctcttgggtcaaaatgtccttttaccaaattgacctttttatgagttctttgggtaaatactttaaagctttttatattttaaagaatacttgtaaaagcatatcacatcttaaaccagtggtgcacatgtggatttacagctcatggactctactgttcagctttaatttataaaacatatcacacatttaatgttatacagtatttacatatagtggaacatagggataactcagttttatgtaaatttttgttaagtgttgtagcctgcccagagtgacttctattttttcttctttgtctccaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttccccaatg

SEQ ID NO: 42 - protein, 279 aa, Homo sapiens
  ref <308>: DATABASE ACCESSION NUMBER: NP_852000
  ref <309>: DATABASE ENTRY DATE: 2004-10-27
  ref <313>: RELEVANT RESIDUES: (1)..(279)
MPCRREEEEEAGEEAEGEEEEEDSFLLLQQSVALGSSGEVDRLVAQIGETLQLDAAQHSPASPCGPPGAPLRAPGPLAAAVPADKARSPAVPLLLPPALAETVGPAPPGVLRCALGDRGRVRGRAAPYCVAELATGPSALSPLPPQADLDGPPGAGKQGIPQPLSGPCRRGWLRGAAASRRLQQRRGSQPETRTGDDDPHRLLQQLVLSGNLIKEAVRRLHSRRLQLRAKLPQRPLLGPLSAPVHEPPSPRSPRAACSDPGASGRAQLRTGDGVLVPGS

SEQ ID NO: 43 - DNA, 2653 bp, Homo sapiens
  ref <308>: DATABASE ACCESSION NUMBER: NM_005479
  ref <309>: DATABASE ENTRY DATE: 2004-10-26
  ref <313>: RELEVANT RESIDUES: (1)..(2653)
ggattccggctcccgcggctgcaggcgcgcggctagagtgcctggcgggctccggcttccgcgtccgccccggccccggtccagacttagtcttcagctccgcgcccgctccgccgcggcccaccgcgcccgccggcagccgagcccccagcgacgcccgcacagctccgggtgcccagacagggggccatgccgtgccggagggaggaggaagaggaagccggcgaggaggcggagggggaggaagaggaggaggacagcttcctcctactgcagcagtcagtggcgctgggcagctcgggcgaggtggaccggctggtggcccagatcggcgagacgctgcagctggacgcggcgcagcacagcccggcctcgccgtgcgggcccccgggggcgccgctgcgggccccggggcccctggctgcggcggtgccggcggacaaggccaggtccccggcggtgccgctgctgctgccgcccgcgttggcggagactgtgggcccggcgccccctggggtcctgcgctgcgccctgggggaccgcggccgcgtgcggggccgcgctgcgccctactgcgtggccgagctcgccacaggccccagcgcgctgtccccactgccccctcaggccgaccttgatgggcctccgggagctggcaagcagggcatcccgcagccgctgtcgggtccgtgccggcgaggatggctccggggcgccgccgcctcccgccgcctgcagcagcgacgcgggtcccaaccagaaacccgcacaggcgacgacgacccgcaccggcttctgcagcagctagtgctctctggaaacctcatcaaggaggccgtgcgaaggcttcattcgcgacggctgcagttacgtgcaaagcttccccaacgcccgctcctgggacctctgtcggccccggtgcatgaacccccttcgcctcgcagccctcgcgcggcctgcagtgaccctggcgcctccgggagggcgcagctcagaactggcgacggcgttcttgtgcctggcagctaacacgcccggggtggccacagcgccagcctcagactggagggcaaggggttcccttgagggctgcagttctactcaggctggtggagaactctggcttttggaagcgagagtaaaaagctaatgacgaggaaccgaaaaatcgcgagtgtttcgcgggtaactggggttgagggccaaaatatttggaatgaaggacttggccctatttaaggcagattttacagagcgcacctcaaacgtacaagtcagtaggactccttatttggcgtgacccgacctggccgcggagcctgcatttcctcgcagcctctcagtgccctccagccccgcgaccatgtggccacaatccacgcttctccggatcgcggtgcgccggaaccacggaggatgatgccagttacttgctttaccttttcagggctggctcctgatccactttgggggaggagaacatgagtagataatttcagggtgcagcccaatctgccagacttaaaaaaaccatcttgtgtctttggaggtgctgcttaataccaaacatgcggtgccatgaagggaccctttgggggttgaataggagttaacccctgcgctctctttgcaactgtctctcttctcagagtggtgggggaaggctgtacgacacgggtggggaaaggaggtgggggcggggagtattgaatggtggtggaagggtagagaggcgcggagtgaaccccacgccctgtctaaagtgtattttcagagccggcccgcctctcctcggttcaaggtcactgtttcctgggcacgcactgggttgcgggacagagtagccaggttctgccggtgctcggagaagagcgcagtgttttgcaagtgctggagtctcctgaggacacgcgcgtcgccgccaccgcgggtgtgggaaagcgcggacgtgctgggcggctgtgcttcggtaggcgaccaccgcccctggccgcgctccgggctttcacggaaactcccgagaccgggccctgggttcctcctctcctactcggctctgcagtcctactcaagcgggtggctctgggatcctgggggcctgggttgggggctagggagacgccatgtgatggacactccagggacacacagcctagcacagcagcttataatgggctctccggggccatttgcaataacagctgcaattccctggatagacgagttgatttcctccctctgcccctcccccagccatgccagctggcctttgtaagtgcaggaaaccgagtagaaaatgtgaccctccaaatggagaagctgcaggctttgccattgtgaaccatggtgaagtgcttggaacatactgttcactcactctaaaggcgctgagactgtgctgttgttctcgtttttatagtcaatggcttgttcatcatccagatgtggctactgacatatctacacttcgcaccggagtgtctggaattgtggctatcctgattataggattttaacttaactgaaatgcctgctttgaataaatgtgttgggttttttgtttggttttattttatacttgccatcagtgaaaaagatgtacagaacacatttctctgatctccataaacatgaaaacacttgaaatctcaaa

SEQ ID NO: 44 - DNA, 2203 bp, Homo sapiens
  ref <308>: DATABASE ACCESSION NUMBER: NM_181355
  ref <309>: DATABASE ENTRY DATE: 2004-10-27
  ref <313>: RELEVANT RESIDUES: (1)..(2203)
ggattccggctcccgcggctgcaggcgcgcggctagagtgcctggcgggctccggcttccgcgtccgccccggccccggtccagacttagtcttcagctccgcgcccgctccgccgcggcccaccgcgcccgccggcagccgagcccccagcgacgcccgcacagctccgggtgcccagacagggggccatgccgtgccggagggaggaggaagaggaagccggcgaggaggcggagggggaggaagaggaggaggacagcttcctcctactgcagcagtcagtggcgctgggcagctcgggcgaggtggaccggctggtggcccagatcggcgagacgctgcagctggacgcggcgcagcacagcccggcctcgccgtgcgggcccccgggggcgccgctgcgggccccggggcccctggctgcggcggtgccggcggacaaggccaggtccccggcggtgccgctgctgctgccgcccgcgttggcggagactgtgggcccggcgccccctggggtcctgcgctgcgccctgggggaccgcggccgcgtgcggggccgcgctgcgccctactgcgtggccgagctcgccacaggccccagcgcgctgtccccactgccccctcaggccgaccttgatgggcctccgggagctggcaagcagggcatcccgcagccgctgtcgggtccgtgccggcgaggatggctccggggcgccgccgcctcccgccgcctgcagcagcgacgcgggtcccaaccagaaacccgcacaggcgacgacgacccgcaccggcttctgcagcagctagtgctctctggaaacctcatcaaggaggccgtgcgaaggcttcattcgcgacggctgcagttacgtgcaaagcttccccaacgcccgctcctgggacctctgtcggccccggtgcatgaacccccttcgcctcgcagccctcgcgcggcctgcagtgaccctggcgcctccgggagggcgcagctcagaactggcgacggcgttcttgtgcctggcagctaacacgcccggggtggccacagcgccagcctcagactggagggcaaggggttcccttgagggctgcagttctactcaggctggtggagaactctggcttttggaagcgagagtaaaaagctaatgacgaggaaccgaaaaatcgcgagtgtttcgcgggtaactggggttgagggccaaaatatttggaatgaaggacttggccctatttaaggcagattttacagagcgcacctcaaacgtacaagtcagtaggactccttatttggcgtgacccgacctggccgcggagcctgcatttcctcgcagcctctcagtgccctccagccccgcgaccatgtggccacaatccacgcttctccggatcgcggtgcgccggaaccacggaggatgatgccagttacttgctttaccttttcagggctggctcctgatccactttgggggaggagaacatgagtagataatttcagggtgcagcccaatctgccagacttaaaaaaaccatcttgtgtctttggaggtgctgcttaataccaaacatgcggtgccatgaagggaccctttgggggttgaataggagttaacccctgcgctctctttgcaactgtctctcttctcagagtggtgggggaaggctgtacgacacgggtggggaaaggaggtgggggcggggagtattgaatggtggtggaagggtagagaggcgcggagtgaaccccacgccctgtctaaagtgtattttcagagccggcccgcctctcctcggttcaaggtcactgtttcctgggcacgcactgggttgcgggacagagtagccaggttctgccggtgctcggagaagagcgcagtgttttgcaagtgctggagtctcctgaggacacgcgcgtcgccgccaccgcgggtgtgggaaagcgcggacgtgctgggcggctgtgcttcgtcaatggcttgttcatcatccagatgtggctactgacatatctacacttcgcaccggagtgtctggaattgtggctatcctgattataggattttaacttaactgaaatgcctgctttgaataaatgtgttgggttttttgtttggttttattttatacttgccatcagtgaaaaagatgtacagaacacatttctctgatctccataaacatgaaaacacttgaaatctcaaa

SEQ ID NO: 45 - protein, 1845 aa, Homo sapiens
  ref <308>: DATABASE ACCESSION NUMBER: NP_444254
  ref <309>: DATABASE ENTRY DATE: 2004-10-28
  ref <313>: RELEVANT RESIDUES: (1)..(1845)
MGDVKLVASSHISKTSLSVDPSRVDSMPLTEAPAFILPPRNLCIKEGATAKFEGRVRGYPEPQVTWHRNGQPITSGGRFLLDCGIRGTFSLVIHAVHEEDRGKYTCEATNGSGARQVTVELTVEGSFAKQLGQPVVSKTLGDRFSASAVETRPSIWGECPPKFATKLGRVVVKEGQMGRFSCKITGRPQPQVTWLKGNVPLQPSARVSVSEKNGMQVLEIHGVNQDDVGVYTCLVVNGSGKASMSAELSIQGLDSANRSFVRETKATNSDVRKEVTNVISKESKLDSLEAAAKSKNCSSPQRGGSPPWAANSQPQPPRESKLESCKDSPRTAPQTPVLQKTSSSITLQAARVQPEPRAPGLGVLSPSGEERKRPAPPRPATFPTRQPGLGSQDVVSKAANRRIPMEGQRDSAFPKFESKPQSQEVKENQTVKFRCEGLAVMEVAPSFSSVLKDCAVIEGQDFVLQCSVRGTPVPRITWLLNGQPIQYARSTCEAGVAELHIQDALPEDHGTYTCLAENALGQVSCSAWVTVHEKKSSRKSEYLLPVAPSKPTAPIFLQGLSDLKVMDGSQVTMTVQVSGNPPPEVIWLHNGNEIQESEDFHFEQRGTQHSLCIQEVFPEDTGTYTCEAWNSAGEVRTQAVLTVQEPHDGTQPWFISKPRSVTASLGQSVLISCAIAGDPFPTVHWLRDGKALCKDTGHFEVLQNEDVFTLVLKKVQPWHAGQYEILLKNRVGECSCQVSLMLQNSSARALPRGREPASCEDLCGGGVGADGGGSDRYGSLRPGWPARGQGWLEEEDGEDVRGVLKRRVETRQHTEEAIRQQEVEQLDFRDLLGKKVSTKTLSEDDLKEIPAEQMDFRANLQRQVKPKTVSEEERKVHSPQQVDFRSVLAKKGTSKTPVPEKVPPPKPATPDFRSVLGGKKKLPAENGSSSAETLNAKAVESSKPLSNAQPSGPLKPVGNAKPAETLKPMGNAKPAETLKPMGNAKPDENLKSASKEELKKDVKNDVNCKRGHAGTTDNEKRSESQGTAPAFKQKLQDVHVAEGKKLLLQCQVSSDPPATIIWTLNGKTLKTTKFIILSQEGSLCSVSIEKALPEDRGLYKCVAKNDAGQAECSCQVTVDDAPASENTKAPEMKSRRPKSSLPPVLGTESDATVKKKPAPKTPPKAAMPPQIIQFPEDQKVRAGESVELFGKVTGTQPITCTWMKFRKQIQESEHMKVENSENGSKLTILAARQEHCGCYTLLVENKLGSRQAQVNLTVVDKPDPPAGTPCASDIRSSSLTLSWYGSSYDGGSAVQSYSIEIWDSANKTWKELATCRSTSFNVQDLLPDHEYKFRVRAINVYGTSEPSQESELTTVGEKPEEPKDEVEVSDDDEKEPEVDYRTVTINTEQKVSDFYDIEERLGSGKFGQVFRLVEKKTRKVWAGKFFKAYSAKEKENIRQEISIMNCLHHPKLVQCVDAFEEKANIVMVLEIVSGGELFERIIDEDFELTERECIKYMRQISEGVEYIHKQGIVHLDLKPENIMCVNKTGTRIKLIDFGLARRLENAGSLKVLFGTPEFVAPEVINYEPIGYATDMWSIGVICYILVSGLSPFMGDNDNETLANVTSATWDFDDEAFDEISDDAKDFISNLLKKDMKNRLDCTQCLQHPWLMKDTKNMEAKKLSKDRMKKYMARRKWQKTGNAVRAIGRLSSMAMISGLSGRKSSTGSPTSPLNAEKLESEEDVSQAFLEAVAEEKPHVKPYFSKTIRDLEVVEGSAARFDCKIEGYPDPEVVWFKDDQSIRESRHFQIDYDEDGNCSLIISDVCGDDDAKYTCKAVNSLGEATCTAELIVETMEEGEGEGEEEEE

SEQ ID NO: 46 - DNA, 5719 bp, Homo sapiens
  ref <308>: DATABASE ACCESSION NUMBER: AF069601
  ref <309>: DATABASE ENTRY DATE: 2000-03-14
  ref <313>: RELEVANT RESIDUES: (1)..(5719)
ccggctgcctctgctgcagttcagagcaacttcaggagcttcccagccgagagcttcaggacgcctttcctgtcccactggcccagttgccacaacaaacaacagagaagacggtgaccatgggggatgtgaagctggttgcctcgtcacacatttccaaaacctccctcagtgtggatccctcaagagttgactccatgcccctgacagaggcccctgctttcattttgccccctcggaacctctgcatcaaagaaggagccaccgccaagttcgaagggcgggtccggggttacccagagccccaggtgacatggcacagaaacgggcaacccatcaccagcgggggccgcttcctgctggattgcggcatccgggggactttcagccttgtgattcatgctgtccatgaggaggacaggggaaagtatacctgtgaagccaccaatggcagtggtgctcgccaggtgacagtggagttgacagtagaaggaagttttgcgaagcagcttggtcagcctgttgtttccaaaaccttaggggatagattttcagcttcagcagtggagacccgtcctagcatctggggggagtgcccaccaaagtttgctaccaagctgggccgagttgtggtcaaagaaggacagatgggacgattctcctgcaagatcactggccggccccaaccgcaggtcacctggctcaagggaaatgttccactgcagccgagtgcccgtgtgtctgtgtctgagaagaacggcatgcaggttctggaaatccatggagtcaaccaagatgacgtgggagtgtacacgtgcctggtggtgaacgggtcggggaaggcctcgatgtcagctgaactttccatccaaggtttggacagtgccaataggtcatttgtgagagaaacaaaagccaccaattcagatgtcaggaaagaggtgaccaatgtaatctcaaaggagtcgaagctggacagtctggaggctgcagccaaaagcaagaactgctccagcccccagagaggtggctccccaccctgggctgcaaacagccagcctcagcccccaagggagtccaagctggagtcatgcaaggactcgcccagaacggccccgcagaccccggtccttcagaagacttccagctccatcaccctgcaggccgcaagagttcagccggaaccaagagcaccaggcctgggggtcctatcaccttctggagaagagaggaagaggccagctcctccccgtccagccaccttccccaccaggcagcctggcctggggagccaagatgttgtgagcaaggctgctaacaggagaatccccatggagggccagagggattcagcattccccaaatttgagagcaagccccaaagccaggaggtcaaggaaaatcaaactgtcaagttcagatgtgaagggcttgccgtgatggaggtggccccctccttctccagtgtcctgaaggactgcgctgttattgagggccaggattttgtgctgcagtgctccgtacgggggaccccagtgccccggatcacttggctgctgaatgggcagcccatccagtacgctcgctccacctgcgaggccggcgtggctgagctccacatccaggatgccctgccggaggaccatggcacctacacctgcctagctgagaatgccttggggcaggtgtcctgcagcgcctgggtcaccgtccatgaaaagaagagtagcaggaagagtgagtaccttctgcctgtggctcccagcaagcccactgcacccatcttcctgcagggcctctctgatctcaaagtcatggatggaagccaggtcactatgactgtccaagtgtcagggaatccaccccctgaagtcatctggctgcacaatgggaatgagatccaagagtcagaggacttccactttgaacagagaggaactcagcacagcctttggatccaggaagtgttcccggaggacacgggcacgtacacctgcgaggcctggaacagcgctggagaggtccgcacccaggccgtgctcacggtacaagagcctcacgatggcacccagccctggttcatcagtaagcctcgctcagtgacagcctccctgggccagagtgtcctcatctcctgcgccatagctggtgacccctttcctaccgtgcactggctcagagatggcaaagccctctgcaaagacactggccacttcgaggtgcttcagaatgaggacgtgttcaccctggttctaaagaaggtgcagccctggcatgccggccagtatgagatcctgctcaagaaccgggttggcgaatgcagttgccaggtgtcactgatgctacagaacagctctgccagagcccttccacgggggagggagcctgccagctgcgaggacctctgtggtggaggagttggtgctgatggtggtggtagtgaccgctatgggtccctgaggcctggctggccagcaagagggcagggttggctagaggaggaagacggcgaggacgtgcgaggggtgctgaagaggcgcgtggagacgaggcagcacactgaggaggcgatccgccagcaggaggtggagcagctggacttccgagacctcctggggaagaaggtgagtacaaagaccctatcggaagacgacctgaaggagatcccggccgagcagatggatttccgtgccaacctgcagcggcaagtgaagccaaagactgtgtctgaggaagagaggaaggtgcacagcccccagcaggtcgattttcgctctgtcctggccaagaaggggacttccaagacccccgtgcctgagaaggtgccaccgccaaaacctgccaccccggattttcgctcagtgctgggtggcaagaagaaattaccagcagagaatggcagcagcagtgccgagaccctgaatgccaaggcagtggagagttccaagcccctgagcaatgcacagccttcagggcccttgaaacccgtgggcaacgccaagcctgctgagaccctgaagccaatgggcaacgccaagcctgccgagaccctgaagcccatgggcaatgccaagcctgatgagaacctgaaatccgctagcaaagaagaactcaagaaagacgttaagaatgatgtgaactgcaagagaggccatgcagggaccacagataatgaaaagagatcagagagccaggggacagccccagccttcaagcagaagctgcaagatgttcatgtggcagagggcaagaagctgctgctccagtgccaggtgtcttctgaccccccagccaccatcatctggacgctgaatggaaagaccctcaagaccaccaagttcatcatcctctcccaggaaggctcactctgctccgtctccatcgagaaggcactgcctgaggacagaggcttatacaagtgtgtagccaagaatgacgctggccaggcggagtgctcctgccaagtcaccgtggatgatgctccagccagtgagaacaccaaggccccagagatgaaatcccggaggcccaagagctctcttcctcccgtgctaggaactgagagtgatgcgactgtgaaaaagaaacctgcccccaagacacctccgaaggcagcaatgccccctcagatcatccagttccctgaggaccagaaggtacgcgcaggagagtcagtggagctgtttggcaaagtgacaggcactcagcccatcacctgtacctggatgaagttccgaaagcagatccaggaaagcgagcacatgaaggtggagaacagcgagaatggcagcaagctcaccatcctggccgcgcgccaggagcactgcggctgctacacactgctggtggagaacaagctgggcagcaggcaggcccaggtcaacctcactgtcgtggataagccagaccccccagctggcacaccttgtgcctctgacattcggagctcctcactgaccctgtcctggtatggctcctcatatgatgggggcagtgctgtacagtcctacagcatcgagatctgggactcagccaacaagacgtggaaggaactagccacatgccgcagcacctctttcaacgtccaggacctgctgcctgaccacgaatataagttccgtgtacgtgcaatcaacgtgtatggaaccagtgagccaagccaggagtctgaactcacaacggtaggagagaaacctgaagagccgaaggatgaagtggaggtgtcagatgatgatgagaaggagcccgaggttgattaccggacagtgacaatcaatactgaacaaaaagtatctgacttctacgacattgaggagagattaggatctgggaaatttggacaggtctttcgacttgtagaaaagaaaactcgaaaagtctgggcagggaagttcttcaaggcatattcagcaaaagagaaagagaatatccggcaggagattagcatcatgaactgcctccaccaccctaagctggtccagtgtgtggatgcctttgaagaaaaggccaacatcgtcatggtcctggagatcgtgtcaggaggggagctgtttgagcgcatcattgacgaggactttgagctgacggagcgtgagtgcatcaagtacatgcggcagatctcggagggagtggagtacatccacaagcagggcatcgtgcacctggacctcaagccggagaacatcatgtgtgtcaacaagacgggcaccaggatcaagctcatcgactttggtctggccaggaggctggagaatgcggggtctctgaaggtcctctttggcaccccagaatttgtggctcctgaagtgatcaactatgagcccatcggctacgccacagacatgtggagcatcggggtcatctgctacatcctagtcagtggcctttcccccttcatgggagacaacgataacgaaaccttggccaacgttacctcagccacctgggacttcgacgacgaggcattcgatgagatctccgacgatgccaaggatttcatcagcaatctgctgaagaaagatatgaaaaaccgcctggactgcacgcagtgccttcagcatccatggctaatgaaagataccaagaacatggaggccaagaaactctccaaggaccggatgaagaagtacatggcaagaaggaaatggcagaaaacgggcaatgctgtgagagccattggaagactgtcctctatggcaatgatctcagggctcagtggcaggaaatcctcaacagggtcaccaaccagcccgctcaatgcagaaaaactagaatctgaagaagatgtgtcccaagctttccttgaggctgttgctgaggaaaagcctcatgtaaaaccctatttctctaagaccattcgcgatttagaagttgtggagggaagtgctgctagatttgactgcaagattgaaggatacccagaccccgaggttgtctggttcaaagatgaccagtcaatcagggagtcccgccacttccagatagactacgatgaggacgggaactgctctttaattattagtgatgtttgcggggatgacgatgccaagtacacctgcaaggctgtcaacagtcttggagaagccacctgcacagcagagctcattgtggaaacgatggaggaaggtgaaggggaaggggaagaggaagaagagtgaaacaaagccagagaaaagcagtttctaagtcatattaaaaggactatttctctcaaaatcca

SEQ ID NO: 47 - protein, 284 aa, Homo sapiens
  ref <308>: DATABASE ACCESSION NUMBER: AAH07433
  ref <309>: DATABASE ENTRY DATE: 2004-06-29
  ref <313>: RELEVANT RESIDUES: (1)..(284)
MDAIKKKMQMLKLDKENALDRAEQAEADKKAAEDRSKQLEDELVSLQKKLKGTEDELDKYSEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGQVRQLEEQLRIMDQTLKALMAAEDKYSQKEDRYEEEIKVLSDKLKEAETRAEFAERSVTKLEKSIDDLEDELYAQKLKYKAISEELDHALNDMTSM

SEQ ID NO: 48 - DNA, 1256 bp, Homo sapiens
  ref <308>: DATABASE ACCESSION NUMBER: NM_000366
  ref <309>: DATABASE ENTRY DATE: 2004-12-18
  ref <313>: RELEVANT RESIDUES: (1)..(1256)
gaggaatgcggtcgcccccttgggaaagtacatatctgggagaagcaggcggctccgcgctcgcactcccgctcctccgcccgaccgcgcgctcgccccgccgctcctgctgcagccccagggcccctcgccgccgccaccatggacgccatcaagaagaagatgcagatgctgaagctcgacaaggagaacgccttggatcgagctgagcaggcggaggccgacaagaaggcggcggaagacaggagcaagcagctggaagatgagctggtgtcactgcaaaagaaactcaagggcaccgaagatgaactggacaaatattctgaggctctcaaagatgcccaggagaagctggagctggcagagaaaaaggccaccgatgctgaagccgacgtagcttctctgaacagacgcatccagctggttgaggaagagttggatcgtgcccaggagcgtctggcaacagctttgcagaagctggaggaagctgagaaggcagcagatgagagtgagagaggcatgaaagtcattgagagtcgagcccaaaaagatgaagaaaaaatggaaattcaggagatccaactgaaagaggcaaagcacattgctgaagatgccgaccgcaaatatgaagaggtggcccgtaagctggtcatcattgagagcgacctggaacgtgcagaggagcgggctgagctctcagaaggccaagtccgacagctggaagaacaattaagaataatggatcagaccttgaaagcattaatggctgcagaggataagtactcgcagaaggaagacagatatgaggaagagatcaaggtcctttccgacaagctgaaggaggctgagactcgggctgagtttgcggagaggtcagtaactaaattggagaaaagcattgatgacttagaagacgagctgtacgctcagaaactgaagtacaaagccatcagcgaggagctggaccacgctctcaacgatatgacttccatgtaaacgttcatccactctgcctgcttacaccctgccctcatgctaatataagtttctttgcttcacttctcccaagactccctcgtcgagctggatgtcccacctctctgagctctgcatttgtctattctccagctgaccctggttctctctcttagcatcctgccttagagccaggcacacactgtgctttctattgtacagaagctcttcgtttcagtgtcaaataaacactgtgtaagctaaaaaaaaaaaaaaaaaaa

SEQ ID NO: 49 - protein, 11 aa, Homo sapiens
EITALAPSTMK

SEQ ID NO: 50 - protein, 7 aa, Homo sapiens
MLTELEK

SEQ ID NO: 51 - protein, 11 aa, Homo sapiens
ALNSIIDVYHK

SEQ ID NO: 52 - protein, 7 aa, Homo sapiens
GADVWFK

(19) United States
(12) Patent Application Publication
(10) Pub. No.: US 2008/0226554 A1
(43) Pub. Date: Sep. 18, 2008

(54) Methods For Detecting Markers Associated With Endometrial Disease or Phase

(75) Inventors: Terence J. Colgan, Toronto (CA); Michael K.W. Siu, Toronto (CA); Alexander D. Romaschin, Toronto (CA); Eric C.C. Yang, Toronto (CA)

Correspondence Address: HOWSON AND HOWSON, SUITE 210, 501 OFFICE CENTER DRIVE, FT WASHINGTON, PA, 19034, US

(73) Assignees: MOUNT SINAI HOSPITAL, TORONTO (CA); YORK UNIVERSITY, TORONTO (CA); UNIVERSITY HEALTH NETWORK, TORONTO (CA)

(21) Appl. No.: 10/584,207

(22) PCT Filed: Dec. 21, 2004
(86) PCT No.: PCT/CA04/02172

§ 371 Date: Nov. 28, 2007

Related U.S. Application Data
(60) Provisional application No. 60/532,601, filed on Dec. 23, 2003.
(60) Provisional application No. 60/630,990, filed on Nov. 24, 2004.

(51) Int. Cl. A61K 49/00; C12Q 1/00; C12Q 1/68; C07K 2/00; G01N 33/567

(52) U.S. Cl. 424/9.1; 435/4; 435/6; 435/7.21; 530/300

(57) ABSTRACT

Methods for detecting endometrial diseases or an endometrium phase in a subject are described comprising measuring endometrial markers or polynucleotides encoding the markers in a sample from the subject. The invention also provides localization or imaging methods for endometrial diseases, and kits for carrying out the methods of the invention. The invention also contemplates therapeutic applications for endometrial diseases employing endometrial markers, polynucleotides encoding the markers, and/or binding agents for the markers.

### FIELD OF THE INVENTION

The invention relates to methods for identifying markers associated with endometrium, endometrial markers, methods for assessing the status of an endometrial tissue, and methods for the detection, diagnosis, prediction, and therapy of an endometrial disease.

### BACKGROUND OF THE INVENTION

The endometrium, the tissue lining the uterus, is a glandular layer of variable thickness that is very sensitive to the hormones estrogen and progesterone. During the menstrual cycle the endometrium undergoes cyclic variation with a proliferation phase where the endometrium grows under the influence of estrogen, an ovulation phase where the endometrium is exposed to estrogen and progesterone, and a secretory or progesterone dominated phase where the endometrium shows signs of increased gland growth and secretion due largely to the influence of progesterone [1]. The secretory phase is followed by the shedding of the endometrium during menstruation. The histologic changes in the endometrium have been used to detect the stage or status of the endometrium, which is important for example, in determining the receptivity of patients to fertility procedures, and in determining responses to estrogen and/or progesterone therapies.

Endometrial carcinoma is a common malignancy in women, being exceeded in incidence only by that of breast, lung, and colorectal cancers [10, 11, 48, 125-131]. The lifetime probability of a Canadian woman developing endometrial carcinoma is 2.2% [10]. Although the case-fatality rate for cancer of the endometrium is lower than that of many other cancer sites, this rate does not fully reflect the health care burden posed by endometrial carcinoma. Investigation of women with perimenopausal and postmenopausal bleeding for the presence of endometrial carcinoma is one of the most common gynecologic investigations and requires invasive endometrial sampling. Yet only a small proportion of these investigations will result in a diagnosis of endometrial carcinoma [12].

At present, no methods for screening or early detection of endometrial carcinoma are available nor are there any serum tumor markers available for the monitoring of endometrial carcinoma patients [13, 14]. Consequently, patients are diagnosed following the development of symptoms, and patients with recurrent disease are detected only following the development of recurrent symptoms, or abnormalities in imaging assessments. Sensitive and specific tumor marker(s) for endometrial carcinoma are urgently needed for screening and diagnosis. There is also a need for reliable endometrial markers for determining the stage/phase, and status of the endometrium.

### SUMMARY OF THE INVENTION

Applicants have developed a method for identifying markers associated with the endometrium, and in particular with proliferative endometrium, secretory endometrium, and diseased endometrial tissue. Using the method they analyzed normal endometrial tissue homogenates and endometrial tumor tissue homogenates, and identified novel endometrial markers, in particular markers of secretory and proliferative endometrium and endometrial cancer markers.

The invention relates to a method of characterizing or classifying a sample of endometrium by detecting or quantitating in the sample one or more polypeptides extracted from the sample that are characteristic of endometrium, a phase thereof or an endometrial disease, the method comprising assaying for differential expression of polypeptides in the sample. Differential expression of polypeptides can be assayed using separation techniques known in the art, an antibody microarray, or mass spectroscopy of polypeptides extracted from the sample. The invention includes polypeptides identified using a method of the invention.

In an aspect the invention provides a method of characterizing a sample of endometrium by detecting or quantitating in the sample one or more polypeptides extracted from the sample that are characteristic of a proliferative, secretory or an endometrial disease the method comprising assaying for differential expression of proteins in the sample by mass spectroscopy of proteins extracted from the sample. In an embodiment, differential expression of the proteins is carried out using surface enhanced laser desorption/ionization (SELDI-TOF MS).

In an embodiment, the invention provides a method for identifying markers associated with the endometrium or a phase thereof, or associated with an endometrial disease comprising:
(a) obtaining a sample of endometrium from a subject;
(b) extracting proteins from the sample and producing a profile of the proteins by subjecting the proteins to mass spectrometry; and
(c) comparing the profile with a profile for normal endometrial tissue or for a known phase of endometrium to identify proteins associated with an endometrial disease or with the endometrium phase or.

In another aspect the invention is directed to bioinformatic methods for analyzing differential expression data generated from the methods of the invention to identify further markers associated with the endometrium or phase thereof, or associated with endometrial disease.

The invention relates to novel markers for the endometrium, and in particular markers of an endometrial disease, and compositions comprising same.

The invention provides marker sets that distinguish the endometrium or phases thereof, or endometrial diseases, and uses therefor. A marker set may comprise a plurality of polypeptides and/or polynucleotides encoding such polypeptides comprising or consisting of at least 2, 3, 4, 5, 6, 7, 8, 9, 10, 11, 12, 13, 14, or 15 of the markers listed in Table 1, 4, 5, or 6. In specific aspects, the markers consist of at least 5, 6, 7, 8, 9, or 10 polypeptides listed in Table 1, 4, 5, or 6. In an aspect the protein marker sets comprise or consist of protein clusters, or proteins in pathways comprising markers listed in Table 1, 4, 5, and 6.

In embodiments of the invention, a marker is provided which is selected from the group consisting of the polypeptides set forth in Table 1, which polypeptides are up-regulated biomarkers in endometrial cancer.

In embodiments of the invention, a marker is provided which is selected from the group consisting of the polypeptides set forth in Table 1, which polypeptides are down-regulated biomarkers in endometrial cancer.

The markers identified in accordance with a method of the invention, in particular the markers identified in Table 1, 4, 5, or 6, including but not limited to native-sequence polypeptides, isoforms, chimeric polypeptides, all homologs, fragments, and precursors of the markers, including modified forms of the polypeptides and derivatives are referred to herein as “endometrial marker(s)”. Polynucleotides encoding endometrial markers are referred to herein as “endometrial polynucleotide marker(s)”, “polynucleotides encoding endometrial markers”, or “polynucleotides encoding the marker(s)”. The endometrial markers and endometrial polynucleotide markers are sometimes collectively referred to herein as “marker(s)”. Markers of endometrial cancer are referred to herein as “endometrial cancer markers”, “endometrial cancer polynucleotide markers”, and “polynucleotides encoding endometrial cancer markers”.

Endometrial markers identified in accordance with a method of the invention, (including the endometrial cancer markers listed in Table 1, 4, 5, or 6), and polynucleotides encoding the markers, have application in the determination of the status or phase of the endometrium and in the detection of an endometrial disease such as endometrial cancer. Thus, the markers can be used for diagnosis, monitoring (i.e. monitoring progression or therapeutic treatment), prognosis, treatment, or classification of an endometrial disease (e.g. endometrial cancer), or as markers before surgery or after relapse. The invention also contemplates methods for assessing the status of an endometrial tissue, and methods for the diagnosis and therapy of an endometrial disease.

The markers characteristic of different stages or phases of endometrium identified by a method of the invention may be used to identify the physiologic stage or phase of the endometrium within the physiologic cycle. In an aspect, the endometrial markers may be used to assess and manage reproductive disorders and infertility. In particular, endometrial markers associated with the secretory phase or proliferative phase identified by a method of the invention may be used to determine if an endometrium is at the optimum stage or phase for embryo implantation. In an embodiment, the endometrial markers are characteristic of the secretory phase, and include the markers glutamate receptor subunit zeta 1 [SEQ ID NO. 26] or a tryptic fragment thereof [e.g. SEQ ID NO. 28], macrophage migration inhibitory factor [SEQ ID NO. 18], FRAT1 [SEQ ID NO. 42], myosin light chain kinase 2 [SEQ ID NO. 45], and tropomyosin 1 alpha chain [SEQ ID NO. 47], and polynucleotides encoding the polypeptides.

In accordance with methods of the invention, endometrium can be assessed or characterized, for example, by detecting the presence in the sample of (a) an endometrial marker or fragment thereof; (b) a metabolite which is produced directly or indirectly by an endometrial marker; (c) a transcribed nucleic acid or fragment thereof having at least a portion with which an endometrial polynucleotide marker is substantially identical; and/or (c) a transcribed nucleic acid or fragment thereof, wherein the nucleic acid hybridizes with an endometrial polynucleotide marker.

The levels of endometrial markers or endometrial polynucleotide markers in a sample may be determined by methods as described herein and generally known in the art. The expression levels may be determined by isolating and determining the level of nucleic acid transcribed from each endometrial polynucleotide. Alternatively or additionally, the levels of endometrial markers translated from mRNA transcribed from an endometrial polynucleotide marker may be determined.

In an aspect, the invention provides a method for characterizing or classifying an endometrial sample comprising detecting a difference in the expression of a first plurality of endometrial markers or endometrial polynucleotide markers relative to a control, the first plurality of markers consisting of at least 2, 3, 4, 5, 6, 7, 8, 9, 10, 11, 12, 13, 14, or 15 of the markers corresponding to the markers listed in Table 1, 4, 5, or 6. In specific aspects, the plurality of markers consists of at least 5 of the markers listed in Table 1, 4, 5, or 6.

In an aspect, a method is provided for characterizing an endometrium by detecting endometrial markers or endometrial polynucleotide markers associated with an endometrium stage or phase, or endometrial disease in a patient comprising:
(a) obtaining a sample from a subject;
(b) detecting or identifying in the sample endometrial markers or endometrial polynucleotide markers; and
(c) comparing the detected amount with an amount detected for a standard.

In an embodiment of the invention, a method is provided for detecting endometrial cancer markers or endometrial cancer polynucleotide markers associated with endometrial cancer in a patient comprising:
(a) obtaining a sample from a patient;
(b) detecting in the sample endometrial cancer markers or endometrial cancer polynucleotide markers; and
(c) comparing the detected amount with an amount detected for a standard.

The term “detect” or “detecting” includes assaying, imaging or otherwise establishing the presence or absence of the target endometrial markers or polynucleotides encoding the markers, subunits thereof, or combinations of reagent bound targets, and the like, or assaying for, imaging, ascertaining, establishing, or otherwise determining one or more factual characteristics of an endometrium phase or endometrial disease including cancer, metastasis, stage, or similar conditions. The term encompasses diagnostic, prognostic, and monitoring applications for the endometrial markers and polynucleotides encoding the markers.

The invention also provides a method of assessing whether a patient is afflicted with or has a pre-disposition for endometrial disease, in particular endometrial cancer, the method comprising comparing:
(a) levels of endometrial markers or polynucleotides encoding endometrial markers associated with the endometrial disease in a sample from the patient; and
(b) normal levels of endometrial markers or polynucleotides encoding endometrial markers associated with the endometrial disease in samples of the same type obtained from control patients not afflicted with the disease, wherein altered levels of the endometrial markers or the polynucleotides relative to the corresponding normal levels of endometrial markers or polynucleotides is an indication that the patient is afflicted with endometrial disease.

In an aspect of a method of the invention for assessing whether a patient is afflicted with or has a pre-disposition for endometrial cancer, higher levels of endometrial cancer markers in a sample relative to the corresponding normal levels is an indication that the patient is afflicted with endometrial cancer.

In another aspect of a method of the invention for assessing whether a patient is afflicted with or has a pre-disposition for endometrial cancer, lower levels of endometrial cancer markers in a sample relative to the corresponding normal levels is an indication that the patient is afflicted with endometrial cancer.

In a further aspect, a method for screening a subject for endometrial disease is provided comprising (a) obtaining a biological sample from a subject; (b) detecting the amount of endometrial markers associated with the disease in said sample; and (c) comparing said amount of endometrial markers detected to a predetermined standard, where detection of a level of endometrial markers that differs significantly from the standard indicates endometrial disease.

In an embodiment, a significant difference between the levels of endometrial marker levels in a patient and normal levels is an indication that the patient is afflicted with or has a predisposition to endometrial disease.

In a particular embodiment the amount of endometrial marker(s) detected is greater than that of a standard and is indicative of endometrial disease, in particular endometrial cancer. In another particular embodiment the amount of endometrial marker(s) detected is lower than that of a standard and is indicative of endometrial disease, in particular endometrial cancer.

In aspects of the methods of the invention, the methods are non-invasive for detecting endometrium phase or endometrial disease which in turn allow for diagnosis of a variety of conditions or diseases associated with the endometrium.

In particular, the invention provides a non-invasive non-surgical method for detection, diagnosis or prediction of endometrial disease in a subject comprising: obtaining a sample of blood, plasma, serum, urine or saliva or a tissue sample from the subject; subjecting the sample to a procedure to detect endometrial markers or endometrial polynucleotide markers in the blood, plasma, serum, urine, saliva or tissue; detecting, diagnosing, and predicting endometrial disease by comparing the levels of endometrial markers or endometrial polynucleotide markers to the levels of marker(s) or polynucleotide(s) obtained from a control subject with no endometrial disease.

In an embodiment, endometrial disease is detected, diagnosed, or predicted by determination of increased levels of markers (e.g Table 1 up-regulated markers) when compared to such levels obtained from the control.

In another embodiment, endometrial disease is detected, diagnosed, or predicted by determination of decreased levels of markers (e.g. Table 1 down-regulated markers) when compared to such levels obtained from the control.

The invention also provides a method for assessing the aggressiveness or indolence of an endometrial disease in particular cancer (e.g. staging), the method comprising comparing:
(a) levels of endometrial markers or polynucleotides encoding endometrial markers associated with the endometrial disease in a patient sample; and
(b) normal levels of the endometrial markers or the polynucleotides in a control sample.

In an embodiment, a significant difference between the levels in the sample and the normal levels is an indication that the endometrial disease, in particular cancer, is aggressive or indolent. In a particular embodiment, the levels of endometrial markers are higher than normal levels. In another particular embodiment, the levels of endometrial markers are lower than normal levels.

In an aspect, the invention provides a method for determining whether a cancer has metastasized or is likely to metastasize in the future, the method comprising comparing:
(a) levels of endometrial cancer markers or polynucleotides encoding endometrial cancer markers in a patient sample; and
(b) normal levels (or non-metastatic levels) of the endometrial cancer markers or polynucleotides in a control sample.

In an embodiment, a significant difference between the levels in the patient sample and the normal levels is an indication that the cancer has metastasized or is likely to metastasize in the future.

In another aspect, the invention provides a method for monitoring the progression of endometrial disease, in particular endometrial cancer in a patient the method comprising:
(a) detecting endometrial markers or polynucleotides encoding the markers associated with the disease in a sample from the patient at a first time point;
(b) repeating step (a) at a subsequent point in time; and
(c) comparing the levels detected in (a) and (b), and therefrom monitoring the progression of the endometrial disease.

The invention contemplates a method for determining the effect of an environmental factor on the endometrium or phase thereof, or endometrial disease comprising comparing endometrial polynucleotide markers or endometrial markers in the presence and absence of the environmental factor.

The invention also provides a method for assessing the potential efficacy of a test agent for inhibiting endometrial disease, and a method of selecting an agent for inhibiting endometrial disease.

The invention contemplates a method of assessing the potential of a test compound to contribute to an endometrial disease comprising:
(a) maintaining separate aliquots of endometrial diseased cells in the presence and absence of the test compound; and
(b) comparing the levels of endometrial markers or polynucleotides encoding the markers associated with the disease in each of the aliquots.

A significant difference between the levels of endometrial markers or polynucleotides encoding the markers in an aliquot maintained in the presence of (or exposed to) the test compound relative to the aliquot maintained in the absence of the test compound, indicates that the test compound potentially contributes to endometrial disease.

The invention further relates to a method of assessing the efficacy of a therapy for inhibiting endometrial disease in a patient. A method of the invention comprises comparing: (a) levels of endometrial markers or polynucleotides encoding the markers associated with disease in a first sample from the patient obtained from the patient prior to providing at least a portion of the therapy to the patient; and (b) levels of endometrial markers or polynucleotides encoding the markers associated with disease in a second sample obtained from the patient following therapy.

In an embodiment, a significant difference between the levels of endometrial markers or polynucleotides encoding the markers in the second sample relative to the first sample is an indication that the therapy is efficacious for inhibiting endometrial disease.

In a particular embodiment, the method is used to assess the efficacy of a therapy for inhibiting endometrial disease (e.g. endometrial cancer), where lower levels of endometrial markers or polynucleotides encoding same in the second sample relative to the first sample, is an indication that the therapy is efficacious for inhibiting the disease.

The “therapy” may be any therapy for treating endometrial disease, in particular endometrial cancer, including but not limited to therapeutics, radiation, immunotherapy, gene therapy, and surgical removal of tissue. Therefore, the method can be used to evaluate a patient before, during, and after therapy.

Certain methods of the invention employ binding agents (e.g. antibodies) that specifically recognize endometrial markers.

In an embodiment, the invention provides methods for determining the presence or absence of endometrial disease, in particular endometrial cancer, in a patient, comprising the steps of (a) contacting a biological sample obtained from a patient with one or more binding agent that specifically binds to one or more endometrial markers associated with the disease; and (b) detecting in the sample an amount of marker that binds to the binding agent, relative to a predetermined standard or cut-off value, and therefrom determining the presence or absence of endometrial disease in the patient.

In another embodiment, the invention relates to a method for diagnosing and monitoring an endometrial disease, in particular endometrial cancer, in a subject by quantitating one or more endometrial markers associated with the disease in a biological sample from the subject comprising (a) reacting the biological sample with one or more binding agent specific for the endometrial markers (e.g. an antibody) that are directly or indirectly labelled with a detectable substance; and (b) detecting the detectable substance.

In another aspect the invention provides a method for using an antibody to detect expression of one or more endometrial marker in a sample, the method comprising: (a) combining antibodies specific for one or more endometrial marker with a sample under conditions which allow the formation of antibody marker complexes; and (b) detecting complex formation, wherein complex formation indicates expression of the marker in the sample. Expression may be compared with standards and is diagnostic of an endometrial disease, in particular endometrial cancer.

Embodiments of the methods of the invention involve (a) reacting a biological sample from a subject with antibodies specific for one or more endometrial markers which are directly or indirectly labelled with an enzyme; (b) adding a substrate for the enzyme wherein the substrate is selected so that the substrate, or a reaction product of the enzyme and substrate forms fluorescent complexes; (c) quantitating one or more endometrial markers in the sample by measuring fluorescence of the fluorescent complexes; and (d) comparing the quantitated levels to levels obtained for other samples from the subject patient, or control subjects.

In another embodiment the quantitated levels are compared to levels quantitated for control subjects (e.g. normal or benign) without an endometrial disease (e.g. cancer) wherein an increase in endometrial marker levels compared with the control subjects is indicative of endometrial disease.

In a further embodiment the quantitated levels are compared to levels quantitated for control subjects (e.g. normal or benign) without an endometrial disease (e.g. cancer) wherein a decrease in endometrial marker levels compared with the control subjects is indicative of endometrial disease.

A particular embodiment of the invention comprises the following steps
(a) incubating a biological sample with first antibodies specific for one or more endometrial cancer markers which are directly or indirectly labeled with a detectable substance, and second antibodies specific for one or more endometrial cancer markers which are immobilized;
(b) detecting the detectable substance thereby quantitating endometrial cancer markers in the biological sample; and
(c) comparing the quantitated endometrial cancer markers with levels for a predetermined standard.

The standard may correspond to levels quantitated for samples from control subjects without endometrial cancer (normal or benign), with a different disease stage, or from other samples of the subject. In an embodiment, increased levels of endometrial cancer markers as compared to the standard may be indicative of endometrial cancer. In another embodiment, lower levels of endometrial cancer markers as compared to a standard may be indicative of endometrial cancer.

Endometrial marker levels can be determined by constructing an antibody microarray in which binding sites comprise immobilized, preferably monoclonal, antibodies specific to a substantial fraction of marker-derived endometrial marker proteins of interest.

Other methods of the invention employ one or more polynucleotides capable of hybridizing to one or more polynucleotides encoding endometrial markers. Thus, methods for detecting endometrial markers can be used to monitor an endometrial disease (e.g. cancer) by detecting endometrial polynucleotide markers associated with the disease.

Thus, the present invention relates to a method for diagnosing and monitoring an endometrial disease (e.g. endometrial cancer) in a sample from a subject comprising isolating nucleic acids, preferably mRNA, from the sample; and detecting endometrial marker polynucleotides associated with the disease in the sample. The presence of different levels of endometrial marker polynucleotides in the sample compared to a standard or control may be indicative of endometrium phase, disease, disease stage, and/or a positive prognosis i.e. longer progression-free and overall survival.

In embodiments of the invention, endometrial cancer marker polynucleotide positive tumors (e.g. higher levels of the polynucleotides compared to a control normal or benign tissue) are a negative diagnostic indicator. Positive tumors can be indicative of endometrial cancer, advanced stage disease, lower progression-free survival, and/or overall survival.

In other embodiments of the invention, endometrial cancer marker polynucleotide negative tumors (e.g. lower levels of the polynucleotides compared to a control normal or benign tissue) are a negative diagnostic indicator. Negative tumors can be indicative of endometrial cancer, advanced stage disease, lower progression-free survival, and/or overall survival.

The invention provides methods for determining the presence or absence of an endometrial disease in a subject comprising detecting in the sample levels of nucleic acids that hybridize to one or more polynucleotides encoding endometrial markers associated with the disease, comparing the levels with a predetermined standard or cut-off value, and therefrom determining the presence or absence of endometrial disease in the subject. In an embodiment, the invention provides methods for determining the presence or absence of endometrial cancer in a subject comprising (a) contacting a sample obtained from the subject with oligonucleotides that hybridize to one or more polynucleotides encoding endometrial cancer markers; and (b) detecting in the sample a level of nucleic acids that hybridize to the polynucleotides relative to a predetermined cut-off value, and therefrom determining the presence or absence of endometrial cancer in the subject.

Within certain embodiments, the amount of polynucleotides that are mRNA are detected via polymerase chain reaction using, for example, oligonucleotide primers that hybridize to one or more polynucleotides encoding endometrial markers, or complements of such polynucleotides. Within other embodiments, the amount of mRNA is detected using a hybridization technique, employing oligonucleotide probes that hybridize to one or more polynucleotides encoding endometrial markers, or complements thereof.

When using mRNA detection, the method may be carried out by combining isolated mRNA with reagents to convert to cDNA according to standard methods; treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of one or more endometrial polynucleotide markers in the sample. For mRNA the analyzing step may be accomplished using Northern Blot analysis to detect the presence of endometrial polynucleotide markers. The analysis step may be further accomplished by quantitatively detecting the presence of endometrial polynucleotide markers in the amplification product, and comparing the quantity of marker detected against a panel of expected values for the known presence or absence of the markers in normal and malignant tissue derived using similar primers.

Therefore, the invention provides a method wherein mRNA is detected by (a) isolating mRNA from a sample and combining the mRNA with reagents to convert it to cDNA; (b) treating the converted cDNA with amplification reaction reagents and nucleic acid primers that hybridize to one or more endometrial polynucleotide markers to produce amplification products; (d) analyzing the amplification products to detect an amount of mRNA encoding the endometrial markers; and (e) comparing the amount of mRNA to an amount detected against a panel of expected values for normal and diseased tissue (e.g. malignant tissue) derived using similar nucleic acid primers.

In particular embodiments of the invention, the methods described herein utilize the endometrial polynucleotide markers placed on a microarray so that the expression status of each of the markers is assessed simultaneously.

In a particular aspect, the invention provides an endometrial microarray comprising a defined set of genes whose expression is significantly altered by endometrium phase or endometrial disease. The invention further relates to the use of the microarray as a prognostic tool to predict endometrium phase or endometrial disease. In an embodiment, the endometrial microarray discriminates between endometrial disease resulting from different etiologies.

In an embodiment, the invention provides for oligonucleotide arrays comprising marker sets described herein. The microarrays provided by the present invention may comprise probes to markers able to distinguish endometrium phase or disease. In particular, the invention provides oligonucleotide arrays comprising probes to a subset or subsets of at least 5 or 10 gene markers up to a full set of markers which distinguish endometrium phase or endometrial disease.

The invention also contemplates a method comprising administering to cells or tissues imaging agents that carry labels for imaging and bind to endometrial markers and optionally other markers of an endometrial disease, and then imaging the cells or tissues.

In an aspect the invention provides an in vivo method comprising administering to a subject an agent that has been constructed to target one or more endometrial markers.

In a particular embodiment, the invention contemplates an in vivo method comprising administering to a mammal one or more agent that carries a label for imaging and binds to one or more endometrial marker, and then imaging the mammal.

According to a particular aspect of the invention, an in vivo method for imaging endometrial cancer is provided comprising:
(a) injecting a patient with an agent that binds to one or more endometrial cancer marker, the agent carrying a label for imaging the endometrial cancer;
(b) allowing the agent to incubate in vivo and bind to one or more endometrial cancer marker associated with the endometrial cancer; and
(c) detecting the presence of the label localized to the endometrial cancer.

In an embodiment of the invention the agent is an antibody which recognizes an endometrial cancer marker. In another embodiment of the invention the agent is a chemical entity which recognizes an endometrial cancer marker.

An agent carries a label to image an endometrial marker and optionally other markers. Examples of labels useful for imaging are radiolabels, fluorescent labels (e.g fluorescein and rhodamine), nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed.

The invention also contemplates the localization or imaging methods described herein using multiple markers for an endometrial disease (e.g. endometrial cancer).

The invention also relates to kits for carrying out the methods of the invention. In an embodiment, a kit is for assessing whether a patient is afflicted with an endometrial disease (e.g. endometrial cancer) and it comprises reagents for assessing one or more endometrial markers or polynucleotides encoding the markers.

The invention further provides kits comprising marker sets described herein. In an aspect the kit contains a microarray ready for hybridization to target endometrial oligonucleotide markers, plus software for the data analyses.

The invention also provides a diagnostic composition comprising an endometrial marker or a polynucleotide encoding the marker. A composition is also provided comprising a probe that specifically hybridizes to endometrial polynucleotide markers, or a fragment thereof, or an antibody specific for endometrial markers or a fragment thereof. In another aspect, a composition is provided comprising one or more endometrial polynucleotide marker specific primer pairs capable of amplifying the polynucleotides using polymerase chain reaction methodologies. The probes, primers or antibodies can be labeled with a detectable substance.

Still further the invention relates to therapeutic applications for endometrial diseases, in particular endometrial cancer, employing endometrial markers and polynucleotides encoding the markers, and/or binding agents for the markers.

In an aspect, the invention relates to compositions comprising markers or parts thereof associated with an endometrial disease, or antibodies specific for endometrial markers associated with an endometrial disease, and a pharmaceutically acceptable carrier, excipient, or diluent. A method for treating or preventing an endometrial disease, in particular endometrial cancer, in a patient is also provided comprising administering to a patient in need thereof, markers or parts thereof associated with an endometrial disease, antibodies specific for endometrial markers associated with an endometrial disease, or a composition of the invention. In an aspect the invention provides a method of treating a patient afflicted with or at risk of developing an endometrial disease (e.g. endometrial cancer) comprising inhibiting expression of endometrial markers.

In an aspect, the invention provides antibodies specific for endometrial markers associated with a disease (e.g. endometrial cancer) that can be used therapeutically to destroy or inhibit the disease (e.g. the growth of endometrial cancer marker expressing cancer cells), or to block endometrial marker activity associated with a disease. In an aspect, endometrial cancer markers may be used in various immunotherapeutic methods to promote immune-mediated destruction or growth inhibition of tumors expressing endometrial cancer markers.

The invention also contemplates a method of using endometrial markers or parts thereof, or antibodies specific for endometrial markers in the preparation or manufacture of a medicament for the prevention or treatment of an endometrial disease e.g. endometrial cancer.

Another aspect of the invention is the use of endometrial markers, peptides derived therefrom, or chemically produced (synthetic) peptides, or any combination of these molecules, for use in the preparation of vaccines to prevent an endometrial disease and/or to treat an endometrial disease.

The invention contemplates vaccines for stimulating or enhancing in a subject to whom the vaccine is administered production of antibodies directed against one or more endometrial markers.

The invention also provides a method for stimulating or enhancing in a subject production of antibodies directed against one or more endometrial marker. The method comprises administering to the subject a vaccine of the invention in a dose effective for stimulating or enhancing production of the antibodies.

The invention further provides a method for treating, preventing, or delaying recurrence of an endometrial disease (e.g. endometrial cancer). The method comprises administering to the subject a vaccine of the invention in a dose effective for treating, preventing, or delaying recurrence of an endometrial disease (e.g. endometrial cancer).

The invention contemplates the methods, compositions, and kits described herein using additional markers associated with an endometrial disease (e.g. endometrial cancer). The methods described herein may be modified by including reagents to detect the additional markers, or polynucleotides for the markers.

In particular, the invention contemplates the methods described herein using multiple markers for an endometrial cancer. Therefore, the invention contemplates a method for analyzing a biological sample for the presence of endometrial cancer markers and polynucleotides encoding the markers, and other markers that are specific indicators of cancer, in particular endometrial cancer. The methods described herein may be modified by including reagents to detect the additional markers, or nucleic acids for the additional markers.

In embodiments of the invention the methods, compositions and kits use one or more of the markers listed in Table 1, 4, 5, or 6. In another embodiment, the method uses a panel of markers selected from the markers listed in Table 1, 4, 5, or 6 in particular a panel comprising two or more of the markers in Table 1, 4, 5 or 6.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1 shows a schematic representation of the ICAT procedure including labeling, digestion, SCX fractionation, affinity isolation, cleavage of the biotin moiety and LC-MS/MS analysis.

FIG. 2 shows histologic sections of human endometrium, stained with hematoxylin and eosin. A) proliferative endometrium, sample PRO 2, and B) secretory endometrium, sample SEC2.

FIG. 3 shows a Nano LC-MS TIC of SCX fraction 16, ICAT sample A.

FIG. 4 shows the distribution of proteins identified from the middle ten SCX fractions of all three samples.

FIG. 5 shows A) a mass spectral window of Sample A, SCX fraction 19, showing enhanced expression of glutamate receptor subunit zeta 1 precursor in the secretory endometrium. The line to the left of the isotope envelope of the heavy-labeled version of the protein denotes where the light-labeled version is expected. B) Resulting MS/MS spectra of the heavy labelled series showing sequence coverage.

FIG. 6 shows A) a mass spectral window of Sample A, SCX fraction 13, showing enhanced expression of MIF in the secretory endometrium. B) Resulting MS/MS spectra of the heavy labelled series sequence coverage.

FIG. 7 (a+b). STEP ONE Mass spectra obtained from five whole endometrial tissue homogenates on Proteinchip WCX2 using (1a) SELDI-TOF-MS, PBSIIc, and (1b) QqTOF-MS, QSTAR XL. Note, using either MS method, the three malignant cases (38, 39, 40) show a distinct protein peak, which is low in the two non-malignant cases [18, 19]. The QSTARXL MS determined the weight of this peak to be 10,843 Da. FIG. 7 STEP TWO Outline of protein purification. The target protein eluted in one of the early fractions of the size exclusion HPLC, was concentrated by Proteospin and then subjected to MALDI and SDS-PAGE (see FIG. 8).

FIG. 8 (A to E). STEP THREE (8A). Molecular weight verification of target protein after SEC. MALDI-TOF MS of samples from Proteospin was used to locate the fraction with the 10,843 Da weight of the target protein in a malignant case (40). The corresponding fraction from normal sample was used in parallel for all the following experiments. (8B) Molecular weight verification of the target protein fraction following DTT treatment. MALDI-TOF analysis of two samples indicates that the 10,843 Da peak remained in the malignant sample even after DTT treatment. (8C) SDS-PAGE analysis of the DTT treated fractions. The gel was stained by colloidal coomassie blue (Gel-Code, Pierce). The region containing the target protein was excised according to the molecular weight marker, and the intact proteins were extracted. (8D) Tryptic peptide profiles of the SDS-PAGE protein extract. MALDI-TOF MS analysis identified six “unique” tryptic peptides in the malignant samples. Three of them with mass of 907.54, 1036.58, and 1529.82 Da matched with the tryptic peptides from chaperonin 10. The remaining three peaks, marked with *, were from Keratin. (insert) Amino acid sequence of chaperonin 10. The locations of the three peptides were underlined. (8E) Fragmentation of peptide by collision induced dissociation. The 1529.82 Da peptide was fragmented. A series of y ions confirmed that the 1529.82 Da peptide comes from chaperonin 10.

FIG. 9. Western blot analysis against chaperonin 10. The malignant endometrial cases (EmCa) demonstrate a higher expression of chaperonin 10 than non-malignant cases (Normal). The level of ERK1 was used to demonstrate similar loading among all lanes.

FIG. 10. Immunohistochemical staining for chaperonin 10. Note granular cytoplasmic positivity in several malignant endometrioid glands, while adjacent endometrial stroma and a portion of one benign endometrial gland (right) shows much reduced staining.

FIG. 11 shows mass spectral windows from isotope-coded affinity tag (ICAT) experiments for three pairs of endometrial cancer/normal samples, demonstrating the over expression of calgizzarin in the cancer samples.

FIG. 12: Number of peptides/protein identified. The first (front) series corresponds to results obtained from cICAT, the second series is from the first iTRAQ run, and the third (rearmost) series is from the second iTRAQ run.

FIG. 13: Cytoplasmic actin sequencing and quantification using iTRAQ. A: MS/MS spectrum of the doubly protonated peptide, EITALAPSTMK [SEQ ID NO. 49], at 725.4 Th. Residues in black are sequenced from the b-ion series, while those in purple from the y-ion series. Residue ‘J’ in the y-ion series is the iTRAQ-modified lysine residue. B: Expanded view of the low-m/z end of the MS/MS spectrum in ‘A’, showing relative abundances of the signature iTRAQ ions at 114.1, 115.1, 116.1 and 117.1 Th.

FIG. 14: Relative abundances of differentially expressed proteins. The ion assignments are as follows: 114 Th, proliferative endometrium; 115 Th, secretory endometrium; 116 Th, first endometrial carcinoma; and 117Th, second endometrial carcinoma. A: Tryptic peptide from chaperonin 10, showing higher abundances in the 116 and 117 Th ions relative to the 114 and 115 Th ions; B: tryptic peptide from alpha-1-antitrypsin precursor, showing lower abundances in the 116 and 117 Th ions relative to the 114 and 115 Th ions; C: tryptic peptide from creatine kinase B, showing lower abundances in the 116 and 117 Th ions relative to the 114 and 115 Th ions; D: tryptic peptide from transgelin, showing once more lower abundances in the 116 and 117 Th ions relative to the 114 and 115 Th ions.

FIG. 15: Relative abundances of calgizzarin. cICAT analysis shows overexpression in all three cancer samples. The dotted line to the left of the heavy-label series, which originates from the cancerous sample, indicates the monoisotopic peak of the light-label series, which originates from the normal sample.

FIG. 16: Distribution of proteins identified by A: iTRAQ and B: cICAT analyses.

FIG. 17 shows histologic sections of human endometrium stained with hematoxylin and eosin: (a) proliferative endometrium, sample PRO2, and (b) secretory endometrium, sample SEC2.

FIG. 18 is a nano LC/MS total ion chromatogram of strong cation exchange fraction 16 of sample A.

FIG. 19 shows the distribution of 119 confirmed proteins. The legend shows the protein categories starting with “Structural” at 18% at the 2 o'clock position and continuing clockwise.

FIG. 20 shows the appearance of selected proteins in the strong cation exchange fractions.

FIG. 21 shows the (a) mass spectral window of sample A, fraction 19, showing enhanced expression of glutamate receptor subunit zeta 1 precursor in the secretory endometrium. The line to the left of the isotope envelope of the heavy-labeled version of the protein denotes where the light-labeled version is expected; (b) resulting MS/MS spectrum showing a partial sequence for the peptide LLTLALLFSCSVAR [SEQ ID NO. 28].

FIG. 22 shows the mass spectral windows of the three sample pairs, fraction 15, showing enhanced expression of FRAT1.

FIG. 23 shows the mass spectral windows of sample A, fraction 4, showing enhanced expression of glycodelin in the secretory endometrium.

FIG. 24 shows the mass spectral windows showing the isotopic clusters of cathepsin B. Sample A shows enhanced cathepsin B expression in the secretory endometrium; sample B shows the opposite trend.

FIG. 25 shows SELDI MS spectral windows acquired on the QqTOF mass spectrometer (QSTAR XL): (a) typical tissue homogenate; (b) homogenate treated with 0% hydrogen peroxide; (c) homogenate in (b) treated incubated with 3% hydrogen peroxide overnight; (d) laser fluence at 34.6 μJ; and (e) laser fluence at 76.9 μJ.

FIG. 26 shows SELDI MS spectra of four malignant (designated by “C”) and two endometrial tissue homogenates (designated by “N”): (a) full scan spectra collected on the linear QTOF mass spectrometer (PBS IIc); and (b) mass spectral windows collected on the PBS IIc, left panel, and on the QqTOF mass spectrometer (QSTAR XL), right panel.

FIG. 27 shows SELDI MS spectral windows showing binding of both (a) chaperonin 10 and (b) the unknown protein at pH 6.0; and selective binding of the unknown protein at pH 7.0.

FIG. 28 shows MS/MS spectra of three tryptic peptides from the unknown protein that identify it as calgranulin A. Peptide identities: (a) MLTELEK [SEQ ID NO. 50], (b) ALNSIIDVYHK [SEQ ID NO. 51], and (c) GADVWFK [SEQ ID NO. 52].

FIG. 29 shows Calgranulin A immunohistochemical stain of endometrial TMA (US Biochem 1:150): (A) Endometrioid adenocarcinoma exhibits diffuse, immunostaining of numerous cells (3+) of the glandular component of the adenocarcinoma. Intense immunostaining of macrophages/granulocytes within the stroma and glandular lumina is also evident. No stromal staining is observed. (B) Endometrioid adenocarcinoma shows strong positive immunostaining of occasional malignant glands (1+). Intense individual cell staining is also apparent in macrophages/granulocytes within gland lumina and stroma.

FIG. 30 shows Calgranulin A immunohistochemical stain of endometrial TMA (US Biochem 1:150): Note intense (3+) cytoplasmic and nuclear immunostaining in squamous areas of this endometrioid adenocarcinoma. Glandular or columnar areas of the adenocarcinoma and adjacent stroma do not demonstrate any staining in this particular case.

FIG. 31 shows Calgranulin A immunohistochemical stain of endometrial TMA (US Biochem 1:150): The glands and stroma of this proliferative endometrium show no immunostaining. However, intense individual macrophage/granulocyte immunostaining is evident within the stroma.

### DETAILED DESCRIPTION OF THE INVENTION

Methods are provided for characterizing the stage or phase of endometrium, detecting the presence of an endometrial disease (e.g. endometrial cancer) in a sample, the absence of a disease (e.g. endometrial cancer) in a sample, the stage of a disease, the stage or grade of the disease, and other characteristics of endometrial diseases that are relevant to prevention, diagnosis, characterization, and therapy of endometrial diseases such as cancer in a patient, for example, the benign or malignant nature of an endometrial cancer, the metastatic potential of an endometrial cancer, assessing the histological type of neoplasm associated with an endometrial cancer, the indolence or aggressiveness of an endometrial cancer, and other characteristics of endometrial diseases that are relevant to prevention, diagnosis, characterization, and therapy of endometrial diseases such as cancer in a patient. Methods are also provided for assessing the efficacy of one or more test agents for inhibiting an endometrial disease, assessing the efficacy of a therapy for an endometrial disease, monitoring the progression of an endometrial disease, selecting an agent or therapy for inhibiting an endometrial disease, treating a patient afflicted with an endometrial disease, inhibiting an endometrial disease in a patient, and assessing the disease (e.g. carcinogenic) potential of a test compound.

### Glossary

“Endometrial disease” refers to any disorder, disease, condition, syndrome or combination of manifestations or symptoms recognized or diagnosed as a disorder of the endometrium, including but not limited to hyperplasia and cancer precursors, endometrial cancer or carcinoma, endometriosis, reproductive disorders, and infertility.

“Endometrial cancer” or “endometrial carcinoma” includes malignant endometrial disease including but not limited to endometrioid, mucinous, and serous adenocarcinomas, adenosquamous carcinomas, clear cell carcinomas, uterine sarcomas including stromal sarcomas, malignant mixed Mullerian tumors (carcinosarcomas), and leiomyosarcomas.

The terms “sample”, “biological sample”, and the like mean a material known or suspected of expressing or containing one or more endometrial polynucleotide markers or one or more endometrial markers. A test sample can be used directly as obtained from the source or following a pretreatment to modify the character of the sample. The sample can be derived from any biological source, such as tissues, extracts, or cell cultures, including cells (e.g. tumor cells), cell lysates, and physiological fluids, such as, for example, whole blood, plasma, serum, saliva, ocular lens fluid, cerebral spinal fluid, sweat, urine, milk, ascites fluid, synovial fluid, peritoneal fluid, lavage fluid, and the like. The sample can be obtained from animals, preferably mammals, most preferably humans. The sample can be treated prior to use, such as preparing plasma from blood, diluting viscous fluids, and the like. Methods of treatment can involve filtration: distillation, extraction, concentration, inactivation of interfering components, the addition of reagents, and the like.

In embodiments of the invention the sample is a mammalian tissue sample. In a particular embodiment, the tissue is endometrial tissue.

In another embodiment the sample is a human physiological fluid. In a particular embodiment, the sample is human serum.

The samples that may be analyzed in accordance with the invention include polynucleotides from clinically relevant sources, preferably expressed RNA or a nucleic acid derived therefrom (cDNA or amplified RNA derived from cDNA that incorporates an RNA polymerase promoter). The target polynucleotides can comprise RNA, including, without limitation total cellular RNA, poly(A)⁺ messenger RNA (mRNA) or fraction thereof, cytoplasmic mRNA, or RNA transcribed from cDNA (i.e., cRNA; see, for example, Linsley & Schelter, U.S. patent application Ser. No. 09/411,074, or U.S. Pat. No. 5,545,522, 5,891,636, or 5,716,785). Methods for preparing total and poly(A)⁺ RNA are well known in the art, and are described generally, for example, in Sambrook et al., (1989, Molecular Cloning—A Laboratory Manual (2^{nd} Ed.), Vols. 1-3, Cold Spring Harbor Laboratory, Cold Spring Harbor, N.Y.) and Ausubel et al, eds. (1994, Current Protocols in Molecular Biology, vol. 2, Current Protocols Publishing, New York). RNA may be isolated from eukaryotic cells by procedures involving lysis of the cells and denaturation of the proteins contained in the cells. Additional steps may be utilized to remove DNA. Cell lysis may be achieved with a nonionic detergent, followed by microcentrifugation to remove the nuclei and hence the bulk of the cellular DNA. (See Chirgwin et al., 1979, Biochemistry 18:5294-5299). Poly(A)+RNA can be selected using oligo-dT cellulose (see Sambrook et al., 1989, Molecular Cloning—A Laboratory Manual (2nd Ed.), Vols. 1-3, Cold Spring Harbor Laboratory, Cold Spring Harbor, N.Y.). In the alternative, RNA can be separated from DNA by organic extraction, for example, with hot phenol or phenol/chloroform/isoamyl alcohol.

It may be desirable to enrich mRNA with respect to other cellular RNAs, such as transfer RNA (tRNA) and ribosomal RNA (rRNA). Most mRNAs contain a poly(A) tail at their 3′ end allowing them to be enriched by affinity chromatography, for example, using oligo(dT) or poly(U) coupled to a solid support, such as cellulose or Sephadex™ (see Ausubel et al., eds., 1994, Current Protocols in Molecular Biology, vol. 2, Current Protocols Publishing, New York). Bound poly(A)+mRNA is eluted from the affinity column using 2 mM EDTA/0.1% SDS.

A sample of RNA can comprise a plurality of different mRNA molecules each with a different nucleotide sequence. In an aspect of the invention, the mRNA molecules in the RNA sample comprise at least 100 different nucleotide sequences.

Target polynucleotides can be detectably labeled at one or more nucleotides using methods known in the art. The label is preferably uniformly incorporated along the length of the RNA, and more preferably, is carried out at a high degree of efficiency. The detectable label can be a luminescent label, fluorescent label, bio-luminescent label, chemi-luminescent label, radiolabel, and colorimetric label. In a particular embodiment, the label is a fluorescent label, such as a fluorescein, a phosphor, a rhodamine, or a polymethine dye derivative. Commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.).

Target polynucleotides from a patient sample can be labeled differentially from polynucleotides of a standard. The standard can comprise target polynucleotides from normal individuals (i.e., those not afflicted with or pre-disposed to endometrial disease, in particular pooled from samples from normal individuals. The target polynucleotides can be derived from the same individual, but taken at different time points, and thus indicate the efficacy of a treatment by a change in expression of the markers, or lack thereof, during and after the course of treatment.

The terms “subject”, “individual” or “patient” refer to a warm-blooded animal such as a mammal. In particular, the terms refer to a human. A subject, individual or patient may be afflicted with or suspected of having or being pre-disposed to endometrial disease or a condition as described herein. The term also includes domestic animals bred for food or as pets, including horses, cows, sheep, poultry, fish, pigs, cats, dogs, and zoo animals.

Methods herein for administering an agent or composition to subjects/individuals/patients contemplate treatment as well as prophylactic use. Typical subjects for treatment include persons susceptible to, suffering from or that have suffered a condition or disease described herein. In particular, suitable subjects for treatment in accordance with the invention are persons that are susceptible to, suffering from or that have suffered endometrial cancer.

The term “endometrial marker” includes a marker associated with normal or diseased endometrial tissue identified using a method of the invention. The term includes native-sequence polypeptides isoforms, chimeric polypeptides, complexes, all homologs, fragments, precursors, and modified forms and derivatives of the markers.

An endometrial marker may be associated with a stage or phase of endometrial tissue such as the secretory or proliferative phase. Examples of endometrial markers associated with the secretory phase are glutamate receptor subunit zeta 1 [SEQ ID NO. 26] and parts thereof (e.g. a tryptic fragment of SEQ ID NO. 28), macrophage migration inhibitory factor [SEQ ID NO. 18], FRAT1 [SEQ ID NO. 42], myosin light chain kinase 2 [SEQ ID NO. 45], and tropomyosin 1 alpha chain [SEQ ID NO. 47].

An endometrial marker may be associated with an endometrial disease, in particular it may be an endometrial cancer marker. The term “endometrial cancer marker” includes a marker associated with endometrial cancer identified using a method of the invention, in particular a marker listed in Table 1.

In an aspect of the invention, an endometrial cancer marker is chaperonin 10. The term “chaperonin 10”, “chaperonin 10 polypeptide” or “chaperonin protein” includes human chaperonin 10, in particular the native-sequence polypeptide, isoforms, chimeric polypeptides, all homologs, fragments, precursors, complexes, and modified forms and derivatives of human chaperonin 10. The amino acid sequence for native human chaperonin 10 includes the sequences of GenBank Accession No. Q04984 and AAH23518 shown in SEQ ID NO. 1.

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” means a polypeptide having at least about 45%, 50%, 55%, 60%, 65%, 70%, 75%, 80%, 85%, 90%, 95%, 97%, 98%, or 99% amino acid sequence identity, particularly at least about 70-80%, more particularly at least about 85%, still more particularly at least about 90%, most particularly at least about 95% amino acid sequence identity with a native-sequence polypeptide. Particular polypeptide variants have at least 70-80%, 85%, 90%, 95% amino acid sequence identity to the sequences identified in Table 1, 4, 5, or 6 (see in particular, GenBank Accession Nos. Q04984 and AAH23518, P05109, P06702, P01833 or Q81ZY7, P30086, P39687, P17066, P14174, P31949, P00938 and NP_{—}000356, Q05586, ITHU and P01009, gi/125294 and P12277, P14618, Q01995, Q14103, NP_{—}852000, NP_{—}444254, and P09493 and AAH07433 and SEQ ID NOs. 1, 3, 6, 9, 11, 13, 15, 18, 21, 23, 26, 30, 33, 36, 38, 40, 42, 45, and 47). Such variants include, for instance, polypeptides wherein one or more amino acid residues are added to, or deleted from, the N- or C-terminus of the full-length or mature sequences of the polypeptide, including variants from other species, but excludes a native-sequence polypeptide. In aspects of the invention variants retain the immunogenic activity of the corresponding native-sequence polypeptide.

Percent identity of two amino acid sequences, or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

An allelic variant may also be created by introducing substitutions, additions, or deletions into a polynucleotide encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides comprising amino acid sequences sufficiently identical to or derived from the amino acid sequence of a native polypeptide which include fewer amino acids than the full length polypeptides. A portion of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally occurring allelic variant may contain conservative amino acid substitutions from the native polypeptide sequence or it may contain a substitution of an amino acid from a corresponding position in a polypeptide homolog, for example, a murine polypeptide.

Endometrial markers include chimeric or fusion proteins. A “chimeric protein” or “fusion protein” comprises all or part (preferably biologically active) of an endometrial marker operably linked to a heterologous polypeptide (i.e., a polypeptide other than an endometrial marker). Within the fusion protein, the term “operably linked” is intended to indicate that an endometrial marker and the heterologous polypeptide are fused in-frame to each other. The heterologous polypeptide can be fused to the N-terminus or C-terminus of an endometrial marker. A useful fusion protein is a GST fusion protein in which an endometrial marker is fused to the C-terminus of GST sequences. Another example of a fusion protein is an immunoglobulin fusion protein in which all or part of an endometrial marker is fused to sequences derived from a member of the immunoglobulin protein family. Chimeric and fusion proteins can be produced by standard recombinant DNA techniques.

A modified form of a polypeptide referenced herein includes modified forms of the polypeptides and derivatives of the polypeptides, including post-translationally modified forms such as glycosylated, phosphorylated, acetylated, methylated or lapidated forms of the polypeptides. For example, an N-terminal methionine may be cleaved from a polypeptide, and a new N-terminal residue may or may not be acetylated. In particular, for chaperonin 10 the first residue, methionine, can be cleaved and the second first residue, alanine can be N-acetylated.

Endometrial markers identified in accordance with a method of the invention may be prepared by recombinant or synthetic methods, or isolated from a variety of sources, or by any combination of these and similar techniques.

“Endometrial polynucleotide marker(s)”, polynucleotides encoding the marker(s)”, and “polynucleotides encoding endometrial markers” refer to polynucleotides that encode endometrial markers including native-sequence polypeptides, polypeptide variants including a portion of a polypeptide, an isoform, precursor, complex, a chimeric polypeptide, or modified forms and derivatives of the polypeptides. An endometrial polynucleotide marker includes the polynucleotides encoding the polypeptides listed in Table 1, 4, 5, or 6. In particular, the invention includes the polynucleotides encoding glutamate receptor subunit zeta 1 [e.g. see Accession No. D 13515 and SEQ ID NO. 27], a tryptic peptide thereof [SEQ ID NO. 28], macrophage migration inhibitory factor [e.g. see Accession Nos. NM_{—}002415 and L19686 and SEQ ID NOs. 19 and 20], FRAT1 [e.g. see Accession Nos. NM_{—}005479 and NM_{—}181355 and SEQ ID NOs. 43 and 44], myosin light chain kinase 2 [Accession No. AF069601 and SEQ ID NO. 46], tropomyosin 1 alpha chain e.g. see Accession No. NM_{—}000366 and BC007433 and SEQ ID NO. 48], and the endometrial markers listed in Table 1 [in particular, GenBank Accession Nos. NM_{—}002157 and U07550, A12027, NM_{—}002964, X06233, M21064, NM_{—}002644, NM_{—}002567, NM_{—}006305, NM_{—}002155, X51757, NM_{—}002415, L19686, NM_{—}005620 and D38583, NM_{—}000365, X69723, NM_{—}000295, K02212, NM_{—}001823, X15334, X56494, D84342, AF026126, and SEQ ID NOs. 2, 4, 5, 7, 8, 10, 12, 14, 16, 17, 19, 20, 22, 24, 25, 31, 32, 34, 35, 37, 39, 41]. In an embodiment, a polynucleotide of the invention encodes chaperonin 10, more particularly a polynucleotide sequence of GenBank Accession No. NM_{—}002157 and U07550 [SEQ ID NO 2], or a fragment thereof.

Endometrial polynucleotide markers include complementary nucleic acid sequences, and nucleic acids that are substantially identical to these sequences (e.g. having at least about 45%, 50%, 55%, 60%, 65%, 70%, 75%, 80%, 85%, 90%, 95%, 97%, 98%, or 99% sequence identity).

Endometrial polynucleotide markers also include sequences that differ from a native sequence [e.g. see GenBank Accession Nos. NM_{—}002157 and U07550, A12027, NM_{—}002964, X06233, M21064, NM_{—}002644, NM_{—}002567, NM_{—}006305, NM_{—}002155, X51757, NM_{—}002415, L19686, NM_{—}005620 and D38583, NM_{—}000365, X69723, D13515, NM_{—}000295, K02212, NM_{—}001823, X15334, X56494, D84342, AF026126, NM_{—}005479, NM_{—}181355, AF069601, NM_{—}000366, and BC007433, and SEQ ID NOs. 2, 4, 5, 7, 8, 10, 12, 14, 16, 17, 19, 20, 22, 24, 25, 27, 31, 32, 34, 35, 37, 39, 41, 43, 44, 46, and 48] due to degeneracy in the genetic code. As one example, DNA sequence polymorphisms within the nucleotide sequence of an endometrial marker may result in silent mutations that do not affect the amino acid sequence. Variations in one or more nucleotides may exist among individuals within a population due to natural allelic variation. DNA sequence polymorphisms may also occur which lead to changes in the amino acid sequence of a polypeptide.

Endometrial polynucleotide markers also include nucleic acids that hybridize under stringent conditions, preferably high stringency conditions to an endometrial polynucleotide marker, in particular an endometrial cancer polynucleotide marker [e.g. see GenBank Accession Nos. NM_{—}002157 and U07550, A12027, NM_{—}002964, X06233, M21064, NM_{—}002644, NM_{—}002567, NM_{—}006305, NM_{—}002155, X51757, NM_{—}002415, L19686, NM_{—}005620 and D38583, NM_{—}000365, X69723, D13515, NM_{—}000295, K02212, NM_{—}001823, X15334, X56494, D84342, AF026126, NM_{—}005479, NM_{—}181355, AF069601, NM_{—}000366, and BC007433, and SEQ ID NOs. 2, 4, 5, 7, 8, 10, 12, 14, 16, 17, 19, 20, 22, 24, 25, 27, 31, 32, 34, 35, 37, 39, 41, 43, 44, 46, and 48]. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

Endometrial polynucleotide markers also include truncated nucleic acids or nucleic acid fragments and variant forms of the nucleic acids that arise by alternative splicing of an mRNA corresponding to a DNA.

The endometrial polynucleotide markers are intended to include DNA and RNA (e.g. mRNA) and can be either double stranded or single stranded. A polynucleotide may, but need not, include additional coding or non-coding sequences, or it may, but need not, be linked to other molecules and/or carrier or support materials. The polynucleotides for use in the methods of the invention may be of any length suitable for a particular method. In certain applications the term refers to antisense polynucleotides (e.g. mRNA or DNA strand in the reverse orientation to sense cancer polynucleotide markers).

“Statistically different levels”, “significantly altered levels”, or “significant difference” in levels of markers in a patient sample compared to a control or standard (e.g. normal levels or levels in other samples from a patient) may represent levels that are higher or lower than the standard error of the detection assay. In particular embodiments, the levels may be 1.5, 2, 3, 4, 5, or 6 times higher or lower than the control or standard.

“Microarray” and “array,” refer to nucleic acid or nucleotide arrays or protein or peptide arrays that can be used to detect biomolecules associated with endometrium or phase thereof or endometrial disease, for instance to measure gene expression. A variety of arrays are made in research and manufacturing facilities worldwide, some of which are available commercially. By way of example, spotted arrays and in situ synthesized arrays are two kinds of nucleic acid arrays that differ in the manner in which the nucleic acid materials are placed onto the array substrate. A widely used in situ synthesized oligonucleotide array is GeneChip™ made by Affymetrix, Inc. Oligonucleotide probes that are 20- or 25-base long can be synthesized in silico on the array substrate. These arrays can achieve high densities (e.g., more than 40,000 genes per cm²). Generally spotted arrays have lower densities, but the probes, typically partial cDNA molecules, are much longer than 20- or 25-mers. Examples of spotted cDNA arrays include LifeArray made by Incyte Genomics and DermArray made by IntegriDerm (or Invitrogen). Pre-synthesized and amplified cDNA sequences are attached to the substrate of spotted arrays. Protein and peptide arrays also are known (see for example, Zhu et al., Science 293:2101 (2001).

“Binding agent” refers to a substance such as a polypeptide or antibody that specifically binds to one or more endometrial markers. A substance “specifically binds” to one or more endometrial markers if is reacts at a detectable level with one or more endometrial markers, and does not react detectably with peptides containing an unrelated or different sequence. Binding properties may be assessed using an ELISA, which may be readily performed by those skilled in the art (see for example, Newton et al, Develop. Dynamics 197: 1-13, 1993).

A binding agent may be a ribosome, with or without a peptide component, an aptamer, an RNA molecule, or a polypeptide. A binding agent may be a polypeptide that comprises one or more endometrial marker sequence, a peptide variant thereof, or a non-peptide mimetic of such a sequence. By way of example, a chaperonin 10 sequence may be a peptide portion of a chaperonin 10 that is capable of modulating a function mediated by chaperonin 10.

An aptamer includes a DNA or RNA molecule that binds to nucleic acids and proteins. An aptamer that binds to a protein (or binding domain) of an endometrial marker or an endometrial polynucleotide marker can be produced using conventional techniques, without undue experimentation. (For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)).

Antibodies for use in the present invention include but are not limited to monoclonal or polyclonal antibodies, immunologically active fragments (e.g. a Fab or (Fab)₂ fragments), antibody heavy chains, humanized antibodies, antibody light chains, genetically engineered single chain Fᵥ molecules (Ladner et al, U.S. Pat. No. 4,946,778), chimeric antibodies, for example, antibodies which contain the binding specificity of murine antibodies, but in which the remaining portions are of human origin, or derivatives, such as enzyme conjugates or labeled derivatives.

Antibodies including monoclonal and polyclonal antibodies, fragments and chimeras, may be prepared using methods known to those skilled in the art. Isolated native or recombinant endometrial markers may be utilized to prepare antibodies. (See, for example, Kohler et al. (1975) Nature 256:495-497; Kozbor et al. (1985) J. Immunol. Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies). Antibodies specific for an endometrial marker may also be obtained from scientific or commercial sources.

In an embodiment of the invention, antibodies are reactive against an endometrial marker if they bind with a Kₐ of greater than or equal to 10⁻⁷ M.

### Markers

The invention provides a set of markers correlated with endometrium or phase thereof, or endometrial disease. A subset of these markers identified as useful for detection, diagnosis, prevention and therapy of endometrial disease is listed in Table 1 or 5. A subset of these markers identified as useful for detection and diagnosis of endometrium phase is listed in Table 4 or 6. The invention also provides a method of using these markers to distinguish endometrium phase or to distinguish endometrial disease.

The invention provides marker sets that distinguish endometrium phase or endometrial disease and uses therefor. In an aspect, the invention provides a method for classifying an endometrium phase or endometrial disease comprising detecting a difference in the expression of a first plurality of endometrial markers or endometrial polynucleotide markers relative to a control, the first plurality of endometrial markers or endometrial polynucleotide markers consisting of at least 5 of the markers listed in Table 1, 4, 5, or 6. In specific aspects, the plurality of markers consists of at least 10 of the markers listed in Table 1, 4, 5, or 6. In another specific aspect, the control comprises markers derived from a pool of samples from individual patients with no endometrial disease, or individuals with a known endometrium phase.

Any of the markers provided herein may be used alone or with other markers of endometrium phase or endometrial disease, or with markers for other phenotypes or conditions.

### Identification of Endometrial Markers

The invention relates to a method for identifying markers associated with the endometrium or a phase thereof, or associated with a disease of the endometrium comprising:
(a) obtaining a sample of endometrium from a subject;
(b) extracting proteins from the sample and producing a profile of the proteins by subjecting the proteins to mass spectrometry; and
(c) comparing the profile with a profile for normal endometrial tissue or for a known endometrium phase to identify proteins associated with the endometrium phase or an endometrial disease.

Proteins may be extracted from the samples in a manner known in the art. For example, proteins may be extracted by first digesting or disrupting cell membranes by standard methods such as detergents or homogenization in an isotonic sucrose solution, followed by ultra-centrifugation or other standard techniques.

The separated proteins may be digested into peptides, in particular using proteolytic enzymes such as trypsin, pepsin, subtilisin, and proteinase. For example, proteins may be treated with trypsin which cleaves at the sites of lysine and arginine, to provide doubly-charged peptides with a length of from about 5 to 50 amino acids. Such peptides may be particularly appropriate for mass spectrometry analysis, especially electrospray ionization mass spectrometry. Chemical reagents including cyanogens bromide may also be utilized to digest proteins.

Mass spectrometers that may be used to analyze the peptides or proteins include a Matrix-Assisted Laser Desorptioon/Ioniation Time-of-Flight Mass Spectrometer (“MALDI-TOF”) (e.g. from PerSeptive Biosystems, Framingham, Mass.); an Electrospray Ionization (“ESI”) ion trap spectrometer, (e.g. from Finnigan MAT, San Jose, Calif.), an ESI quadrupole mass spectrometer (e.g. from Finnigan or Perkin-Elmer Corporation, Foster City, Calif.), a quadrupole/TOF hybrid tandem mass spectrometer, QSTAR XL (Applied Biosystems/MDS Sciex), or a Surface Enhanced Laser Desorption/Ionization (SELDI-TOF) Mass Spectrometer (e.g. from Ciphergen Biosystems Inc.).

### Detection Methods

A variety of methods can be employed for the diagnostic and prognostic evaluation of endometrial disease or endometrial status involving one or more endometrial markers and polynucleotides encoding the markers, and the identification of subjects with a predisposition to endometrial diseases or that are receptive to in vitro fertilization and embryo transfer procedures. Such methods may, for example, utilize endometrial polynucleotide markers, and fragments thereof, and binding agents (e.g. antibodies) against one or more endometrial markers, including peptide fragments. In particular, the polynucleotides and antibodies may be used, for example, for (1) the detection of the presence of endometrial polynucleotide marker mutations, or the detection of either over- or under-expression of endometrial marker mRNA relative to a non-disorder state or different endometrium phase, or the qualitative or quantitative detection of alternatively spliced forms of endometrial polynucleotide marker transcripts which may correlate with certain conditions or susceptibility toward such conditions; and (2) the detection of either an over- or an under-abundance of one or more endometrial markers relative to a non-disorder state or a different endometrium phase or the presence of a modified (e.g., less than full length) endometrial marker which correlates with a disorder state or a progression toward a disorder state, or a particular endometrium phase.

The invention contemplates a method for detecting the phase of an endometrial tissue, in particular a secretory endometrial tissue, comprising producing a profile of levels of one or more endometrial marker associated with a known endometrium phase and/or polynucleotides encoding the markers, and optionally other markers associated with the endometrium phase in cells from a patient, and comparing the profile with a reference to identify a profile for the test cells indicative of the endometrium phase. In an aspect, the endometrial markers are one or more of glutamate receptor subunit zeta 1, macrophage migration inhibitory factor, FRAT1, myosin light chain kinase 2, tropomyosin 1 alpha chain, and fragments thereof.

The invention also contemplates a method for detecting an endometrial disease, in particular an endometrial cancer, comprising producing a profile of levels of one or more endometrial marker associated with an endometrial disease and/or polynucleotides encoding the markers, and other markers associated with endometrial disease in cells from a patient, and comparing the profile with a reference to identify a profile for the test cells indicative of disease. In an aspect, the endometrial markers are one or more of chaperonin 10, calgranulin A, calgranulin B, polymeric-immunoglobulin receptor (precursor), phosphatidylethanolamine-binding protein, acidic leucine-rich nuclear phosphoprotein 32 family member A, heat shock 70 kDa protein 6, macrophage migration inhibitory factor, calgizzarin (S100C protein), triosephosphate isomerase, alpha-1-antitrypsin precursor, creatine kinase, B chain, (B-CK), pyruvate, M1 or M2 isozyme, transgelin (smooth muscle protein 22-alpha), and heterologous nuclear ribonucleoprotein D0.

The methods described herein may be used to evaluate the probability of the presence of malignant or pre-malignant cells, for example, in a group of cells freshly removed from a host. Such methods can be used to detect tumors, quantitate their growth, and help in the diagnosis and prognosis of endometrial disease. The methods can be used to detect the presence of cancer metastasis, as well as confirm the absence or removal of all tumor tissue following surgery, cancer chemotherapy, and/or radiation therapy. They can further be used to monitor cancer chemotherapy and tumor reappearance.

The methods described herein can be adapted for diagnosing and monitoring endometrial tissue status or an endometrial disease by detecting one or more endometrial markers or polynucleotides encoding the markers in biological samples from a subject. These applications require that the amount of markers or polynucleotides quantitated in a sample from a subject being tested be compared to a predetermined standard or cut-off value. The standard may correspond to levels quantitated for another sample or an earlier sample from the subject, or levels quantitated for a control sample. Levels for control samples from healthy subjects, different endometrial tissue phases, or subjects with an endometrial disease may be established by prospective and/or retrospective statistical studies. Healthy subjects who have no clinically evident disease or abnormalities may be selected for statistical studies. Diagnosis may be made by a finding of statistically different levels of detected endometrial markers associated with disease or polynucleotides encoding same, compared to a control sample or previous levels quantitated for the same subject.

The methods described herein may also use multiple markers for an endometrial disease, in particular endometrial cancer. Therefore, the invention contemplates a method for analyzing a biological sample for the presence of one or more endometrial markers and polynucleotides encoding the markers, and other markers that are specific indicators of an endometrial disease. The methods described herein may be modified by including reagents to detect the additional markers, or polynucleotides for the markers.

### Nucleic Acid Methods/Assays

As noted herein an endometrial disease or phase may be detected based on the level of endometrial polynucleotide markers in a sample. Techniques for detecting polynucleotides such as polymerase chain reaction (PCR) and hybridization assays are well known in the art.

Probes may be used in hybridization techniques to detect endometrial polynucleotide markers. The technique generally involves contacting and incubating nucleic acids (e.g. recombinant DNA molecules, cloned genes) obtained from a sample from a patient or other cellular source with a probe under conditions favorable for the specific annealing of the probes to complementary sequences in the nucleic acids. After incubation, the non-annealed nucleic acids are removed, and the presence of nucleic acids that have hybridized to the probe if any are detected.

Nucleotide probes for use in the detection of nucleic acid sequences in samples may be constructed using conventional methods known in the art. Suitable probes may be based on nucleic acid sequences encoding at least 5 sequential amino acids from regions of an endometrial polynucleotide marker, preferably they comprise 10-200, more particularly 10-30, 10-40, 20-50, 40-80, 50-150, 80-120 nucleotides in length.

The probes may comprise DNA or DNA mimics (e.g., derivatives and analogues) corresponding to a portion of an organism's genome, or complementary RNA or RNA mimics. Mimics are polymers comprising subunits capable of specific, Watson-Crick-like hybridization with DNA, or of specific hybridization with RNA. The nucleic acids can be modified at the base moiety, at the sugar moiety, or at the phosphate backbone.

DNA can be obtained using standard methods such as polymerase chain reaction (PCR) amplification of genomic DNA or cloned sequences. (See, for example, in innis et al., eds., 1990, PCR Protocols: A Guide to Methods and Applications, Academic Press Inc., San Diego, Calif.). Computer programs known in the art can be used to design primers with the required specificity and optimal amplification properties, such as Oligo version 5.0 (National Biosciences). Controlled robotic systems may be useful for isolating and amplifying nucleic acids.

A nucleotide probe may be labeled with a detectable substance such as a radioactive label that provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect endometrial polynucleotide markers, preferably in human cells. The nucleotide probes may also be useful in the diagnosis of an endometrial disease involving one or more endometrial polynucleotide markers, in monitoring the progression of such disorder, or monitoring a therapeutic treatment.

The detection of endometrial polynucleotide markers may involve the amplification of specific gene sequences using an amplification method such as polymerase chain reaction (PCR), followed by the analysis of the amplified molecules using techniques known to those skilled in the art. Suitable primers can be routinely designed by one of skill in the art.

By way of example, at least two oligonucleotide primers may be employed in a PCR based assay to amplify a portion of a polynucleotide encoding one or more endometrial marker derived from a sample, wherein at least one of the oligonucleotide primers is specific for (i.e. hybridizes to) a polynucleotide encoding the endometrial marker. The amplified cDNA is then separated and detected using techniques well known in the art, such as gel electrophoresis.

In order to maximize hybridization under assay conditions, primers and probes employed in the methods of the invention generally have at least about 60%, preferably at least about 75%, and more preferably at least about 90% identity to a portion of a polynucleotide encoding an endometrial marker; that is, they are at least 10 nucleotides, and preferably at least 20 nucleotides in length. In an embodiment the primers and probes are at least about 10-40 nucleotides in length.

Hybridization and amplification techniques described herein may be used to assay qualitative and quantitative aspects of endometrial polynucleotide marker expression. For example, RNA may be isolated from a cell type or tissue known to express an endometrial polynucleotide marker and tested utilizing the hybridization (e.g. standard Northern analyses) or PCR techniques referred to herein.

The primers and probes may be used in the above-described methods in situ i.e directly on tissue sections (fixed and/or frozen) of patient tissue obtained from biopsies or resections.

In an aspect of the invention, a method is provided employing reverse transcriptase-polymerase chain reaction (RT-PCR), in which PCR is applied in combination with reverse transcription. Generally, RNA is extracted from a sample tissue using standard techniques (for example, guanidine isothiocyanate extraction as described by Chomcynski and Sacchi, Anal. Biochem. 162:156-159, 1987) and is reverse transcribed to produce cDNA. The cDNA is used as a template for a polymerase chain reaction. The cDNA is hybridized to a set of primers, at least one of which is specifically designed against an endometrial marker sequence. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with a suspected endometrial disease and an individual who is not afflicted with an endometrial disease. The reaction may be performed on several dilutions of cDNA spanning at least two orders of magnitude. A statistically significant difference in expression in several dilutions of the subject sample as compared to the same dilutions of the non-disease sample may be considered positive for the presence of an endometrial disease.

In an embodiment, the invention provides methods for determining the presence or absence of an endometrial disease in a subject comprising (a) contacting a sample obtained from the subject with oligonucleotides that hybridize to endometrial polynucleotide markers; and (b) detecting in the sample a level of nucleic acids that hybridize to the polynucleotides relative to a predetermined cut-off value, and therefrom determining the presence or absence of an endometrial disease in the subject. In an aspect, the endometrial disease is cancer and the endometrial markers are one or more of chaperonin 10, calgranulin A, calgranulin B, polymeric-immunoglobulin receptor (precursor), phosphatidylethanolamine-binding protein, acidic leucine-rich nuclear phosphoprotein 32 family member A, heat shock 70 kDa protein 6, macrophage migration inhibitory factor, calgizzarin (S100C protein), triosephosphate isomerase, alpha-1-antitrypsin precursor, creatine kinase, B chain, (B-CK), pyruvate, M1 or M2 isozyme, transgelin (smooth muscle protein 22-alpha), and heterologous nuclear ribonucleoprotein D0.

In another embodiment, the invention provides methods for determining uterine receptivity of a subject to in vitro fertilization comprising (a) contacting a sample obtained from the subject with oligonucleotides that hybridize to endometrial polynucleotide markers associated with an endometrial tissue phase (e.g. secretory phase); and (b) detecting in the sample a level of nucleic acids that hybridize to the polynucleotides relative to a predetermined cut-off value, wherein the presence or absence of the endometrial marker polynucleotides as compared to non-receptive controls indicates uterine receptivity. In an aspect, the endometrial markers are one or more of glutamate receptor subunit zeta 1, macrophage migration inhibitory factor, FRAT1, myosin light chain kinase 2, tropomyosin 1 alpha chain, and fragments thereof.

The invention provides a method wherein an endometrial marker mRNA is detected by (a) isolating mRNA from a sample and combining the mRNA with reagents to convert it to cDNA; (b) treating the converted cDNA with amplification reaction reagents and nucleic acid primers that hybridize to one or more endometrial marker polynucleotides, to produce amplification products; (d) analyzing the amplification products to detect amounts of mRNA encoding endometrial polynucleotide markers; and (e) comparing the amount of mRNA to an amount detected against a panel of expected values for normal and malignant tissue derived using similar nucleic acid primers.

Endometrial cancer marker-positive samples or alternatively higher levels in patients compared to a control (e.g. non-cancerous tissue) may be indicative of late stage disease, and/or that the patient is not responsive to chemotherapy. Alternatively, negative samples or lower levels compared to a control (e.g. non-cancerous tissue or negative samples) may also be indicative of progressive disease and shorter overall survival.

In another embodiment, the invention provides methods for determining the presence or absence of endometrial cancer in a subject comprising (a) contacting a sample obtained from the subject with oligonucleotides that hybridize to one or more endometrial cancer polynucleotide markers; and (b) detecting in the sample levels of nucleic acids that hybridize to the polynucleotides relative to a predetermined cut-off value, and therefrom determining the presence or absence of endometrial cancer in the subject. In an embodiment, the endometrial cancer polynucleotide markers encode one or more polypeptides listed in Table 1. In particular, the endometrial markers are one or more of chaperonin 10, calgranulin A, calgranulin B, polymeric-immunoglobulin receptor (precursor), phosphatidylethanolamine-binding protein, acidic leucine-rich nuclear phosphoprotein 32 family member A, heat shock 70 kDa protein 6, macrophage migration inhibitory factor, calgizzarin (S100C protein), triosephosphate isomerase, alpha-1-antitrypsin precursor, creatine kinase, B chain, (B-CK), pyruvate, M1 or M2 isozyme, transgelin (smooth muscle protein 22-alpha), and heterologous nuclear ribonucleoprotein D0, and fragments thereof.

In particular, the invention provides a method wherein chaperonin 10 mRNA is detected by (a) isolating mRNA from a sample and combining the mRNA with reagents to convert it to cDNA; (b) treating the converted cDNA with amplification reaction reagents and nucleic acid primers that hybridize to a polynucleotide encoding chaperonin 10, to produce amplification products; (d) analyzing the amplification products to detect an amount of mRNA encoding chaperonin 10; and (e) comparing the amount of mRNA to an amount detected against a panel of expected values for normal and malignant tissue derived using similar nucleic acid primers.

Endometrial cancer marker-positive samples or alternatively higher levels, in particular significantly higher levels of chaperonin 10 polynucleotides in patients compared to a control (e.g. normal or benign) are indicative of endometrial cancer. Negative samples or lower levels compared to a control (e.g. normal or benign) may also be indicative of progressive disease and poor overall survival.

Oligonucleotides or longer fragments derived from an endometrial cancer polynucleotide marker may be used as targets in a microarray. The microarray can be used to simultaneously monitor the expression levels of large numbers of genes and to identify genetic variants, mutations, and polymorphisms. The information from the microarray may be used to determine gene function, to understand the genetic basis of a disorder, to diagnose a disorder, and to develop and monitor the activities of therapeutic agents.

The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (1995) PCT application WO95/35505; Heller, R. A. et al. (1997) Proc. Natl. Acad. Sci. 94:2150-2155; and Heller, M. J. et al. (1997) U.S. Pat. No. 5,605,662.)

Thus, the invention also includes an array comprising one or more endometrial polynucleotide markers (in particular the markers listed in Table 1, 4, 5, or 6) and optionally other markers. The array can be used to assay expression of endometrial polynucleotide markers in the array. The invention allows the quantitation of expression of one or more endometrial polynucleotide markers.

Microarrays typically contain at separate sites nanomolar quantities of individual genes, cDNAs, or ESTs on a substrate (e.g. nitrocellulose or silicon plate), or photolithographically prepared glass substrate. The arrays are hybridized to cDNA probes using conventional techniques with gene-specific primer mixes. The target polynucleotides to be analyzed are isolated, amplified and labeled, typically with fluorescent labels, radiolabels or phosphorous label probes. After hybridization is completed, the array is inserted into the scanner, where patterns of hybridization are detected. Data are collected as light emitted from the labels incorporated into the target, which becomes bound to the probe array. Probes that completely match the target generally produce stronger signals than those that have mismatches. The sequence and position of each probe on the array are known, and thus by complementarity, the identity of the target nucleic acid applied to the probe array can be determined.

Microarrays are prepared by selecting polynucleotide probes and immobilizing them to a solid support or surface. The probes may comprise DNA sequences, RNA sequences, copolymer sequences of DNA and RNA, DNA and/or RNA analogues, or combinations thereof. The probe sequences may be full or partial fragments of genomic DNA, or they may be synthetic oligonucleotide sequences synthesized either enzymatically in vivo, enzymatically in vitro (e.g., by PCR), or non-enzymatically in vitro.

The probe or probes used in the methods of the invention can be immobilized to a solid support or surface which may be either porous or non-porous. For example, the probes can be attached to a nitrocellulose or nylon membrane or filter covalently at either the 3′ or the 5′ end of the polynucleotide probe. The solid support may be a glass or plastic surface. In an aspect of the invention, hybridization levels are measured to microarrays of probes consisting of a solid support on the surface of which are immobilized a population of polynucleotides, such as a population of DNA or DNA mimics, or, alternatively, a population of RNA or RNA mimics. A solid support may be a nonporous or, optionally, a porous material such as a gel.

In accordance with embodiments of the invention, a microarray is provided comprising a support or surface with an ordered array of hybridization sites or “probes” each representing one of the markers described herein. The microarrays can be addressable arrays, and in particular positionally addressable arrays. Each probe of the array is typically located at a known, predetermined position on the solid support such that the identity of each probe can be determined from its position in the array. In preferred embodiments, each probe is covalently attached to the solid support at a single site.

Microarrays used in the present invention are preferably (a) reproducible, allowing multiple copies of a given array to be produced and easily compared with each other; (b) made from materials that are stable under hybridization conditions; (c) small, (e.g., between 1 cm² and 25 cm², between 12 cm² and 13 cm², or 3 cm²; and (d) comprise a unique set of binding sites that will specifically hybridize to the product of a single gene in a cell (e.g., to a specific mRNA, or to a specific cDNA derived therefrom). However, it will be appreciated that larger arrays may be used particularly in screening arrays, and other related or similar sequences will cross hybridize to a given binding site.

In accordance with an aspect of the invention, the microarray is an array in which each position represents one of the markers described herein. Each position of the array can comprise a DNA or DNA analogue based on genomic DNA to which a particular RNA or cDNA transcribed from a genetic marker can specifically hybridize. A DNA or DNA analogue can be a synthetic oligomer or a gene fragment. In an embodiment, probes representing each of the endometrial markers and endometrial polynucleotide markers is present on the array. In a preferred embodiment, the array comprises at least 5 of the endometrial polynucleotide markers.

Probes for the microarray can be synthesized using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 10 and about 500 bases, 20-100 bases, or 40-70 bases in length. Synthetic nucleic acid probes can include non-natural bases, such as, without limitation, inosine. Nucleic acid analogues such as peptide nucleic acid may be used as binding sites for hybridization. (see, e.g., Egholm et al., 1993, Nature 363:566-568; U.S. Pat. No. 5,539,083).

Probes can be selected using an algorithm that takes into account binding energies, base composition, sequence complexity, cross-hybridization binding energies, and secondary structure (see Friend et al., International Patent Publication WO 01/05935, published Jan. 25, 2001).

Positive control probes, (e.g., probes known to be complementary and hybridizae to sequences in the target polynucleotides), and negative control probes, (e.g., probes known to not be complementary and hybridize to sequences in the target polynucleotides) are typically included on the array. Positive controls can be synthesized along the perimeter of the array or synthesized in diagonal stripes across the array. A reverse complement for each probe can be next to the position of the probe to serve as a negative control.

The probes can be attached to a solid support or surface, which may be made from glass, plastic (e.g., polypropylene, nylon), polyacrylamide, nitrocellulose, gel, or other porous or nonporous material. The probes can be printed on surfaces such as glass plates (see Schena et al., 1995, Science 270:467-470). This method may be particularly useful for preparing microarrays of cDNA (See also, DeRisi et al., 1996, Nature Genetics 14:457-460; Shalon et al., 1996, Genome Res. 6:639-645; and Schena et al., 1995, Proc. Natl. Acad. Sci. U.S.A. 93:10539-11286).

High-density oligonucleotide arrays containing thousands of oligonucleotides complementary to defined sequences, at defined locations on a surface can be produced using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). Using these methods oligonucleotides (e.g., 60-mers) of known sequence are synthesized directly on a surface such as a derivatized glass slide. The array produced may be redundant, with several oligonucleotide molecules per RNA.

Microarrays can be made by other methods including masking (Maskos and Southern, 1992, Nuc. Acids. Res. 20:1679-1684). In an embodiment, microarrays of the present invention are preduced by synthesizing polynucleotide probes on a support wherein the nucleotide probes are attached to the support covalently at either the 3′ or the 5′ end of the polynucleotide.

The invention provides microarrays comprising a disclosed marker set. In one embodiment, the invention provides a microarray for distinguishing endometrial disease samples comprising a positionally-addressable array of polynucleotide probes bound to a support, the polynucleotide probes comprising a plurality of polynucleotide probes of different nucleotide sequences, each of the different nucleotide sequences comprising a sequence complementary and hybridizable to a plurality of genes, the plurality consisting of at least 2, 3, 4, 5, 6, 7, 8, 9, 10, 11, 12, 13, 14, or 15 of the genes corresponding to the markers listed in Table 1, 4, 5, or 6. An aspect of the invention provides microarrays comprising at least 5, 10, or 15 of the polynucleotides encoding the markers listed in Table 1, 4, 5, or 6.

The invention provides gene marker sets that distinguish endometrium phase or endometrial disease and uses therefor. In an aspect, the invention provides a method for classifying an endometrium phase or disease comprising detecting a difference in the expression of a first plurality of genes relative to a control, the first plurality of genes consisting of at least 5 of the genes encoding the markers listed in Table 1, 4, 5, or 6. In specific aspects, the plurality of genes consists of at least 10 or 15 of the genes encoding the markers listed in Table 1, 4, 5, or 6. In another specific aspect, the control comprises nucleic acids derived from a pool of samples from individual control patients.

The invention provides a method for classifying an endometrium phase or endometrial disease by calculating the similarity between the expression of at least 5 polynucleotides encoding markers listed in Table 1, 4, 5, or 6 in a sample to the expression of the same markers in a control pool, comprising the steps of:
(a) labeling nucleic acids derived from a sample, with a first fluorophore to obtain a first pool of fluorophore-labeled nucleic acids;
(b) labeling with a second fluorophore a first pool of nucleic acids derived from two or more endometrial disease samples, and a second pool of nucleic acids derived from two or more control samples;
(c) contacting the first fluorophore-labeled nucleic acid and the first pool of second fluorophore-labeled nucleic acid with a first microarray under conditions such that hybridization can occur, and contacting the first fluorophore-labeled nucleic acid and the second pool of second fluorophore-labeled nucleic acid with a second microarray under conditions such that hybridization can occur, detecting at each of a plurality of discrete loci on the first microarray a first fluorescent emission signal from the first fluorophore-labeled nucleic acid and a second fluorescent emission signal from the first pool of second fluorophore-labeled genetic matter that is bound to the first microarray and detecting at each of the marker loci on the second microarray the first fluorescent emission signal from the first fluorophore-labeled nucleic acid and a third fluorescent emission signal from the second pool of second fluorophore-labeled nucleic acid;
(d) determining the similarity of the sample to patient and control pools by comparing the first fluorescence emission signals and the second fluorescence emission signals, and the first emission signals and the third fluorescence emission signals; and
(e) classifying the sample as endometrial disease where the first fluorescence emission signals are more similar to the second fluorescence emission signals than to the third fluorescent emission signals, and classifying the sample as non-endometrial disease where the first fluorescence emission signals are more similar to the third fluorescence emission signals than to the second fluorescent emission signals, wherein the first microarray and the second microarray are similar to each other, exact replicas of each other, or are identical, and wherein the similarity is defined by a statistical method such that the cell sample and control are similar where the p value of the similarity is less than 0.01.

In aspects of the invention, the array can be used to monitor the time course of expression of one or more endometrial polynucleotide markers in the array. This can occur in various biological contexts such as tumor progression.

The array is also useful for ascertaining differential expression patterns of endometrial polynucleotide markers, and optionally other markers, in normal and abnormal cells. This may provide a battery of nucleic acids that could serve as molecular targets for diagnosis or therapeutic intervention.

### Protein Methods

Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of an endometrial disease (e.g. cancer) or an endometrium phase in a subject may be determined by (a) contacting a sample from the subject with a binding agent; (b) detecting in the sample a level of polypeptide that binds to the binding agent; and (c) comparing the level of polypeptide with a predetermined standard or cut-off value.

In particular embodiments of the invention, the binding agent is an antibody. Antibodies specifically reactive with one or more endometrial marker, or derivatives, such as enzyme conjugates or labeled derivatives, may be used to detect one or more endometrial marker in various samples (e.g. biological materials). They may be used as diagnostic or prognostic reagents and they may be used to detect abnormalities in the level of expression of one or more endometrial marker, or abnormalities in the structure, and/or temporal, tissue, cellular, or subcellular location of one or more endometrial marker. Antibodies may also be used to screen potentially therapeutic compounds in vitro to determine their effects on disorders (e.g. endometrial cancer) involving one or more endometrial markers, and other conditions. In vitro immunoassays may also be used to assess or monitor the efficacy of particular therapies.

In an aspect, the invention provides a method for monitoring or diagnosing an endometrial disease (e.g. cancer) in a subject by quantitating one or more endometrial markers in a biological sample from the subject comprising reacting the sample with antibodies specific for one or more endometrial markers, which are directly or indirectly labeled with detectable substances and detecting the detectable substances. In a particular embodiment of the invention, endometrial markers are quantitated or measured.

In an aspect of the invention, a method for detecting an endometrial disease (e.g. cancer) is provided comprising:
(a) obtaining a sample suspected of containing one or more endometrial markers associated with an endometrial disease;
(b) contacting said sample with antibodies that specifically bind to the endometrial markers under conditions effective to bind the antibodies and form complexes;
(c) measuring the amount of endometrial markers present in the sample by quantitating the amount of the complexes; and
(d) comparing the amount of endometrial markers present in the samples with the amount of endometrial markers in a control, wherein a change or significant difference in the amount of endometrial markers in the sample compared with the amount in the control is indicative of an endometrial disease.

In an embodiment, the invention contemplates a method for monitoring the progression of an endometrial disease (e.g. cancer) in an individual, comprising:
(a) contacting antibodies which bind to one or more endometrial markers with a sample from the individual so as to form complexes comprising the antibodies and one or more endometrial markers in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of disease, disease stage, and/or progression of the disease in said individual.

The amount of complexes may also be compared to a value representative of the amount of the complexes from an individual not at risk of, or afflicted with, an endometrial disease at different stages. A significant difference in complex formation may be indicative of advanced disease e.g. advanced endometrial cancer, or an unfavourable prognosis.

In aspects of the invention for diagnosis and monitoring of endometrial cancer, the endometrial markers are one or more of chaperonin 10, calgranulin A, calgranulin B, polymeric-immunoglobulin receptor (precursor), phosphatidylethanolamine-binding protein, acidic leucine-rich nuclear phosphoprotein 32 family member A, heat shock 70 kDa protein 6, macrophage migration inhibitory factor, calgizzarin (S100C protein), triosephosphate isomerase, alpha-1-antitrypsin precursor, creatine kinase, B chain, (B-CK), pyruvate, M1 or M2 isozyme, transgelin (smooth muscle protein 22-alpha), and heterologous nuclear ribonucleoprotein D0, or fragments thereof.

In embodiments of the methods of the invention, chaperonin 10 is detected in samples and higher levels, in particular significantly higher levels compared to a control (normal or benign) is indicative of endometrial cancer.

In aspects of the invention for characterizing endometrium phase the endometrial markers are one or more of glutamate receptor subunit zeta 1, macrophage migration inhibitory factor, FRAT1, myosin light chain kinase 2, tropomyosin 1 alpha chain, and fragments thereof.

In another embodiment, the invention provides methods for determining uterine receptivity of a subject to in vitro fertilization comprising (a) contacting a sample obtained from the subject with antibodies that bind to one or more endometrial marker associated with a certain endometrium phase (e.g. secretory phase); and (b) detecting in the sample a level of endometrial marker relative to a predetermined cut-off value, wherein the presence or absence of the endometrial marker as compared to non-receptive controls indicates uterine receptivity. In a particular embodiment, the marker is one or more of glutamate receptor subunit zeta 1, macrophage migration inhibitory factor, FRAT1, myosin light chain kinase 2, tropomyosin 1 alpha chain, and fragments thereof, more particularly glutamate receptor subunit zeta 1 or a fragment thereof, and/or macrophage migration inhibitory factor.

Antibodies may be used in any known immunoassays that rely on the binding interaction between antigenic determinants of one or more endometrial marker and the antibodies. Immunoassay procedures for in vitro detection of antigens in fluid samples are also well known in the art. [See for example, Paterson et al., Int. J. Can. 37:659 (1986) and Burchell et al., Int. J. Can. 34:763 (1984) for a general description of immunoassay procedures]. Qualitative and/or quantitative determinations of one or more endometrial marker in a sample may be accomplished by competitive or non-competitive immunoassay procedures in either a direct or indirect format. Detection of one or more endometrial marker using antibodies can be done utilizing immunoassays which are run in either the forward, reverse or simultaneous modes. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. These terms are well understood by those skilled in the art. A person skilled in the art will know, or can readily discern, other immunoassay formats without undue experimentation.

According to an embodiment of the invention, an immunoassay for detecting one or more endometrial markers in a biological sample comprises contacting binding agents that specifically bind to endometrial markers in the sample under conditions that allow the formation of first complexes comprising a binding agent and endometrial markers and determining the presence or amount of the complexes as a measure of the amount of endometrial markers contained in the sample. In a particular embodiment, the binding agents are labeled differently or are capable of binding to different labels.

Antibodies may be used to detect and quantify one or more endometrial markers in a sample in order to diagnose and treat pathological states. In particular, the antibodies may be used in immunohistochemical analyses, for example, at the cellular and sub-subcellular level, to detect one or more endometrial markers, to localize them to particular endometrial cells and tissues (e.g. tumor cells and tissues), and to specific subcellular locations, and to quantitate the level of expression.

Immunohistochemical methods for the detection of antigens in tissue samples are well known in the art. For example, immunohistochemical methods are described in Taylor, Arch. Pathol. Lab. Med. 102:112 (1978). Briefly, in the context of the present invention, a tissue sample obtained from a subject suspected of having an endometrial-related problem is contacted with antibodies, preferably monoclonal antibodies recognizing one or more endometrial markers. The site at which the antibodies are bound is determined by selective staining of the sample by standard immunohistochemical procedures. The same procedure may be repeated on the same sample using other antibodies that recognize one or more endometrial markers. Alternatively, a sample may be contacted with antibodies against one or more endometrial markers simultaneously, provided that the antibodies are labeled differently or are able to bind to a different label. The tissue sample may be normal endometrial tissue, a cancer tissue or a benign tissue.

An antibody microarray in which binding sites comprise immobilized, preferably monoclonal, antibodies specific to a substantial fraction of marker-derived endometrial markers of interest can be utilized in the present invention. Antibody arrays can be prepared using methods known in the art [(see for example, Zhu et al., Science 293:2101 (2001) and reference 20].

Antibodies specific for one or more endometrial marker may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

One of the ways an antibody can be detectably labeled is to link it directly to an enzyme. The enzyme when later exposed to its substrate will produce a product that can be detected. Examples of detectable substances that are enzymes are horseradish peroxidase, beta-galactosidase, luciferase, alkaline phosphatase, acetylcholinesterase, malate dehydrogenase, ribonuclease, urease, catalase, glucose-6-phosphate, staphylococcal nuclease, delta-5-steriod isomerase, yeast alcohol dehydrogenase, alpha-glycerophosphate, triose phosphate isomerase, asparaginase, glucose oxidase, and acetylcholine esterase.

For increased sensitivity in an immunoassay system a fluorescence-emitting metal atom such as Eu (europium) and other lanthanides can be used. These can be attached to the desired molecule by means of metal-chelating groups such as DTPA or EDTA.

A bioluminescent compound may also be used as a detectable substance. Bioluminescence is a type of chemiluminescence found in biological systems where a catalytic protein increases the efficiency of the chemiluminescent reaction. The presence of a bioluminescent molecule is determined by detecting the presence of luminescence. Examples of bioluminescent detectable substances are luciferin, luciferase and aequorin.

Indirect methods may also be employed in which the primary antigen-antibody reaction is amplified by the introduction of a second antibody, having specificity for the antibody reactive against one or more endometrial markers. By way of example, if the antibody having specificity against one or more endometrial markers is a rabbit IgG antibody, the second antibody may be goat anti-rabbit gamma-globulin labelled with a detectable substance as described herein.

Methods for conjugating or labelling the antibodies discussed above may be readily accomplished by one of ordinary skill in the art. (See for example Inman, Methods In Enzymology, Vol. 34, Affinity Techniques, Enzyme Purification Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988 re methods for conjugating or labelling the antibodies with enzyme or ligand binding partner).

Cytochemical techniques known in the art for localizing antigens using light and electron microscopy may be used to detect one or more endometrial markers. Generally, antibodies may be labeled with detectable substances and one or more endometrial markers may be localised in tissues and cells based upon the presence of the detectable substances.

In the context of the methods of the invention, the sample, binding agents (e.g. antibodies specific for one or more endometrial markers), or one or more endometrial markers may be immobilized on a carrier or support. Examples of suitable carriers or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well, beads, disc, sphere, etc. The immobilized antibody may be prepared by reacting the material with a suitable insoluble carrier using known chemical or physical methods, for example, cyanogen bromide coupling. An antibody may be indirectly immobilized using a second antibody specific for the antibody. For example, mouse antibody specific for an endometrial marker may be immobilized using sheep anti-mouse IgG Fc fragment specific antibody coated on the carrier or support.

Where a radioactive label is used as a detectable substance, one or more endometrial marker may be localized by radioautography. The results of radioautography may be quantitated by determining the density of particles in the radioautographs by various optical methods, or by counting the grains.

Time-resolved fluorometry may be used to detect a signal. For example, the method described in Christopoulos T K and Diamandis E P Anal Chem 1992:64:342-346 may be used with a conventional time-resolved fluorometer.

In accordance with an embodiment of the invention, a method is provided wherein one or more endometrial marker antibodies are directly or indirectly labelled with enzymes, substrates for the enzymes are added wherein the substrates are selected so that the substrates, or a reaction product of an enzyme and substrate, form fluorescent complexes with a lanthanide metal (e.g. europium, terbium, samarium, and dysprosium, preferably europium and terbium). A lanthanide metal is added and one or more endometrial cancer markers are quantitated in the sample by measuring fluorescence of the fluorescent complexes. Enzymes are selected based on the ability of a substrate of the enzyme, or a reaction product of the enzyme and substrate, to complex with lanthanide metals such as europium and terbium. Suitable enzymes and substrates that provide fluorescent complexes are described in U.S. Pat. No. 5,3112,922 to Diamandis. Examples of suitable enzymes include alkaline phosphatase and β-galactosidase. Preferably, the enzyme is alkaline phosphatase.

Examples of enzymes and substrates for enzymes that provide such fluorescent complexes are described in U.S. Pat. No. 5,312,922 to Diamandis. By way of example, when the antibody is directly or indirectly labelled with alkaline phosphatase the substrate employed in the method may be 4-methylumbelliferyl phosphate, 5-fluorosalicyl phosphate, or diflunisal phosphate. The fluorescence intensity of the complexes is typically measured using a time-resolved fluorometer e.g. a CyberFluor 615 Immunoanalyzer (Nordion International, Kanata, Ontario).

One or more endometrial marker antibodies may also be indirectly labelled with an enzyme. For example, the antibodies may be conjugated to one partner of a ligand binding pair, and the enzyme may be coupled to the other partner of the ligand binding pair. Representative examples include avidin-biotin, and riboflavin-riboflavin binding protein. In an embodiment, the antibodies are biotinylated; and the enzyme is coupled to streptavidin. In another embodiment, an antibody specific for endometrial marker antibody is labeled with an enzyme.

In accordance with an embodiment, the present invention provides means for determining one or more endometrial markers in a sample by measuring one or more endometrial markers by immunoassay. It will be evident to a skilled artisan that a variety of immunoassay methods can be used to measure one or more endometrial markers. In general, an immunoassay method may be competitive or noncompetitive. Competitive methods typically employ an immobilized or immobilizable antibody to one or more endometrial marker and a labeled form of one or more endometrial marker. Sample endometrial markers and labeled endometrial markers compete for binding to antibodies to endometrial markers. After separation of the resulting labeled endometrial markers that have become bound to antibodies (bound fraction) from that which has remained unbound (unbound fraction), the amount of the label in either bound or unbound fraction is measured and may be correlated with the amount of endometrial markers in the test sample in any conventional manner, e.g., by comparison to a standard curve.

In an aspect, a non-competitive method is used for the determination of one or more endometrial markers, with the most common method being the “sandwich” method. In this assay, two antibodies to endometrial markers are employed. One of the antibodies to endometrial markers is directly or indirectly labeled (sometimes referred to as the “detection antibody”) and the other is immobilized or immobilizable (sometimes referred to as the “capture antibody”). The capture and detection antibodies can be contacted simultaneously or sequentially with the test sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally it is measured in the capture antibody phase since it comprises endometrial cancer markers bound by (“sandwiched” between) the capture and detection antibodies. In an embodiment, the label may be measured without separating the capture antibodies and liquid test mixture.

In a typical two-site immunometric assay for endometrial markers, one or both of the capture and detection antibodies are polyclonal antibodies or one or both of the capture and detection antibodies are monoclonal antibodies (i.e. polyclonal/polyclonal, monoclonal/monoclonal, or monoclonal/polyclonal). The label used in the detection antibody can be selected from any of those known conventionally in the art. The label may be an enzyme or a chemiluminescent moiety, but it can also be a radioactive isotope, a fluorophor, a detectable ligand (e.g., detectable by a secondary binding by a labeled binding partner for the ligand), and the like. In a particular aspect, the antibody is labelled with an enzyme which is detected by adding a substrate that is selected so that a reaction product of the enzyme and substrate forms fluorescent complexes. The capture antibody may be selected so that it provides a means for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody may comprise an antibody covalently or noncovalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, the capture antibody may be immobilized using a species specific antibody for the capture antibody that is bound to the solid phase.

The above-described immunoassay methods and formats are intended to be exemplary and are not limiting.

### Computer Systems

Analytic methods contemplated herein can be implemented by use of computer systems and methods described below and known in the art. Thus, the invention provides computer readable media comprising one or more endometrial markers, and/or polynucleotides encoding one or more endometrial markers, and optionally other markers (e.g. markers of endometrial cancer). “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on one or more endometrial markers, and optionally other markers.

A variety of data processor programs and formats can be used to store information on one or more endometrial markers, and/or polynucleotides encoding one or more endometrial markers, and other markers on computer readable medium. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of data processor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining uterine endometrial receptivity of a patient, or whether a patient has an endometrial disease (e.g. endometrial cancer) or a pre-disposition to an endometrial disease (e.g. cancer), comprising determining the presence or absence of one or more endometrial markers, and/or polynucleotides encoding one or more endometrial markers, and optionally other markers, and based on the presence or absence of the one or more endometrial markers, and/or polynucleotides encoding one or more endometrial markers, and optionally other markers, determining uterine endometrial receptivity, endometrial disease (e.g. cancer) or a pre-disposition to an endometrial disease (e.g. cancer), and optionally recommending a procedure or treatment.

The invention also provides in an electronic system and/or in a network, a method for determining uterine endometrial receptivity of a patient, whether a subject has an endometrial disease (e.g. cancer) or a pre-disposition to an endometrial disease (e.g. cancer), comprising determining the presence or absence of one or more endometrial markers, and/or polynucleotides encoding one or more endometrial markers, and optionally other markers (e.g. cancer markers), and based on the presence or absence of the one or more endometrial markers, and/or polynucleotides encoding one or more endometrial markers, and optionally other markers, determining the uterine endometrial receptivity of the patient, whether the subject has an endometrial disease (e.g. cancer) or a pre-disposition to an endometrial disease (e.g. cancer), and optionally recommending a procedure or treatment.

The invention further provides in a network, a method for determining whether a subject is receptive to in vitro fertilization, has an endometrial disease (e.g. cancer) or a pre-disposition to an endometrial disease (e.g. cancer) comprising: (a) receiving phenotypic information on the subject and information on one or more endometrial markers, and/or polynucleotides encoding one or more endometrial markers, and optionally other markers associated with samples from the subject; (b) acquiring information from the network corresponding to the one or more endometrial markers, and/or polynucleotides encoding one or more endometrial markers, and optionally other markers; and (c) based on the phenotypic information and information on the one or more endometrial markers, and/or polynucleotides encoding one or more endometrial markers, and optionally other markers, determining whether the subject is receptive to in vitro fertilization, has an endometrial disease (e.g. cancer) or a pre-disposition to an endometrial disease (e.g. cancer); and (d) optionally recommending a procedure or treatment.

The invention still further provides a system for identifying selected records that identify a diseased endometrial cell or tissue (e.g. cancer cell or tissue) or an endometrium phase. A system of the invention generally comprises a digital computer; a database server coupled to the computer; a database coupled to the database server having data stored therein, the data comprising records of data comprising one or more endometrial markers, and/or polynucleotides encoding one or more endometrial markers, and optionally other endometrial markers, and a code mechanism for applying queries based upon a desired selection criteria to the data file in the database to produce reports of records which match the desired selection criteria.

In an aspect of the invention a method is provided for detecting endometrial cancer tissue or cells using a computer having a processor, memory, display, and input/output devices, the method comprising the steps of:
(a) creating records of one or more endometrial cancer markers, and/or polynucleotides encoding one or more endometrial cancer markets, and optionally other markers of cancer identified in a sample suspected of containing endometrial cancer cells or tissue;
(b) providing a database comprising records of data comprising one or more endometrial cancer markers, and/or polynucleotides encoding one or more endometrial cancer markers, and optionally other markers of cancer; and
(c) using a code mechanism for applying queries based upon a desired selection criteria to the data file in the database to produce reports of records of step (a) which provide a match of the desired selection criteria of the database of step (b) the presence of a match being a positive indication that the markers of step (a) have been isolated from cells or tissue that are endometrial cancer cells or tissue.

The invention contemplates a business method for determining whether a subject is receptive to in vitro fertilization, has an endometrial disease (e.g. cancer) or a pre-disposition to endometrial cancer comprising: (a) receiving phenotypic information on the subject and information on one or more endometrial markers, and/or polynucleotides encoding the markers, and optionally other markers, associated with samples from the subject; (b) acquiring information from a network corresponding to one or more endometrial markers, and/or polynucleotides encoding the markers, and optionally other markers; and (c) based on the phenotypic information, information on one or more endometrial markers, and/or polynucleotides encoding the markers, and optionally other markers, and acquired information, determining whether the subject is receptive to in vitro fertilization, has an endometrial disease (e.g. cancer) or a pre-disposition to an endometrial disease (e.g. cancer); and (d) optionally recommending a procedure or treatment.

In an aspect of the invention, the computer systems, components, and methods described herein are used to monitor disease or determine the stage of disease, or determine uterine endometrial receptivity.

### Imaging Methods

Binding agents, in particular antibodies, specific for one or more endometrial markers may also be used in imaging methodologies in the management of an endometrial disease or determining uterine endometrial receptivity.

In an aspect, the invention provides a method for imaging tumors associated with one or more endometrial cancer markers.

The invention also contemplates imaging methods described herein using multiple markers for an endometrial disease or endometrium phase. Preferably each agent is labeled so that it can be distinguished during the imaging.

In an embodiment the method is an in vivo method and a subject or patient is administered one or more agents that carry an imaging label and that are capable of targeting or binding to one or more endometrial markers. The agent is allowed to incubate in vivo and bind to the endometrial markers associated with endometrial cells or tissues of a particular phase or associated with diseased cells or tissues, (e.g. an endometrial tumor). The presence of the label is localized to the endometrial cells or tissues, and the localized label is detected using imaging devices known to those skilled in the art.

The agent may be an antibody or chemical entity that recognizes the endometrial markers. In an aspect of the invention the agent is a polyclonal antibody or monoclonal antibody, or fragments thereof, or constructs thereof including but not limited to, single chain antibodies, bifunctional antibodies, molecular recognition units, and peptides or entities that mimic peptides. The antibodies specific for the endometrial markers used in the methods of the invention may be obtained from scientific or commercial sources, or isolated native endometrial markers or recombinant endometrial markers may be utilized to prepare antibodies etc. as described herein.

An agent may be a peptide that mimics the epitope for an antibody specific for an endometrial marker and binds to the marker. The peptide may be produced on a commercial synthesizer using conventional solid phase chemistry. By way of example, a peptide may be prepared that includes either tyrosine, lysine, or phenylalanine to which N₂S₂ chelate is complexed (See U.S. Pat. No. 4,897,255). An anti-endocrine marker peptide conjugate is then combined with a radiolabel (e.g. sodium ^{99m}Tc pertechnetate or sodium ¹⁸⁵Re perrhenate) and it may be used to locate an endometrial marker producing cell or tissue (e.g. tumor).

The agent carries a label to image the endometrial markers. The agent may be labelled for use in radionuclide imaging. In particular, the agent may be directly or indirectly labelled with a radioisotope. Examples of radioisotopes that may be used in the present invention are the following: ²⁷⁷Ac, ²¹¹At, ¹²⁸Ba, ¹³¹Ba, ⁷Be, ²⁰⁴Bi, ²⁰⁵Bi, ²⁰⁶Bi, ⁷⁶Br, ⁷⁷Br, ⁸²Br, ¹⁰⁹Cd, ⁴⁷Ca, ¹¹C, ¹⁴C, ³⁶Cl, ⁴⁸Cr, ⁵¹Cr, ⁶²Cu, ⁶⁴Cu, ⁶⁷Cu, ¹⁶⁵Dy, ¹⁵⁵Eu, ¹⁸F, ¹⁵³Gd, ⁶⁶Ga, ⁶⁷Ga, ⁶⁸Ga, ⁷²Ga, ¹⁹⁸Au, ³H, ¹⁶⁶Ho, ¹¹¹In, ^{113m}In, ^{115m}In, ¹²³I, ¹²⁵I, ¹³¹I, ¹⁸⁹Ir, ^{191m}Ir, ¹⁹²Ir, ¹⁹⁴Ir, ⁵²Fe, ⁵⁵ Fe, ⁵⁹Fe, ¹⁷⁷Lu, ¹⁵O, ^{191m-191}Os, ¹⁰⁹Pd, ³²P, ³³P, ⁴²K, ²²⁶Ra, ¹⁸⁶Re, ¹⁸⁸Re, ^{82m}Rb, ¹⁵³Sm, ⁴⁶Sc, ⁴⁷Sc, ⁷²Se, ⁷⁵Se, ¹⁰⁵Ag, ²²Na, ²⁴Na, ⁸⁹Sr, ³⁵S, ³⁸S, ¹⁷⁷Ta, ⁹⁶Tc, ^{99m}Tc, ²⁰¹Tl, ²⁰²Tl, ¹¹³Sn, ^{117m}Sn, ¹²¹Sn, ¹⁶⁶Yb, ¹⁶⁹Yb, ¹⁷⁵Yb, ⁸⁸Y, ⁹⁰Y, ⁶²Zn and ⁶⁵Zn. Preferably the radioisotope is ¹³¹I, ¹²⁵I, ¹²³I, ¹¹¹I, ^{99m}Tc, ⁹⁰Y, ¹⁸⁶Re, ¹⁸⁸Re, ³²P, ¹⁵³Sm, ⁶⁷Ga, ²⁰¹Tl, ⁷⁷Br, or ¹⁸F, and is imaged with a photoscanning device.

Procedures for labeling biological agents with the radioactive isotopes are generally known in the art. U.S. Pat. No. 4,302,438 describes tritium labeling procedures. Procedures for iodinating, tritium labeling, and ³⁵S labeling especially adapted for murine monoclonal antibodies are described by Goding, J. W. (supra, pp 124-126) and the references cited therein. Other procedures for iodinating biological agents, such as antibodies, binding portions thereof, probes, or ligands, are described in the scientific literature (see Hunter and Greenwood, Nature 144:945 (1962), David et al., Biochemistry 13:1014-1021 (1974), and U.S. Pat. Nos. 3,867,517 and 4,376,110). Iodinating procedures for agents are described by Greenwood, F. et al., Biochem. J. 89:114-123 (1963); Marchalonis, J., Biochem. J. 113:299-305 (1969); and Morrison, M. et al., Immunochemistry, 289-297 (1971). ^{99m}Tc-labeling procedures are described by Rhodes, B. et al. in Burchiel, S. et al. (eds.), Tumor Imaging: The Radioimmunochemical Detection of Cancer, New York: Masson 111-123 (1982) and the references cited therein. Labelling of antibodies or fragments with technetium-99m are also described for example in U.S. Pat. No. 5,317,091, U.S. Pat. No. 4,478,815, U.S. Pat. No. 4,478,818, U.S. Pat. No. 4,472,371, U.S. Pat. No. Re 32,417, and U.S. Pat. No. 4,311,688. Procedures suitable for ¹¹¹In-labeling biological agents are described by Hnatowich, D. J. et al., J. Immul. Methods, 65:147-157 (1983), Hnatowich, D. et al., J. Applied Radiation, 35:554-557 (1984), and Buckley, R. G. et al., F.E.B.S. 166:202-204 (1984).

An agent may also be labeled with a paramagnetic isotope for purposes of an in vivo method of the invention. Examples of elements that are useful in magnetic resonance imaging include gadolinium, terbium, tin, iron, or isotopes thereof. (See, for example, Schaefer et al., (1989) JACC 14, 472-480; Shreve et al., (1986) Magn. Reson. Med. 3, 336-340; Wolf, G L., (1984) Physiol. Chem. Phys. Med. NMR 16, 93-95; Wesbey et al., (1984) Physiol. Chem. Phys. Med. NMR 16, 145-155; Runge et al., (1984) Invest. Radiol. 19, 408-415 for discussions on in vivo nuclear magnetic resonance imaging.)

In the case of a radiolabeled agent, the agent may be administered to the patient, it is localized to the cell or tissue (e.g. tumor) having an endometrial marker with which the agent binds, and is detected or “imaged” in vivo using known techniques such as radionuclear scanning using e.g., a gamma camera or emission tomography. [See for example, A. R. Bradwell et al., “Developments in Antibody Imaging”, Monoclonal Antibodies for Cancer Detection and Therapy, R. W. Baldwin et al., (eds.), pp. 65-85 (Academic Press 1985)]. A positron emission transaxial tomography scanner, such as designated Pet VI located at Brookhaven National Laboratory, can also be used where the radiolabel emits positrons (e.g., ¹¹C, ¹⁸F, ¹⁵O, and ¹³N).

Whole body imaging techniques using radioisotope labeled agents can be used for locating diseased cells and tissues (e.g. primary tumors and tumors which have metastasized). Antibodies specific for endometrial markers, or fragments thereof having the same epitope specificity, are bound to a suitable radioisotope, or a combination thereof, and administered parenterally. For endometrial cancer, administration preferably is intravenous. The bio-distribution of the label can be monitored by scintigraphy, and accumulations of the label are related to the presence of endometrial cancer cells. Whole body imaging techniques are described in U.S. Pat. Nos. 4,036,945 and 4,311,688. Other examples of agents useful for diagnosis and therapeutic use that can be coupled to antibodies and antibody fragments include metallothionein and fragments (see, U.S. Pat. No. 4,732,864). These agents are useful in diagnosis staging and visualization of cancer, in particular endometrial cancer, so that surgical and/or radiation treatment protocols can be used more efficiently.

An imaging agent may carry a bioluminescent or chemiluminescent label. Such labels include polypeptides known to be fluorescent, bioluminescent or chemiluminescent, or, that act as enzymes on a specific substrate (reagent), or can generate a fluorescent, bioluminescent or chemiluminescent molecule. Examples of bioluminescent or chemiluminescent labels include luciferases, aequorin, obelin, mnemiopsin, berovin, a phenanthridinium ester, and variations thereof and combinations thereof. A substrate for the bioluminescent or chemiluminescent polypeptide may also be utilized in a method of the invention. For example, the chemiluminescent polypeptide can be luciferase and the reagent luciferin. A substrate for a bioluminescent or chemiluminescent label can be administered before, at the same time (e.g., in the same formulation), or after administration of the agent.

An imaging agent may comprise a paramagnetic compound, such as a polypeptide chelated to a metal, e.g., a metalloporphyrin. The paramagnetic compound may also comprise a monocrystalline nanoparticle, e.g., a nanoparticle comprising a lanthanide (e.g., Gd) or iron oxide; or, a metal ion comprising a lanthanide. “Lanthanides” refers to elements of atomic numbers 58 to 70, a transition metal of atomic numbers 21 to 29, 42 or 44, a Gd(III), a Mn(II), or an element comprising a Fe element. Paramagnetic compounds can also comprise a neodymium iron oxide (NdFeO₃) or a dysprosium iron oxide (DyFeO₃). Examples of elements that are useful in magnetic resonance imaging include gadolinium, terbium, tin, iron, or isotopes thereof. (See, for example, Schaefer et al., (1989) JACC 14, 472-480; Shreve et al., (1986) Magn. Reson. Med. 3, 336-340; Wolf, G L., (1984) Physiol. Chem. Phys. Med. NMR 16, 93-95; Wesbey et al., (1984) Physiol. Chem. Phys. Med. NMR 16, 145-155; Runge et al., (1984) Invest. Radiol. 19, 408-415 for discussions on in vivo nuclear magnetic resonance imaging.)

An image can be generated in a method of the invention by computer assisted tomography (CAT), magnetic resonance spectroscopy (MRS) image, magnetic resonance imaging (MRI), positron emission tomography (PET), single-photon emission computed tomography (SPECT), or bioluminescence imaging (BLI) or equivalent.

Computer assisted tomography (CAT) and computerized axial tomography (CAT) systems and devices well known in the art can be utilized in the practice of the present invention. (See, for example, U.S. Pat. Nos. 6,151,377; 5,946,371; 5,446,799; 5,406,479; 5,208,581; 5,109,397). The invention may also utilize animal imaging modalities, such as MicroCAT™ (ImTek, Inc.).

Magnetic resonance imaging (MRI) systems and devices well known in the art can be utilized in the practice of the present invention. In magnetic resonance methods and devices, a static magnetic field is applied to a tissue or a body in order to define an equilibrium axis of magnetic alignment in a region of interest. A radio frequency field is then applied to the region in a direction orthogonal to the static magnetic field direction to excite magnetic resonance in the region. The resulting radio frequency signals are then detected and processed, and the exciting radio frequency field is applied. The resulting signals are detected by radio-frequency coils that are placed adjacent to the tissue or area of the body of interest. (For a description of MRI methods and devices see, for example, U.S. Pat. Nos. 6,151,377; 6,144,202; 6,128,522; 6,127,825; 6,121,775; 6,119,032; 6,115,446; 6,111,410; 602,891; 5,555,251; 5,455,512; 5,450,010; 5,378,987; 5,214,382; 5,031,624; 5,207,222; 4,985,678; 4,906,931; 4,558,279). MRI and supporting devices are commercially available for example, from Bruker Medical GMBH; Caprius; Esaote Biomedica; Fonar; GE Medical Systems (GEMS); Hitachi Medical Systems America; Intermagnetics General Corporation; Lunar Corp.; MagneVu; Marconi Medicals; Philips Medical Systems; Shimadzu; Siemens; Toshiba America Medical Systems; including imaging systems, by, e.g., Silicon Graphics. The invention may also utilize animal imaging modalities such as micro-MRIs.

Positron emission tomography imaging (PET) systems and devices well known in the art can be utilized in the practice of the present invention. For example, a method of the invention may use the system designated Pet VI located at Brookhaven National Laboratory. For descriptions of PET systems and devices see, for example, U.S. Pat. Nos. 6,151,377; 6,072,177; 5,900,636; 5,608,221; 5,532,489; 5,272,343; 5,103,098. Animal imaging modalities such as micro-PETs (Corcorde Microsystems, Inc.) can also be used in the invention.

Single-photon emission computed tomography (SPECT) systems and devices well known in the art can be utilized in the practice of the present invention. (See, for example, U.S. Pat. Nos. 6,115,446; 6,072,177; 5,608,221; 5,600,145; 5,210,421; 5,103,098.) The methods of the invention may also utilize animal imaging modalities, such as micro-SPECTs.

Bioluminescence imaging includes bioluminescence, fluorescence or chemiluminescence or other photon detection systems and devices that are capable of detecting bioluminescence, fluorescence or chemiluminescence. Sensitive photon detection systems can be used to detect bioluminescent and fluorescent proteins externally; see, for example, Contag (2000) Neoplasia 2:41-52; Zhang (1994) Clin. Exp. Metastasis 12:87-92. The methods of the invention can be practiced using any such photon detection device, or variation or equivalent thereof, or in conjunction with any known photon detection methodology, including visual imaging. By way of example, an intensified charge-coupled device (ICCD) camera coupled to an image processor may be used in the present invention. (See, e.g., U.S. Pat. No. 5,650,135). Photon detection devices are also commercially available from Xenogen, Hamamatsue.

### Screening Methods

The invention also contemplates methods for evaluating test agents or compounds for their ability to inhibit an endometrial disease (e.g. cancer), potentially contribute to an endometrial disease (e.g. cancer), or inhibit or enhance an endometrium phase. Test agents and compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The agents or compounds may be endogenous physiological compounds or natural or synthetic compounds.

The invention provides a method for assessing the potential efficacy of a test agent for inhibiting an endometrial disease (e.g. cancer) in a patient, the method comprising comparing:
(a) levels of one or more endometrial markers, and/or polynucleotides encoding endometrial markers, and optionally other markers in a first sample obtained from a patient and exposed to the test agent; and
(b) levels of one or more endometrial markers and/or polynucleotides encoding endometrial markers, and optionally other markers, in a second sample obtained from the patient, wherein the sample is not exposed to the test agent, wherein a significant difference in the levels of expression of one or more endometrial markers, and/or polynucleotides encoding one or more endometrial markers, and optionally the other markers, in the first sample, relative to the second sample, is an indication that the test agent is potentially efficacious for inhibiting an endometrial disease (e.g. cancer) in the patient.

The first and second samples may be portions of a single sample obtained from a patient or portions of pooled samples obtained from a patient.

In an aspect, the invention provides a method of selecting an agent for inhibiting an endometrial disease (e.g. cancer) in a patient comprising:
(a) obtaining a sample from the patient;
(b) separately maintaining aliquots of the sample in the presence of a plurality of test agents;
(c) comparing one or more endometrial markers, and/or polynucleotides encoding endometrial markers, and optionally other markers, in each of the aliquots; and
(d) selecting one of the test agents which alters the levels of one or more endometrial markers, and/or polynucleotides encoding endometrial markers, and optionally other markers in the aliquot containing that test agent, relative to other test agents.

In a further aspect, the invention provides a method of selecting an agent for inhibiting or enhancing an endometrium phase in a patient comprising:
(a) obtaining a sample of endometrium in a selected phase (e.g. secretory or proliferative phase);
(b) separately maintaining aliquots of the sample in the presence of a plurality of test agents;
(c) comparing one or more endometrial markers, and/or polynucleotides encoding endometrial markers, and optionally other markers, in each of the aliquots; and
(d) selecting one of the test agents which alters the levels of one or more endometrial markers, and/or polynucleotides encoding endometrial markers, and optionally other markers in the aliquot containing that test agent, relative to other test agents.

Still another aspect of the present invention provides a method of conducting a drug discovery business comprising:
(a) providing one or more methods or assay systems for identifying agents that inhibit an endometrial disease (e.g. endometrial cancer) or affect an endometrium phase in a patient;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The invention also contemplates a method of assessing the potential of a test compound to contribute to an endometrial disease (e.g. endometrial cancer) comprising:
(a) maintaining separate aliquots of cells or tissues from a patient with an endometrial disease (e.g. cancer) in the presence and absence of the test compound; and
(b) comparing one or more endometrial markers, and/or polynucleotides encoding endometrial markers, and optionally other markers in each of the aliquots.

A significant difference between the levels of the markers in the aliquot maintained in the presence of (or exposed to) the test compound relative to the aliquot maintained in the absence of the test compound, indicates that the test compound possesses the potential to contribute to an endometrial disease (e.g. endometrial cancer).

### Kits

The invention also contemplates kits for carrying out the methods of the invention. Kits may typically comprise two or more components required for performing a diagnostic assay. Components include but are not limited to compounds, reagents, containers, and/or equipment.

The methods described herein may be performed by utilizing pre-packaged diagnostic kits comprising one or more specific endometrial marker polynucleotide or antibody described herein, which may be conveniently used, e.g., in clinical settings to screen and diagnose patients and to screen and identify those individuals exhibiting a predisposition to developing an endometrial disease.

In an embodiment, a container with a kit comprises a binding agent as described herein. By way of example, the kit may contain antibodies or antibody fragments which bind specifically to epitopes of one or more endometrial markers and optionally other markers, antibodies against the antibodies labelled with an enzyme; and a substrate for the enzyme. The kit may also contain microtiter plate wells, standards, assay diluent, wash buffer, adhesive plate covers, and/or instructions for carrying out a method of the invention using the kit.

In an aspect of the invention, the kit includes antibodies or fragments of antibodies which bind specifically to an epitope of one or more protein listed in Table 1, 4, 5, or 6 and means for detecting binding of the antibodies to their epitope associated with tumor cells, either as concentrates (including lyophilized compositions), which may be further diluted prior to use or at the concentration of use, where the vials may include one or more dosages. Where the kits are intended for in vivo use, single dosages may be provided in sterilized containers, having the desired amount and concentration of agents. Containers that provide a formulation for direct use, usually do not require other reagents, as for example, where the kit contains a radiolabelled antibody preparation for in vivo imaging.

A kit may be designed to detect the level of polynucleotides encoding one or more endometrial polynucleotide markers in a sample. In an embodiment, the polynucleotides encode one or more polynucleotides encoding a polypeptide listed in Table 1, 4, 5 or 6. Such kits generally comprise at least one oligonucleotide probe or primer, as described herein, that hybridizes to a polynucleotide encoding one or more endometrial cancer markers. Such an oligonucleotide may be used, for example, within a PCR or hybridization procedure. Additional components that may be present within the kits include a second oligonucleotide and/or a diagnostic reagent or container to facilitate detection of a polynucleotide encoding one or more endometrial cancer markers.

The invention provides a kit containing a microarray described herein ready for hybridization to target endometrial polynucleotide markers, plus software for the data analysis of the results. The software to be included with the kit comprises data analysis methods, in particular mathematical routines for marker discovery, including the calculation of correlation coefficients between clinical categories and marker expression. The software may also include mathematical routines for calculating the correlation between sample marker expression and control marker expression, using array-generated fluorescence data, to determine the clinical classification of the sample.

The reagents suitable for applying the screening methods of the invention to evaluate compounds may be packaged into convenient kits described herein providing the necessary materials packaged into suitable containers.

The invention contemplates a kit for assessing the presence of endometrial cells, wherein the kit comprises antibodies specific for one or more endometrial markers, or primers or probes for polynucleotides encoding same, and optionally probes, primers or antibodies specific for other markers associated with an endometrial disease (e.g. cancer).

The invention relates to a kit for assessing the suitability of each of a plurality of test compounds for inhibiting an endometrial disease (e.g. endometrial cancer) in a patient. The kit comprises reagents for assessing one or more endometrial markers or polynucleotides encoding same, and optionally a plurality of test agents or compounds.

Additionally the invention provides a kit for assessing the potential of a test compound to contribute to an endometrial disease (e.g. cancer). The kit comprises endometrial diseased cells (e.g. cancer cells) and reagents for assessing one or more endometrial markers, polynucleotides encoding same, and optionally other markers associated with an endometrial disease.

### Therapeutic Applications

One or more endometrial markers may be targets for immunotherapy. Immunotherapeutic methods include the use of antibody therapy, in vivo vaccines, and ex vivo immunotherapy approaches.

In one aspect, the invention provides one or more endometrial marker antibodies that may be used systemically to treat an endometrial disease associated with the marker. In particular, the endometrial disease is endometrial cancer and one or more endometrial marker antibodies may be used systemically to treat endometrial cancer. Preferably antibodies are used that target the tumor cells but not the surrounding non-tumor cells and tissue.

Thus, the invention provides a method of treating a patient susceptible to, or having a disease (e.g. cancer) that expresses one or more endometrial marker (in particular a marker up-regulated in endometrial cancer, for example, an up-regulated marker in Table 1), comprising administering to the patient an effective amount of an antibody that binds specifically to one or more endometrial marker.

In another aspect, the invention provides a method of inhibiting the growth of tumor cells expressing one or more endometrial cancer markers, comprising administering to a patient an antibody which binds specifically to one or more endometrial cancer markers in an amount effective to inhibit growth of the tumor cells.

One or more endometrial marker antibodies may also be used in a method for selectively inhibiting the growth of, or killing a cell expressing one or more endometrial marker (e.g. tumor cell expressing one or more endometrial cancer marker) comprising reacting one or more endometrial marker antibody immunoconjugate or immunotoxin with the cell in an amount sufficient to inhibit the growth of, or kill the cell.

By way of example, unconjugated antibodies to endometrial cancer markers may be introduced into a patient such that the antibodies bind to endometrial cancer marker expressing cancer cells and mediate growth inhibition of such cells (including the destruction thereof), and the tumor, by mechanisms which may include complement-mediated cytolysis, antibody-dependent cellular cytotoxicity, altering the physiologic function of one or more endometrial cancer markers, and/or the inhibition of ligand binding or signal transduction pathways. In addition to unconjugated antibodies to endometrial cancer markers, one or more endometrial cancer marker antibodies conjugated to therapeutic agents (e.g. immunoconjugates) may also be used therapeutically to deliver the agent directly to one or more endometrial cancer marker expressing tumor cells and thereby destroy the tumor. Examples of such agents include abrin, ricin A, Pseudomonas exotoxin, or diphtheria toxin; proteins such as tumor necrosis factor, alpha-interferon, beta-interferon, nerve growth factor, platelet derived growth factor, tissue plasminogen activator; and biological response modifiers such as lymphokines, interleukin-1, interleukin-2, interleukin-6, granulocyte macrophage colony stimulating factor, granulocyte colony stimulating factor, or other growth factors.

Cancer immunotherapy using one or more endometrial cancer marker antibodies may utilize the various approaches that have been successfully employed for cancers, including but not limited to colon cancer (Arlen et al., 1998, Crit. Rev Immunol 18: 133-138), multiple myeloma (Ozaki et al., 1997, Blood 90: 3179-3186; Tsunenati et al., 1997, Blood 90: 2437-2444), gastric cancer (Kasprzyk et al., 1992, Cancer Res 52:2771-2776), B-cell lymphoma (Funakoshi et al., 1996, J Immunther Emphasis Tumor Immunol 19: 93-101), leukemia (Zhong et al., 1996, Leuk Res 20: 581-589), colorectal cancer (Moun et al., 1994, Cancer Res 54:6160-6166); Velders et al., 1995, Cancer Res 55: 4398-4403), and breast cancer (Shepard et al., 1991, J Clin Immunol 11: 117-127).

In the practice of a method of the invention, endometrial cancer marker antibodies capable of inhibiting the growth of cancer cells expressing endometrial cancer markers are administered in a therapeutically effective amount to cancer patients whose tumors express or overexpress one or more endometrial cancer markers. The invention may provide a specific, effective and long-needed treatment for endometrial cancer. The antibody therapy methods of the invention may be combined with other therapies including chemotherapy and radiation.

Patients may be evaluated for the presence and level of expression or overexpression of one or more endometrial markers in diseased cells and tissues (e.g. tumors), in particular using immunohistochemical assessments of tissue, quantitative imaging as described herein, or other techniques capable of reliably indicating the presence and degree of expression of one or more endometrial markers. Immunohistochemical analysis of tumor biopsies or surgical specimens may be employed for this purpose.

Endometrial marker antibodies useful in treating disease (e.g. cancer) include those that are capable of initiating a potent immune response against the disease (e.g. tumor) and those that are capable of direct cytotoxicity. In this regard, endometrial marker antibodies may elicit cell lysis by either complement-mediated or antibody-dependent cell cytotoxicity (ADCC) mechanisms, both of which require an intact Fc portion of the immunoglobulin molecule for interaction with effector cell Fc receptor sites or complement proteins.

Endometrial marker antibodies that exert a direct biological effect on tumor growth may also be useful in the practice of the invention. Such antibodies may not require the complete immunoglobulin to exert the effect. Potential mechanisms by which such directly cytotoxic antibodies may act include inhibition of cell growth, modulation of cellular differentiation, modulation of tumor angiogenesis factor profiles, and the induction of apoptosis. The mechanism by which a particular antibody exerts an anti-tumor effect may be evaluated using any number of in vitro assays designed to determine ADCC, antibody-dependent macrophage-mediated cytotoxicity (ADMMC), complement-mediated cell lysis, and others known in the art.

The anti-tumor activity of a particular endometrial cancer marker antibody, or combination of endometrial cancer marker antibodies, may be evaluated in vivo using a suitable animal model. Xenogenic cancer models, where human cancer explants or passaged xenograft tissues are introduced into immune compromised animals, such as nude or SCID mice, may be employed.

The methods of the invention contemplate the administration of single endometrial marker antibodies as well as combinations, or “cocktails”, of different individual antibodies such as those recognizing different epitopes of other markers. Such cocktails may have certain advantages inasmuch as they contain antibodies that bind to different epitopes of endometrial markers and/or exploit different effector mechanisms or combine directly cytotoxic antibodies with antibodies that rely on immune effector functionality. Such antibodies in combination may exhibit synergistic therapeutic effects. In addition, the administration of one or more endometrial marker specific antibodies may be combined with other therapeutic agents, including but not limited to chemotherapeutic agents, androgen-blockers, and immune modulators (e.g., IL2, GM-CSF). The endometrial marker specific antibodies may be administered in their “naked” or unconjugated form, or may have therapeutic agents conjugated to them.

The endometrial marker specific antibodies used in the methods of the invention may be formulated into pharmaceutical compositions comprising a carrier suitable for the desired delivery method. Suitable carriers include any material which when combined with the antibodies retains the function of the antibody and is non-reactive with the subject's immune systems. Examples include any of a number of standard pharmaceutical carriers such as sterile phosphate buffered saline solutions, bacteriostatic water, and the like (see, generally, Remington's Pharmaceutical Sciences 16.sup.th Edition, A. Osal., Ed., 1980).

One or more endometrial marker specific antibody formulations may be administered via any route capable of delivering the antibodies to the a disease (e.g. tumor) site. Routes of administration include, but are not limited to, intravenous, intraperitoneal, intramuscular, intratumor, intradermal, and the like. Preferably, the route of administration is by intravenous injection. Antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration such as intravenous injection (IV), at an effective dose. Dosages will depend upon various factors generally appreciated by those of skill in the art, including the type of disease and the severity, grade, or stage of the disease, the binding affinity and half life of the antibodies used, the degree of endometrial marker expression in the patient, the extent of circulating endometrial markers, the desired steady-state antibody concentration level, frequency of treatment, and the influence of any chemotherapeutic agents used in combination with the treatment method of the invention. Daily doses may range from about 0.1 to 100 mg/kg. Doses in the range of 10-500 mg antibodies per week may be effective and well tolerated, although even higher weekly doses may be appropriate and/or well tolerated. A determining factor in defining the appropriate dose is the amount of a particular antibody necessary to be therapeutically effective in a particular context. Repeated administrations may be required to achieve disease inhibition or regression. Direct administration of one or more endometrial marker antibodies is also possible and may have advantages in certain situations.

Patients may be evaluated for serum cancer markers in order to assist in the determination of the most effective dosing regimen and related factors. The endometrial cancer assay methods described herein, or similar assays, may be used for quantitating circulating endometrial marker levels in patients prior to treatment. Such assays may also be used for monitoring throughout therapy, and may be useful to gauge therapeutic success in combination with evaluating other parameters such as serum levels of endometrial markers.

The invention further provides vaccines formulated to contain one or more endometrial marker or fragment thereof.

In an embodiment, the invention provides a method of vaccinating an individual against one or more endometrial marker listed in Table 1 comprising the step of inoculating the individual with the marker or fragment thereof that lacks activity, wherein the inoculation elicits an immune response in the individual thereby vaccinating the individual against the marker.

The use in anti-cancer therapy of a tumor antigen in a vaccine for generating humoral and cell-mediated immunity is well known and, for example, has been employed in prostate cancer using human PSMA and rodent PAP immunogens (Hodge et al., 1995, Int. J. Cancer 63: 231-237; Fong et al., 1997, J. Immunol. 159: 3113-3117). These and similar methods can be practiced by employing one or more endometrial markers, or fragment thereof, or endometrial polynucleotide markers and recombinant vectors capable of expressing and appropriately presenting endometrial marker immunogens.

By way of example, viral gene delivery systems may be used to deliver one or more endometrial polynucleotide markers. Various viral gene delivery systems which can be used in the practice of this aspect of the invention include, but are not limited to, vaccinia, fowlpox, canarypox, adenovirus, influenza, poliovirus, adeno-associated virus, lentivirus, and sindbus virus (Restifo, 1996, Curr. Opin. Immunol. 8: 658-663). Non-viral delivery systems may also be employed by using naked DNA encoding one or more endometrial cancer marker or fragment thereof introduced into the patient (e.g., intramuscularly) to induce an anti-tumor response.

Various ex vivo strategies may also be employed. One approach involves the use of cells to present one or more endometrial marker to a patient's immune system. For example, autologous dendritic cells which express MHC class I and II, may be pulsed with one or more endometrial marker or peptides thereof that are capable of binding to MHC molecules, to thereby stimulate the patients' immune systems (See, for example, Tjoa et al., 1996, Prostate 28: 65-69; Murphy et al., 1996, Prostate 29: 371-380).

Anti-idiotypic endometrial marker specific antibodies can also be used in therapy as a vaccine for inducing an immune response to cells expressing one or more endometrial marker. The generation of anti-idiotypic antibodies is well known in the art and can readily be adapted to generate anti-idiotypic endometrial cancer marker specific antibodies that mimic an epitope on one or more endometrial cancer markers (see, for example, Wagner et al., 1997, Hybridoma 16: 33-40; Foon et al., 1995, J Clin Invest 96: 334-342; Herlyn et al., 1996, Cancer Immunol Immunother 43: 65-76). Such an antibody can be used in anti-idiotypic therapy as presently practiced with other anti-idiotypic antibodies directed against antigens associated with disease (e.g. tumor antigens).

Genetic immunization methods may be utilized to generate prophylactic or therapeutic humoral and cellular immune responses directed against cells expressing one or more endometrial cancer marker. One or more DNA molecules encoding endometrial markers, constructs comprising DNA encoding one or more endometrial markers/immunogens and appropriate regulatory sequences may be injected directly into muscle or skin of an individual, such that the cells of the muscle or skin take-up the construct and express the encoded endometrial markers/immunogens. The endometrial markers/immunogens may be expressed as cell surface proteins or be secreted. Expression of one or more endometrial markers results in the generation of prophylactic or therapeutic humoral and cellular immunity against the disease (e.g. cancer). Various prophylactic and therapeutic genetic immunization techniques known in the art may be used.

The invention further provides methods for inhibiting cellular activity (e.g., cell proliferation, activation, or propagation) of a cell expressing one or more endometrial marker. This method comprises reacting immunoconjugates of the invention (e.g., a heterogeneous or homogenous mixture) with the cell so that endometrial markers form complexes with the immunoconjugates. A subject with a neoplastic or preneoplastic condition can be treated when the inhibition of cellular activity results in cell death.

In another aspect, the invention provides methods for selectively inhibiting a cell expressing one or more endometrial marker by reacting any one or a combination of the immunoconjugates of the invention with the cell in an amount sufficient to inhibit the cell. Amounts include those that are sufficient to kill the cell or sufficient to inhibit cell growth or proliferation.

Vectors derived from retroviruses, adenovirus, herpes or vaccinia viruses, or from various bacterial plasmids, may be used to deliver polynucleotides encoding endometrial cancer markers to a targeted organ, tissue, or cell population. Methods well known to those skilled in the art may be used to construct recombinant vectors that will express antisense polynucleotides for endometrial markers. (See, for example, the techniques described in Sambrook et al (supra) and Ausubel et al (supra)).

Methods for introducing vectors into cells or tissues include those methods discussed herein and which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art.

Genes encoding endometrial markers can be turned off by transfecting a cell or tissue with vectors that express high levels of a desired endometrial marker-encoding fragment. Such constructs can inundate cells with untranslatable sense or antisense sequences. Even in the absence of integration into the DNA, such vectors may continue to transcribe RNA molecules until all copies are disabled by endogenous nucleases:

Modifications of gene expression can be obtained by designing antisense molecules, DNA, RNA or PNA, to the regulatory regions of a gene encoding an endometrial marker, i.e., the promoters, enhancers, and introns. Preferably, oligonucleotides are derived from the transcription initiation site, e.g. between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA were reviewed by Gee J E et al. (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of sequences encoding an endometrial marker.

Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

One or more endometrial markers and polynucleotides encoding the markers, and fragments thereof, may be used in the treatment of an endometrial disease (e.g. endometrial cancer) in a subject. In an aspect the endometrial markers and polynucleotides encoding the markers are endometrial cancer markers that are down-regulated in endometrial cancer, for example, a down-regulated marker in Table 1. The markers or polynucleotides may be formulated into compositions for administration to subjects suffering from an endometrial disease. Therefore, the present invention also relates to a composition comprising one or more endometrial markers or polynucleotides encoding the markers, or a fragment thereof, and a pharmaceutically acceptable carrier, excipient or diluent. A method for treating or preventing an endometrial disease in a subject is also provided comprising administering to a patient in need thereof, one or more endometrial markers or polynucleotides encoding the markers, or a composition of the invention.

The invention further provides a method of inhibiting an endometrial disease (e.g. endometrial cancer) in a patient comprising:
(a) obtaining a sample comprising diseased cells from the patient;
(b) separately maintaining aliquots of the sample in the presence of a plurality of test agents;
(c) comparing levels of one or more endometrial markers, and/or polynucleotides encoding one or more endometrial markers in each aliquot;
(d) administering to the patient at least one of the test agents which alters the levels of the endometrial markers, and/or polynucleotides encoding one or more endometrial markers in the aliquot containing that test agent, relative to the other test agents.

Endometrial markers in uterine biopsy tissue or fluid and sera may vary between known fertile and infertile women during the window of implantation, deviate in women undergoing ovarian hyperstimulation/ovulation induction, and correlate with successful initiation of pregnancy. Therefore, endometrial markers of the invention may serve as minimally or noninvasive markers of uterine receptivity for implantation.

The present invention further provides a method of determining uterine endometrial receptivity by first obtaining a serum, uterine fluid or endometrial biopsy sample from a patient and detecting the presence of an endometrial marker associated with a certain endometrium phase, wherein the presence or absence of an endometrial marker as compared to controls indicates uterine receptivity. In an embodiment, the endometrium phase is the secretory phase. Where necessary for the evaluation, repetitive samples may be collected throughout the menstrual cycle. Non-receptive controls are both women who are in the non-fertile stage of the menstrual cycle and women with known uterine dysfunction where an endometrial marker is not present or present on the endometrium throughout the menstrual cycle or certain endometrium phases.

The present invention further provides a method of monitoring the effects of ovarian hyperstimulation and/or ovulation induction protocols on uterine receptivity either for individual women receiving the treatment or for the evaluation of new protocols. In an embodiment, the method comprises: (a) obtaining a serum, uterine or fluid or endometrial biopsy sample from a patient receiving the treatments; and (b) detecting the presence of an endometrial marker of the invention present in the endometrium at the time of fertilization, early embryogenesis, and implantation; wherein presence or absence of an endometrial marker indicates receptivity. A disruption of the normal cyclic presence of an endometrial marker indicates that the treatment may adversely affect uterine receptivity. This disruption may include non-cyclic presence of an endometrial marker or an aberrant presence of an endometrial marker as compared to controls.

In an aspect the invention provides a method of determining a probability of successful implantation with an ovarian stimulation in vitro fertilization and embryo transfer procedure, comprising:
(a) determining a level of an endometrial marker identified in accordance with a method of the invention in a sample obtained from a patient who has undergone an ovarian stimulation in vitro fertilization and embryo transfer procedure; and
(b) determining a probability of successful implantation based on the patient's determined endometrial marker level;
   wherein a significantly different endometrial marker level relative to a standard level is associated with a decreased or increased probability of successful implantation.

The present invention further provides a method of contraception by interrupting the cyclic presence of an endometrial marker. The interruption can be to reduce or eliminate a marker present during the uterine receptivity window for implantation of the menstrual cycle and to thereby alter the cyclic presence/pattern of a marker. The interruption can utilize an antagonist of a marker. The term antagonist or antagonizing is used in its broadest sense. Antagonism can include any mechanism or treatment that results in inhibition, inactivation, blocking or reduction or alteration of cyclic presence of an endometrial marker.

An active therapeutic substance described herein may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance. Solutions of an active compound as a free base or pharmaceutically acceptable salt can be prepared in an appropriate solvent with a suitable surfactant. Dispersions may be prepared in glycerol, liquid polyethylene glycols, and mixtures thereof, or in oils.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment. The compositions of the invention may be administered concurrently, separately, or sequentially with other therapeutic agents or therapies.

The therapeutic activity of compositions and agents/compounds identified using a method of the invention and may be evaluated in vivo using a suitable animal model.

The following non-limiting examples are illustrative of the present invention:

### EXAMPLE 1

Isotope-coded affinity tag (ICAT) analysis with a cleavable tag was used to examine differentially expressed proteins in proliferative and secretory endometria. Sample complexity of the tissue homogenates was reduced by strong cation exchange (SCX) fractionation and subsequent affinity cleanup. Analysis of ten SCX fractions in triplicate by nanobore liquid chromatography—tandem mass spectrometry (nanoLC-MS/MS) resulted in the identification of approximately 400 labeled proteins and the discovery of potential biomarkers for the secretory phase of the endometrial cycle. This study demonstrated the feasibility of using this approach for identifying markers at a proteomics level for different stages of the endometrial cycle.

### Tissue Samples

Endometrium tissue was retrieved from an in-house dedicated, research endometrial tissue bank. All tissues were snap frozen in liquid nitrogen within 15-20 minutes of devitalization at the time of hysterectomy, and were obtained with patient consent. In each case, the endometrium was classified as proliferative or secretory by a pathologist. The histological classification was verified by examination of a histopathologic section from the frozen research tissue. Tissue was taken for proteomic analysis from the mirror-face of the residual block. After addition of 1 ml Hanks' Balanced Salt Solution containing protease inhibitors (leupeptin, aprotinin, pepstatin at 1 μg/mL), the tissue was mechanically homogenized at 30,000 rpms using a Polytron PT 1300D handheld homogenizer (Brinkmann, Westbury, USA). The samples were stored in aliquots at −80° C. and/or submitted for protein profiling. These whole tissue homogenates contain endometrial epithelium, supportive stroma and vessels, as well as any secretions. Tissue samples from six different individuals were selected for the study. Three of these tissues were classified as proliferative endometria (PRO1, PRO2 and PRO3) and the other three as secretory endometria (SEC1, SEC2, SEC3).

### Chemicals

Acetonitrile, formic acid, potassium chloride, monobasic potassium phosphate, leupeptin, aprotinin, pepstatin and Hanks' Balanced Salt Solution were obtained from Sigma-Aldrich (Oakville, Canada). All reagents and buffers for the cleavable ICAT sample preparation procedure were from Applied Biosystems (Foster City, USA).

### ICAT Sample Preparation Procedure

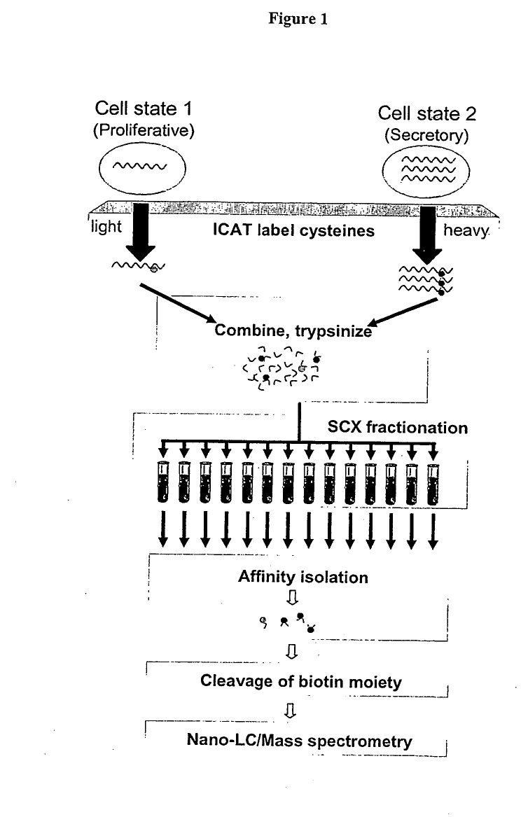

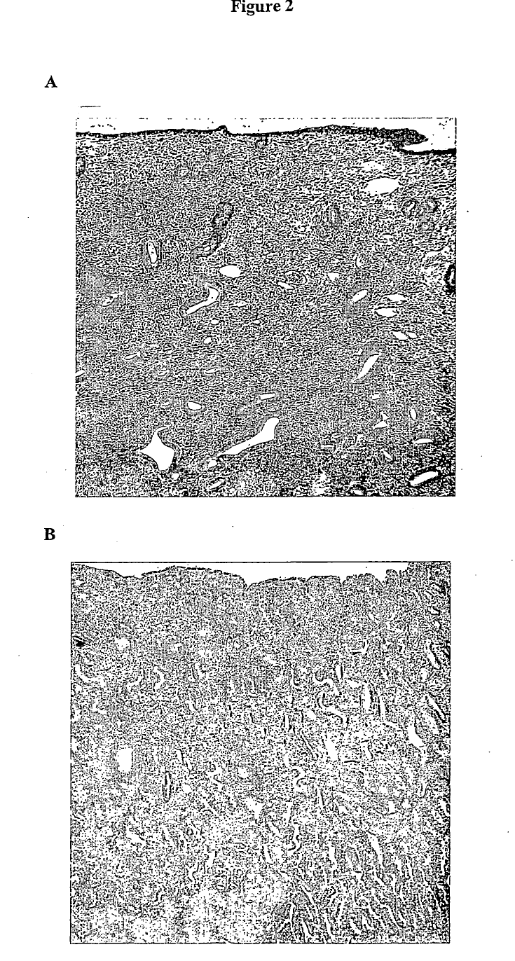

After removal of cell debris by centrifugation, the total protein content for each of the six clarified homogenates was measured using a commercially available Bradford protein assay reagent (Bio-Rad, Hercules, USA). ICAT sample preparation procedure was carried out according to the cleavable ICAT protocol (Applied Biosystems, Foster City, USA) and is illustrated in FIG. 1. Following denaturing and reducing steps, 100 μg total protein of each of the samples was labeled with either the light ICAT reagent (proliferative samples) or the heavy reagent (secretory samples). The labeled PRO1 and SEC1 samples were combined to form ICAT Sample A, PRO2 and SEC2 form ICAT Sample B, and lastly PRO3 and SEC3 form ICAT Sample C. Mixing of the labeled proliferative and secretory samples in pairs in this manner ensures that any protein or peptide losses during subsequent processing steps is the same for both samples in a pair. Since the peptides are differentially labeled, they can be traced to specific samples in the pair. Any difference detected in the levels of individual peptides can then be ascribed solely to initial differences in expression level. Chromatographic separation using a strong cation exchange (SCX) column was performed after trypsin digestion to fractionate the ICAT samples. Selected SCX fractions were purified by affinity chromatography according to the ICAT protocol, and subsequently analyzed by nanobore liquid chromatography-tandem mass spectrometry (nanoLC-MS/MS).

### Instrumentation for SCX Fractionation

SCX fractionation was performed on an HP 1050 LC system (Agilent, Palo Alto, USA) using a 1.5 ml injection loop and a SF-2120 Super Fraction Collector (Advantec MFS, Dublin, USA).

### LC/MS/MS Instrumentation

The LC system from LC Packings (Amsterdam, The Netherlands) consisted of a Famos autosampler and Ultimate Nano LC system. It was interfaced to an API QSTAR Pulsar QqTOF mass spectrometer (Applied Biosystems/MDS Sciex, Foster City, USA) using a Protana NanoES ion source (Protana Engineering A/S, Odense, Denmark). PicoTip's SilicaTip emitters with a 10 μm tip i.d. (New Objective, Woburn, USA) were used as spray capillaries. All data were acquired using Analyst QS SP5 with Bioanalyst Extension 1.1 and analyzed with ProICAT SP2 software (Applied Biosystems/MDS Sciex, Foster City, USA).

### LC Conditions

Strong cation exchange chromatography (SCX) was performed using a PolyLC Polysulfoethyl A column (The Nest Group, Southborough, USA) equipped with a guard column of the same material with the following dimensions: 5-μm particle size, 300-Å bead, 2.1-mm i.d., 10-mm length (guard column) and 100-mm length (analytical column). Eluent A of the mobile phase consisted of a 10 mM KH₂PO₄ solution in 25% acetonitrile and 75% deionized water (pH=3.0). Eluent B consisted of a 10 mM KH₂PO₄ and 350 mM KCl solution in 25% acetonitrile and 75% deionized water (pH=3.0). In each case, 1.5 ml of the total 2.4 ml sample (after acidifying with 2 ml Eluent A) was injected. Fractions were collected every 2 mins at a flow rate of 0.2 ml/min. using a binary gradient with the following profile:

t [min]

| | 0.01 | 2 | 58 | 60 |
|---|---|---|---|---|
| c (Eluent B) [%] | 0 | 0 | 100 | stop |

All reversed-phase separations were performed using PepMap C18 nano capillary columns (LC Packings, Amsterdam, The Netherlands) with the following dimensions: 3-μm particle size, 100-Å bead, 75-μm i.d. and 150-mm length. Eluent A of the mobile phase consisted of 950 ml deionized water, 50 ml acetonitrile and 1 ml formic acid (pH 3). Eluent B consisted of 50 ml deionized water, 950 ml acetonitrile and 1 ml formic acid. A binary gradient at a flow rate of approximately 200 nl/min with the following profile was used:

| | t [min] | | | | | | | |
|---|---|---|---|---|---|---|---|---|
| | 0.01 | 5 | 125 | 135 | 157 | 160 | 163 | 190 |
| c (Eluent B) [%] | 5 | 5 | 30 | 60 | 80 | 80 | 5 | Stop |

Injections of 1 μl of sample were performed in full loop mode.

### MS Conditions

The source conditions were a curtain-gas setting of 20 and an ionspray voltage (in the range of 2000-3800 V) that was optimized daily. All data were obtained in the positive-ion detection mode. In the Q0 region, the instrument parameters were a declustering potential (DP) of 65 V and a focusing potential (FP) of 265 V. Nitrogen was used as the collision gas at a setting of CAD5 for both TOF-MS and MS/MS scans.

All LC-MS/MS data were acquired in information-dependent acquisition (IDA) mode. A TOF-MS survey scan with a mass range of m/z=400-1500 and Is scan time was followed by two product ion scans with a mass range of m/z=70-2000 and 2 s scan time. The collision energy (CE) was automatically controlled by the IDA CE Parameters script. The switching criteria were set to ions greater than m/z=400 and smaller than m/z=1500 with a charge state of 2 to 5 and an intensity of ≧10 counts/s. Former target ions were excluded for 60 s and peaks within a 4 Th window were ignored. In addition, the IDA Extensions II script was set to 2 repetitions before dynamic exclusion and to select a precursor ion nearest to a threshold of 15 count/s every 4 cycles.

### Results and Discussion

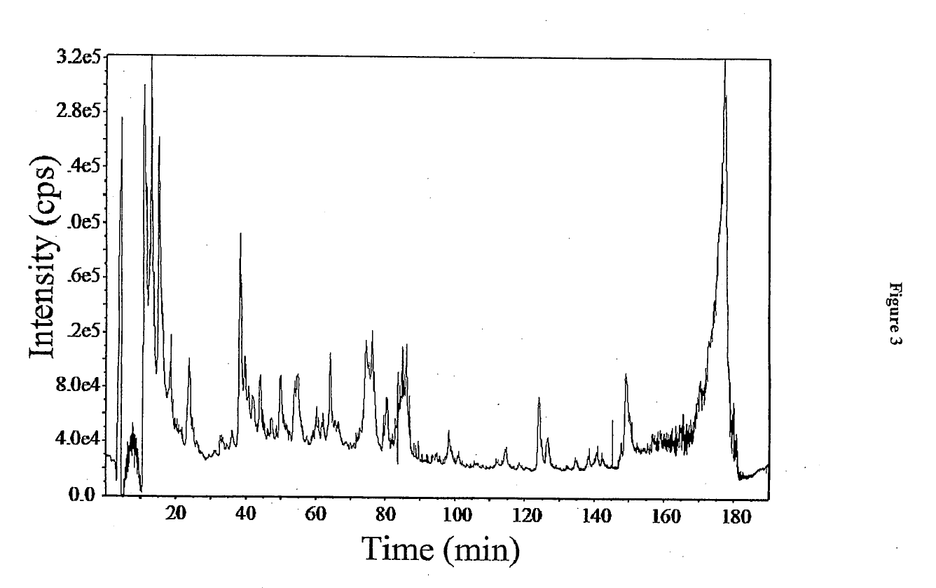

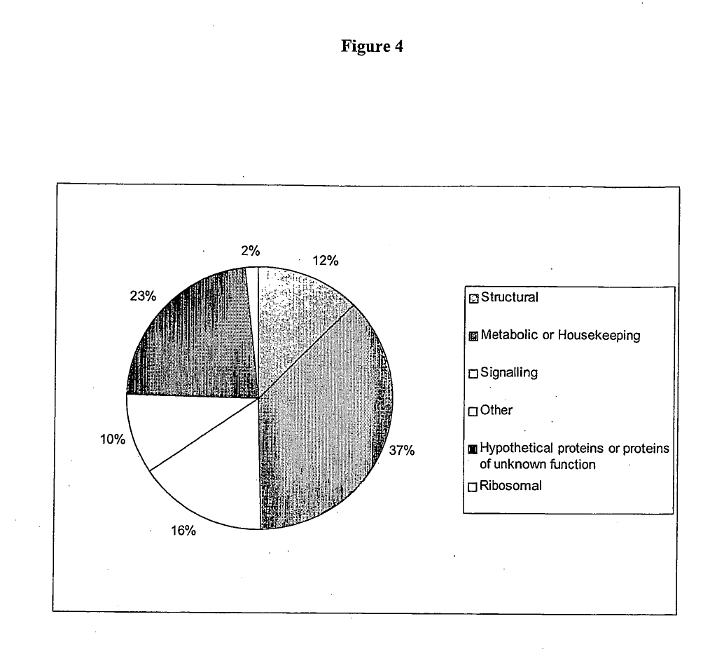

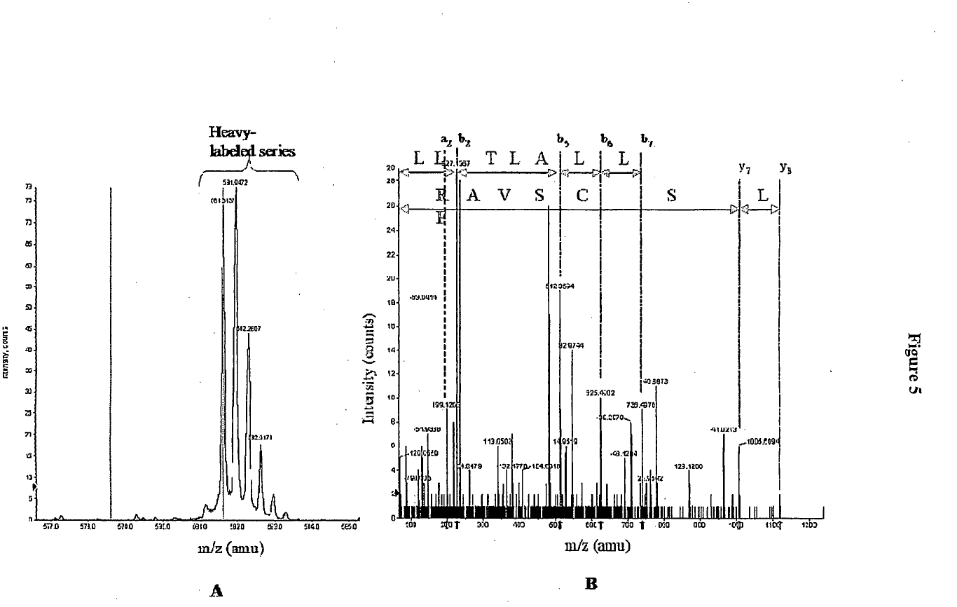

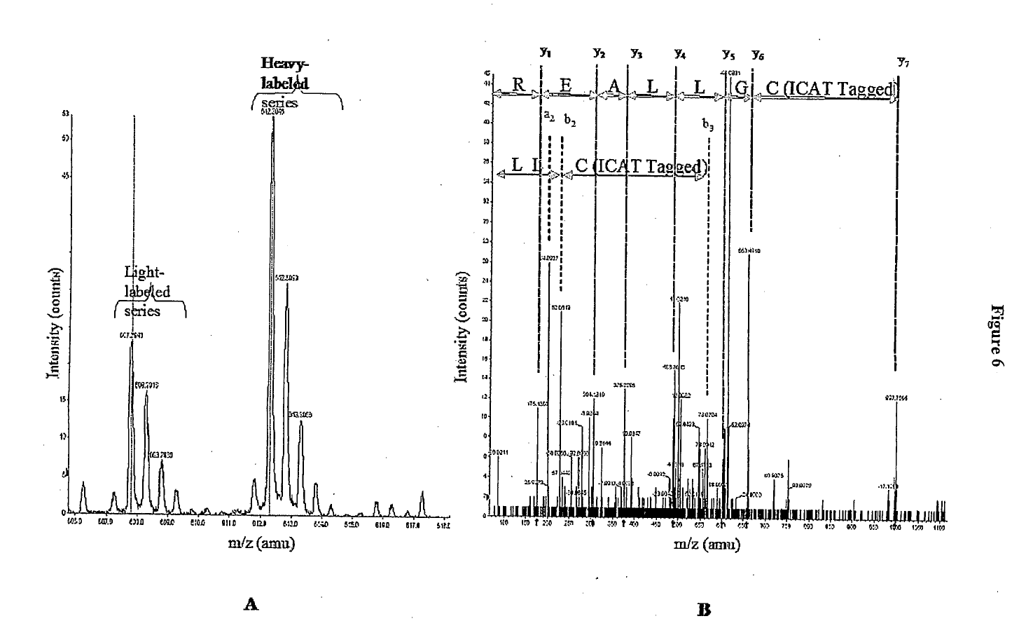

FIG. 2 shows examples of the histologic appearance of proliferative (FIG. 2A) and secretory (FIG. 2B) endometrium. In both endometria the stratum basalis is characterized by a denser stroma than the physiologic responsive stratum functionalis above. Across the top of the stratum functionalis is the surface epithelium, which lines the endometrial cavity. The proliferative endometrium (PRO2) shows small, coiled glands with lining columnar epithelium reaching to the surface. In contrast, the secretory endometrium (SEC2) is thicker, and contains more tortuous glands with intra-luminal secretions. The endometrium of both types of physiologic phases has abundant supportive stroma and vessels among the epithelial glands. In this study homogenates from the mirror faces of these tissues were used for quantitative analysis. After performing the procedure described above, SCX fractions 11-20 (of the 30 fractions) from each of the samples were chosen for further processing. This choice was based on the UV trace generated during fractionation. The 10 chosen fractions from each sample were affinity-purified, cleaved as per the ICAT protocol and analyzed using nanoLC-MS/MS. FIG. 3 is an example of a nano LC-MS total ion chromatogram (TIC) from one of the ICAT Sample A SCX fractions. As the samples were run in IDA mode (See above), each such TIC resulted in hundreds of MS/MS spectra. A ProICAT confidence value of 75 was adopted after trial-and-error for reliable protein detection and identification for this initial investigation. This has resulted in identification of approximately 400 distinct proteins. A preliminary classification of these proteins based on function, is seen in FIG. 4. As expected the majority of proteins fall under one of the metabolic, housekeeping or structural categories. The proteins classified under “other” are proteins like antibodies, which could not be included with the previously mentioned categories. There were also a significant number of proteins for which function could not be assigned or were identified from cDNA matches and are therefore classified as hypothetical. This last category often contains the most interesting cases for biologists. Adopting a less stringent confidence value resulted in apparent identification of many more proteins; the reliability of such identifications, however, was judged much poorer after manual inspection. In addition, manual inspection of the spectra was also found necessary to confirm automated quantifications in ProICAT.

The results show that expression levels of the majority of proteins identified were not consistently different between the two phases of the endometrial cycle. This similarity in expression levels of proteins is not surprising since most of the abundant proteins detected tend to be housekeeping or structural in nature. In addition, similar or identical, housekeeping or structural proteins such as actin and tubulin are expressed in many tissue types. This is significant since whole tissue homogenates consist not only of epithelial cells, but also supportive stromal cells and interstitium, blood, vessels and any glandular secretions. These sources provide a major contribution to the protein profile of the overall tissue. These high abundance proteins from these adjacent cellular types may mask differential protein expression by a cellular component of the endometrium. Thirdly the differential expression of low abundance proteins in any one cellular type within proliferative and secretory endometrium may not have been detectable by the methodology used in this study.

Despite these limitations, some instances of differential protein expression was noted. One such example is given in FIG. 5, which shows very significant enhancement of expression of a protein, identified as glutamate receptor subunit zeta 1 precursor [SEQ ID NO. 26], in all three secretory samples. The triply charged series of peaks, starting at 581.6 Th, was MS/MS-analyzed and identified as the heavy-labeled version of the tryptic peptide LLTLALLFSCSVAR [SEQ ID NO. 28], which maps to the N-terminal region of the protein. The absence of the light-labeled analogue of this protein, which would have manifested as a series of peaks starting at 578.6 Th, suggests a significantly lower level of the same protein in the proliferative samples. This is the first evidence of this protein being a marker for the endometrial secretory phase.

A second example is shown in FIG. 6 for the protein, macrophage migration inhibitory factor (MIF). Again, the protein's expression is enhanced in the secretory endometrium (1.71±0.38 times) versus the proliferative endometrium. This relative quantification is based on the three sets of samples and is calculated from the ratios of the total area of the ion peaks within the heavy-labeled series to that of the corresponding light-labeled series. Previous studies have demonstrated that MIF is expressed by the human endometrium throughout the menstrual cycle and that this expression is predominantly in the glandular epithelial cells [9]. It was found that MIF localized throughout the glandular epithelial cytoplasm in the proliferative phase, but that this distribution changed during the secretory phase, when it localized to the apical portion of the glandular epithelial cells, and was also detected in glandular secretions. Macrophages are common in female reproductive tissues. In the endometrium, they play an important role in defense. Macrophage degradation of cellular debris and foreign material may play an important role in endometrial shedding and repair. Quantification of MIF levels using ELISA assays suggested a slight increase in the mean concentration in the secretory phase (from approximately 15 to 18 ng/mg of protein based on a total of 25 samples), although the increase was not statistically significant [9]. By contrast, ICAT analysis of the six-sample set shows a significant enhancement (1.71±0.38 times) of MIF expression in the secretory endometrium. The results of this study serve to illustrate the power of the ICAT method for detecting and quantifying gross as well as subtle differences in expression levels over traditional quantitative methods relied on thus far.

### Conclusions

The ICAT technology employing the new, cleavable ICAT reagent is a powerful tool that can be used for discovering differentially expressed proteins, which could potentially be significant biomarkers of different histological cell states. The results demonstrate that for the human endometrium the expression levels of the majority of proteins do not vary significantly between the proliferative and the secretory phases. Two examples of differentially expressed proteins have been discussed in this study. Further investigation of the remainder of the SCX fractions from each of the three pairs of samples can be expected to yield more markers some of which might have implications for fertility/infertility.

### EXAMPLE 2

The ICAT analysis as described in Example 1 was used to examine differentially expressed proteins in cancer and normal endometrial tissue. The results of the analysis are illustrated in FIG. 11 which shows mass spectral windows from the ICAT experiments for three pairs of endometrial cancer/normal samples, demonstrating the over expression of calgizzarin in the cancer samples.

### EXAMPLE 3

### Material and Methods

### i) Tissue Preparation and Histologic Classification

Endometrium and endometrial cancer tissues were retrieved from a dedicated, research in-house endometrial tissue bank. The consenting and tissue banking procedures for this tissue bank were approved by the relevant institutions. All tissues had been snap frozen in liquid nitrogen within 15-20 minutes of devitalization at the time of hysterectomy, and were obtained with patient consent. In each case, the endometrium was classified as non-malignant, or malignant by a pathologist. (26). Non-malignant endometrial cases included both normal physiologic states (atrophic, proliferative, secretory, menstrual) and pathologic states (benign endometrial polyp, disordered proliferative). Malignant endometrial cases included endometrioid, mucinous, and serous adenocarcinomas and malignant mixed Mullerian tumors (carcinosarcomas). This classification was performed using the routine surgical pathology sections. Study cases included only benign or malignant cases; cases of endometrial hyperplasia, some of which could be considered to represent an intermediate phenotype, were not included in this study. The histologic classification was verified by examination of a histopathologic section from the frozen research tissue. Tissue was taken for proteomic analysis from the mirror-face of the residual block.

Tissue was thawed in Hanks' balanced salt solution (HBSS, Sigma) containing protease inhibitors (leupeptine, aprotinin, pepstatin in 1 μg/mL) and followed by mechanical homogenation. The specimens were then stored in aliquots at −80° C. and/or submitted for protein profiling. These whole tissue homogenates contain both endometrial epithelium or carcinoma, supportive stroma and vessels, and any secretions.

Immunohistochemical staining of selected malignant endometrial tissue was done using a polyclonal (rabbit) antibody against the putative tumor marker available from Calbiochem (San Diego, Calif.). Sections were cut from the paraffin embedded tissue, antibody applied in a 1:2000 dilution in Universal Strepavidin System, and immunohistochemical completed using a diaminobenzidine (DAB) chromogen.

### ii) Protein Profiling

Tissue lysate was fractionated to reduce the sample complexity before protein profiling. An identical quantity of proteins was used for all samples within a method; HBSS was used to compensate the initial volume to ensure equal volumes for all samples. For C18 Zip-tip (Millipore) fractionation, 2 μg of proteins from endometrial tissue homogenate in the presence of 0.3% trifluoroacetic acid (TFA) were loaded. After washing with water containing 0.3% TFA, 1 μL of 60% acetonitrile with 0.3% TFA were used to elute proteins from C18 directly onto a MALDI target containing pre-dried 1 μL 10 mg/mL sinapinic acid in 60% acetonitrile. The dried protein spots were analyzed by a MALDI-TOF (Voyager DE-STR, Applied Biosystems) mass spectrometer.

For protein profiling using SELDI-TOF MS, 1 μg proteins from endometrial tissue homogenate were incubated with WCX2, SAX2, IMAC, H50 surfaces according to the manufacturer's instructions. In brief, samples were diluted to 55 μL with the corresponding binding buffer, spotted onto the appropriate ProteinChip surface, and incubated in a sealed BioProcessor for one hour at room temperature. The ProteinChip surface was washed twice with the appropriate buffer for five-minutes, briefly rinsed with water and air-dried. Two times 0.5 μL of 50% saturated sinapinic acid in 50% acetonitrile was applied on the samples to form crystals. The ProteinChips were analyzed using a linear TOF analyzer, PBSIIc (Protein Biology System IIc, Ciphergen), or a quadrupole/TOF hybrid tandem mass spectrometer, QSTAR XL (Applied Biosystems/MDS Sciex).

iii) Protein Purification and Identification

A normal and an EmCa sample were subject to chromatographic separation in parallel to yield partially purified protein for identification. 500 μg proteins from the whole tissue homogenate were fractionated using size exclusion (BioSep 2000, Phenomenex) at 1 mL/min flow with phosphate buffer (pH7.9) and 0.05% (w/v) sodium azide. One-millilitre fractions were collected; the eluates were then concentrated to 50 μL with a silicon carbide-based spincolumn, (ProteoSpin, MDS Sciex). Five microlitres (10%) of concentrate was desalted by C18 zip-tip and analyzed with MALDI-TOF MS to locate the fractions containing the protein marker of interest (10,843 Da) in the EmCa sample. The fractions with the enriched 10,843 Da protein were diluted to 100 μL with freshly prepared dithiothreitol (DTT) (5 mM final) in 150 mM Tris pH 8.5 buffer, and incubated at 60 C for one hour. Ten microlitres (10%) of the reaction mixture was desalted by C18 zip-tip and analyzed with MALDI-TOF MS to assess the effect of DTT on the protein of interest (see Result for detail). The remaining 90 μL was precipitated by acetone (80% (v/v) final), resuspended in SDS sample buffer, and the proteins were resolved by sodium dodecyl sulfate polyacrylamide gel electrophoresis (SDS-PAGE). Protein molecular weight markers (New England Biolabs) and cytochrome C(C-2506 Sigma) were included to guide the excision of gel portions containing the protein of interest. Intact proteins were extracted from the gel by 50 μL extraction solution (formic acid/acetonitrile/isopropanol/water in a ratio of 50/25/15/10) at room temperature for four hours. The extracts were completely dried by SpeedVac and resuspended in 40 μL 100 mM ammonium bicarbonate. Half of the resuspended proteins was desalted by C18 zip-tip and analyzed with MALDI-TOF, the other half was digested in solution with 100 ng trypsin (Promega). The resulted tryptic peptides were analyzed by MALDI-QqTOF MS. The identity of the 10,843Da protein, chaperonin 10, was determined using amino acid sequence-tag analysis (Mascot, Matrix Science).

Identification of chaperonin 10 was verified by western blot, 12 μg of proteins was resolved by 8-16% gradient SDS-PAGE and electrophoretically transferred to nitrocellulose membrane (MSI). Membranes were incubated for two hours with 1:1000 dilution of anti-chaperonin 10 antiserum (Stressgen) or ERK1 antibodies (Santa Cruz) diluted in 5% milk with 0.2% NP-40 and one hour with secondary antibodies. Chaperonin 10 and ERK1 were detected by chemiluminescence reagent (NEN) and X-ray film according to the manufacturer's instructions.

### Results:

A total of 44 malignant and non-malignant endometrial tissue samples were submitted for proteomic analysis. Twenty-three of these cases were non-malignant, and the remaining 21 cases were malignant. The exact histopathologic diagnoses are shown in Tables 2 and 3.

### Protein Profiling

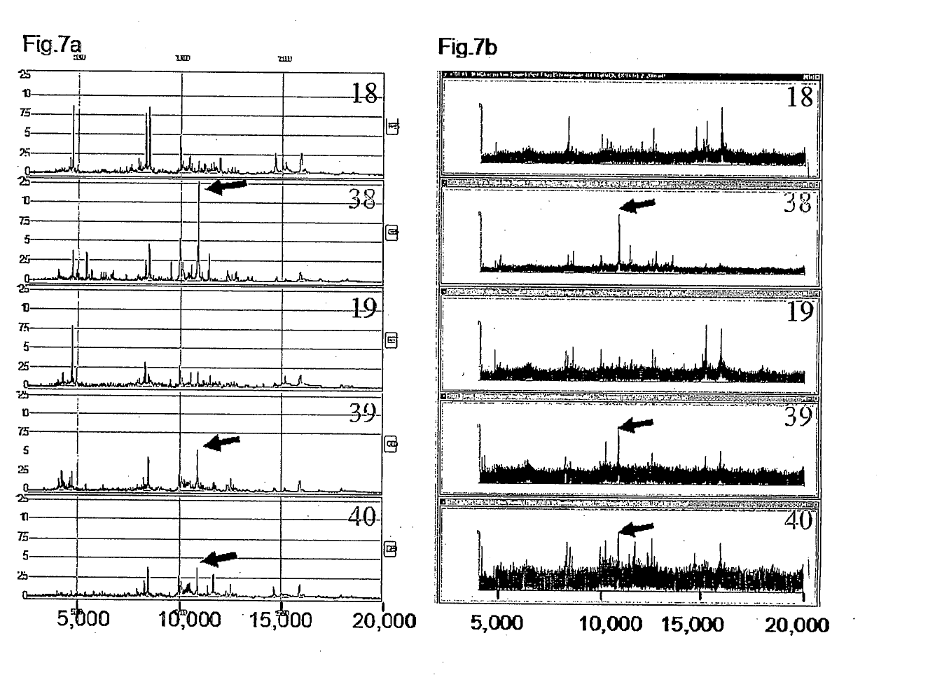

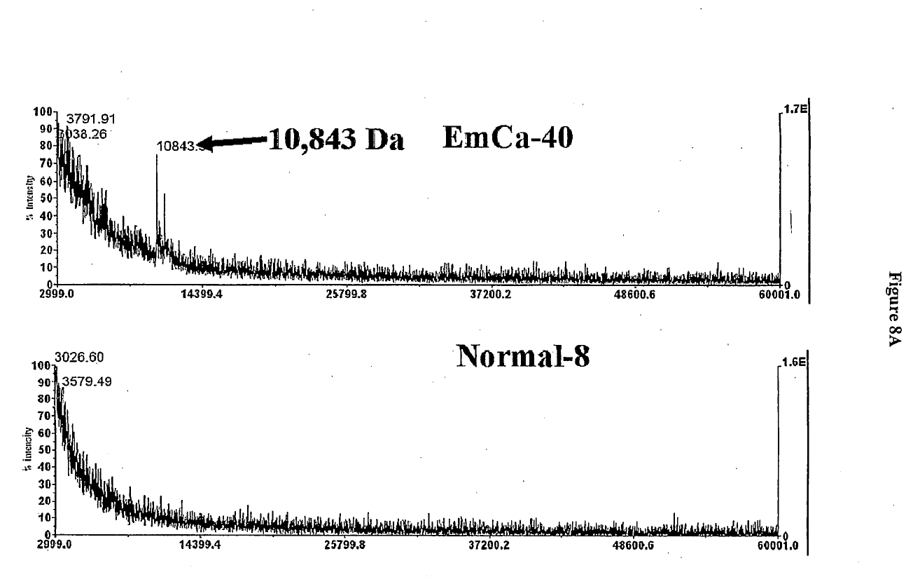

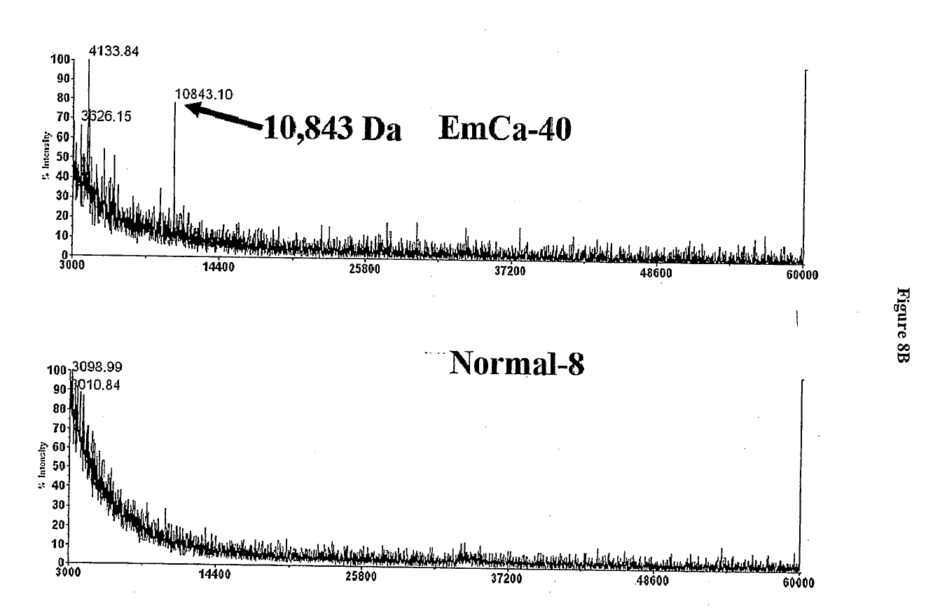

Both C18 zip-tip purification and retentive separation on ProteinChip WCX2 were effective in generating protein profiles that permit differentiation between normal and tumorous epithelium samples. Distinguishing features include both appearances and disappearances of proteins. One protein at approximately 10,840±10 Da was present in all EmCa samples and absent or greatly diminished in all normal samples tested. FIG. 7 shows the mass spectra obtained on the ProteinChip WCX2 using (a) the linear TOF mass spectrometer, PBSIIc, and (b) the QqTOF mass spectrometer, QSTAR XL. The superior mass accuracy and resolution of the latter afforded determination of the molecular weight of the marker protein as 10,843 Da.

### Marker Protein Purification and Identification

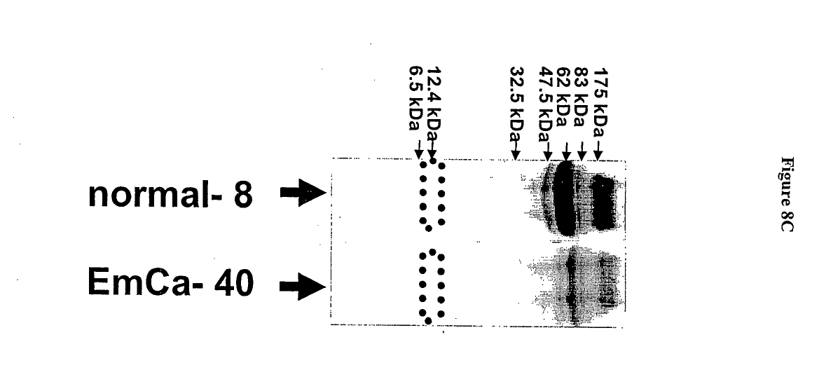

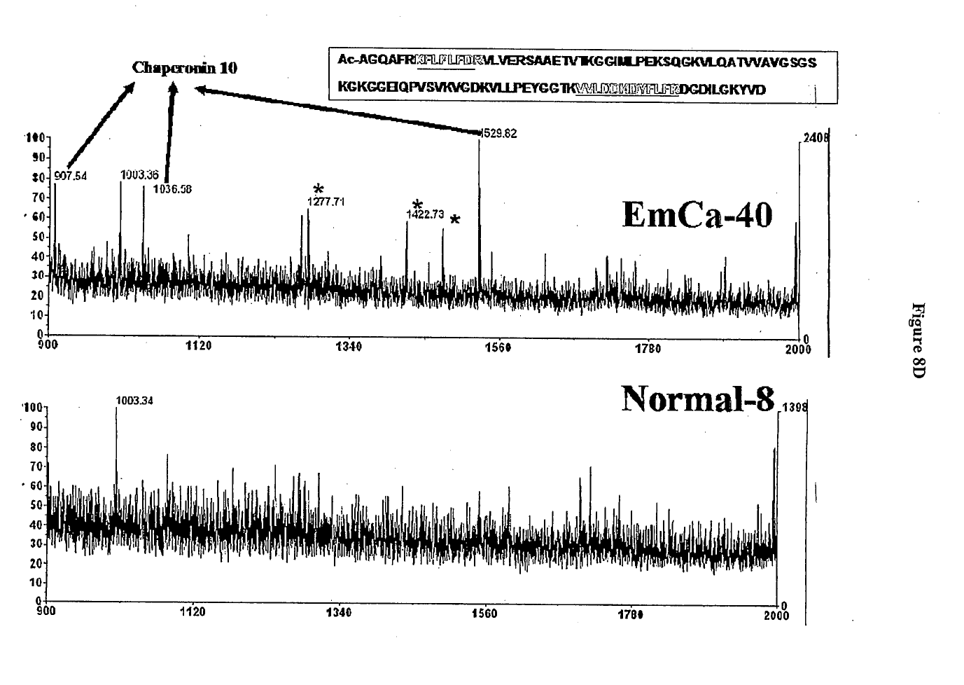

As detailed above and outlined in FIG. 8 size-exclusion LC was employed to fractionate the tissue homogeneates. The target 10,843 Da protein was found to elute in one of the early fractions from size exclusion column which suggests that this protein is a part of a large protein complex (FIG. 8A). After concentration on the spin column, the proteins were treated with DTT to break the tertiary structure and reduce any disulfide bonds. The molecular weight of the target protein was verified to remain as 10,843 Da after DTT treatment and before SDS-PAGE (FIG. 8B). This result suggests that the 10,843 Da target protein contains no intra- or inter-polypeptide disulfide bonds. The remaining protein concentrates were further separated by SDS-PAGE, and the gel was stained with colloidal Coomassie Blue (FIG. 8C). There were, however, no visible bands at the region around 10-11 kDa, probably a consequence of low protein concentration and relatively low sensitivity of the Coomassie stain. The gel portions covering approximate 7,000-16,000 Da were excised as guided by the molecular weight markers (FIG. 8C). After protein extraction, digestion with trypsin and MALDI analysis, six “unique” tryptic peptides were detected in the EmCa sample vs. the control (FIG. 8D). All six peptides were sequenced by MALDI-QqTOF MS; three were traced to keratin and three to chaperonin 10 (FIGS. 8D and 8E). The average molecular weight of chaperonin 10 was calculated to be 10842.5 Da after considering two putative posttranslational modifications, the removal of the N-terminal methionine and acetylation of the alanine residue. This is consistent with the measurement of the target protein and the reported molecular weight of chaperonin 10 purified from human platelet (18).

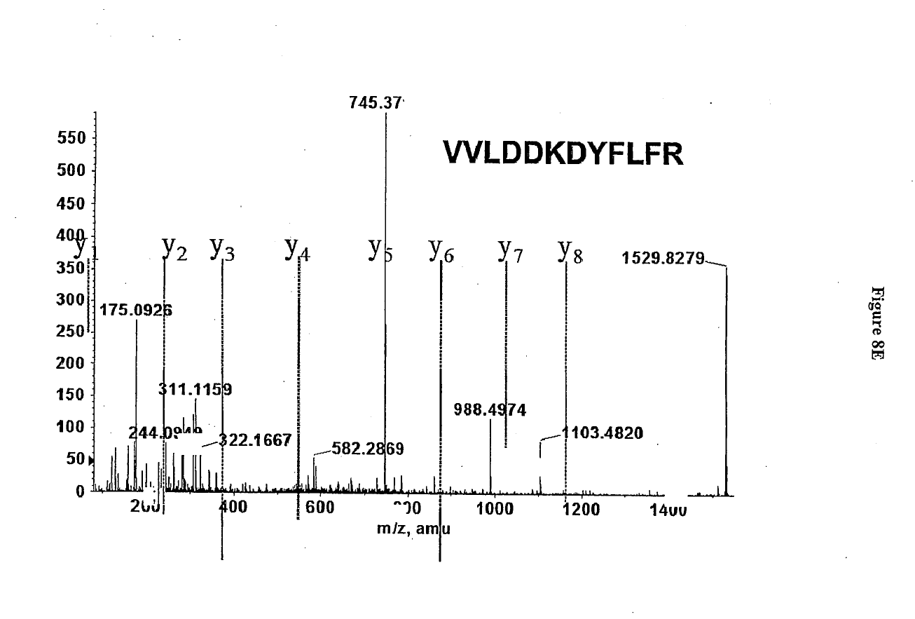

The differential expression of chaperonin 10 among cancerous and normal tissue was re-tested by western blot analysis. The signal of chaperonin 10 is higher in all EmCa specimens that also display a relative high 10,843 Da peak in their corresponding protein profiles (FIG. 9).

Tables 2 and 3 summarizes the results in identifying chaperonin 10 by MS and western blot analyses in non-malignant and malignant endometrial tissue respectively. The results for both MS and western blotting have been reported using a semi-quantitative system ranging from absent (0) to 5+ (for high intensity). These two independent methodologies demonstrate consistency in the detection of chaperonin 10.

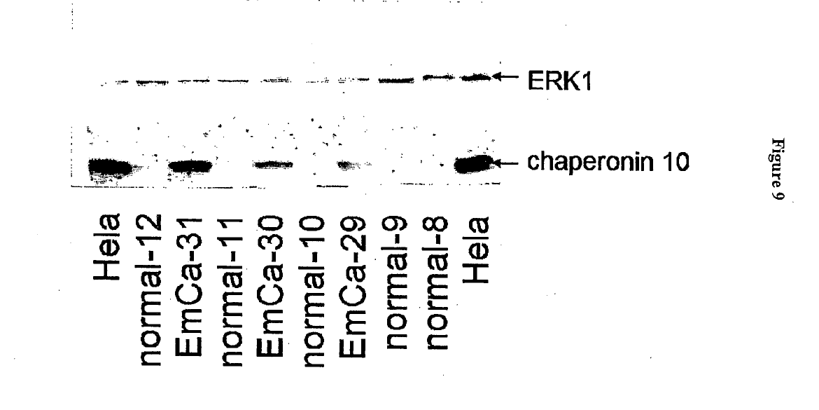

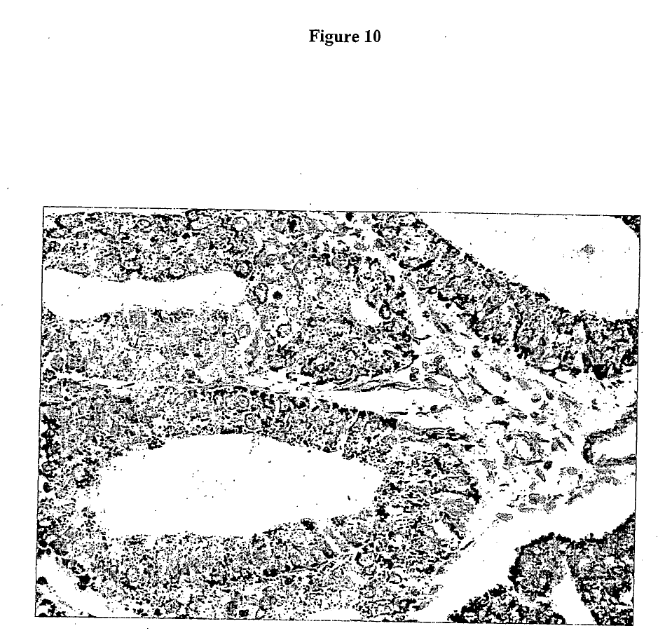

Moderately strong immunohistochemical staining for chaperonin 10 was noted in the cytoplasm of an endometrioid adenocarcinoma, as compared to adjacent stroma and benign endometrial gland. (FIG. 10). This result demonstrates the association of chaperonin 10 with malignant endometrial tissues, but much less with normal endometrial epithelium or supportive stroma and vessels, which were also present in the whole tissue homogeneates.

### Discussion:

Genomic studies of endometrial carcinoma have revealed that there are two, and possibly three, types of endometrial carcinoma that are each characterized by multistep pathogenetic pathways characterized by different molecular profiles [28]. A wide variety of genetic and enzymatic markers characterize the initiation, promotion and progression toward each type of endometrial carcinoma. Large-scale messenger RNA expression analysis of the endometrioid type of endometrial carcinoma has identified 50 genes that are capable of discriminating normal from malignant endometrial tissues [29]. Many genes that are constitutively expressed in normal endometrium show either diminished or increased expression in endometrial carcinomas. In addition, there is aberrant expression of the one hundred hormonally regulated genes that are variably expressed in normal endometrial tissues, although finally endometrial carcinoma resembles proliferative endometrium more than secretory endometrium [29].

In contrast to the genomic studies of the endometrium, no studies of the endometrial proteome are available, even though proteome analysis may offer information about protein expression, functions, and modifications which might not be fully reflected by gene expression analysis. Thus, protein expression profiling of the endometrium offers a new opportunity to identify and classify endometrial phenotypes, including carcinoma. Among the available methodologies for protein profiling, SELDI-TOF based method has the advantage of requiring only miniscule samples and high throughput capability, although the determination of any protein identity requires much more work [30].

This proteomic study of endometrium used lysates of whole tissue homogenates from both control endometrial tissues and endometrial carcinomas. Such tissues include not only the epithelial cells of interest, but also supportive stroma (including both endometrial stromal cells or fibroblasts and extracellular matrix), blood vessels (including smooth muscle cells and endothelium), any secretions, and possibly small amounts of adjacent myometrium. While the use of such whole tissue homogenates is technically straight forward, it does have inherent limitations. The heterogeneous nature of the constituent tissue will lead to a similarly heterogeneous protein profiling [17]. The resultant proteomic analysis reflects not only the cells of interest, but also the presence of contaminating cells [15, 17, 31]. Successful proteomic analysis of some types of tumors (e.g. hepatoma) may still be productive even with the limitation of whole tissue homogenization, since there is an abundance of tumor cells and minimal associated contaminating stroma [32].

In some tumor types, the inherent limitation of whole tissue homogenates must be surmounted by utilizing cell purification techniques. In the endometrium LCM may be optimal in achieving cellular purification for proteomic analysis since there is a relative abundance of stroma in endometrial carcinoma tissues [32]. Studies using laser capture microdissection (LCM) of melanoma have clearly revealed a different protein profile for melanoma than that of the surrounding epithelium, and thus supports the use of LCM in proteomic profiling studies [21, 33-35].

Despite the limitations of using whole tissue homogenates for protein expression profiling, this preliminary study of endometrial carcinoma has shown that proteomic analysis of endometrial carcinoma can detect differences from that of normal endometrium. Furthermore, a specific protein (Chaperonin 10) was strongly associated with endometrial carcinoma cases. This potential marker might be clinically useful on tissue aspirates since its differential expression pattern can be detected without the LCM procedure.

Chaperonin 10 was not identified exclusively in malignant endometrial tissues; low levels were detected in non-malignant endometrial tissues by both mass spectrometry and western blotting techniques (Table 2). Furthermore, there is no apparent association between any particular histopathologic classification and the detection of these low levels of chaperonin 10. In contrast, high levels of chaperonin 10 were detected in 17 of 22 malignant endometrial tissues by either mass spectrometry and/or western blotting techniques (Table 3). The apparent absence of chaperonin 10 in the remaining five of the 22 malignant cases may be due to either true absence or technical factors in pre-analytic processing or proteomic analysis. In two of these five cases, re-examination of the corresponding mirror image histologic section revealed minimal tumor in one case (case 28), or abundant necrosis of tumor (case 44). Furthermore, specific protein peaks of interest may be obscured in less-than-optimal mass spectromectric analysis or by adjacent protein peaks.

Chaperonin 10 (Cpn 10) is a heat shock protein (HSP) that functions intra-cellularly as a molecular chaperone for nascent proteins [36, 37]. HSP's are ubiquitous intra-cellular proteins that ensure homeostasis of metabolism [37]. Aberrations of HSP function, including chaperonin 10, have been described in a variety of pathologic conditions, including neoplasia [37, 38]. Furthermore, HSP's are differentially expressed in a variety of neoplasms. For example, immunohistochemical studies of both primary and secondary brain tumours have shown production of other HSP's [39], and that the expression of some HSP's may depend upon proliferating potential. Furthermore, the modulation of HSP expression profile has been shown to reflect the stage of prostatic carcinoma [37].

Immunohistochemical studies have identified HSP's within the nuclei and cytoplasm of epithelium, stroma, endothelium, and lymphocytes of the endometrium, with certain types of HSP's showing preferential localization to certain cell types [40]. Whereas the expression of some HSP's occurs independent of the stage of the endometrial cycle (e.g. HSP90), the expression of other HSP's is cycle dependent. The expression of chaperonin 10 can be determined among proliferative, secretory, and menstrual endometria using proteomic, western blotting, and immunohistochemical methods. It is known that the amount of HSP27 and 60 is increased during late proliferative and early secretory phases, and subsequently reduced during mid- and late secretory and menstrual phases [40]. Studies of both decidualized endometrium (decidua) and placenta have shown that there are striking differences in the cellular localization of HSP's during normal human gestation.

There have been no studies regarding the role of HSP's or Cpn10 in endometrial carcinoma. The eutopic endometrial glands from women with endometriosis and adenomyosis shows significantly increased expression of HSP's as compared to endometria from control women—regardless of the menstrual phase [41]. The abnormal expression of HSP's may play a role in the pathophysiology of both endometriosis and adenomyosis.

Early pregnancy factor (EPF) is an extra-cellular homologue of Cpn 10 that appears within 24 hours of fertilization and persists throughout the first half of gestation [36]. It is necessary for embryonic development [42-44] and is immunosuppressive [45]. EPF is also detectable in animal models of liver regeneration and in the development of cancer [38, 46, 47]. An association between cellular growth and the appearance of extracellular EPF has been shown [38, 46, 47]. These findings suggest a role for EPF in neoplastic growth and that the detection of Cpn 10 in the serum of endometrial carcinoma has diagnostic potential.

In conclusion, this protein profiling study of non-malignant and malignant endometrial tissues has identified chaperonin 10 as a tumor marker for endometrial malignancies.

### EXAMPLE 4

The work described in the previous example using solid-phase extraction followed by matrix-assisted laser desorption/ionization mass spectrometry (MALDI MS) as well as selective surface binding and surface-enhanced laser desorption/ionization (SELDI MS, Ciphergen Biosystems Inc, CA, USA) has succeeded in identifying individual markers that show significant enhanced expression in EmCa tissues. The MALDI/SELDI MS strategy relies on side-by-side comparison of spectra from proteins that have been selected via fast separation [120-124]. Using this methodology the identities of the proteins are unknown and identifying a given protein marker typically involves offline multidimensional chromatography, concentration, trypsin digestion and MS/MS. A second available strategy that highlights differentially expressed proteins involves differential tagging of proteins from samples that are being compared using isotope-coded affinity tag (ICAT) in an isotope-dilution mass spectrometry experiment [6]. This strategy has recently been applied to discover differentially expressed proteins between the proliferative and secretory phases of the human endometrium [49] using a cleavable, second-generation ICAT reagent (cICAT, Applied Biosystems Inc, CA, USA) (see above). Recently, a variation of the ICAT technology, iTRAQ (also from the Applied Biosystems Inc, CA, USA), has been introduced. Both cICAT and iTRAQ tagging permit online identification of multiple markers and relative quantification of these proteins. Although similar in their basic concepts, the two tagging reagents and methodologies differ in significant areas. The cICAT method relies on tagging cysteine residues and isolating peptides containing these tagged residues by affinity chromatography. The net result is a reduction in the complexity of peptide pools generated by digestion with proteases including trypsin [6]. In the case of the new iTRAQ method, tagging is on primary amines. This difference in labeling strategy eliminates the dependence on relatively nonabundant cysteine containing peptides intrinsic to ICAT-based methods, thus potentially allowing the tagging of most tryptic peptides. Other noteworthy features of the iTRAQ technology are that relative quantification is performed via MS/MS and that there are four possible tags, which permit multiplexing of up to four samples (tissue states) in a single experiment. Quantification is performed via the differences in abundances of four product ions, 114, 115, 116 and 117 Th that are each cleaved from one of the four possible tags. The tags have an identical mass, a result of differences in other parts of the iTRAQ tag structure, with the consequence that an identical peptide in the four samples will have an identical mass and LC retention time after tagging. This strategy simplifies analysis and will potentially increase analytical accuracy and precision. The multi-sample capability of the iTRAQ technology is ideally suited for this study, as it now provides a means to perform a proteomic analysis of both the major phases of the normal endometrium, while simultaneously comparing them against cancer samples.

This example describes a feasibility study that compares protein expression profiles between normal and cancerous endometria using iTRAQ as well as contrasts it against a similar feasibility study using the cICAT technology. Both iTRAQ and cICAT labeling afforded discovery of a number of differentially expressed proteins that are potential cancer markers (PCMs). There is little overlap in the PCMs discovered and identified, thus pointing to the complementary nature of the two technologies. It is noteworthy that application of the iTRAQ methodology permits confirmation of the overexpression of chaperonin 10 in EmCa tissues [50]; chaperonin 10 is an approximately 10 kDa heat shock protein that does not contain the cysteine residue, which is the tagging site for cICAT. Thus, in addition to an interest in the discovery of markers for endometrial carcinoma, these experiments have also helped to illustrate the relative strengths and differences of the two tagging techniques, when applied to studies of clinical samples.

### Materials and Methods

Sample Preparation. Endometrial tissue was retrieved from an in-house dedicated, research endometrial tissue bank. All tissues were snap frozen in liquid nitrogen within 15-20 minutes of devitalization at the time of hysterectomy, and were obtained with patient consent. The patient consent forms and tissue-banking procedures were approved by the Research Ethics Boards of York University, Mount Sinai Hospital, University Health Network, and North York General Hospital. In each case, the endometrium was classified by a pathologist (TJC). Histological classification was verified by examination of a section from the frozen research tissue. Tissue for proteomic analysis was taken from the mirror-face of the residual block. In the case of samples used for iTRAQ analysis, 0.5 ml phosphate buffered saline (PBS) containing protease inhibitors (1 mM AEBSF, 10 μM leupeptin, 1 μg/ml aprotinin and 1 μM pepstatin) was added. The tissue was then mechanically homogenized at 30,000 rpm using a Polytron PT 1300D handheld homogenizer (Brinkmann, Westbury, USA). For samples used for cICAT analysis, tissues were similarly homogenized in 1 ml Hanks' Balanced Salt Solution with the same concentration of protease inhibitors as listed above. The samples were stored in aliquots at −70° C. until used for further processing. These whole tissue homogenates contained not only endometrial epithelium, but supportive stroma and vessels, as well as secretions. The iTRAQ analysis involved one normal proliferative, one normal secretory, and two cancer homogenates, while the cICAT analysis combined one normal proliferative homogenate with three different cancer homogenates in pair-wise comparisons.
Chemicals. Reagent grade chemicals were purchased from Sigma Aldrich (Oakville, ON, Canada), or Fisher Scientific (Nepean, ON, Canada). All iTRAQ and cICAT reagents and buffers were obtained from Applied Biosystems (Foster City, Calif., USA).
iTRAQ Sample Preparation Procedure. Cell debris from each of the homogenates was removed by centrifugation in a microfuge at 4° C. for 30 min at 14,000 rpm. The clarified supernatant was then transferred to fresh microfuge tubes and the total protein content determined using a commercial Bradford assay reagent (Bio-Rad, Mississauga, ON, Canada). A standard curve for the Bradford assay was made using γ-globulin as a control. 100 μg of each sample was then denatured and the cysteines blocked as described in the iTRAQ protocol (Applied Biosystems, Foster City, Calif., USA). Each sample was then digested with 0.2 mL of a 50 μg/mL trypsin (Promega) solution at 37° C. overnight and labeled with the iTRAQ tags as follows: normal proliferative endometrium, iTRAQ 114; normal secretory endometrium, iTRAQ 115; and the two EmCa samples, iTRAQ 116 and iTRAQ 117. The labeled samples were then pooled and acidified by mixing with Eluent A (see later) to a total volume of 2.0 mL for strong cation exchange (SCX) chromatography. 1.5 mL of this acidified labeled sample was injected into an HP1050 LC system (Agilent, Palo Alto, Calif., USA) with a 1.5 mL injection loop and a 2.1 mm internal diameter (ID)×100 mm length PolyLC Polysulfoethyl A column packed with 5 μm beads with 300 Å pores (The Nest Group, Southborough, Mass., USA). A 2.1 mm ID×10 mm length guard column of the same material was plumbed upstream from the analytical column. Fractionation was effected by a binary mobile-phase gradient at a total flow rate of 0.2 mL/min. Eluent A consisted of a 10 mM KH₂PO₄ solution in 25% acetonitrile and 75% deionized water acidified to a pH of 3.0 with phosphoric acid. Eluent B consisted of a 10 mM KH₂PO₄ and 350 mM KCl solution in 25% acetonitrile and 75% deionized water acidified to a pH of 3.0 with phosphoric acid. Initially, the gradient comprised 100% Eluent A. At the 2^{nd} minute, the % Eluent B was changed linearly from 0 to 100% at the 58^{th} min. The run was terminated at the ₆₀^{th} min. A total of 30 fractions were collected for the sample, one every two minutes, using an SF-2120 Super Fraction Collector (Advantec MFS, Dublin, Calif., USA). Following fractionation, the samples were dried by speed vacuuming and stored at −20° C. Prior to reverse phase nanobore liquid chromatography-tandem mass spectrometric (nanoLC MS/MS) analysis, these fractions were redissolved in an aqueous buffer containing 5% acetonitrile and 0.1% formic acid for nanoLC MS/MS.
cICAT Sample Preparation Procedure. Samples were clarified and their total protein content determined as described above. cICAT sample preparation procedure was carried out according to the cleavable ICAT protocol (Applied Biosystems, Foster City, Calif., USA). Three 100 μg aliquots of the clarified normal proliferative homogenate were paired with 100 μg each of the three clarified cancer homogenates separately. After denaturing and reduction, the normal homogenates were labeled with the light reagent while the EmCa homogenates were labeled with the heavy reagent. A light labeled sample was then mixed with one of the heavy labeled samples to form in total three ICAT sample pairs: A, B and C. The final volume of each sample was 0.2 mL. The sample pairs were then digested by incubating each pair with 0.2 mL of a 50 μg/mL trypsin solution at 37° C. overnight. Afterwards each sample pair was mixed with 2.0 mL of Eluent A and fractionated into 30 fractions using SCX chromatography as described above. The fractions were screened by MALDI MS analysis. Those showing the signature ICAT peak pairs separated by 9 Da were further processed by affinity purification using the avidin cartridge provided with the ICAT kit. The affinity-purified sample was then dried, treated with the cleavage reagent to eliminate biotin, and dried again by speed vacuuming. The resulting solids were re-dissolved in an aqueous buffer containing 5% acetonitrile and 0.1% formic acid for nanoLC MS/MS.
Nanobore LC/MS/MS. The nanobore LC system was from LC Packings (Amsterdam, The Netherlands) and consisted of a Famos autosampler and an Ultimate Nano LC system. It was interfaced to an API QSTAR Pulsar QqTOF mass spectrometer (Applied Biosystems/MDS Sciex, Foster City, Calif., USA) using a Protana NanoES ion source (Protana Engineering A/S, Odense, Denmark). The spray capillary was a PicoTip SilicaTip emitter with a 10 μm ID tip (New Objective, Woburn, Mass., USA). The nanobore LC column was a 75 μm ID×150 mm length reverse-phase PepMap C18 nano capillary column (LC Packings, Amsterdam, The Netherlands) packed with 3 μm beads with 100 Å pores. One μL of sample was injected via the full-loop mode. Separation was performed using a binary mobile-phase gradient at a total flow rate of 200 mL/min. Eluent A consisted of 94.9% deionized water, 5.0% acetonitrile and 0.1% formic acid (pH 3). Eluent B consisted of 5.0% deionized water, 94.9% acetonitrile and 0.1% formic acid.

The following binary gradient was used for the iTRAQ experiments:

| | Time (min) | | | | | | | | |
|---|---|---|---|---|---|---|---|---|---|
| | 0 | 5 | 10 | 125 | 135 | 155 | 160 | 162 | 188 |
| % Eluent B | 5 | 5 | 15 | 35 | 60 | 80 | 80 | 5 | Stop |

While the binary gradient used for cICAT experiments was:

| | Time (min) | | | | | | | |
|---|---|---|---|---|---|---|---|---|
| | 0 | 5 | 125 | 135 | 157 | 160 | 163 | 190 |
| % Eluent B | 5 | 5 | 30 | 60 | 80 | 80 | 5 | Stop |

For nanospray, the source conditions were a curtain-gas setting of 20 and an ionspray voltage in the range of 1800-3000 V that was optimized daily. In the Q0 region, the instrument parameters were a declustering potential (DP) of 65 V and a focusing potential (FP) of 265 V. Nitrogen was used as the collision gas at a setting of CAD=5 for both TOF-MS and MS/MS scans. All nanoLC MS/MS data were acquired in information-dependent acquisition (IDA) mode in Analyst QS SP8 with Bioanalyst Extension 1.1 (Applied Biosystems/MDS Sciex). Two sets of runs for the iTRAQ fractions were performed. For the first set, MS cycles comprised a TOF MS survey scan with an m/z range of 400-1500 Th for 2 s, followed by three product ion scans with an m/z range of 70-2000 Th for 10 s each. For the second, a 1 s TOF MS survey scan, followed by three product ion scans of 3 s each were used. For cICAT experiments, the MS cycles consisted of a TOF MS survey scan for 1 s, followed by two product ion scans of 2 s each. The ranges for the TOF MS and product ion scans were the same as those of the iTRAQ experiments. Collision energy (CE) was automatically controlled by the IDA CE Parameters script. Switching criteria were set to ions greater than m/z=400 Th and smaller than m/z=1500. Th with a charge state of 2 to 5 and an abundance of ≧10 counts/s. Former target ions were excluded for 60 s and ions within a 4 Th window were ignored. In addition, for iTRAQ runs, the IDA Extensions II script was set to no repetitions before dynamic exclusion for the first run, and one repetition for the second. While for the ICAT experiments, the IDA Extensions II script was set to two repetitions before dynamic exclusion. In all three experiments, the script was set to select a precursor ion nearest to a threshold of 15 count/s every 4 cycles. These settings ensured examination of not only high abundance ions, but low abundance ones as well.

Data Analysis. Data analysis for the iTRAQ experiments was performed with ProQUANT 1.0, while that for cICAT experiments was with ProICAT 1.0 SP3. The cut off for the confidence settings for both analyses was at 75; that for the score was at 20. The tolerance set for peptide identification in ProQUANT searches were 0.15 Da for MS and 0.1 Da for MS/MS, while those for ProICAT searches were, 0.2 Da and 0.1 Da, respectively. All identifications were manually inspected for correctness. Relative quantification of proteins in the case of iTRAQ is performed on the MS/MS scans and is the ratio of the areas under the peaks at 114 Da, 115 Da, 116 Da and 117 Da which are the masses of the tags that correspond to the iTRAQ reagents. Relative quantification of proteins in the case of cICAT was performed on the TOF-MS scans by calculating the relative areas between the pairs of light and heavy label series of peaks. In both cases, the results of the quantification were normalized using the overall ratio obtained for all tagged peptide pairs in the sample. On the basis of previous studies utilizing cICAT reagents to compare secretory to proliferative endometrium, which involved paired tissue analysis of samples labeled with heavy and light isotopes, it was found that many proteins were expressed within a standard deviation (SD) of <0.3 and an expression ratio close to 1.0, when protein expression was normalized against a housekeeping protein (n=6) [49]. Using this SD as an approximate benchmark for variation of protein expression among proteins whose expression is not altered when utilizing this technique, it was considered that a difference of 3 SDs i.e. 1.0 plus 0.9, or about a 2 fold difference, in normalized expression levels would be outside of the variation expected by chance, with a confidence limit of >95% using a simple Gaussian approximation. This approximation would, therefore, apply to normalized expression levels >2.0 or in reciprocal form <0.5, as no assumption was made with regard to which tissue showed greater abundance. Using this approach, therefore, a two-fold expression change was chosen as an initial benchmark for potentially significant changes. In small sample sizes used for these initial studies, i.e. one sample in each group, at an alpha value of 0.05 (two-sided) and a beta value of 0.2 (80% power), the ratio of expression increase to SD must be at least 4 in order to achieve significance. This means that for minimal statistical significance to be achieved using the above alpha and beta parameters for a single paired-tissue comparison, a 2-fold mean increase in expression must be associated with an SD less than 0.5 for replicate measurements in order to satisfy the sample size equation [n=2 SD²(Z_{α}+Z_{β})²/(expression increase)²]=1, when the power index (Z_{α}+Z_{β})²=8 (α=0.05, β=0.2). For the sample size of three paired comparisons (one normal tissue and three tumor tissues were used in the cICAT experiment), the sample-size approximation of n=2 in each group was used to compensate for the fact that one normal tissue was used (i.e. n=1 in the control group, and n=3 in the tumor group). The ratio of expression increase to SD must be greater than 2.8 in order to satisfy the above equation for an n=2 (using the same α and β error limits of 0.05 and 0.2, respectively).

### Results

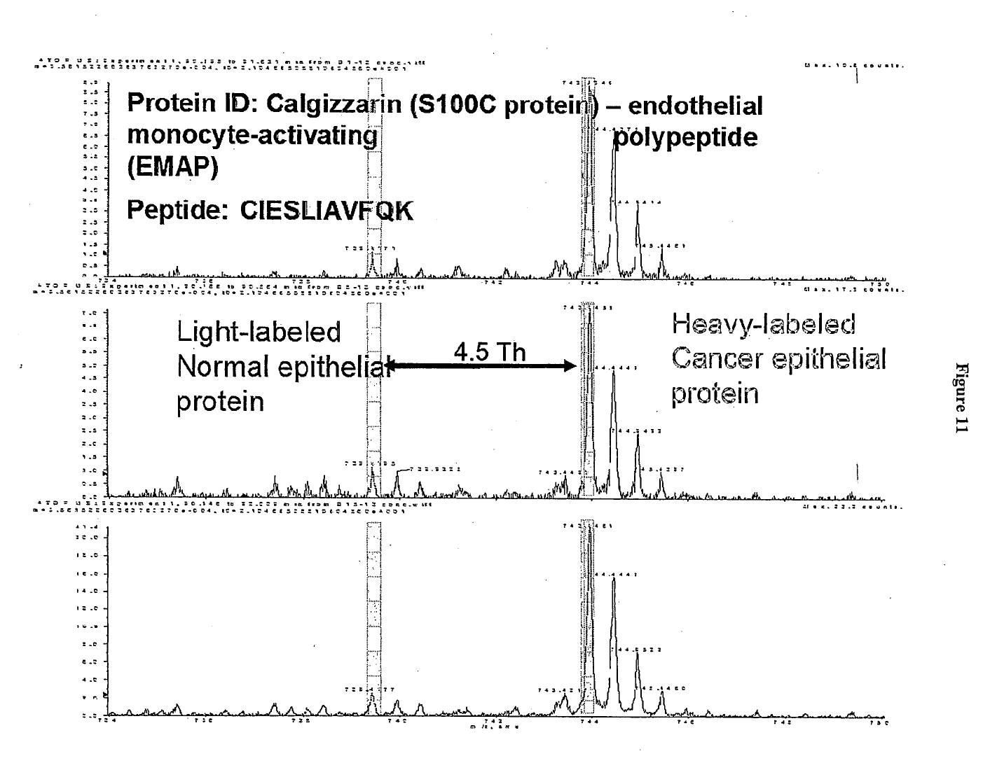

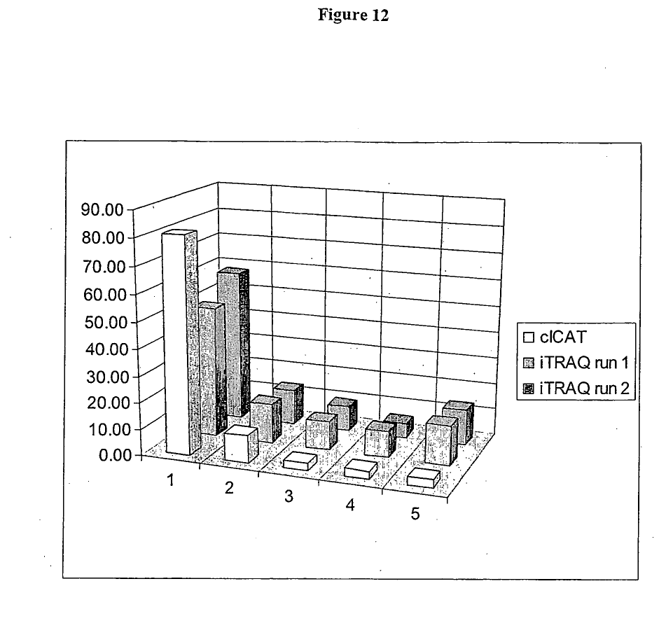

iTRAQ runs 1 and 2 led to sequencing and identification of 645 and 1,026 peptides, respectively. The previous studies (see above) showed that a confidence setting of 75 and a score setting of 20 were optimal. Manual inspection of hundreds MS/MS spectra concluded that while these conditions were sufficiently stringent in that most identifications were correct, they also did not exclude too many peptides that could have been identified based on spectral quality. The MS/MS spectra of all peptides that scored above the cut offs were manually inspected to verify proper identifications. The shorter MS and MS/MS cycles used for run 2 resulted in a higher number of peptides identified. A significant number of these peptides were identified more than once, thus the number of unique peptides dropped to 292 and 312 for runs 1 and 2, respectively. Many of the more abundant proteins were identified by several peptides (FIG. 12); the numbers of proteins identified were 101 and 126, respectively. 63 proteins were identified in both runs. The numbers are modest, which were likely due to the small amount of starting materials. Five proteins have abundance ratios that show more than a two-fold change (≧2.0 or ≦0.5) in both cancer samples relative to the proliferative as well as the secretory endometrium, and meet the criteria of differential expression. These are shown in Table 4.

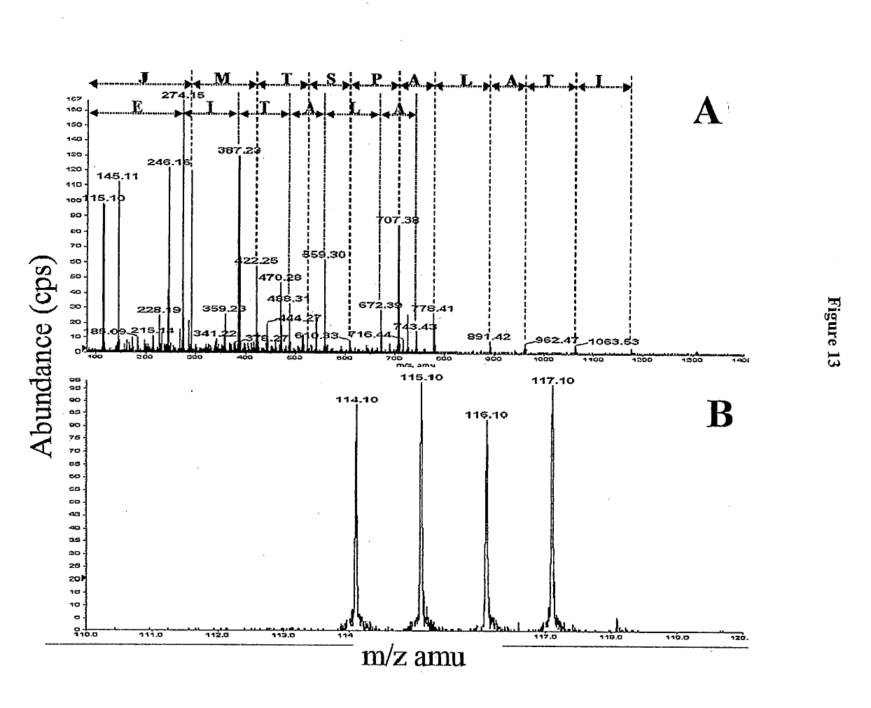

Relative quantification is expressed as three pair-wise ratios against the proliferative endometrium (iTRAQ 114) for the secretory endometrium (iTRAQ 115) and the two EmCa samples (iTRAQ 116 and 117). To account for small differences in protein loadings across the samples, these ratios have been normalized using the overall ratios for all proteins in the samples, as recommended by Applied Biosystems. The rationale for this choice is based on the assumption that the relative abundances for the majority of proteins are close to one. This assumption is exemplified in this study in terms of the abundance ratios in secretory versus proliferative endometrium for the following abundant proteins: cytoplasmic actin, 0.98; alpha enolase, 0.91; alpha filamin, 1.04; serum albumin precursor, 1.56; and tropomyosin alpha 4 chain, 0.99. FIG. 13a shows the MS/MS spectrum for the doubly charged cytoplasmic actin peptide at 725.4 Th in one of the runs. The cluster of peals around 115 Th is better shown in the mass spectral window 110-120 Th in FIG. 13b, demonstrating near identical abundances in the four samples: proliferative (iTRAQ 114), secretory (iTRAG 115), EmCa 1 (iTRAQ 116), and EmCa 2 (iTRAQ 117).

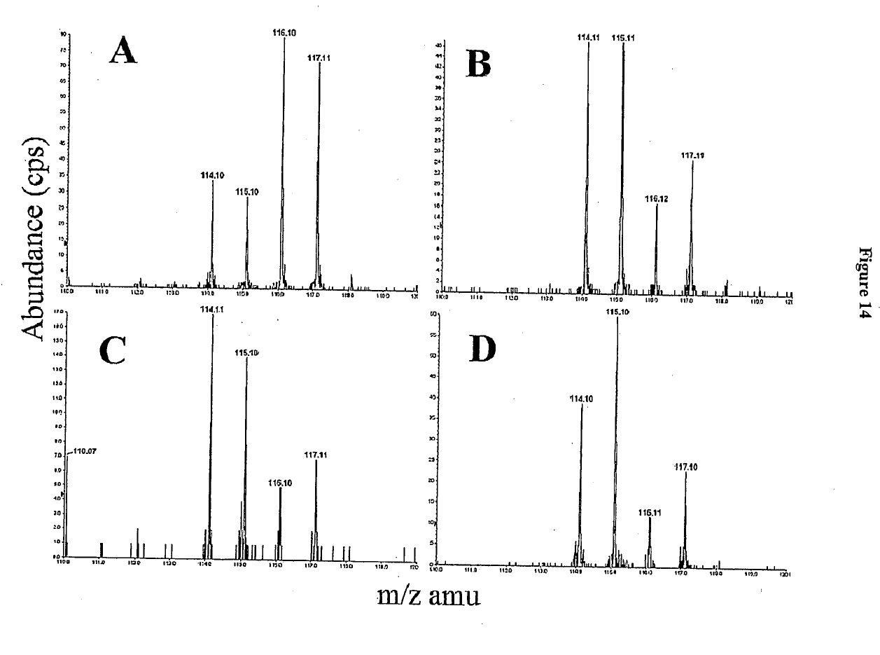

Typical MS/MS windows (10-120 Th) for tryptic peptides in four differentially expressed proteins, (A) chaperonin 10, (B) alpha-1-antitrypsin precursor, (C) creatine kinase B, and (D) transgelin, are shown in FIG. 14. Chaperonin 10 is overexpressed in both EmCa samples, whereas alpha-1-antitrypsin precursor, creatine kinase B, and transgelin are underexpressed. The remaining differentially expressed protein, pyruvate kinase M1 or M2 isozyme, is also overexpressed in the two EmCa samples.

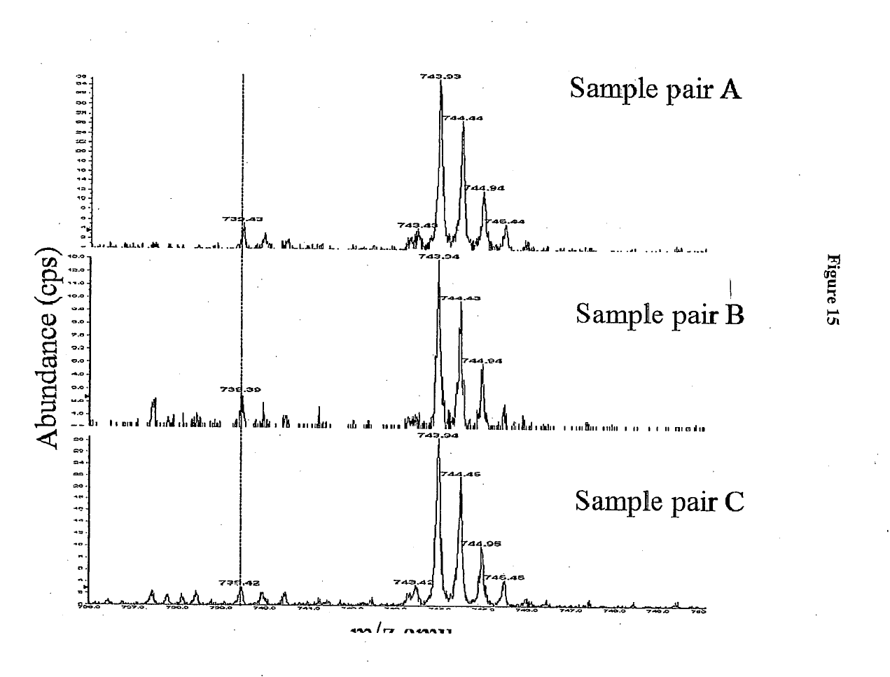

The cICAT experiments led to identification and quantification of 68 proteins, all were manually verified. Again, the modest number stemmed from the small sample size. Fewer proteins were identified by multiple peptides relative to iTRAQ (FIG. 12), in accordance with the more selective nature of cICAT labeling on only cysteine residues. Five proteins that meet the two-fold differential expression criterion in all three EmCa versus proliferative endometrium pairs are: calgizzarin, heterogeneous nuclear ribonucleoprotein (hnRNP) D0, macrophage migration inhibitory factor (MIF), polymeric immunoglobulin receptor (PIGR) precursor, and pyruvate kinase M1 or M2 isozyme. These results are summarized in Table 5. All five proteins are overexpressed in EmCa tissues; pyruvate kinase is also shown to be overexpressed with iTRAQ. FIG. 15 shows an example of overexpressed protein, calgizzarin A. Again, the relative abundance ratios are normalized to the overall ratio of all proteins in a given sample pair to account for small differences in protein loadings in the two samples. Cytoplasmic actin exhibits a heavy/light label ratio of 0.95±0.24 (standard deviation of three samples).

### Discussion

The combination of iTRAQ and cICAT labeling results in the discovery and identification of nine differentially expressed proteins that are potential cancer markers (PCMs) for EmCa. Six of the nine PCMs are overexpressed, whereas three are underexpressed in EmCa.

PCMs Overexpressed in EmCa. Chaperonin 10 is a heat shock protein that was identified as a PCM using MALDI/SELDI MS and identified by offline separation, preconcentration, trypsinization and MS/MS (see above). Overexpression of chaperonin 10 in EmCa tissues was verified independently by Western analysis; chaperonin 10 was localized to the cancerous epithelium by means of immunohistochemistry (IHC). Elevation of levels of chaperonin 10 has been associated with large bowel and cervical carcinomas [53]. The level of chaperonin 10 in serum was demonstrated to be an indicator of trophoblastic tumor [54]. The observation of chaperonin 10 overexpression in EmCa samples in this study with iTRAQ confirms the previous finding using MALDI/SELDI MS, Western analysis and IHC. Thus, chaperonin 10 may be a serum marker protein for EmCa.

Overexpression of pyruvate kinase M1 or M2 isozyme was demonstrated by both the iTRAQ and cICAT applications. Pyruvate kinase was found to be expressed at elevated levels in both plasma and fecal samples in patients of gastrointestinal cancer [55,56]. The ability to detect pyruvate kinase in plasma samples is of particular interest as this might indicate that screening plasma for elevated pyruvate kinase levels in endometrial cancer cases may also be possible. An investigation into the activity of 12 enzymes related to the glycolytic pathway in cervical and endometrial cancers found that only two, pyruvate kinase and phosphoglucose isomerase, were significantly higher in both cancers [57].

Calgizzarin was found to be overexpressed in EmCa tissues using cICAT labeling. Calgizzarin was one of two proteins that exhibited significant upregulation in colorectal and lung carcinoma cell lines over normal colorectal mucosal cells [58]. Calgizzarin was one of three proteins that have been identified as tumor markers in mouse colon cancer [59]. The other two tumor markers were calgranulins A and B, the former of which was also identified as a protein marker in human endometrial carcinoma using SELDI MS. Identification was made possible by size-exclusion chromatography, trypsinization and online nanoLC MS/MS; confirmation of calgranulin A overexpression in EmCa was rendered by IHC in a tissue microarray format.

HnRNP D0, also known as AU-rich element RNA-binding protein 1, binds to the AU rich 3′ UTR of many proto-oncogenes. One study has shown that this protein is more abundant in murine neoplastic lung epithelial cell lines and that this abundance decreases when non-tumorigenic cells reach confluence or growth arrest [60]. Conversely, it was also found that the abundance was unaffected in spontaneous transformants from this cell line. Another more recent study using transgenic mice showed that overexpression in an isoform of this protein altered mRNA levels of several oncogenes, including c-myc, c-jun, c-fos and TNF-alpha [61]. The mouse line with the highest level of this isoform developed sarcomas [61].

Macrophage Migratory Inhibitory Factor is another protein that has been well documented as being involved with cancer [62]. This ranges from hepatocellular carcinomas [63], to non small cell lung cancer [64], to brain tumors [65] and gliblastomas [66].

PIGR has previously been detected in serum from patients with lung cancer and was shown to be significantly upregulated in the secretory component by an ELISA study involving 45 lung cancer patients compared with 45 control subjects [68]. Another study has also demonstrated a possible linkage between PIGR and bladder carcinoma with protein levels in serum being significantly increased in patients with transitional cell carcinoma [69].

PCMs Underexpressed in EmCa. Alpha-1-antitrypsin precursor is one of the three proteins that are underexpressed in EmCa tissues. Recent studies have shown downregulation of alpha-1-antitrypsin to be associated with malignant lymphoma as well as liver, lung, stomach, bladder and gall bladder cancer [70,71]. There is also evidence for upregulation contributing to enhanced cell migration and metastases of human colon cells in a rat model [72]. Such a contradiction might be explained by the effect of alpha-1-antitrypsin being tissue specific which would prove useful for distinguishing between forms of cancer.

Creatine kinase B shown here as being underexpressed in EmCa has likewise been demonstrated as being downregulated in cervical, colon, and lung cancers but not in hepatocarcinoma [73,74].

Transgelin is another protein observed to be underexpressed in this study. It has also been implicated as being downregulated in breast and colon carcinomas as well as in a lung epithelial cell line [75,76]. Transgelin was isolated and identified as an antigen from renal cell carcinoma; subsequent in-situ hybridization experiments, however, found that the malignant cells were negative with respect to transgelin and that the transgelin source was from the mesenchymal cells of the stroma [77]. Thus, downregulation of transgelin appears to be true for renal cell carcinoma as well.

Proteins Slowing Possible Differential Expression. There are four proteins that showed differential expression by cICAT, but a smaller than critical (two-fold) change by iTRAQ labeling. One such protein is cyclophilin A, which was observed as being overexpressed by approximately four-fold in the ICAT analysis; however, the iTRAQ experiments showed a smaller overexpression of 1.47±0.29 across both cancer samples in both runs compared with the normal proliferative sample, which is not considered as significant according to our criterion of a two-fold change. Cyclophilin A has recently been reported as overexpressed in lung cancer [76].

Triosephosphate isomerase is another protein showing overexpression by cICAT tagging. However, the relative standard deviation for this protein at ±72% was also large, suggesting a variable expression level. There is a recent study that suggests triosephosphate isomerase to be upregulated by two-fold in lung cancer [78]. Triosephosphate isomerase level was found to be highly variable in renal cell carcinoma [79]. iTRAQ labeling shows an overexpression of 1.55±0.28.

Superoxide dismutase [Cu—Zn] is another protein that showed a large variation, ±50%, in the cICAT results. This protein has been implicated in pancreatic adenocarcinoma [80]. iTRAQ results at 1.46±0.08 is below the criterion of overexpression. Phosphatidylethanolamine binding protein showed a cICAT overexpression level of 3.7±1.0 and an iTRAQ overexpression level of 1.39±0.13. There is evidence for upregulation in rat hepatoma cell lines [81].

The use of isotope-coded affinity tags and nanoLC/MS/MS afforded examination of a large number of proteins for differential expression. This method is a lot more efficient and powerful in terms of the number of proteins that can be examined in a given experiment than SELDI MS. However, complex data analysis and the need for manual examination of MS/MS data limit the number of samples that can be examined within a given period of time. As a consequence, iTRAQ tagging involved two normal endometrial tissues versus two EmCa tissues, whereas cICAT pairing involved only one normal endometrial versus three EmCa tissues. By contrast, PCM discovery using SELDI MS involved in excess of 40 samples [see above]. A contribution to the uncertainties of the aforementioned four proteins may simply be individual variations in protein expression, both within the normal group as well as the EmCa group. The experience with the results of chaperonin 10 and calgranulin A shows that, while there is overexpression in EmCa tissues, there are considerable variations in protein abundances, possibly reflecting variability in the cellular subpopulations of the whole tissue homogenates or in the type or nature of the EmCa. In addition, there is variation in the abundances of these proteins across individual normal endometrial samples.

Selectivity of PCMs. Literature data show that the nine differentially expressed proteins discovered and identified here are associated with cancers and are, indeed, potential cancer markers. Individually, these nine PCMs are nonspecific for endometrial carcinoma, as each has been linked with cancers other than EmCa. As the principal concern in screening, diagnosis, and monitoring of EmCa is the exclusion or omission of any malignant endometrial disease, the fact that these PCMs have been noted in other cancers would rarely be a potential clinical drawback. In other words, for envisaged clinical use, the sensitivity is of far more concern than specificity for EmCa. It is probable that different cancers will share similar pathways in tumorigenesis, which will induce over- and underexpression of similar proteins [57]. Nevertheless, it is unlikely that the differential expression pattern involving many proteins will be identical for all or some primary tumor sites. Their collective use as a panel may be more effective in indicating the site of origin of a cancer [82,84], despite the association of individual markers with a variety of primary sites.

The absolute expression level of PCMs will be an important issue. Knowing protein abundances in normal and diseased states will allow establishment of threshold levels beyond which EmCa is signaled. iTRAQ permits simultaneous investigation of up to four samples, thus facilitating the inclusion of synthetic tryptic peptides of known amounts in absolute isotope-dilution experiments

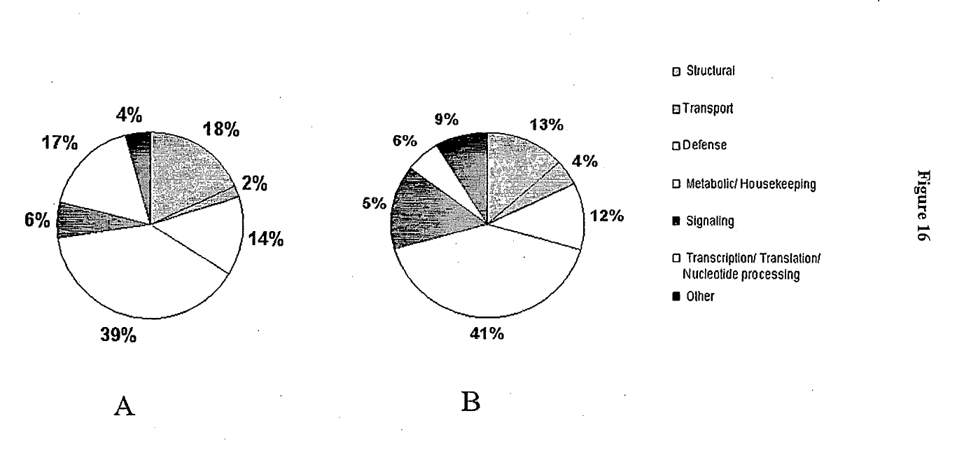

iTRAQ versus cICAT. The results obtained by iTRAQ and ICAT analyses suggest that the information generated by the two methods is complementary. There are, however, a few aspects on which each of these methods has advantages over the other. Quantification by cICAT can be compromised by overlapping peaks in the MS spectrum; this complication is resolved in iTRAQ as quantification is performed on the MS/MS spectrum. iTRAQ on the other hand requires processing samples separately until after the tryptic digestion. This increases the potential for errors introduced, as a result of sample handling or different extents of tryptic digestion. Another aspect on which iTRAQ differs is complexity because of the relatively nonspecific nature of labeling. Many more proteins are identified by multiple peptides. This is not necessarily a disadvantage, as it permits quantification of multiple peptides, thereby increasing the confidence in the ratios report. After classifying identified proteins into broad categories (FIG. 16), it is apparent that there is a higher proportion of signaling proteins identified by the cICAT method. Conversely, iTRAQ analysis identified a larger percentage of the more abundant ribosomal proteins and transcription factors.

### EXAMPLE 5

### Summary

Proteomic analyses of the proliferative and secretory phases of the human endometrium were carried out to identify proteins and discover differentially expressed proteins using isotope-coded affinity tags and three stages of chromatographic separation and online MS/MS. From an initial list of 346 proteins identified by ProICAT, manual inspection of MS/MS spectra and confirmatory searches pared the list down to 119 positively identified proteins. Only five of the proteins showed consistent differential expression. The two proteins with unquestionable differential expressions in the secretory endometrium are: glutamate NMDA receptor subunit zeta 1 precursor and FRAT1. Some of the proteins that show no differential expression have previously been examined in gene-expression studies with similar conclusions.

Below are reported results of a study to identify proteins in the human endometrium, and especially proteins that are differentially expressed in the proliferative and secretory phases. Differences in protein expression levels are highlighted and determined by the use of the cleavable ICAT reagent [8]. Proteins that are identified are compared with genes that have been examined for regulatory changes.

The following materials and methods were used in the study.

### Materials and Methods

### Tissue Samples

Endometrial tissues were retrieved from an in-house dedicated endometrial tissue bank. All tissues had been snap-frozen in liquid nitrogen within 15-20 minutes of devitalization at the time of hysterectomy. All tissue procurements were carried out after informed patient consents. The consent, procurement and banking procedures were approved by the Research Ethics Boards of York University, Mount Sinai Hospital, University Health Network, and North York General Hospital. In every case, the endometrium was classified as proliferative or secretory by a pathologist. The histological classification was verified by examination of a histopathologic section from the frozen tissue. The mirror-face of the residual block was used for proteomic analysis. After addition of 1 ml of Hanks' Balanced Salt Solution containing protease inhibitors (1 mM 4-(2-aminoethyl)benzenesulfonyl fluoride, 10 μM leupeptin, 1 μg/ml aprotinin and 1 μM pepstatin), the tissue sample was mechanically homogenized at 30,000 rpm using a Polytron PT 1300D handheld homogenizer (Brinkmann, Westbury, USA). The whole tissue homogenate contained not only endometrial epithelium, but supportive stroma, vessels as well as secretions. The homogenate was stored in aliquot at −80° C. and/or submitted for proteomic analysis. Samples from six different individuals were selected for this study. Three of these tissues had been classified as proliferative endometria (randomly designated as PRO1, PRO2 and PRO3), and the other three as secretory endometria (randomly designated as SEC1, SEC2, SEC3).

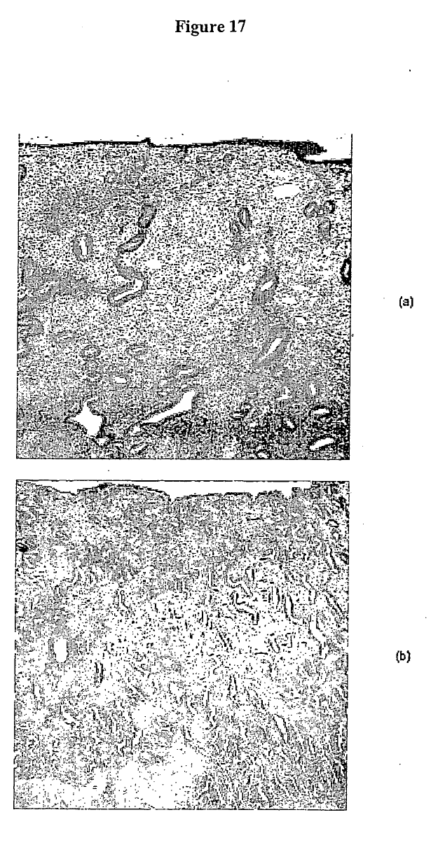

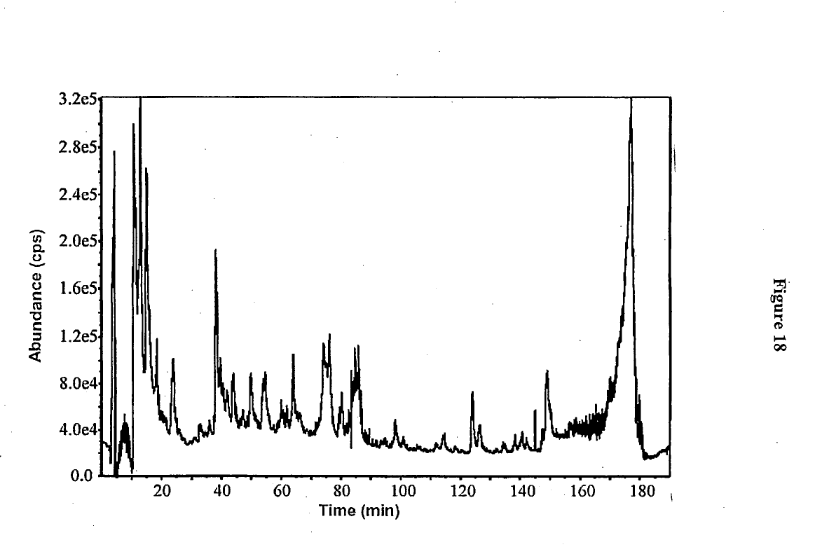

FIG. 17 shows an example of the histologic appearances of (a) a proliferative (PRO2) and (b) a secretory (SEC2) endometrium. In both endometria, the stratum basalis is characterized by a denser stroma than the physiologic responsive stratum functionalis above. Across the top of the stratum functionalis is the surface epithelium, which lines the endometrial cavity. The proliferative endometrium shows small, coiled glands with lining columnar epithelium reaching to the surface. By contrast, the secretory endometrium is thicker, and contains more tortuous glands with intra-luminal secretions. The endometria of both types of physiologic phases have abundant supportive stroma and vessels among the epithelial glands.

### Chemicals

Solvents, chemicals (except otherwise noted), and Hanks' Balanced Salt Solution were obtained from Sigma-Aldrich (Oakville, ON, Canada). All reagents and buffers for the cleavable ICAT sample preparation procedure were from Applied Biosystems (Foster City, Calif., USA).

### Sample Preparation

After removal of cell debris by centrifugation, the total protein content for each of the six clarified homogenates was measured using a commercially available Bradford protein assay (Bio-Rad, Mississauga, ON, Canada). ICAT sample preparation procedure was carried out according to the cleavable ICAT protocol (Applied Biosystems, Foster City, Calif., USA). One hundred micrograms of proteins was used per sample. The proteins were denatured with the Denaturing Buffer supplied with the ICAT reagent kit. Afterwards, disulfide bonds were cleaved by adding the reducing reagent supplied, which contained 50 mM tris-(carboxyethyl)phosphine, and boiling for 10 min. The proliferative samples were then labeled with the light ICAT reagent and the secretory samples with the heavy reagent by incubating with the respective ICAT label for 2 h at 37° C. The labeled PRO1 and SEC1 samples were combined to form ICAT sample A, PRO2 and SEC2 to form ICAT sample B, and PRO3 and SEC3 to form ICAT sample C. The final volume of each sample was 0.2 ml. Mixing of the labeled proliferative and secretory samples in pairs in this manner ensures that any protein or peptide losses during subsequent processing steps is the same for both samples in a pair. The sample pairs were then digested by incubating each pair with 0.2 ml of a 50 μg/ml trypsin solution at 37° C. overnight. The resulting peptides were fractionated by means of strong cation exchange chromatography using an HP1050 LC system (Agilent, Palo Alto, Calif., USA) with a 1.5 ml injection loop and a 2.1 mm internal diameter (ID)×100 mm length PolyLC Polysulfoethyl A column packed with 5 μm beads with 300 Å pores (The Nest Group, Southborough, Mass., USA). A 2.1 mm ID×10 mm length guard column of the same material was plumbed upstream from the analytical column. Fractionation was effected by a binary mobile-phase gradient at a total flow rate of 0.2 ml/min. Eluent A consisted of a 10 mM KH₂PO₄ solution in 25% acetonitrile and 75% deionized water acidified to a pH of 3.0 with phosphorus acid. Eluent B consisted of a 10 mM KH₂PO₄ and 350 mM KCl solution in 25% acetonitrile and 75% deionized water acidified to a pH of 3.0 with phosphorus acid. The trypsinized ICAT sample pair (0.4 ml) was mixed with 2.0 ml of Eluent A. 1.5 ml of the 2.4 ml sample was injected. Initially, the gradient comprised 100% Eluent A. At the 2^{nd} minute, the % Eluent B was changed linearly from 0 to 100% at the 58^{th} min. The run was terminated at the 60^{th} min. A total of 30 fractions were collected, one every two minutes, using an SF-2120 Super Fraction Collector (Advantec MFS, Dublin, Calif., USA). UV monitoring of the chromatographic eluent revealed abundant peptides in fractions 11-20. These 10 fractions were further purified by affinity chromatography according to the ICAT protocol recommended by Applied Biosystems. Eluted labeled peptides were treated with the Cleaving Reagent, which contains TFA in order to remove the biotin label, and resolved in a third stage of chromatographic separation using reverse-phase nanobore LC with online MS/MS.

### Nanobore LC/MS/MS

The nanobore LC system was from LC Packings (Amsterdam, The Netherlands) and consisted of a Famos autosampler and an Ultimate Nano LC system. It was interfaced to an API QSTAR Pulsar QqTOF mass spectrometer (Applied Biosystems/MDS Sciex, Foster City, Calif., USA) using a Protana NanoES ion source (Protana Engineering A/S, Odense, Denmark). The spray capillary was a PicoTip SilicaTip emitter with a 10 μm ID tip (New Objective, Woburn, Mass., USA). The nanobore LC column was a 75 μm ID×150 mm length reverse-phase PepMap C18 nano capillary column (LC Packings, Amsterdam, The Netherlands) packed with 3 μm beads with 100 Å pores. One μl of sample was injected via the full-loop mode. Separation was performed using a binary mobile-phase gradient at a total flow rate of 200 nl/min. Eluent A consisted of 94.9% deionized water, 5.0% acetonitrile and 0.1% formic acid (pH≈3). Eluent B consisted of 5.0% deionized water, 94.9% acetonitrile and 0.1% formic acid. The following binary gradient was used:

| | Time (min) | | | | | | | |
|---|---|---|---|---|---|---|---|---|
| | 0 | 5 | 125 | 135 | 157 | 160 | 163 | 190 |
| % Eluent B | 5 | 5 | 30 | 60 | 80 | 80 | 5 | Stop |

For nanospray, the source conditions were a curtain-gas setting of 20 and an ionspray voltage in the range of 1800-3800 V that was optimized daily. In the Q0 region, the instrument parameters were a declustering potential (DP) of 65 V and a focusing potential (FP) of 265 V. Nitrogen was used as the collision gas at a setting of CAD=5 for both TOF-MS and MS/MS scans.

All LC-MS/MS data were acquired in information-dependent acquisition (IDA) mode in Analyst QS SP5 with Bioanalyst Extension 1.1 (Applied Biosystems/MDS Sciex). A TOF-MS survey scan with an m/z range of 400-1500 and 1 s scan time was followed by two product ion scans with an m/z range of 70-2000 and 2 s scan time. The collision energy (CE) was automatically controlled by the IDA CE Parameters script. The switching criteria were set to ions greater than m/z=400 and smaller than m/z=1500 with a charge state of 2 to 5 and an abundance of ≧10 counts/s. Former target ions were excluded for 60 s and ions within a 4 Th window were ignored. In addition, the IDA Extensions II script was set to 2 repetitions before dynamic exclusion and to select a precursor ion nearest to a threshold of 15 count/s every 4 cycles. These settings ensured examination of not only high abundance ions, but low abundance ones as well. Each of the three sample pairs, A, B and C, was injected twice, thus yielding a total of six sets of runs.

Data analysis was performed using ProICAT 1.0 SP3 software (Applied Biosystems/MDS Sciex). An initial setting of a confidence value of 75 and a score of 15 was used. Relative quantification of proteins between the light and heavy labels was performed on the TOF-MS scans by calculating the relative areas under the series of peaks. Differential levels of expression were based upon measured protein expression ratios in proliferative versus secretory endometrium normalized to glyceraldehyde-3-phosphate dehydrogenase (GAPDH) expression and were considered significant when they exceeded three SDs from the mean expression of housekeeping proteins (actin, tubulin α-chain and triose phosphate isomerase). All expression ratios were based on a mean of two essays. The sample size calculation for determination of minimum numbers of tissue samples for ICAT analysis (N) was based upon the difference between two means, N=8 (SD/precision) [85]. Based on preliminary experiments, an SD value of 0.3 was observed with a target precision of 0.5, yielding a minimum sample size of 3 for each tissue group. The significance of differential protein expression between proliferative and secretory endometrium was evaluated using the Wilcoxon rank sum test.

### Results

FIG. 18 shows a typical nano LC/MS total ion chromatogram (TIC) from fraction 16 of sample A. As a total of 10 fractions were analyzed per sample and each sample was injected twice, there were a total of 60 TICs. The use of the IDA mode generated hundreds of MS/MS spectra per TIC.

Using the initial setting of a confidence value of 75 (ProICAT recommends a lower confidence value of 50) and a score of 15 in ProICAT 1.0 SP3 results in the identification of 346 non-redundant proteins. Manual inspection of the MS/MS spectra reveals that identification of some proteins that score between 15 and 20 may be problematic. A random selection of 50 spectra from proteins scoring between 15 and 20 shows that 15 were analyzed and sequenced correctly, 13 were probably correct, and 22 were incorrect. Manual inspection of the MS/MS spectra of the 145 proteins that score 20 or higher also reveals occasional errors in assignment, but at a rate considerably lower than that between scores 15 and 20. Unfortunately, the use of ICAT-labeling results in a reduced opportunity for corroborating identification based on a second tryptic peptide from the same protein. Although the majority of human proteins do contain more than one cysteine residue per protein, some of these residues may be located within tryptic peptides that are too small or large, others may fall into peptides that have poor ionization efficiencies. Of the 346 proteins, only 23 were identified with more than one peptide; 15 of these 23 proteins were identified with two peptides.

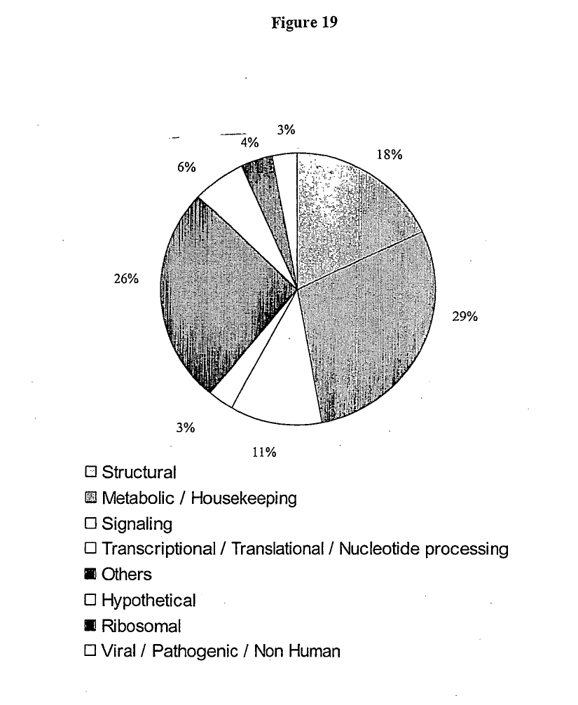

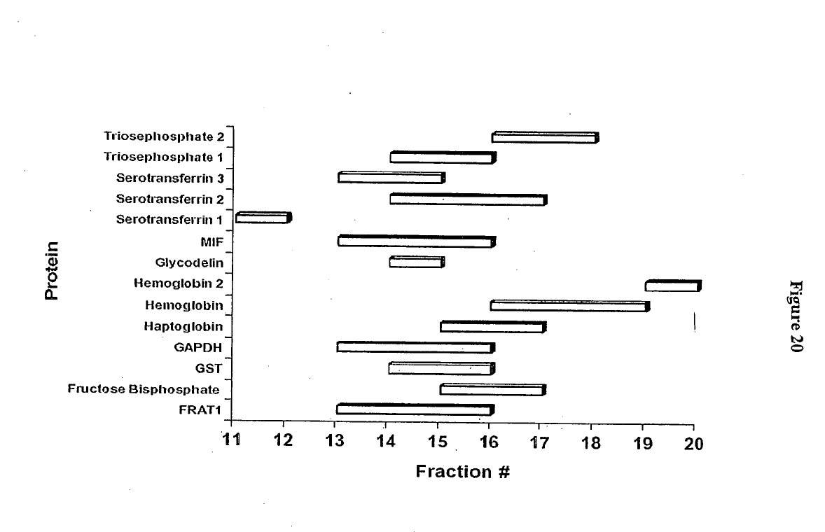

Table 6 lists the 119 proteins that have been identified by ProICAT and verified by manual inspection of MS/MS data. FIG. 19 shows the distributions of the proteins in the form of a pie chart. Of the 119 proteins, 15 were added after manual inspection of 50 randomly selected proteins' MS/MS spectra. If the MS/MS spectra of the remaining 151 proteins was inspected, it would have been expected to confirm the identifications of an additional ˜45 proteins. This, however, was considered impractical, especially after it was known that none of the 15 additional proteins were observed in all three runs and would contribute towards knowledge of differential expression.

The results show that expression levels of the majority of proteins identified are not consistently different between the proliferative and secretory phase of the endometrial cycle. As expected, the majority of identified proteins fall under the metabolic/housekeeping and structural categories. The proteins classified under “Others” include antibodies. A small percentage of the proteins are viral or pathogenic in origin, reflecting the possibility of infection in the biopsied endometria. A few proteins have no known functions or were identified from cDNA matches; these are, therefore, classified as hypothetical.

To normalize small variations of protein amounts, relative quantification of the protein levels is normalized to the ratio of GAPDH. Gene expression studies involving different stages in the endometrial cycle have used GAPDH expression to normalize differential mRNA expressions [88-90]. The assumption is that the constant mRNA level of GAPDH through the endometrial cycle translate to constant protein level (no variation caused by translational controls). The GAPDH ratio of 1.5±0.5 (one SD) before self-normalization in the six runs is in accordance with this assumption.

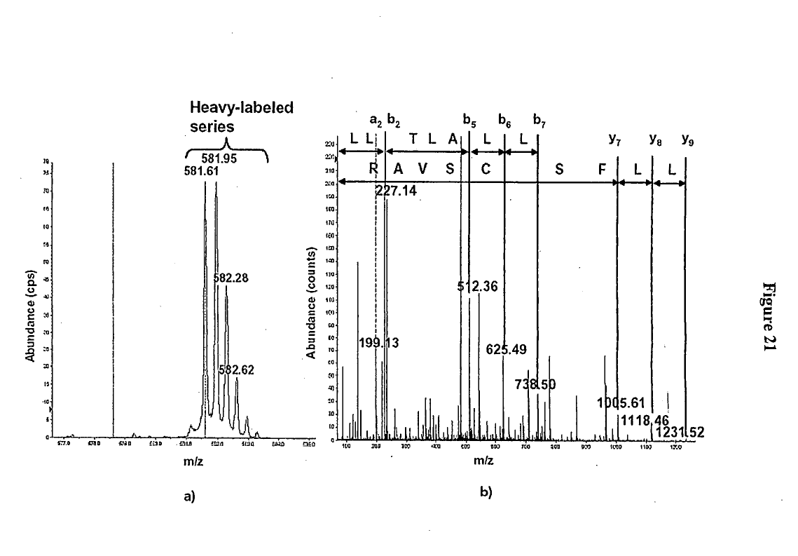

The ratios of the proteins in the secretory/proliferative phase (heavy/light label) listed in Table 6 are the averages ±SDs of the peptides in all the identified runs. In the case of more abundant proteins, e.g. serum albumin and serotransferrin, individual peptides were detected in as many as 3-4 fractions. Less abundant proteins, however, were frequently detected only in 1-2 fractions (see FIG. 20). In cases of multiple fractions, multiple peptides, and/or multiple runs, the ratios listed in Table 6 are the averages and the SDs of all contributing elements. Twenty four proteins show “extreme” differential expression in that only one labeled form was observable (these are noted as “Singleton H or L”. However, only five of these 24 proteins were observed in all three samples (and an additional one in two samples). All five proteins were identified by single tryptic peptides. The expression levels of three housekeeping proteins (actin, tubulin α-chain and triose phosphate isomerase) normalized against GADPH were pooled to establish a mean expression level of 1.07˜1.1 and an SD of 0.3. The SD of these expression levels was calculated from expression data normalized to a value of 1 for any expression ratios less than 1 to generate linear data. (This was done by taking the reciprocal of expression values less than 1 in the original data set; for example, an expression ratio of 0.7 was converted to its reciprocal 1.42.) This data was then used to calculate the SD of housekeeping protein expression. A significant differential expression value was, therefore, defined as a normalized expression ratio that was three SDs above the ratio of 1.0. On the basis of the observed data, this ratio was set as 2.0 (approximately 1.0+3(0.3)). Consequently, only two-fold or larger changes in expression ratios were considered to be of significance, based on the analysis of the variation in measured expression of housekeeping proteins. An additional stringency requirement of this level of expression in all three sample pairs was imposed due to the small initial sample set. No additional proteins in Table 6 meet the differential expression criteria of abundance ratios larger than 2.0 or smaller than 0.5 in all three sample pairs.

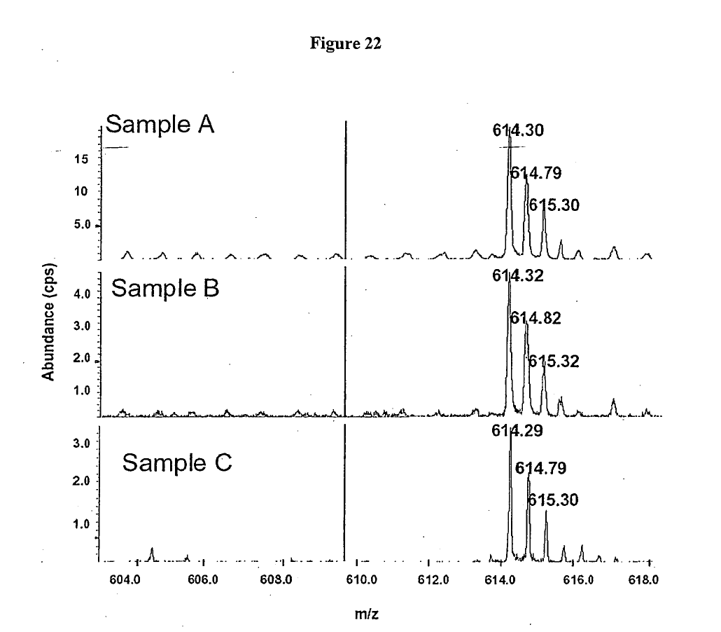

An example of extreme differential expression observable in all three sample pairs is shown in FIG. 21, which shows very significant expression enhancement of glutamate NMDA receptor subunit zeta 1 precursor in the secretory endometrium. The absence of the light-labeled analogue, which would have manifested as a cluster of peaks beginning at 578.6 Th, demonstrates the significantly lower expression of this protein in the proliferative endometrium. The triply charged tryptic peptide at 581.6 Th was identified as LLTLALLFSCSVAR [SEQ ID NO. 28], which maps to the N-terminal region of the protein. A second example is shown in FIG. 22, again showing extreme differential expression in the secretory phase. This protein was identified as FRAT1. For all five proteins that met the differential expression criteria stipulated above, a minimum p value of less than 0.04 was obtained on the basis of the Wilcoxon rank sum test, suggesting that the differences observed were of statistical significance.

### Discussion

The observation of similar expression levels in the majority of proteins shown in Table 6 is not surprising, as many proteins are structural and housekeeping in nature. In addition, structural and housekeeping proteins, e.g. tubulin and actin, are expressed in many tissue types. This is of significance as the biopsies that were examined comprise not only epithelial cells, but also supportive stroma cells, blood and vessels that are not expected to be affected by the endometrial cycle.

In this initial examination of three sample pairs, a conservative strategy was adopted of recognizing proteins as differentially expressed only when their expression ratios are larger than 2.0 or smaller than 0.5 in all three sample pairs. Of the 119 proteins listed in Table 6, five proteins are in this category. In fact, differential expression is in the extreme form that only single labels are apparent. There is one protein (POL polyprotein) that was seen only in the secretory endometrium, but was observed in only two sample pairs and does not meet the criteria of differential expression in this study (see later).

One of the five differentially expressed proteins is glutamate NMDA receptor subunit zeta 1 precursor, which was only observed in the secretory endometrium. This protein is a subunit of a ligand gated ion channel and is known to be involved with synaptic plasticity in neurons. A recent paper suggests that it may also play a role in glutamate-mediated toxicity to mitochondria, eventually leading to apotosis [91]. As stated earlier, the peptide maps to the N-terminal region of the precursor, which is normally cleaved to form the mature protein.

Similarly, FRAT1 is detected only in the secretory endometrial samples under the experimental conditions. There is no direct connection between FRAT1 and the endometrium reported in the literature; however, FRAT1 is expressed in a wide range of human tissues [92]. The gene expressing FRAT1 is a known proto-oncogene that activates the WNT pathway [93,94]. This pathway is important in the endometrial cycle; FRAT1 is known to inhibit c-Jun activity, thereby inhibiting subsequent apotosis [95]. c-Jun, in turn, has been shown to be expressed in the endometrium and this expression is at a higher level during the proliferative phase than the secretory phase [96].

Myosin light chain kinase 2 is another protein that was observed exclusively in the secretory endometrium in all three sample pairs. While there are no studies on the relative amounts of this enzyme during the endometrial cycle, previous studies showed that it is present in the human myometrium at a level that is four-to five-fold higher than that in endometrial stromal cells [97]. It was also demonstrated that the specific activity of myosin light chain kinase, which is believed to be essential for smooth muscle contraction, was 20-fold higher in the myometrium than that in non-muscle cells, e.g. skin fibroblasts [97].

Isopentenyl diphosphate delta-isomerase, was present only in the secretory endometrium. This protein was identified via the peptide ITMLCTGSRTLK [SEQ ID NO. 29]. In addition to this identification with ProICAT, a subsequent search with Mascot (Matrix Science) verified this assignment. This peptide, and therefore the protein, however, is unique and found only in the bacterium Rickettsia conorii. BLASTing the human and the R. conorii isopentenyl disphosphate delta-isomerase proteins showed no region of homology. In addition, MS BLASTing the peptide ITMLCTGSRTLK against non-redundant protein databases returned no other matches.

A protein that appeared in all three sample pairs was initially identified by ProICAT as a simian immunodeficient viral protein, envelope polyprotein GP160 precursor. However, searching Mascot with the same MS/MS spectra returned a different protein from clostridium, putative phosphatidylserine decarboxylase proenzyme (gi 28209853). Endometrial receptors for a clostridium toxin have been documented [98]; thus the identification of a clostridium protein is not entirely surprising.

Another protein initially identified by ProICAT as POL polyprotein from Rous sarcoma virus was observed only in the secretory endometrium in two of the three sample pairs. A subsequent Mascot search, however, returned a positive hit for an unlabeled peptide from serum albumin, and strongly suggested a wrong initial identification.

Tropomyosin 1 alpha chain was identified in two out of three sample pairs to be selectively expressed in the secretory phase at a ratio of 2.3±0.7. An enhanced expression of this protein in the secretory endometrium is in accordance with the result of a previous gene expression study that reported an up-regulation of 3.7 times [85].

A protein that showed no difference in expression levels (1.2±0.7 in three samples) is macrophage migration inhibitory factor, MIF. This observation is consistent with those in previous studies that demonstrated MIF as being expressed by the human endometrium throughout the menstrual cycle and, in particular, predominantly in the glandular epithelial cells [9]. MIF was found to localize throughout the glandular epithelial cytoplasm in the proliferative phase; however, this distribution changes in the secretory phase, when MIF localizes to the apical portion of the glandular epithelial cells and is also present in glandular secretions. Macrophages are common in female reproductive tissues. In the endometrium, they play an important role in defense. In addition, macrophage degradation of cellular debris and foreign material may play an important role in endometrial shedding and repair [9].

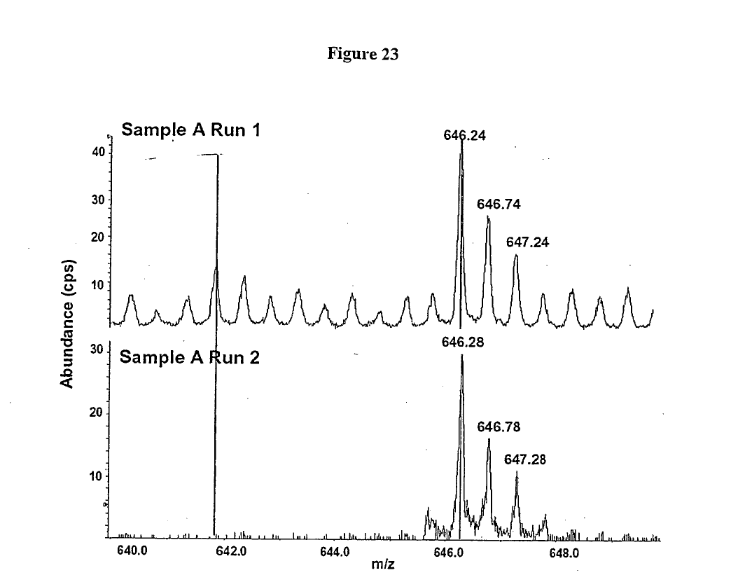

Another protein observed in this study and for which there was precedence in the literature is glycodelin. On the basis of gene expression studies [85], glycodelin was believed to be up-regulated during the secretory phase. In this study, the precursor of this protein was identified in only one sample pair, but it was identified in both runs in only the secretory endometrium (FIG. 23).

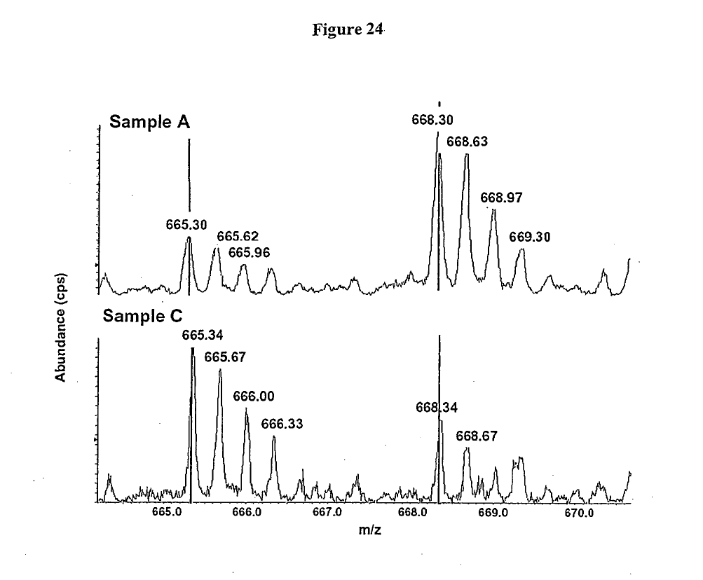

Cathepsin B which, according to a previous study, does not appear to be differentially regulated during the menstrual cycle [99], was seen in two of the sample pairs with opposite trends (FIG. 24). Glutathione S transferase is known to show large variability which is believed to result from individual differences, rather than endometrial cycling [100]. The relative expression level observed in this study is 1.3±0.4. Lactate dehydrogenase is another protein not believed to have any cyclic trend [101]; the relative level measured in this study, 1.4±0.3, is in accordance with this expectation. Peptidyl-propyl cis-trans isomerase A or cyclophilin A has been observed in placental and decidual tissues, but there was no study on relative amounts between the phases of the endometrial cycle [102]. The relative expression results of 1.2±0.2 show a constant level of expression. Haptoglobin is thought to be up-regulated during the secretory phase [103]. The average relative expression level of 2.2±1.6 is based on two enhanced secretory expression level measurements in two sample pairs and one reduced secretory expression level measurement in another sample pair that account for the very large SD. Haptoglobin is a liver protein secreted into blood. As the amounts of blood in the biopsied tissues could not be controlled prior to homogenizing, the large SD might simply reflect that the samples contain different amounts of blood as opposed to truly representing increased or decreased haptoglobin expression.

It should perhaps be emphasized that protein identifications using ICAT-labeled peptides are reliable as the identification of one labeled peptide in a pair corroborates the identification of the other in the pair. This offsets somewhat the reduced opportunities in seeing multiple peptides because ICAT targets only cysteine-containing peptides. The chance of a missed identification increases when only one member of the labeled peptide pair is observable in the form of an extreme differential expression and there is uncertainty as to whether the observable peptide is labeled with the light or the heavy ICAT reagent. In addition, the presence of an unlabeled peptide may be mistakenly interpreted as a labeled peptide that is differentially expressed in the extreme. An unlabeled peptide will need to have a combination of “right” chemical properties and very high initial concentrations (e.g. a serum albumin peptide) to “survive” the affinity chromatography stage intended to select only labeled peptides. The above is intended to be a cautionary note in pointing out potential shortcomings in a powerful technique that readily highlights and quantifies differential protein expressions, and not as a negative opinion on the suitability and utility of ICAT in protein discovery or quantification.

Of the 46 studies published to date that centered on ICAT, the vast majority were on methods development that compared differential protein expressions in two different cell states. Most of these studies were on yeast cultured under different medium conditions and they compared protein levels of one sample pair. Only one recent study [104] targeted clinical samples, and even in this study only one sample pair was analyzed for differential protein expression. The present study is the only work in which multiple sample pairs have been examined. It is, of course, possible that a given observed deviation in protein ratio from 1, i.e. a heavy/light label ratio ≠1, is due to individual differences, and that different pairing of the proliferative and secretory samples may produce a different ratio. It is because of this possibility that the criterion for differential expression was stringently set to a ratio of larger than 2.0 and smaller than 0.5 in all three sample pairs. It turns out that for the five proteins that met the above criterion of differential expression, the differences were in the extreme in that the proteins were observed in only one endometrial phase in a pair. Expression differences at this extreme level in all three sample pairs are highly unlikely to have originated from individual differences.

### Conclusion

From an initial list of 346 proteins identified by ProICAT 1.0 SP3, 119 proteins were identified after manual inspection of MS/MS data and additional searches using Mascot. The expression levels of the majority of proteins do not vary significantly between the proliferative and the secretory phases. Only five proteins satisfy the criteria on differential expression in having an expression ratio larger than 2.0 or smaller than 0.5 in all three sample pairs. In fact, the differential expressions observed are extreme in that the proteins are present in only one of the two endometrial phases. The two proteins with unquestionable differential expressions in the secretory endometrium are: glutamate NMDA receptor subunit zeta 1 precursor and FRAT1. Some of the proteins that show no differential expression have previously been examined in gene-expression studies with similar conclusions.

### EXAMPLE 6

### Abstract

SELDI MS has conventionally been practiced on linear TOF which has low mass accuracy and resolution. Here in an examination of both malignant and nonmalignant endometrial tissue homogenates it is demonstrated that high mass accuracy and resolution in the MS stage are crucial. Using a commercially available QqTOF, two potential cancer markers were resolved and subsequently identified offline as chaperonin 10 and calgranulin A, that differ by 8 Da in mass. Two offline protein identification protocols were developed: the first was based on SEC, SDS PAGE, protein extraction, trypsin digestion and MALDI-MS/MS; the second on SEC and shotgun nanoLC/MS/MS. Analyses on a cohort of 44 endometrial homogenates showed 22 out of 23 nonmalignant samples had nondetectable to very low abundance of chaperonin 10 and calgranulin A; 17 of the 21 malignant samples had detectable to abundant levels of both proteins. Immunohistochemical staining of a tissue microarray of 32 samples showed that approximately half of malignant endometrial tissues exhibited positive staining for calgranulin A in the malignant epithelium, while 9 out of 10 benign tissues exhibited negative epithelial staining. In addition, macrophages/granulocytes in malignant as well as nonmalignant tissues showed positive staining. No immunostaining occurred in stroma or myometrium.

The following materials and methods were used in the study.

### Materials and Methods

### Tissue Samples and Sample Preparation

Endometrium and EmCa tissues were retrieved from a dedicated, research in-house endometrial tissue bank. The consenting and tissue banking procedures for this tissue bank were approved by the Research Ethics Boards of York University, Mount Sinai Hospital, University Health Network, and North York General Hospital. All tissues had been snap frozen in liquid nitrogen within 15-20 minutes of devitalization at the time of hysterectomy, and were obtained with patients' consent. In each case, the endometrium was classified as nonmalignant or malignant (EmCa) by one of the authors (TJC) [Kurman, R. J., Ed., Blaustein's Pathology of the Female Genital Tract, 5^{th} ed. New York, Springer-Verlag, 2002]. Nonmalignant endometrium included both benign physiologic endometrial cases (atrophic, proliferative, secretory, and menstrual) and pathologic states (benign endometrial polyp and disordered proliferative). EmCa cases included adenocarcinomas and rarer malignant mixed Mullerian tumors (carcinosarcomas). This classification was performed using routine surgical pathology sections. The histologic classification of the research tissue was verified by examination of a histopathologic section from the frozen research tissue. Tissue was taken for proteomic analysis from the mirror-face of the residual block. Cases of atypical endometrial hyperplasia, which could be considered to represent an intermediate phenotype, were not available for frozen-tissue banking and MS analysis because all tissue was required for histopathologic examination, but tissue was available for tissue microarray (TMA) preparation.

Tissue was thawed in Hanks' balanced salt solution (HBSS, Sigma) containing protease inhibitors (1 mM 4-(2-aminoethyl)benzenesulfonyl fluoride, 10 μM leupeptide, 1 μg/ml aprotinin and 1 μM pepstatin) and followed by mechanical homogenation at 30,000 rpm using a Polytron PT 1300D handheld homogenizer (Brinkmann, Westbury, USA). These whole tissue homogenates contained not only endometrial epithelium, but supportive stroma, vessels, as well as secretions. The homogenate was then stored in aliquot at −80° C. until protein profiling.

### Chemicals

Solvents, chemicals (except otherwise noted), and Hanks' Balanced Salt Solution were obtained from Sigma-Aldrich (Oakville, ON, Canada).

### Protein Profiling Using SELD-MS

Tissue lysate was fractionated to reduce sample complexity before protein profiling. An identical quantity of proteins was used for all samples; protein amounts were measured using Bradford Assay (Bio-Rad) on a DU-65 spectrometer (Beckman) at 595 nm. HBSS or urea/thiourea 2-D lysis buffer (7 M urea, 2 M thiourea) was used to compensate the initial volume to ensure equal volumes for all samples. Two micrograms of proteins from endometrial tissue homogenate was incubated with WCX2 or CM10 ProteinChips (Ciphergen) according to the manufacturer's instructions. In brief, samples were diluted to 55 μl with the corresponding binding buffer, spotted onto the appropriate ProteinChip, and incubated in a sealed BioProcessor (Ciphergen) for 1 h at room temperature. The ProteinChip surface was washed twice with the appropriate buffer for 5 minutes, briefly rinsed with water and air-dried. Two times 0.5 μl of 50% saturated sinapinic acid in 50% acetonitrile was applied on the samples to form crystals. The ProteinChips were analyzed using a linear TOF analyzer, PBS IIc (Protein Biology System IIc, Ciphergen), or a hybrid QqTOF mass spectrometer, QSTAR XL (Applied Biosystems/MDS Sciex) fitted with the SELDI interface (Ciphergen). For accurate molecular-weight measurements, on-chip, internal mass calibration was performed using insulin (5,733 Da), cytochrome c (12,361 Da) and myoglobin (16,951 Da) as calibrants.

### Protein Purification and Identification

Selected EmCa samples were subject to chromatographic separation to yield purified proteins for identification. Five hundred micrograms of proteins from a whole tissue homogenate was first fractionated using SEC (BioSep 2000, Phenomenex) at 1 ml/min flow with phosphate buffer (pH 7.9) containing 0.05% (w/v) sodium azide. One-millilitre fractions were collected; the eluates were then concentrated to 50 μl with a silicon carbide-based spin column (ProteoSpin, MDS Sciex). Five microlitres of a concentrate was desalted by C18 Zip-Tip and analyzed with MALDI-MS to locate the fractions containing the protein markers of interest in the EmCa sample. Two different approaches were developed and used to identify the target proteins. Some proteins were more amenable to identification with one and others with the second approach. The first approach used SDS PAGE technique to further purify the target proteins. The fractions with the enriched proteins were diluted to 100 μl with freshly prepared DTT (5 mM final) in 150 mM Tris pH 8.5 buffer, and incubated at 60° C. for 1 h. Ten microlitres of the reaction mixture was desalted by C18 Zip-Tip and analyzed with MALDI-MS to assess the effect of DTT on the protein of interest. The remaining 90 μl was precipitated by acetone (80% (v/v) final), resuspended in SDS sample buffer, and the proteins were resolved by SDS PAGE. Protein molecular-weight markers (New England Biolabs) and cytochrome C(C-2506 Sigma) were included to guide the excision of gel portions containing the protein of interest. Intact proteins were extracted from the gel by 50 μl extraction solution (formic acid/acetonitrile/isopropanol/water in a ratio of 50/25/15/10) at room temperature for 4 h. The extracts were completely dried by SpeedVac and resuspended in 40 μl 100 mM ammonium bicarbonate. Half of the resuspended proteins was desalted by C18 Zip-Tip and analyzed with MALDI-MS, the other half was digested in solution with 100 ng trypsin (Promega). The identity of the target protein was determined using MS/MS ion search (ProID, Applied Biosystems). The second approach used nanoLC/MS/MS technology. After concentration using ProteoSpin, the fractions with the enriched target proteins were further cleaned up using C18 Zip-Tips. Four C18 Zip-Tips were used for each 45 μl of concentrated fractions. The sorbed proteins were eluted by 100 μl of 60% acetonitrile with 0.3% TFA. The eluates were completely dried by SpeedVac and resuspended in 100 μl of 100 mM ammonium bicarbonate. The proteins were then digested in solution with 100 ng trypsin (Promega). The resulted tryptic peptides were analyzed by MALDI-MS and online nanoLC/MS/MS. The identity of the target protein was determined using MS/MS ion search (ProID, Applied Biosystems).

### NanoLC/MS/MS

The nanoLC system was from LC Packings (Amsterdam, The Netherlands) and consisted of a Famos autosampler and an Ultimate nanoLC system. It was interfaced to a second QSTAR Pulsar hybrid QqTOF mass spectrometer using a Protana NanoES ion source (Protana Engineering A/S, Odense, Denmark). The spray capillary was a PicoTip SilicaTip emitter with a 10 μm ID tip (New Objective, Woburn, Mass., USA). The nanoLC column was a 75 μm ID×150 mm length reverse-phase PepMap C18 nano capillary column (LC Packings, Amsterdam, The Netherlands) packed with 3 μm beads with 100 Å pores. One microliter of desalted sample was injected via the full-loop mode. Separation was performed using a binary mobile-phase gradient at a total flow rate of 100 nl/min. Eluent A consisted of 94.9% deionized water, 5.0% acetonitrile and 0.1% formic acid (pH≈3). Eluent B consisted of 5.0% deionized water, 94.9% acetonitrile and 0.1% formic acid. The following binary gradient was used:

| | Time (min) | | | | | | | |
|---|---|---|---|---|---|---|---|---|
| | 0 | 10 | 40 | 42 | 44 | 45 | 70 | 72 |
| % Eluent B | 5.0 | 5.0 | 30.0 | 80.0 | 80.0 | 5.0 | 5.0 | Stop |

The ion source conditions were a curtain-gas setting of 20 and an ionspray voltage in the range of 1800-3800 V that was optimized daily. In the Q0 region, the instrument parameters were a declustering potential (DP) of 65 V and a focusing potential (FP) of 265 V. Nitrogen was used as the collision gas at a setting of CAD=5 for both TOF-MS and MS/MS scans.

All LC-MS/MS data were acquired in information-dependent acquisition (IDA) mode in Analyst QS SP5 with Bioanalyst Extension 1.1 (Applied Biosystems/MDS Sciex). A TOF-MS survey scan with an m/z range of 400-1500 and 1 s scan time was followed by two product ion scans with an m/z range of 70-2000 and 2 s scan time. The collision energy (CE) was automatically controlled by the IDA CE Parameters script. The switching criteria were set to ions greater than m/z=400 and smaller than m/z=1500 with a charge state of 2 to 5 and an abundance of ≧10 counts/s. Former target ions were excluded for 60 s and ions within a 4 Th window were ignored. In addition, the DA Extensions II script was set to 2 repetitions before dynamic exclusion and to select a precursor ion nearest to a threshold of 15 count/s every 4 cycles. These settings ensured examination of not only high abundance ions, but low abundance ones as well.

### Tissue Microarray and Immunohistochemical Protein Localization

A 32-core TMA was prepared to confirm the presence of an identified protein marker (10, 835 Th, see later sections), and to determine its localization. Following fixation in 10% buffered formalin, benign, hyperplastic and malignant endometrial tissues were histologically processed and embedded in paraffin blocks. A single 2-mm core of tissue was removed from the paraffin block using a Manual Tissue Arrayer (Beecher Instruments). The 26 endometrial study cases included 11 benign endometrium (five secretory, four proliferative, one atrophic and one menstrual), 13 malignant endometrium (12 adenocarcinomas and one carcinosarcoma), and two cases of atypical endometrial hyperplasia. Six other control tissues were also embedded into the TMA, including four non-endometrial carcinomas and 2 benign tumors (salivary gland pleomorphic adenoma and ovarian thecoma). Following MS identification, monoclonal mouse antibodies against the potential cancer marker were procured from two sources: Research Diagnostics, Inc. (Flanders, N.J.), labeled as RDI, and US Biological (Swampscott, Mass.), labeled as USB. Sections were cut from the TMA. The antibodies were applied in an appropriate dilution determined through a pilot study and immunohistochemically visualized using a diaminobenzidine chromogen.

Interpretations of the two immunohistochemically stained TMA sections were conducted using a standardized method; microscopic reviews were conducted blinded to the findings of the other TMA section. Positive staining (brown) for the potential marker was determined for three cellular components: macrophage/granulocyte, epithelium/carcinoma, and endometrial stroma and myometrium. Immunostaining of these three cell types was semi-quantitatively graded as: 0 (no immunostaining), 1+ (weak positivity and/or positivity in only occasional cells), 2+ (moderate positivity and/or positivity in more abundant cells), and 3+ (strong positivity and/or positivity in numerous cells).

### Results and Discussion

### SELDI-MS Details

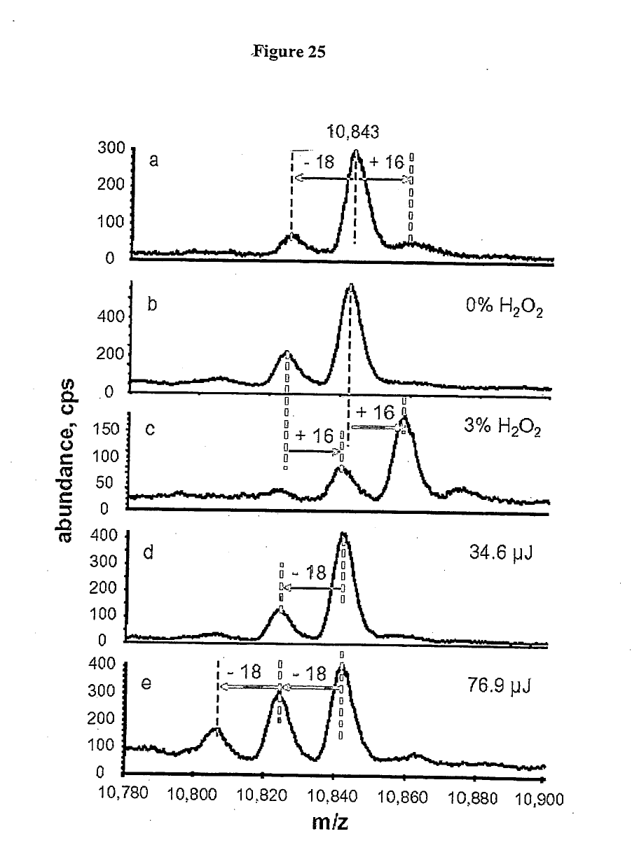

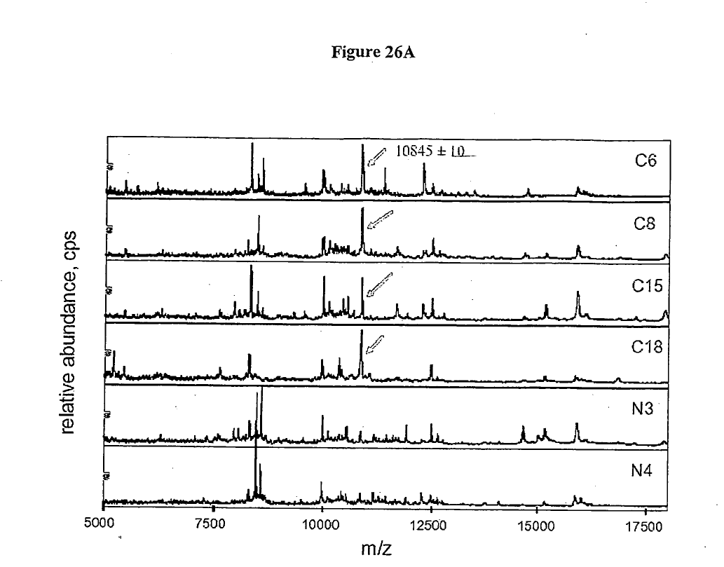

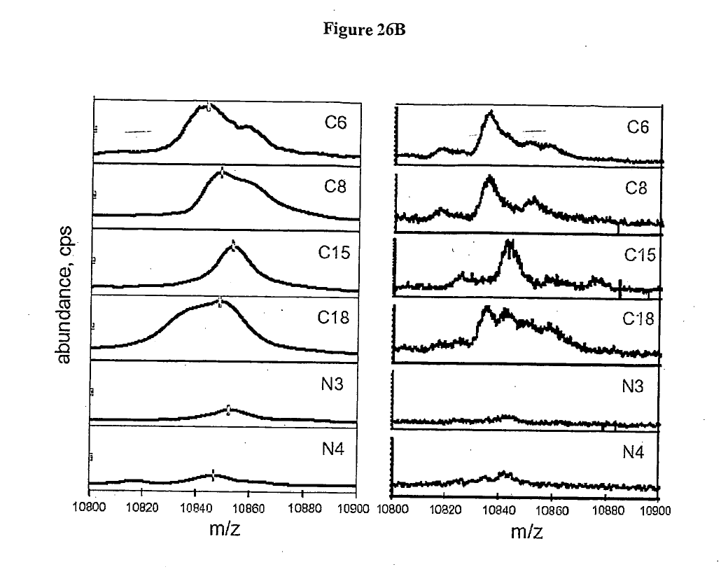

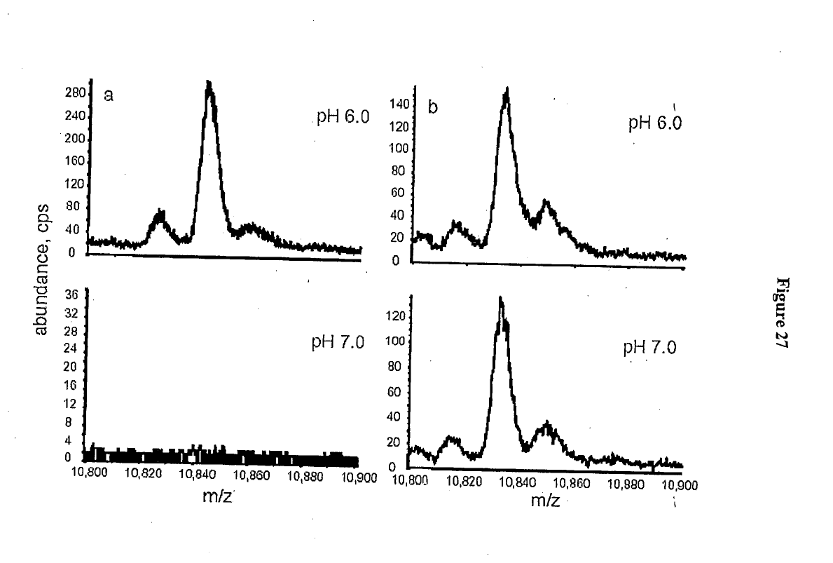

The use of SELDI-MS profiling in discovering a protein marker, chaperonin 10, for EmCa is described above. Chaperonin 10 has a molecular weight of 10,843 Da. Performing SELDI-MS on the higher resolution QqTOF mass spectrometer (QSTAR XL) was revealing: chaperonin 10 frequently appeared as three instead of a single peak (FIG. 25a). On the lower resolution PBS Ic TOF mass spectrometer, which is much more widely available and used, the peaks are unresolved. Incubating the tissue homogenate with hydrogen peroxide overnight reduced the abundance of chaperonin 10 while increased the abundance of the protein that is heavier by 16 Da (compare FIG. 25b with 25c). Increasing the fluence of the MALDI laser (from 34.6 to 76.9 μJ) had the effect of increasing the abundance of the protein peak that is 18 Th lower than chaperonin 10 in m/z value. In addition, a new protein peak that is 36 Th lower in m/z value emerged (compare FIG. 25d with 25e). Incubating with hydrogen peroxide enhanced oxidation of chaperonin 10, probably the lone methionine residue; as a result, the molecular weight of chaperonin 10 increased by 16 Da. Increasing the laser intensity probably promoted dehydration of chaperonin 10 within the MALDI source. The sources of the water were likely the five glutamic and eight aspartic acid residues. Laser-induced deamidation (−17 Th) could also occur, but was ruled out after careful and repeated measurements mass-calibrated with cytochrome c. Single measurements for chaperonin 10 on the QSTAR XL with external or internal calibrants carry a typical uncertainty of ±2 Th.

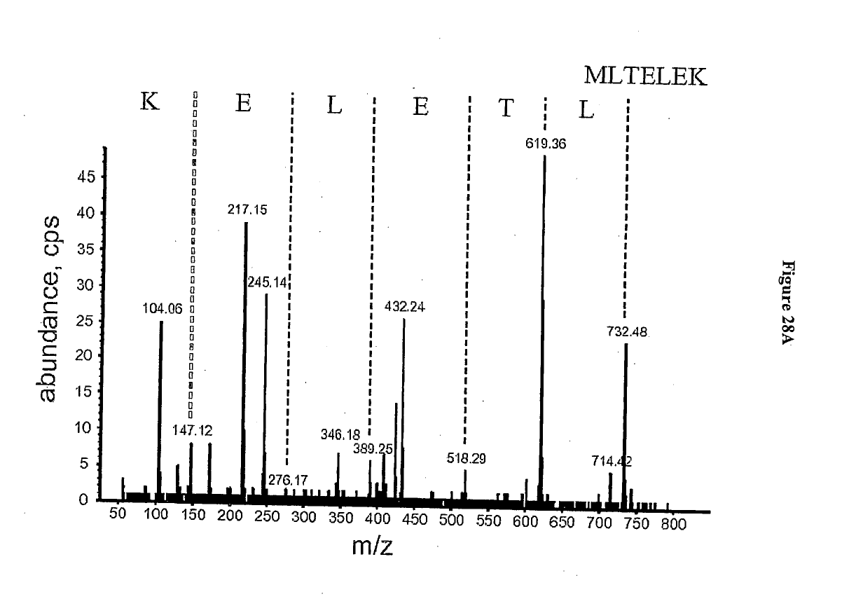

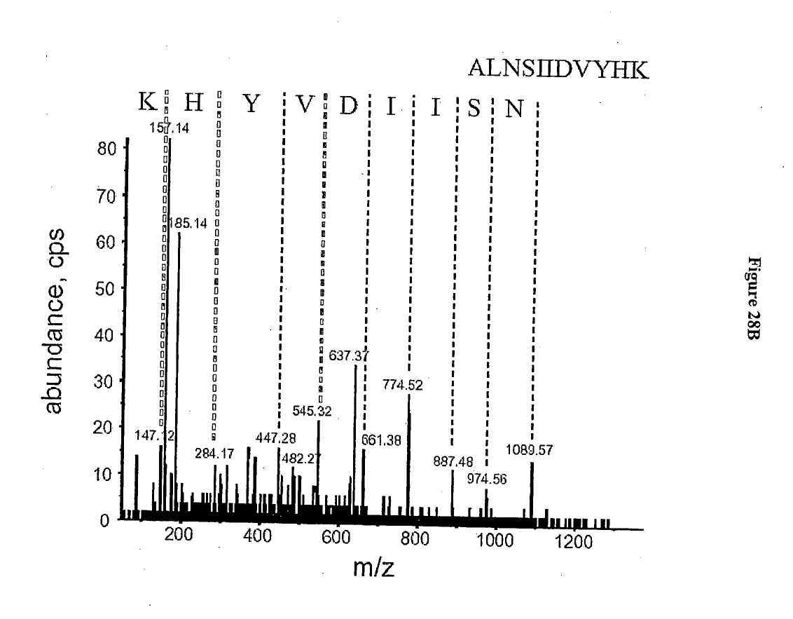

As stated earlier, the aforementioned details were unresolved on the PBS IIc TOF mass spectrometer, which displayed only one broad hump. The poor resolution in this case has no negative consequence as all peaks are different forms of chaperonin 10. However, this deficiency in resolution (and mass accuracy) may be detrimental when the sample contains a second protein of similar molecular weight. Having screened in excess of 40 endometrial homogenates (see later), it was found that a significant fraction of EmCa homogenates carry a protein that is lighter than chaperonin 10 by 8 Da. FIG. 26 shows selected SELDI MS profiles, from four EmCa (designated by “C”) and two normal endometria (designated by “N”), as collected on the PBS IIc TOF and the QSTAR XL QqTOF mass spectrometers. The pH of the washing buffer was 6.0. The PBS instrument exhibited better sensitivity than the QSTAR. However, the profiles generated by the former contain broad humps, while those by the latter exhibit considerable details (FIG. 26B). In addition, the mass accuracy of the PBS is considerably inferior to that of the QSTAR. Sample C15 is the simplest of the four EmCa samples and contains only chaperonin 10, its oxidized (10,859 Th) and dehydrated (10,825 Th) forms (compare FIG. 25). Sample C8 has a little chaperonin 10 and contains mostly this second protein (10,835 Th), its oxidized (10,851 Th) and dehydrated (10,817 Th) forms. Similarly, sample C6 has some chaperonin 10 admixed with the second protein. In sample C18, the relative abundance of chaperonin 10 and the unknown protein is approximately 1 to 1. The normal samples show traces of chaperonin 10. The possibility of having two proteins that differ by 8 Da in EmCa samples was confirmed by repeating some experiments, using pH 7.0 washing buffer. In the latter condition, the ProteinChip binds only the unknown protein (FIG. 27). The abundances of chaperonin 10 and this second protein in nonmalignant and malignant endometrial tissue homogenates are summarized in Tables 7 and 8, respectively. As SELDI/MALDI analyses on the same sample exhibit considerable variation from shot to shot and from spot to spot, the scales adopted, from “0” (absence) to “5+” (S/N ≧10 or higher) are intended to convey semiquantitative rather than quantitative information. For nonmalignant samples, chaperonin 10 and the second protein are absent or present in only very low abundance (“1+”) in 22 out of 23 cases; the exception, however, is different for the two proteins. For the malignant samples, two out of 21 cases show absence of both chaperonin 10 and the second protein; in addition, two cases show absence of chaperonin 10 but high abundances of the second protein, while another two cases show high abundances of chaperonin 10 but absence of the second protein. The absence of the potential cancer markers in some malignant tissue homogenates may be due to nonoptimal MS conditions and/or absence of tumors in the tissue block homogenized and analyzed.

### Identification of the Unknown Protein

The vast majority of SELDI-MS applications use spectral signatures from sera to differentiate normal and diseased states without actual protein identification [22, 25, 30, 105-108]. This strategy has come under criticisms and the clinical effectiveness of serum proteomics is currently being questioned [109-119). The identification of chaperonin 10 in EmCa tissue homogenates as a marker for endometrial cancer is described above. The identification strategy was based on SEC, SDS PAGE isolation, protein extraction, trypsin digestion and MALDI-MS/MS identification. However, the preferred strategy is shotgun nanoLC/MS/MS after initial purification using SEC.

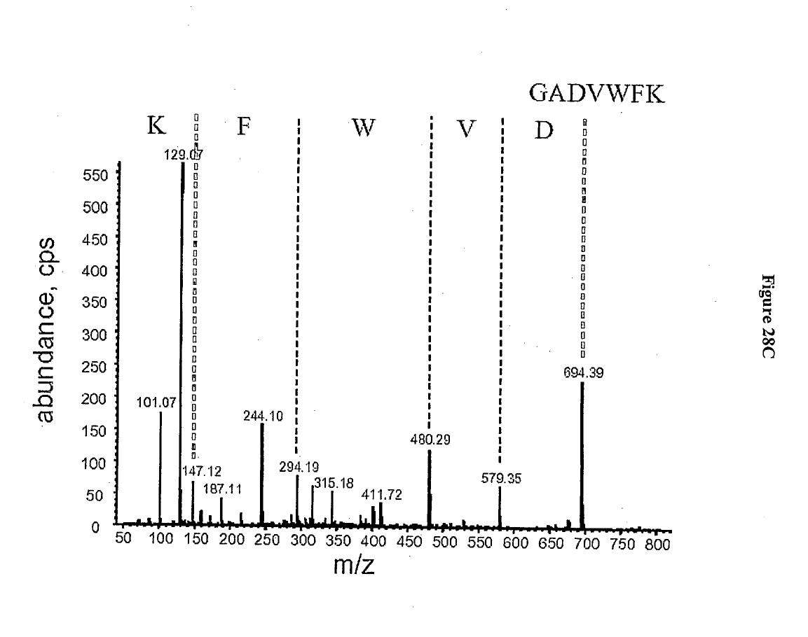

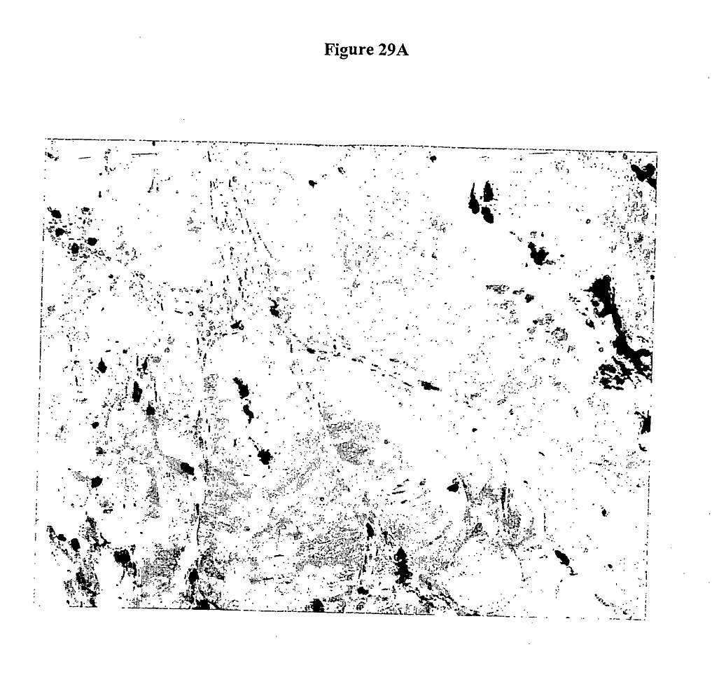

The unknown protein has a molecular weight of 10,835 Da. Using the TagIdent tool available at the ExPASy Website [133] with the following entries: molecular weight, 10,835±2 Da (±0.02%); pI, 7±5; and organism classification, human to search the Swiss-Prot database that contains 151,047 entries, returned only two proteins, calgranulin A (accession number, P05109) and small nuclear ribonuclear protein D homolog (accession number, Q9Y333). A sample was used that contained the 10,835 Da protein in abundance for SEC. This protein was found to have the highest concentration in SEC fraction 7. NanoLC/MS/MS of the tryptic peptides contained in this fraction identified a number of proteins (Table 9). The only protein that has the correct molecular weight is calgranulin A, 10,834 Da. The other proteins all have higher molecular weights (see later). FIG. 28 shows MS/MS spectra of three tryptic peptides, (A) MLTELEK [SEQ ID NO. 50], (B) ALNSIIDVYHK [SEQ ID. NO. 51] and (C) GADVWFK [SEQ ID NO. 52], that unambiguously identify calgranulin A. The first two peptides constitute residues 1-18, the third peptide residues 50-56. The three peptides constitute a sequence coverage of 25 out of a total of 93 residues or 27%.

### Calgranulin

Calgranulin A is a member of the S100 family of calcium-binding proteins [134]. It is also known as S100 calcium-binding protein A8, migration inhibitory factor-related protein 8 (MRP-8), cystic fibrosis antigen (CFAG), P8, leukocyte L1 complex light chain, and calprotectin L1L subunit. It has been reported as a specific protein for several diseases [135-156], but has not been implicated in EmCa. Calgranulin A can form homodimers as well as higher-order oligomers [139]. Calgranulin A has been found to be associated with a second protein in the S100 family, calgranulin B (13,242 Da) [137,139]. This heterodimer is thought to be involved in inflammation, including the transmigration process of leukocytes and transport of arachidonic acid to target cells [134,137]. In the present study, calgranulin B was also identified along with calgranulin A, albeit by only one peptide. The possibility that calgranulin A and B coexist as members of a larger unit is in accordance with the observation that calgranulin eluted early in SEC in fraction 7 along with other proteins of much higher molecular weights (Table 9). However, calgranulin B does not appear to be retained on the ProteinChips under conditions that are favorable for calgranulin A.

### Tissue Microarray and Immunohistochemical Localization of Calgranulin A

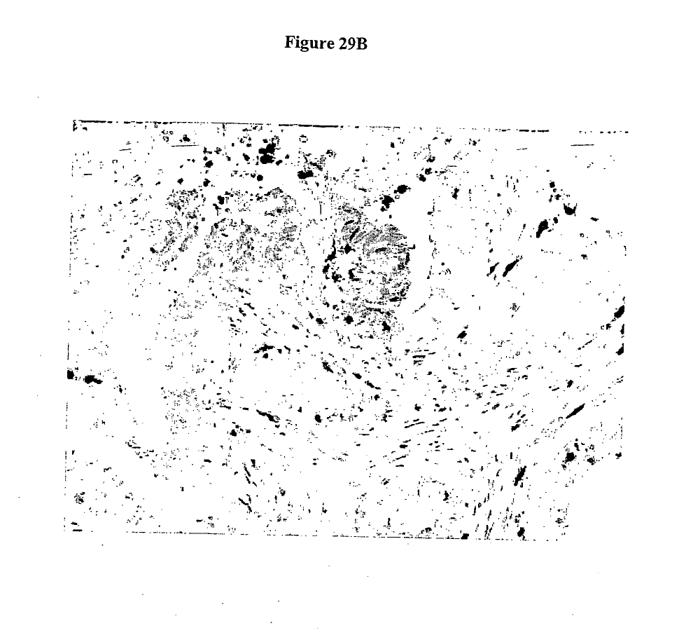

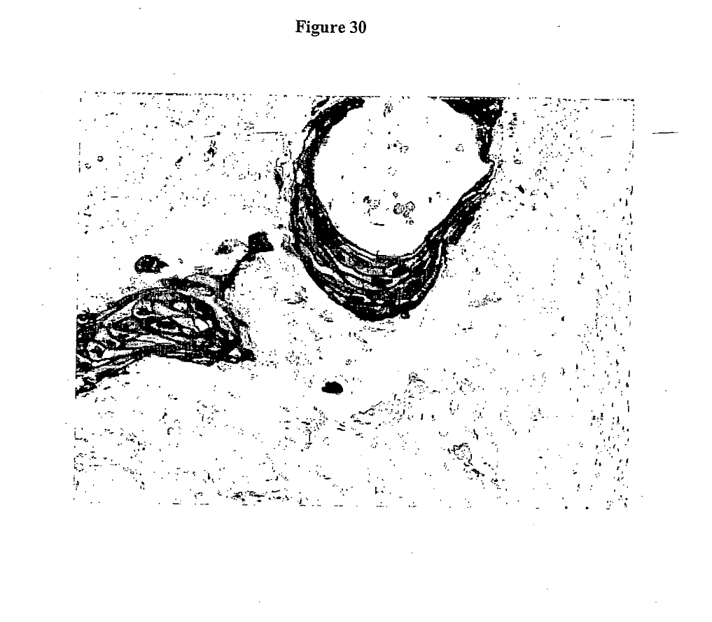

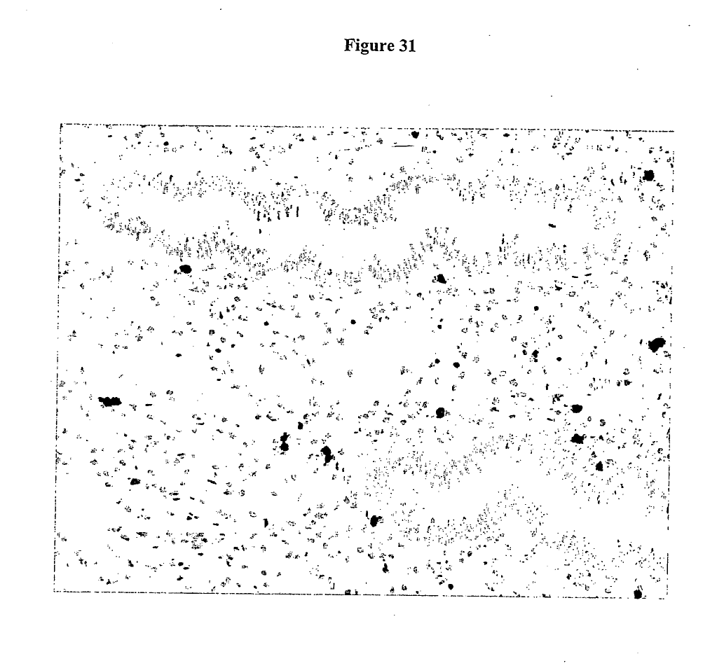

A summary of the TMA immunohistochemical studies using two different monoclonal antibodies (RDI and USB) is shown in Table 10. There is great similarity/identity between the findings from the two immunohistochemical studies. Calgranulin A was localized to the malignant epithelium of endometrial adenocarcinoma and carcinosarcoma. Approximately half of these malignant endometrial tissues showed 1+ to 3+ immunostaining for calgranulin A (FIGS. 29A and B). The most striking positivity was noted in the squamous areas of three endometrioid carcinomas (FIG. 30). Two of four non-endometrial carcinomas also exhibited immunostaining for calgranulin A in the malignant epithelium. Immunohistochemical staining for calgranulin A was absent in 9 of 10 benign endometrial cases. A single case of menstrual endometrium exhibited focal immunostaining (1+). Macrophages/granulocytes in benign endometrium (FIG. 31), malignant endometrium (FIG. 29), and non-endometrial carcinomas showed discrete strong immunostaining of variable numbers of cells with immunostaining ranging from 1+ to 3+. No immunostaining for calgranulin A was noted in stroma or myometrium.

The MS results presented here show that elevated levels of calgranulin A were detectable in about two-thirds of EmCa homogenate cases, with 14 of 21 cases demonstrating 2+ or greater abundance for this protein (Table 8). By contrast, only minor levels were observed in most nonmalignant endometrium, with 22 of 23 cases demonstrating 1+ or lower abundance (Table 7).

The TMA findings not only confirm the presence of calgranulin A, at least as assessed by immunohistochemistry, but also indicate two cellular sources for the MS findings. In EmCa, the sources are both the malignant epithelium (carcinoma) and macrophage/granulocytes, whereas in nonmalignant endometrium, macrophages seem to be the predominate source, and only rarely benign (menstrual) endometrium (Table 10). The TMA analysis identified calgranulin A not only in a greater proportion of the epithelium in the EmCa cases, but also with greater intensity, than in nonmalignant endometrial cases. Both cases of atypical hyperplasia showed 1+ immunostaining of the affected endometrial glands.

Employment of calgranulin A, in combination with other protein markers (including chaperonin 10), may provide a robust endometrial cancer diagnostic and/or screening panel. The utility of any panel will increase with the number of markers employed, and the sensitivity and specificity of individual markers. This study demonstrates the complementary nature of MS and TMA in identifying and localizing sources of potential cancer markers in this search, and a possible utility of TMA in selecting cancer markers.

### CONCLUSION

High mass accuracy and high resolution in the MS stage are crucial in SELDI MS. Using the QSTAR XL, two potential cancer markers were resolved and subsequently identified offline as chaperonin 10 and calgranulin A, that differ by 8 Da in mass. These two proteins were unresolved on the PBS IIc mass spectrometer. Two offline protein identification protocols were developed: the first was based on SEC, SDS PAGE, protein extraction, trypsin digestion and MALDI-MS/MS; the second on SEC and shotgun nanoLC/MS/MS. Analyses on a cohort of 44 endometrial homogenates showed 22 out of 23 nonmalignant samples had nondetectable to very low abundance of chaperonin 10 and calgranulin A; 17 of the 21 malignant samples had detectable to abundant levels of both proteins. Immunohistochemical staining of a tissue microarray of 32 samples showed that approximately half of malignant endometrial tissues exhibited positive staining for calgranulin A in the malignant epithelium, while 9 out of 10 benign tissues exhibited negative epithelial staining. In addition, macrophages/granulocytes in malignant as well as nonmalignant tissues showed positive staining. No immunostaining occurred in stroma or myometrium. Calgranulin A, in combination with chaperonin 10 and other proteins, can constitute a panel of markers to permit diagnosis and screening of endometrial cancer.

TABLE 1
Differentially Expressed Proteins in Endometrial Malignancies/Cancer

| | | | Expression |
|---|---|---|---|
| Protein | Gene name | Swiss-Prot Acc. No. | in EmCa |
| Chaperonin 10 | HSPE1 | Q04984 and AAH23518 | Up |
| | | [SEQ ID 1] | |
| | | NM_002157 and | |
| | | U07550 [SEQ ID 2] | |
| Calgranulin A | S100A8 | P05109 [SEQ ID 3] | Up |
| | | A12027 [SEQ ID 4] | |
| | | NM_002964 | |
| | | [SEQ ID 5] | |
| Calgranulin B | S100A9 | P06702 [SEQ ID 6] | Up |
| | | X06233 [SEQ ID 7] | |
| | | M21064 [SEQ ID 8] | |
| Polymeric-immunoglobulin | PIGR | P01833 or Q81ZY7 [SEQ ID 9] | Up |
| Receptor [precursor] | | NM_002644 [SEQ ID 10] | |
| Phosphatidylethanolamine- | PBP | P30086 [SEQ ID 11] | Up |
| binding protein (PEBP) | | NM_002567[SEQ ID 12] | |
| Acidic leucine-rich nuclear | ANP32A | P39687 [SEQ ID 13] | Up |
| phosphoprotein 32 family | | NM_006305 [SEQ ID 14] | |
| member A | | | |
| Heat shock 70 kDa protein 6 | HSPA6 | P17066 [SEQ ID 15] | Up |
| | | NM_002155 [SEQ ID 16] | |
| | | X51757 [SEQ ID 17] | |
| Macrophage migration | MIF | P14174 [SEQ ID 18] | Up |
| Inhibitory factor (MIF) | | NM_002415 [SEQ ID 19] | |
| | | L19686 [SEQ ID 20] | |
| Calgizzarin (S100C protein) | S100A11 | P31949 [SEQ ID 21] | Up |
| | | NM_005620 and D38583 | |
| | | [SEQ ID 22] | |
| Triosephosphate isomerase | TPI1 | P00938 and NP_000356 | Up |
| | | [SEQ ID 23] | |
| | | NM_000365 [SEQ ID 24] | |
| | | X69723 [SEQ ID 25] | |
| Alpha-1-antitrypsin precursor | SERPINA 1 | gi/1703025 | Under |
| | | ITHU and P01009 [SEQ ID 30] | |
| | | NM_000295[SEQ ID NO. 31] | |
| | | K02212 [SEQ ID 32] | |
| Creatine kinase, B chain (B-CK) | CKB | gi/125294 | Under |
| | | P12277[SEQ ID NO. 33] | |
| | | NM_001823 [SEQ ID NO 34] | |
| | | X15334 [SEQ ID NO 35] | |
| Pyruvate kinase, M1 or M2 isozyme | PKM2 | gi/20178296; gi/125604; | Up |
| | | KPY1_HUMAN (P14618) | |
| | | [SEQ ID NO. 36] | |
| | | X56494 [SEQ ID 37] | |
| Transgelin (smooth muscle | TAGLN | gi/3123283 | Under |
| protein 22-alpha) | | Q01995 [SEQ ID. 38] | |
| | | D84342 [SEQ ID 39] | |
| Heterologous nuclear | (hnRPD | ROD_HUMAN (Q14103) | Up |
| ribonucleoprotein D | | [SEQ ID NO. 40] | |
| | | AF026126 [SEQ ID 41] | |

TABLE 2
The identification of Chaperonin 10 by mass spectrometry and
Western blotting techniques in non-malignant endometrial
tissue homogeneates.

| | | | Western |
|---|---|---|---|
| | Histopathologic | MS results | Blotting Results |
| Case | Classification | (relative intensity) | (relative intensity) |
| 1 | Proliferative | + | ∘ |
| 2 | Proliferative | + | ∘ |
| 3 | Secretory | + | ∘ |
| 4 | Atrophic | + | + |
| 5 | Benign polyp | ∘ | ∘ |
| 6 | Atrophic | + | + |
| 7 | Secretory | ∘ | ∘ |
| 8 | Disordered proliferative | ∘ | + |
| | endometrium | | |
| 9 | Secretory | ∘ | + |
| 10 | Disordered proliferative | ∘ | + |
| 11 | Secretory | ∘ | + |
| 12 | Secretory | + | + |
| 13 | Secretory | ∘ | + |
| 14 | Secretory | + | ∘ |
| 15 | Atrophic | ∘ | ∘ |
| 16 | Secretory | + | + |
| 17 | Menstrual | + | + |
| 18 | Secretory | + | + |
| 19 | Proliferative | + | ∘ |
| 20 | Proliferative | + | + |
| 21 | Menstrual | ∘ | ∘ |
| 22 | Atrophic | ∘ | ∘ |
| 23 | Atrophic | ++ | + |

TABLE 3
The identification of Chaperonin 10 by mass spectrometry and
Western blotting techniques in malignant endometrial tissue
homogeneates.

| | | | Western |
|---|---|---|---|
| | Histopathologic | MS results | Blotting Results |
| Case | Classification | (relative intensity) | (relative intensity) |
| 24 | Em AdCa ⅓ | ++ | ++ |
| 25 | Em AdCa ⅓ | ◯ | + |
| 26 | MMMT | +++++ | +++++ |
| 27 | Em AdCa | ++ | ++ |
| 28 | MMMT* | + | ∘ |
| 29 | Em AdCa ⅔ | + | +++ |
| 30 | Em AdCa ⅔ | + | ++++ |
| 31 | Ser AdCa | + | +++++ |
| 32 | Em AdCa ⅓ | +++++ | +++++ |
| 33 | Em AdCa Grade n.k. | ◯ | ++++ |
| 34 | Em AdCa Grade n.k. | + | +++ |
| 35 | Em AdCa Grade n.k. | ◯ | + |
| 36 | Em AdCa ⅓ | +++++ | ++++ |
| 37 | Em AdCa ⅓ | ++++ | +++ |
| 38 | Em AdCa ⅓ | +++ | +++ |
| 39 | Muc AdCa ⅓ | ++ | ∘ |
| 40 | Em AdCa ⅓ | +++ | +++ |
| 41 | Em AdCa ⅓ | +++ | ++ |
| 42 | Em AdCa-Ser AdCa | +++++ | +++++ |
| 43 | Em** AdCa ⅓ | ∘ | ∘ |
| 44 | Em AdCa ⅔ | ++++ | ++++ |

*Mirror section showed minimal tumor.
**Mirror image showed necrotic tumor only.
Em AdCa = Endometrioid adenocarcinoma
MMMT = Malignant Mixed Mullerian Tumor
Muc AdCa = Mucinous adenocarcinoma
n.k. = not known
Ser AdCa = Serous adenocarcinoma

TABLE 4
Differentially expressed proteins quantified by iTRAQ.
S:P is the ratio of secretory phase relative to the proliferative phase
C1:P and C2:P are the ratios of cancer samples 1 and 2 relative to the
proliferative phase, respectively.

| accession_id | name | S:P (± SD) | C1:P (± SD) | C2:P (± SD) |
|---|---|---|---|---|
| gi\|461730 | 10 kDa heat shock protein, | 1.06 ± 0.12 | 2.71 ± 0.52 | 2.23 ± 0.29 |
| | mitochondrial (Hsp10) (10 kDa | | | |
| | chaperonin) (CPN10) | | | |
| gi\|1703025 | Alpha-1-antitrypsin precursor | 1.25 ± 0.39 | 0.34 ± 0.07 | 0.44 ± 0.16 |
| gi\|125294 | Creatine kinase, B chain (B-CK) | 0.96 ± 0.04 | 0.38 ± 0.16 | 0.52 ± 0.06 |
| gi\|20178296 or | Pyruvate kinase, M1 or M2 | 1.03 ± 0.11 | 2.75 ± 0.03 | 2.02 ± 0.08 |
| gi\|125604 | isozyme | | | |
| gi\|3123283 | Transgelin (Smooth muscle | 1.46 ± 0.26 | 0.26 ± 0.04 | 0.45 ± 0.05 |
| | protein 22-alpha) | | | |

TABLE 5
Differentially expressed proteins quantified using cICAT. Singleton H signifies extreme
Overexpression in the cancer sample relative to the normal proliferative control.
Standard deviations were calculated from all three sample pairs.

| Accession # | Name | Avg. C:N ratio (± SD) |
|---|---|---|
| S111_HUMAN (P31949) | Calgizzarin (S100C protein) (MLN 70). | Singleton H^{a} |
| ROD_HUMAN (Q14103) | Heterogeneous nuclear ribonucleoprotein D0 | 4.62 (± 0.39) |
| MIF_HUMAN (P14174) | Macrophage migration inhibitory factor | 2.74 (± 0.92) |
| | (MIF) | |
| PEBP_HUMAN (P30086) | Phosphatidylethanolamine-binding protein | 3.72 (± 1.03) |
| | (PEBP) | |
| KPY1_HUMAN (P14618) | Pyruvate kinase, M1 isozyme (EC 2.7.1.40) | 4.61 (± 1.40) |
| KPY_HUMAN: | Polymeric-immunoglobulin receptor | 9.83 (+/− 6.61) |
| PIGR_HUMAN (P01833) | precursor | |

^{a}Calculation of SD is not possible

TABLE 6
Proteins Identified and Secretory/Proliferative Expression Levels

| | | Average H:L | | |
|---|---|---|---|---|
| Accession # | Description | ratio | SD | Frequency |
| 143T_HUMAN | 14-3-3 protein tau (14-3-3 protein theta) | 0.81 | | 1 |
| (P27348) | | | | |
| RS12_ORENI | 40S ribosomal protein S12. | 0.52 | | 1 |
| (O13019) | | | | |
| RS21_HUMAN | 40S ribosomal protein S21. | NQ | | 1 |
| (P35265) | | | | |
| HPPD_MYCGR | 4-hydroxyphenylpyruvate dioxygenase | Singleton L | | 1 |
| (O42764) | (EC 1.13.11.27) | | | |
| RL36_VIBCH | 50S ribosomal protein L36. | 0.84 | | 1 |
| (P78001) | | | | |
| ACTA_HUMAN | Actin, aortic smooth muscle or Actin | 1.86 | | 1 |
| (P03996) or | alpha skeletal muscle | | | |
| ACTS_HUMAN | | | | |
| (P02568) | | | | |
| ACTB_HUMAN | Actin, cytoplasmic | 0.93 | 0.04 | 3 |
| (P02570) | | | | |
| SAH1_XENLA | Adenosylhomocysteinase 1 (EC 3.3.1.1) | 0.70 | | 1 |
| (P51893) | | | | |
| AFAM_HUMAN | Afamin precursor (Alpha-albumin) | NQ | | 1 |
| (P43652) | (Alpha-Alb). | | | |
| ADHL_HUMAN | Alcohol dehydrogenase class III L chain | 0.67 | | 1 |
| (P11766) | | | | |
| DHA1_HUMAN | Aldehyde dehydrogenase 1A1 (EC | 0.60 | | 1 |
| (P00352) | 1.2.1.3) | | | |
| A1AG_HUMAN | Alpha-1-acid glycoprotein 1 precursor | 1.1 | 0.5 | 3 |
| (P02763) | (AGP.1) | | | |
| A2HS_HUMAN | Alpha-2-HS-glycoprotein precursor | 0.82 | 0.54 | 2 |
| (P02765) | (Fetuin-A) | | | |
| ALU5_HUMAN | Alu subfamily | 1.1 | | 1 |
| (P39192) | | | | |
| AOP2_HUMAN | Antioxidant protein 2 (1-Cys | NQ | | 1 |
| (P30041) | peroxiredoxin) | | | |
| TRAI_AGRT5 | Autoinducer synthesis protein traI. | 0.55 | | 1 |
| (P33907) | | | | |
| APOH_HUMAN | Beta-2-glycoprotein I precursor | 0.58 | | 1 |
| (P02749) | (Apolipoprotein H) | | | |
| ARK1_HUMAN | Beta-adrenergic receptor kinase 1 (EC | Singleton L | | 1 |
| (P25098) | 2.7.1.126) | | | |
| BPEA_HUMAN | Bullous pemphigoid antigen 1, isoforms | 0.99 | | 1 |
| (O94833) | 6/9/10 | | | |
| CLP1_HUMAN | Calponin H1, smooth muscle (Basic | 1.8 | | 1 |
| (P51911) | calponin) | | | |
| KAP3_HUMAN | cAMP-dependent protein kinase type II- | Singleton L | | 1 |
| (P31323) | beta | | | |
| CABA_MOUSE | CARG-binding factor-A (CBF-A). | 0.97 | 0.33 | 3 |
| (Q99020) | | | | |
| CATB_HUMAN | Cathepsin B precursor (EC 3.4.22.1) | 0.88 | 0.70 | 2 |
| (P07858) | (Cathepsin B1) | | | |
| CB32_YEAST | Centromere DNA-binding protein | NQ | | 1 |
| (P40969) | complex CBF3 subunit | | | |
| BPHB_BURCE | Cis-2,3-dihydrobiphenyl-2,3-diol | Singleton L | | 1 |
| (P47227) | dehydrogenase (EC | | | |
| CLH_BOVIN | Clathrin heavy chain. | 0.62 | | 1 |
| (P49951) | | | | |
| COF1_HUMAN | Cofilin, non-muscle isoform (18 kDa | 0.95 | 0.15 | 3 |
| (P23528) | phosphoprotein) | | | |
| CO3_HUMAN | Complement C3 precursor | 1.1 | 0.1 | 2 |
| (P01024) | | | | |
| CFAH_HUMAN | Complement factor H precursor (H factor | NQ | | 1 |
| (P08603) | 1). | | | |
| AXO1_CHICK | Contactin 2 precursor (Axonin-1). | 0.85 | | 1 |
| (P28685) | | | | |
| KCRB_HUMAN | Creatine kinase, B chain (EC 2.7.3.2) (B- | 6.0 | | 1 |
| (P12277) | CK). | | | |
| CRBH_HUMAN | Crumbs protein homolog 1 precursor. | NQ | | 1 |
| (P82279) | | | | |
| PYRG_STAAM | CTP synthase (EC 6.3.4.2) (UTP-- | Singleton H | | 1 |
| (Q99SD1) | ammonia ligase) (C | | | |
| CYSR_HUMAN | Cysteine-rich protein 1 (CRP1) (CRP). | 4.4 | | 1 |
| (P21291) | | | | |
| DDX4_HUMAN | DEAD-box protein 4 (VASA homolog). | Singleton H | | 1 |
| (Q9NQI0) | | | | |
| DEST_HUMAN | Destrin (Actin-depolymerizing factor) | 2.4 | | 1 |
| (P18282) | (ADF). | | | |
| EF2_HUMAN | Elongation factor 2 | 0.60 | | 1 |
| (P13639) | | | | |
| XYNA_PIRSP | Endo-1,4-beta-xylanase A precursor (EC | 0.32 | | 1 |
| (Q12667) | 3.2.1.8) (1 | | | |
| ENOA_HUMAN | Enolase (EC 4.2.1.11) (2- | 2.6 | 1.9 | |
| (P06733) or | phosphoglycerate dehydrat.) or c-myc | | | |
| MPB1_HUMAN | promoter binding protein (MBP-1) | | | |
| (P22712) | | | | |
| ENV_SIVM1^{a} | Envelope polyprotein GP160 precursor | Singleton L | | 3 |
| (P05885) | | | | |
| LEM2_CANFA | E-selectin precursor (Endothelial | Singleton H | | 1 |
| (P33730) | leukocyte adhesi | | | |
| FIBG_HUMAN | Fibrinogen gamma chain precursor | 1.2 | | 1 |
| (P02679) | | | | |
| FLNA_HUMAN | Filamin A (Alpha-filamin) | 1.6 | 0.8 | 3 |
| (P21333) | (Filamin 1) | | | |
| ALFA_HUMAN | Fructose-bisphosphate aldolase A (EC | 1.2 | 0.2 | 2 |
| (P04075) | 4.1.2.13) (Mu | | | |
| G25P_HUMAN | G25K GTP binding protein (CDC 42 | NQ | | 1 |
| (P25763) or | homolog) or Ras related C3 botulinum | | | |
| RAC1_HUMAN | toxin substrate 1 or 2 or 3 or Rho-related | | | |
| (P15154) or | GTP-binding protein Rho G or RhoJ | | | |
| RHOJ_HUMAN | | | | |
| (Q9H4E5) | | | | |
| LEG1_CRIGR | Galectin-1 (Beta-galactoside-binding | 0.67 | 0.52 | 3 |
| (P48538) | lectin L-14-I) | | | |
| GELS_HUMAN | Gelsolin (Actin-depolymerizing factor) | 1.4 | | 2 |
| (P06396) | (ADF) | | | |
| NMZ1_HUMAN | Glutamate [NMDA] receptor subunit zeta | Singleton H | | 3 |
| (Q05586) | 1 precursor | | | |
| GTP_HUMAN | Glutathione S-transferase P | 1.3 | 0.4 | 3 |
| (P09211) | (EC 2.5.1.18) | | | |
| G3P2_HUMAN^{b} | Glyceraldehyde 3-phosphate | 1.0 | 0.0 | 3 |
| (P04406) | dehydrogenase | | | |
| PAEP_HUMAN | Glycodelin precursor (GD) (Pregnancy- | Singleton H | | 1 |
| (P09466) | associated en | | | |
| HPT1_HUMAN | Haptoglobin-1 precursor. | 2.2 | 1.6 | 2 |
| (P00737) | | | | |
| HS70_HUMAN | Heat shock cognate 71 kDa protein. | 1.3 | 1.1 | 3 |
| (08107) | | | | |
| HS9A_HUMAN | Heat Shock Protein HSP90-alpha | NQ | | 1 |
| (P07900) | | | | |
| HBB_HUMAN | Hemoglobin beta chain. | 0.60 | 0.45 | 3 |
| (P02023) | | | | |
| HEMO_HUMAN | Hemopexin precursor (Beta-1B- | 1.1 | 0.3 | 3 |
| (P02790) | glycoprotein). | | | |
| ROD_HUMAN | Heterogeneous nuclear ribonucleoprotein | 1.0 | 0.2 | 3 |
| (Q14103) | D0 (hnRNP D0) | | | |
| ROK_HUMAN | Heterogeneous nuclear ribonucleoprotein | 0.68 | | 1 |
| (Q07244) | K (hnRNP K) | | | |
| HNT1_HUMAN | Histidine triad nucleotide binding protein 1 | 1.4 | | 1 |
| (P49773) | | | | |
| YF48_HUMAN | Hypothetical protein KIAA1548 | 0.85 | | 1 |
| (Q9HCM4) | | | | |
| Y885_MYCTU | Hypothetical protein Rv0885. | NQ | | 1 |
| (Q10546) | | | | |
| GC1_HUMAN | Ig gamma-1 chain C region. | 0.76 | 0.23 | 3 |
| (P01857) | | | | |
| GC2_HUMAN | Ig gamma-2 chain C region. Or Ig | 1.0 | 0.5 | 3 |
| (P01859) or | gamma-4 chain C region | | | |
| GC4_HUMAN | | | | |
| (P01861) | | | | |
| GC3_HUMAN | Ig gamma-3 chain C region | 0.85 | 0.22 | 3 |
| (P01860) | | | | |
| KAC_HUMAN | Ig kappa chain C region. | 0.88 | 0.37 | 3 |
| (P01834) | | | | |
| HUMAN (P01876) | Ig alpha-1 chain C region or Ig alpha-2 | 1.4 | | 1 |
| or (P01877) | chain C region | | | |
| IDI2_RICCN^{c} | Isopentenyl-diphosphate delta-isomerase | Singleton H | | 3 |
| (Q92HM7) | (EC 5.3.3. | | | |
| TRFL_MOUSE | Lactotransferrin precursor (Lactoferrin). | Singleton H | | 1 |
| (P08071) | | | | |
| LGUL_HUMAN | Lactoylglutathione lyase (EC 4.4.1.5) | 1.0 | 0.2 | 2 |
| (Q04760) | (Methylglyox | | | |
| CSR3_HUMAN | LIM domain protein, cardiac (Muscle | 4.2 | | 1 |
| (P50461) | LIM protein) ( | | | |
| LDH_HUMAN | L-lactate dehydrogenase (EC 1.1.1.27) | 1.4 | 0.3 | 3 |
| (P00338) or | (LDH). | | | |
| (P07195) | | | | |
| MIF_HUMAN | Macrophage migration inhibitory factor | 1.2 | 0.7 | 3 |
| (P14174) | (MIF) | | | |
| MSRE_MOUSE | Macrophage scavenger receptor types I | Singleton H | | 1 |
| (P30204) | and II | | | |
| MDHM_HUMAN | Malate dehydrogenase, mitochondrial | NQ | | 2 |
| (P40926) | precursor | | | |
| MSP1_PLAF3 | Merozoite surface protein 1 precursor | Singleton L | | 1 |
| (P19598) | | | | |
| MU5A_HUMAN | Mucin 5AC (Mucin 5 subtype AC, | NQ | | 1 |
| (P98088) | tracheobronchial) | | | |
| MYH9_HUMAN | Myosin heavy chain non muscle type A or B | 0.29 | | 1 |
| (P35579) or | | | | |
| MYHA_HUMAN | | | | |
| (P35580) | | | | |
| KML2_RABIT | Myosin light chain kinase 2 | Singleton H | | 3 |
| (P07313) | | | | |
| NIR_NEUCR | Nitrite reductase [NAD(P)H] | 1.3 | | 1 |
| (P38681) | (EC 1.7.1.4). | | | |
| NOG3_BRARE | Noggin 3 precursor. | 1.3 | | 1 |
| (Q9YHV3) | | | | |
| NDKA_HUMAN | Nucleotide diphosphate kinase A or B | 1.2 | | 1 |
| (P15531) or | | | | |
| NDKB_HUMAN | | | | |
| (P22392) | | | | |
| CYPH_HUMAN | Peptidyl-prolyl cis-trans isomerase A (EC | 1.2 | 0.2 | 3 |
| (P05092) | 5.2.1.8) | | | |
| PEBP_BOVIN | Phosphatidylethanolamine-binding protein | 1.0 | 0.2 | 2 |
| (P13696) | (PEBP) (B | | | |
| PGK_HUMAN | Phosphoglycerate kinase (EC 2.7.2.3). | 0.99 | | 1 |
| (P00559) | | | | |
| PMG1_HUMAN | Phosphoglycerate mutase 1 (EC 5.4.2.1) | 0.73 | | 1 |
| (P18669) | (EC 5.4.2.4 | | | |
| POL_RSVP^{d} | POL polyprotein | Singleton H | | 2 |
| (P03354) | | | | |
| PIGR_HUMAN | Polymeric-immunoglobulin receptor | 1.1 | | 1 |
| (P01833) | precursor (Poly- | | | |
| AR34_DROME | Probable ARP2/3 complex 34 kDa subunit | 3.5 | | 1 |
| (Q9VIM5) | (P34-ARC). | | | |
| AMPA_CAMJE | Probable cytosol aminopeptidase (EC | 0.93 | | 1 |
| (Q9PP04) | 3.4.11.1) (Leu | | | |
| PRO1_HUMAN | Profilin I | 1.1 | | 1 |
| (P07737) | | | | |
| PROP_HUMAN | Properdin precursor (Factor P). | Singleton H | | 1 |
| (P27918) | | | | |
| PRTH_PORGI | Protease prtH (EC 3.4.22.—). | Singleton L | | 1 |
| (P46071) | | | | |
| PSA5_HUMAN | Proteasome subunit alpha type 5 (EC | 2.1 | | 1 |
| (P28066) | 3.4.25.1) (Pro | | | |
| VN02_VACCC | Protein N2. | Singleton L | | 1 |
| (P20641) | | | | |
| FRT1_HUMAN | Proto-oncogene FRAT1 | Singleton H | | 3 |
| (Q92837) | | | | |
| O22C_DROME | Putative odorant receptor 22c. | 0.90 | 0.31 | 3 |
| (P81911) | | | | |
| KPY1_HUMAN | Pyruvate kinase, M1 isozyme (EC | 1.2 | 0.4 | 3 |
| (P14618) | 2.7.1.40) (Pyruvat | | | |
| CUT1_SCHPO | Separin (EC 3.4.22.—). | NQ | | 1 |
| (P18296) | | | | |
| TRFE_HUMAN | Serotransferrin precursor (Transferrin) | 1.3 | 0.4 | 3 |
| (P02787) | (Siderophi | | | |
| ALBU_HUMAN | Serum albumin precursor. | 1.1 | 0.3 | 3 |
| (P02768) | | | | |
| STC_DROME | Shuttle craft protein. | 1.1 | | 1 |
| (P40798) | | | | |
| SLI1_HUMAN | Skeletal muscle LIM protein 1 | 2.0 | | 1 |
| (Q13642) | | | | |
| PCP1_SCHPO | Spindle pole body protein pcp1. | Singleton H | | 1 |
| (Q92351) | | | | |
| SODC_HUMAN | Superoxide dismutase [Cu—Zn] | 0.92 | 0.33 | 3 |
| (P00441) | | | | |
| THIO_HUMAN | Thioredoxin. | 0.87 | | 1 |
| (P10599) | | | | |
| TPIS_HUMAN | Triosephosphate isomerase (EC 5.3.1.1) | 1.1 | 0.5 | 3 |
| (P00938) | (TIM). | | | |
| TPM1_HUMAN | Tropomyosin 1 alpha chain (Alpha- | 2.3 | 0.7 | 2 |
| (P09493) | tropomyosin). | | | |
| TPM4_HUMAN | Tropomyosin alpha 4 chain or beta 2 | 0.75 | | 1 |
| (P07226) or | chain | | | |
| TPM2_HUMAN | | | | |
| (P07951) | | | | |
| TBA_HUMAN | Tubulin alpha chain. | 1.2 | 0.5 | 3 |
| (P05209) | | | | |
| TBB_HUMAN | Tubulin beta chain (Fragment). | 0.77 | 0.25 | 3 |
| (P07437 or | | | | |
| P05217)) | | | | |
| PTK7_HUMAN | Tyrosine-protein kinase like 7 precursor | 0.35 | | 1 |
| (Q13308) | | | | |
| SPK1_DUGTI | Tyrosine-protein kinase SPK-1 | Singleton L | | 1 |
| (P42687) | (EC 2.7.1.112). | | | |
| VSI1_TRYBB | Variant surface glycoprotein ILTAT 1.21 | Singleton L | | 1 |
| (P26326) | precursor | | | |
| PGCV_MACNE | Versican core protein | 4.3 | | 1 |
| (Q28858) | | | | |
| VIME_HUMAN | Vimentin (Fragment). | 1.6 | 2.2 | 3 |
| (P08670) | | | | |
| VINC_HUMAN | Vinculin (Metavinculin). | 3.1 | | 2 |
| (P18206) | | | | |
| VTDB_HUMAN | Vitamin D-binding protein precursor | 1.3 | 0.4 | 2 |
| (P02774) | (DBP) (Group-s | | | |
| WDR1_HUMAN | WD-repeat protein 1 (Actin interacting | 1.6 | 1.0 | 2 |
| (O75083) | protein 1) | | | |
| Z363_HUMAN | Zinc finger protein 363 | Singleton L | | 1 |
| (Q96PM5) | | | | |

Note:
Frequency is the number of sample pairs in which the protein is observed
NQ = Not quantified
Singleton L = Only observed in Proliferative phase
Singleton H = Only observed in Secretory phase
^{a}Subsequently identified by Mascot as Clostridium putative phosphatidylserine decarboxylase proenzyme (gi: 28209853)
^{b}Internal standard to which other proteins' peak areas are normalized
^{c}Rickettsia conorii protein
^{d}Subsequently identified by Mascot as unlabeled serum albumin

TABLE 7
Protein abundances in nonmalignant endometrial tissue homogenates.

| | | Chaperonin 10 | |
|---|---|---|---|
| | Histopathologic | (relative | Second protein^{‡} |
| Case | Classification | abundance)^{†} | (relative abundance)^{†} |
| 1 | Proliferative | 1+ | 0 |
| 2 | Proliferative | 1+ | 0 |
| 3 | Secretory | 1+ | 0 |
| 4 | Atrophic | 1+ | 0 |
| 5 | Benign polyp | 0 | 0 |
| 6 | Atrophic | 1+ | 1+ |
| 7 | Secretory | 0 | 1+ |
| 8 | Disordered proliferative | 0 | 0 |
| 9 | Secretory | 0 | 1+ |
| 10 | Disordered proliferative | 0 | 1+ |
| 11 | Secretory | 0 | 1+ |
| 12 | Secretory | 1+ | 0 |
| 13 | Secretory/menstrual | 0 | 5+* |
| 14 | Secretory | 1+ | 0 |
| 15 | Atrophic | 0 | 1+ |
| 16 | Secretory | 1+ | 0 |
| 17 | Menstrual | 1+ | 1+ |
| 18 | Secretory | 1+ | 0 |
| 19 | Proliferative | 1+ | 0 |
| 20 | Proliferative | 1+ | 0 |
| 21 | Menstrual | 0 | 0 |
| 22 | Atrophic | 0 | 1+ |
| 23 | Atrophic | 2+ | 1+ |

^{†}0 = absence, 1+ = S/N 2-3, 2+ = S/N 3-5, 3+ = S/N 5-7, 4+ = S/N 7-10, 5+ = S/N ≧ 10.
^{‡}calgranulin A.
*cervical cancer present several centimeters away.

TABLE 8
Protein abundances in malignant endometrial tissue homogenates.

| | | | Second protein^{‡} |
|---|---|---|---|
| | Histopathologic | Chaperonin 10 | (relative |
| Case | Classification | (relative abundance)^{†} | abundance)^{†} |
| 24 | Em AdCa ⅓ | 2+ | 1+ |
| 25 | Em AdCa ⅓ | 0 | 5+ |
| 26 | MMMT | 5+ | 1+ |
| 27 | Em AdCa | 2+ | 5+ |
| 28 | MMMT | 1+ | 4+ |
| 29 | Em AdCa ⅔ | 1+ | 3+ |
| 30 | Em AdCa ⅔ | 1+ | 4+ |
| 31 | Ser AdCa | 1+ | 3+ |
| 32 | Em AdCa ⅓ | 5+ | 2+ |
| 33 | Em AdCa Grade n.k. | 0 | 0 |
| 34 | Em AdCa Grade n.k. | 1+ | 3+ |
| 35 | Em AdCa Grade n.k. | 0 | 0 |
| 36 | Em AdCa ⅓ | 5+ | 0 |
| 37 | Em AdCa ⅓ | 4+ | 2+ |
| 38 | Em AdCa ⅓ | 3+ | 5+ |
| 39 | Muc AdCa ⅓ | 2+ | 3+ |
| 40 | Em AdCa ⅓ | 2+ | 2+ |
| 41 | Em AdCa ⅓ | 3+ | 1+ |
| 42 | Em AdCa-Ser AdCa | 5+ | 0 |
| 43 | Em AdCa ⅓ | 0 | 5+ |
| 44 | Em AdCa ⅔ | 4+ | 4+ |

^{†}0 = absence, + = S/N 2-3, 2+ = S/N 3-5, 3+ = S/N 5-7, 4+ = S/N 7-10, 5+ = S/N ≧ 10.
^{‡}calgranulin A.
Em AdCa = Endometrioid adenocarcinoma
MMMT = Malignant Mixed Mullerian Tumor
Muc AdCa = Mucinous adenocarcinoma
n.k. = not known
Ser AdCa = Serous adenocarcinoma

TABLE 9
Proteins in SEC fraction 7 of Endometrial Cancer Tissue
Identified by nanoLC/MS/MS

| | | Averaged |
|---|---|---|
| | | mol. |
| | | weight |
| Name | Confidence^{a} | (Da) |
| Calgranulin A | 99 | 10,834 |
| Calgranulin B | 99 | 13,242 |
| Heat shock protein 10 | 99 | 10,800 |
| Hemoglobin alpha chain | 90 | 15,126 |
| Hemoglobin beta chain | 99 | 15,867 |
| Putative nucleoside diphosphate kinase | 75 | 15,529 |
| Nucleoside diphosphate kinase A | 75 | 17,149 |
| Nucleoside diphosphate kinase B | 75 | 17,298 |
| Cofilin, non-muscle isoform | 90 | 18,502 |
| Cofilin, muscle isoform | 90 | 18,736 |
| Translationally controlled tumor protein | 90 | 19,595 |
| Peroxiredoxin 1 | 99 | 22,110 |
| Peroxiredoxin 2 | 99 | 21,892 |
| Neutrophil gelatinase-associated lipocalin | 99 | 22,588 |
| Glutathione S-transferase P | 99 | 23,224 |
| Triosephosphate isomerase | 90 | 26,538 |
| Peroxiredoxin 4 | 90 | 30,540 |
| NADPH dehydrogenase | 90 | 30,867 |
| Ig gamma-3 chain C region | 75 | 32,331 |
| Ig gamma-2 chain C region | 75 | 35,884 |
| Ig gamma-4 chain C region | 75 | 35,940 |
| Ig gamma-1 chain C region | 75 | 36,106 |
| C-myc promoter-binding protein | 99 | 37,087 |
| Hetero. nuclear ribonucleoproteins A2 | 99 | 37,429 |
| Cathepsin B precursor | 99 | 37,807 |
| Beta enolase | 75 | 46,855 |
| Alpha enolase | 99 | 47,037 |
| Gamma enolase | 75 | 47,137 |
| Alpha enolase, lung specific | 99 | 49,477 |
| Serum albumin precursor | 99 | 69,366 |
| Polymeric-immunoglobulin receptor precursor | 75 | 83,313 |
| Cone cGMP-specific 3′,5′-cyclic | 75 | 99,102 |
| phosphodiesterase | | |

^{a}“Confidence” value in ProID: the highest possible score is 99.

TABLE 10
Immunohistochemical identification of calgranulinA in tissue microarray
using two monoclonal mouse antibodies (RDI & USB)

| | Macrophage/ | | Epithelium/ | | Stroma/ | |
|---|---|---|---|---|---|---|
| Histologic | Granulocytic | | Carcinoma | | Myometrium | |
| Classification | RDI | USB | RDI | USB | RDI | USB |
| Benign endometrium, n = 10‡ | 0 (1) | 0 (0) | 0 (9) | 0 (9) | 0 (10) | 0 (10) |
| | 1+ (6) | 1+ (10) | 1+ (1) | 1+ (1) | 1+ (0) | 1+ (0) |
| | 2+ (3) | 2+ (0) | 2+ (0) | 2+ (0) | 2+ (0) | 2+ (0) |
| | 3+ (0) | 3+ (0) | 3+ (0) | 3+ (0) | 3+ (0) | 3+ (0) |
| Atypical endometrial | 1+ (2) | 1+ (2) | 1+ (2) | 1+ (2) | 0 (2) | 0 (2) |
| hyperplasia, n = 2 | | | | | | |
| EmCa, n = 13† | 0 (1) | 0 (1) | 0 (6) | 0 (7) | 0 (12) | 0 (13) |
| | 1+ (7) | 1+ (8) | 1+ (4) | 1+ (2) | 1+ (0) | 1+ (0) |
| | 2+ (3) | 2+ (3) | 2+ (2) | 2+ (2) | 2+ (0) | 2+ (0) |
| | 3+ (1) | 3+ (1) | 3+ (0) | 3+ (2) | 3+ (0) | 3+ (0) |
| Non-endometrial | 0 (0) | 0 (1) | 0 (2) | 0 (2) | 0 (4) | 0 (4) |
| carcinomas, n = 4 | 1+ (1) | 1+ (1) | 1+ (1) | 1+ (1) | 1+ (0) | 1+ (0) |
| | 2+ (3) | 2+ (2) | 2+ (1) | 2+ (1) | 2+ (0) | 2+ (0) |
| | 3+ (0) | 3+ (1) | 3+ (0) | 3+ (0) | 3+ (0) | 3+ (0) |
| Benign non-endometrial | 0 (0) | 0 (1) | 0 (0) | 0 (0) | 0 (0) | 0 (0) |
| neoplasms, n = 2 | | | | | | |

( ) = Number of samples
0 = No immunostaining
1+ = Positivity in occasional or few cells
2+ = Positivity in more abundant cells
3+ = Strong positivity in numerous cells
†= In TMA-RDI, one core of EmCa was not displayed
‡= 1 of 11 benign endometrial cores (secretory) did not display in either TMA.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the antibodies, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a cell” includes a plurality of such cells, reference to the “antibody” is a reference to one or more antibodies and equivalents thereof known to those skilled in the art, and so forth.

Below full citations are set out for references.

### FULL CITATIONS FOR REFERENCES

1. Buckley C H: Normal endometrium and non-proliferative conditions of the endometrium. In Obstetrical and Gynaecological Pathology. Fox H, Wells M. Eds. Churchill-Livingstoneo. 5^{th} Ed. London, 2003, Pgs. 391-442.
2. Andersen J S, Mann M (2000) FEBS Lett 480: 25-31.
3. Yao X, Freas A, Ramirez J, Demirev P A, Fenselau C (2001) Anal Chem 73: 2836-2842.
4. Geng M, Ji J, Regnier P (2000) J Chromoatogr A 870: 295-313.
5. Münchbach M, Quadroni M, Miotto G, James P (2000) Anal Chem 72: 4047-4057.
6. Gygi S P, Rist B, Gerber S A, Turecek F, Gelb M H, Aebersold R (1999) Nat Biotechnol 17: 994-999.
7. Goshe M B, Conrads T P, Panisko E A, Angell N H, Veenstra T D, Smith R D (2001) Anal Chem 73: 2578-2586.
8. Williamson B, Marchese J, Juhasz, P, Hatten S, Patterson D, Graber A, Khainovski N, Romeo A, Martin S (2002) Proceedings of the 50th ASMS Conference on Mass Spectrometry and Allied Topics, Orlando.
9. Arcuri F, Ricci C, Ietta F, Cintorino M, Tripodi S A, Cetin I, Garzia E, Schatz F, Klemi P, Santopietro R, Paulesu L (2001) Biol Reprod 64: 1200-1205.
10. Canadian Cancer Statistics 2003. Canadian Cancer Society. 2003. 7-28-0003. Ref Type: Electronic Citation
11. Cancer Facts and Figures. American Cancer Society. 2003.7-25-0003. Ref Type: Electronic Citation
12. Rose P G: Endometrial carcinoma. N Engl J Med 1996, 335:640-649.
13. Agboola O O, Grunfeld E, Coyle D, Perry G A: Costs and benefits of routine follow-up after curative treatment for endometrial cancer. CMAJ 1997, 157:879-886.
14. Duffy M J: Clinical uses of tumor markers: a critical review. Crit. Rev Clin Lab Sci 2001, 38:225-262.
15. Chambers G, Lawrie L, Cash P, Murray G I: Proteomics: a new approach to the study of disease. J Pathol 2000, 192:280-288.
16. Peng J, Gygi S P: Proteomics: the move to mixtures. J Mass Spectrom 2001, 36:1083-1091.
17. Wu W, Hu W, Kavanagh J J: Proteomics in cancer research. Int J Gynecol Cancer 2002, 12:409-423.
18. Bryant-Greenwood P K, Petricoin 3rd EF, Abati A, Liotta L: High-Throughput Proteomic Analysis of Microdissected Cytological Specimens Yields Distinct Expression Profiles of Follicular vs Papillary Thyroid Carcinomas. Mod Pathol 2000, 13:30 A.
19. Jones M B, Krutzsch H, Shu H, Zhao Y, Liotta L A, Kohn E C, Petricoin E F, III: Proteomic analysis and identification of new biomarkers and therapeutic targets for invasive ovarian cancer. Proteomics 2002, 2:76-84.
20: Knezevic V, Leethanakul C, Bichsel V E, Worth J M, Prabhu V V, Gutkind J S, Liotta L A, Munson P J, Petricoin E F, III, Krizman D B: Proteomic profiling of the cancer microenvironment by antibody arrays. Proteomics 2001, 1:1271-1278.
21. Paweletz C P, Gillespie J W, Ornstein D K, Simone N L, Brown M R, Cole K A, Wang Q-H, Huang J, Hu N, Yip T-T, Rich W E, Kohn E C, Linehan W M, Weber T, Taylor P, Emmert-Buck M R, Liotta L A, Petricoin E F, III: Rapid Protein Display Profiling of Cancer Progression Directly From Human Tissue Using a Protein Biochip. Drug Dev Res 2000, 49:3442.
22. Petricoin E F, Ardekani A M, Hitt B A, Levine P J, Fusaro V A, Steinberg S M, Mills G B, Simone C, Fishman D A, Kohn E C, Liotta L A: Use of proteomic patterns in serum to identify ovarian cancer. Lancet 2002, 359:572-577.
23. Vlahou A, Schellhammer P F, Mendrinos S, Patel K, Kondylis F I, Gong L, Nasim S, Wright J G, Jr.: Development of a novel proteomic approach for the detection of transitional cell carcinoma of the bladder in urine. Am J Pathol 2001, 158:1491-1502.
24. Wright Jr G L, Cazares L H, Leung S-M, Nasim S, Adam B-L, Yip T-T, Schellhammer P F, Gong L, Vlahou A: Proteinchip® surface enhanced laser desorption/ionization (SELDI) mass spectrometry: a novel protein biochip technology for detection of prostate cancer biomarkers in complex protein mixtures. Prostate Cancer and Prostatic Diseases 1999, 2:264-276.
25. Wulfkuhle J D, McLean K C, Paweletz C P, Sgroi D C, Trock B J, Steeg P S, Petricoin E F, III: New approaches to proteomic analysis of breast cancer. Proteomics 2001, 1:1205-1215.
26. Blaustein's Pathology of the Female Genital Tract. New York, Springer-Verlag, 2002.
27. Cavanagh A C, Morton H: The purification of early-pregnancy factor to homogeneity from human platelets and identification as chaperonin 10. Eur J Biochem 1994, 222:551-560.
28. Inoue M: Current molecular aspects of the carcinogenesis of the uterine endometrium. Int J Gynecol Cancer 2001, 11:339-348.
29. Mutter G L, Baak J P, Fitzgerald J T, Gray R, Neuberg D, Kust G A, Gentleman R, Gullans S R, Wei L J, Wilcox M: Global expression changes of constitutive and hormonally regulated genes during endometrial neoplastic transformation. Gynecol Oncol 2001, 83:177-185.
30. Wulfkluhle J D, Liotta L A, Petricoin E F: Proteomic applications for the early detection of cancer. Nat Rev Cancer 2003, 3:267-275.
31. Walch A, Komminoth P, Hutzler P, Aubele M, Hofler H, Werner M: Microdissection of tissue sections: application to the molecular genetic characterisation of premalignant lesions. Pathobiology 2000, 68:9-17.
32. Sugiyama Y, Sugiyama K, Hirai Y, Akiyama F, Hasumi K: Microdissection is essential for gene expression profiling of clinically resected cancer tissues. Am J Clin Pathol 2002, 117:109-116.
33. Batorfi J, Ye B, Mok S C, Cseh I, Berkowitz R S, Fulop V: Protein profiling of complete mole and normal placenta using ProteinChip analysis on laser capture microdissected cells. Gynecol Oncol 2003, 88:424-428.
34. Jr G W, Cazares L H, Leung S M, Nasim S, Adam B L, Yip T T, Schellhammer P F, Gong L, Vlahou A: Proteinchip® surface enhanced laser desorption/ionization (SELDI) mass spectrometry: a novel protein biochip technology for detection of prostate cancer biomarkers in complex protein mixtures. Prostate Cancer Prostatic Dis 1999, 2:264-276.
35. von Eggeling F, Davies H, Lomas L, Fiedler W, Junker K, Claussen U, Ernst G: Tissue-specific microdissection coupled with ProteinChip array technologies: applications in cancer research. BioTechniques 2000, 29:1066-1070.
36. Somodevilla-Torres M J, Hillyard N C, Morton H, Alewood D, Halliday J A, Alewood P F, Vesey D A, Walsh M D, Cavanagh A C: Preparation and characterization of polyclonal antibodies against human chaperonin 10. Cell Stress Chaperones 2000, 5:14-20.
37. Cornford P A, Dodson A R, Parsons K F, Desmond A D, Woolfenden A, Fordham M, Neoptolemos J P, Ke Y, Foster C S: Heat shock protein expression independently predicts clinical outcome in prostate cancer. Cancer Res 2000, 60:7099-7105.
38. Quinn K A, Morton H: Effect of monoclonal antibodies to early pregnancy factor (EPF) on the in vivo growth of transplantable murine tumours. Cancer Immunol Immunother 1992, 34:265-271.
39. Kato S, Kato M, Hirano A, Takikawa M, Ohama E: The immunohistochemical expression of stress-response protein (srp) 60 in human brain tumours: relationship of srp 60 to the other five srps, proliferating cell nuclear antigen and p53 protein. Histol Histopathol 2001, 16:809-820.
40. Tabibzadeh S, Kong Q F, Satyaswaroop P G, Babaknia A: Heat shock proteins in human endometrium throughout the menstrual cycle. Hum Reprod 1996, 11:633-640.
41. Ota H, Igarashi S, Hatazawa J, Tanaka T: Distribution of heat shock proteins in eutopic and ectopic endometrium in endometriosis and adenomyosis. Fertil Steril 1997, 68:23-28.
42. Athanasas-Platsis S, Quinn K A, Wong T Y, Rolfe B E, Cavanagh A C, Morton H: Passive immunization of pregnant mice against early pregnancy factor causes loss of embryonic viability. J Reprod Fertil 1989, 87:495-502.
43. Athanasas-Platsis S, Morton H, Dunglison G F, Kaye P L: Antibodies to early pregnancy factor retard embryonic development in mice in vivo. J Reprod Fertil 1991, 92:443-451.
44. Athanasas-Platsis S, Corcoran C M, Kaye P L, Cavanagh A C, Morton H: Early pregnancy factor is required at two important stages of embryonic development in the mouse. Am J Reprod Immunol 2000, 43:223-233.
45. Noonan F P, Halliday W J, Morton H, Clunie G J: Early pregnancy factor is immunosuppressive. Nature 1979, 278:649-651.
46. Quinn I A, Cavanagh A C, Hillyard N C, McKay D A, Morton H: Early pregnancy factor in liver regeneration after partial hepatectomy in rats: relationship with chaperonin 10. Hepatology 1994, 20:1294-1302.
47. Quinn K A: Active immunization with EPF suppresses the formation of immune ascites in BALB/c mice. Immunol Cell Biol 1991, 69 (Pt 1):1-6.
48. National Cancer Institute of Canada: Canadian Cancer Statistics 2003, Toronto, Canada, 2003 [Serial online] April 2003; Available from: URL: http://www.bc.cancer.ca/vgn/images/portal/cit_{—}776/61/38/56158640niw_stats_en.pdf
49. DeSouza, L.; Diehl, G.; Yang, E. C. C.; Guo, J.; Rodrigues, M. J.; Romaschin, A. D.; Colgan, T. J.; Siu, K. W. M. Proteomic Analysis of the Proliferative and Secretory Phases of the Human Endometrium: Protein Identification and Differential Protein Expression. Proteomics. 2004, in press.
50. Yang, E. C. C.; Guo, J.; Diehl, G.; DeSouza, L.; Rodrigues, M. J.; Romaschin, A. D.; Colgan, T. J.; Siu, K. W. M. Expression Profiling of Endometrial Malignancies Reveals a New Tumor Marker: Chaperonin 10.J. Proteome Res. 2004 3, 636-643.
51. Guo, J.; Yang, E. C. C.; DeSouza, L.; Diehl, G.; Rodrigues, M. J.; Romaschin, A. D.; Colgan, T. J.; Siu, K. W. M. A Strategy for High-Resolution and Protein Identification in Surface-Enhanced Laser Desorption/Ionization (SELDI) Mass Spectrometry: Calgranulin A and Chaperonin 10 as Protein Markers for Endometrial Carcinoma. Proteomics.
52. Li, J.; Steen, H.; Gygi, S. P. Protein profiling with cleavable isotope-coded affinity tag (cICAT) reagents. Mol. Cell. Proteomics 2003, 2, 1198-1204.
53. Cappello, F.; Bellafiore, M.; David, S.; Anzalone, R.; Zummo, G. Ten kilodalton heat shock protein (HSP10) is overexpressed during carcinogenesis of large bowel and uterine exocervix. Cancer Lett. 2003, 196, 35-81.
54. Fan, X.; Yan, L.; Jia, S.; Ma, A.; Qiao, C. A study of early pregnancy factor activity in the sera of women with trophoblastic tumour. Am. J. Reprod. Immunol. 1999, 41, 204-208.
55. Kim, C. W.; Kim, J. I.; Park, S. H.; Han, J. Y.; Kim, J. K.; Chung, K. W.; Sun, H. S. Usefulness of plasma tumor M2-pyruvate kinase in the diagnosis of gastrointestinal cancer. Korean J Gastroenterol. 2003, 42, 387-393.
56. Hardt, P. D.; Toepler, M.; Ngoumou, B.; Rupp, J.; Kloer, H. U. Fecal pyruvate kinase concentrations (ELISA based on a combination of clone 1 and clone 3 antibodies) for gastric cancer screening. Anticancer Res. 2003, 23, 855-857.
57. Marshall, M. J.; Goldberg, D. M.; Neal, F. E.; Millar, D. R. Enzymes of glucose metabolism in carcinoma of the cervix and endometrium of the human uterus. Br J Cancer. 1978, 37, 990-1001.
58. Tanaka, M.; Adzuma, K.; Iwami, M.; Yoshimoto, K.; Monden, Y.; Itakura, M. Human calgizzarin; one colorectal cancer-related gene selected by a large scale random cDNA sequencing and northern blot analysis. Cancer Lett. 1995, 89, 195-200.
59. Chaurand, P.; DaGue, B. B.; Pearsall, R. S.; Threadgill, D. W.; Caprioli, R. M. Profiling proteins from azoxymethane-induced colon tumors at the molecular level by matrix-assisted laser desorption/ionization mass spectrometry. Proteomics 2001, 1, 1320-1326.
60. Blaxall, B. C.; Dwyer-Nield, L. D.; Bauer, A. K.; Bohlmeyer, T. J.; Malkinson, A. M.; Port, J. D. Differential expression and localization of the mRNA binding proteins, AU-rich element mRNA binding protein (AUF1) and Hu antigen R (HuR), in neoplastic lung tissue. Mol. Carcinog. 2000, 28, 76-83.
61. Gouble, A.; Grazide, S.; Meggetto, F.; Mercier, P.; Delsol, G.; Morello, D. A new player in oncogenesis: AUF1/hnRNPD overexpression leads to tumorigenesis in transgenic mice. Cancer Res. 2002, 62, 1489-1495.
62. Nishihira, J.; Ishibashi, T.; Fukushima, T.; Sun, B.; Sato, Y.; Todo, S. Macrophage migration inhibitory factor (MIF): Its potential role in tumor growth and tumor-associated angiogenesis. Ann. New York Acad. Sci. 2003, 995, 171-182.
63. Ren, Y.; Tsui, H. T.; Poon, R. T.; Ng, I. O.; Li, Z.; Chen, Y.; Jiang, G.; Lau, C.; Yu, W. C.; Bacher, M.; Fan, S. T. Macrophage migration inhibitory factor: roles in regulating tumor cell migration and expression of angiogenic factors in hepatocellular carcinoma. Int. J. Cancer. 2003, 107, 22-29.
64. Campa, M. J.; Wang, M. Z.; Howard, B.; Fitzgerald, M. C.; Patz, E. F. Jr. Protein expression profiling identifies macrophage migration inhibitory factor and cyclophilin a as potential molecular targets in non-small cell lung cancer. Cancer Res. 2003, 63, 1652-1656.
65. Markert, J. M.; Fuller, C. M.; Gillespie, G. Y.; Bubien, J. K.; McLean, L. A.; Hong, R. L.; Lee, K.; Gullans, S. R.; Mapstone, T. B.; Benos, D. J. Differential gene expression profiling in human brain tumors. Physiol. Genomics. 2001, 555, 21-33.
66. Bacher, M.; Schrader, J.; Thompson, N.; Kuschela, K.; Gemsa, D.; Waeber, G.; Schlegel, J. Up-regulation of macrophage migration inhibitory factor gene and protein expression in glial tumor cells during hypoxic and hypoglycemic stress indicates a critical role for angiogenesis in glioblastoma multiforme. Am. J. Pathol. 2003, 162, 11-17.
67. Mahutte, N. G.; Matalliotakis, I. M.; Goumenou, A. G.; Koumantakis, G. E.; Vassiliadis, S.; Arici, A. Elevations in peritoneal fluid macrophage migration inhibitory factor are independent of the depth of invasion or stage of endometriosis. Fertil. Steril. 2004, 82, 97-101.
68. Rossel, M.; Brambilla, E.; Billaud, M.; Vuitton, D. A.; Blanc-Jouvan, F.; Biichle, S.; Revillard, J. P. Nonspecific increased serum levels of secretory component in lung tumors: relationship to the gene expression of the transmembrane receptor form. Am. J. Respir. Cell. Mol. Biol. 1993, 9, 341-346.
69. Rossel, M.; Billerey, C.; Bittard, H.; Ksiazek, P.; Alber, D.; Revillard, J. P.; Vuitton, D. A. Alterations in polymeric immunoglobulin receptor expression and secretory component levels in bladder carcinoma. Urol. Res. 1991, 19, 361-366.
70. Sun, Z.; Yang, P. Role of imbalance between neutrophil elastase and alpha 1-antitrypsin in cancer development and progression. Lancet Oncol. 2004, 5, 182-190.
71. Ryu, J. W.; Kim, H. J.; Lee, Y. S.; Myong, N. H.; Hwang, C. H.; Lee, G. S.; Yom, H. C. The proteomics approach to find biomarkers in gastric cancer. J. Korean Med. Sci. 2003, 18, 505-509.
72. Nejjari, M.; Berthet, V.; Rigot, V.; Laforest, S.; Jacquier, M. F.; Seidah, N. G.; Remy, L.; Bruyneel, E.; Scoazec, J. Y.; Marvaldi, J.; Luis, J. Inhibition of proprotein convertases enhances cell migration and metastases development of human colon carcinoma cells in a rat model. Am. J. Pathol. 2004, 164, 1925-1933.
73. Choi, H.; Park, C. S.; Kim, B. G.; Cho, J. W.; Park, J. B.; Bae, Y. S.; Bae, D. S. Creatine kinase B is a target molecule of reactive oxygen species in cervical cancer. Mol. Cells. 2001, 12, 412-417.
74. Joseph, J.; Cardesa, A.; Carreras, J. Creatine kinase activity and isoenzymes in lung, colon and liver carcinomas. Br. J. Cancer. 1997, 7776, 600-605.
75. Shields, J. M.; Rogers-Graham, K.; Der, C. J. Loss of transgelin in breast and colon tumors and in RIE-1 cells by Ras deregulation of gene expression through Raf-independent pathways. J. Biol. Chem. 2002, 277, 9790-9799.
76. Chang, J. W.; Jeon, H. B.; Lee, J. H.; Yoo, J. S.; Chun, J. S.; Kim, J. H.; Yoo, Y. J. Augmented expression of peroxiredoxin I in lung cancer. Biochem. Biophys. Res. Commun. 2001, 289, 507-512.
77. Klade, C. S.; Voss, T.; Krystek, E.; Ahom, H.; Zatloukal, K.; Pummer, K; Adolf, G. R. Identification of tumor antigens in renal cell carcinoma by serological proteome analysis. Proteomics 2001, 1, 890-898.
78. Chen, G.; Gharib, T. G.; Huang, C. C.; Thomas, D. G.; Shedden, K. A.; Taylor, J. M.; Kardia, S. L.; Misek, D. E.; Giordano, T. J.; Iannettoni, M. D.; Oninger, M. B.; Hanash, S. M.; Beer, D. G. Proteomic analysis of lung adenocarcinoma: identification of a highly expressed set of proteins in tumors. Clin. Cancer Res. 2002, 8, 2298-2305.
79. Lichtenfels R, Kellner R. Atkins D, Bukur J, Ackermann A, Beck J, Brenner W, Melchior S, Seliger B. Identification of metabolic enzymes in renal cell carcinoma utilizing PROTEOMEX analyses. Biochim. Biophys. Acta. 2003, 1646, 21-31.
80. Crnogorac-Jurcevic, T.; Efthimiou, E.; Nielsen, T.; Loader, J.; Terris, B.; Stamp, G.; Baron, A.; Scarpa, A.; Lemoine, N. R. Expression profiling of microdissected pancreatic adenocarcinomas. Oncogene 2002, 21, 4587-4594.
81. Kuramitsu, Y.; Fujimoto, M.; Tanaka, T.; Ohata, J.; Nakamura, K. Differential expression of phosphatidylethanol-amine-binding protein in rat hepatoma cell lines: analyses of tumor necrosis factor-alpha-resistant cKDH-8/11 and -sensitive KDH-8/YK cells by two-dimensional gel electrophoresis. Electrophoresis 2000, 21, 660-664.
82. Srivastava, S.; Verma, M.; Henson, D. E. Biomarkers for early detection of colon cancer. Clin. Cancer Res. 2001, 7, 1118-1126.
83. Vlahou, A.; Schellhammer, P. F.; Mendrinos, S.; Patel, K.; Kondylis, F. I.; Gong, L.; Nasim, S.; Wright, J. G., Jr. Development of a novel proteomic approach for the detection of transitional cell carcinoma of the bladder in urine. Am. J. Pathol. 2001, 158, 1491-1502.
84. Bundred, N. J. Prognostic and predictive factors in breast cancer. Cancer Treat. Rev. 2001, 27, 137-142.
85. Kao, L. C., Tulac, S. Lobo, S., Imani, B., Yang, J. P., Germeyer, A., Osteen, K., Taylor, R. N., Lessey, B. A., Guidice, L. C., Endocrinology 2002, 143, 2119-2138.
86. Hathout, Y., Riordan, K, Gehrmann, M., Fenselau, C., J. Prot. Res. 2002, 1, 435-442.
87. Rautajoki, K., Nyman, T. A., Lahesmaa, R., Proteomics 2004, 4, 84-92.
88. Bigonnesse, F., Labelle, Y., Akoum, A., Fertility and Sterility 2001, 75, 79-87.
89. Fujiwaki, R., Iida, K, Nakayama, K., Kanasaki, H., Ozaki, T., Hata, K., Sakai, E., Miyazaki, K., Virchows Arch. 2003, 443, 672-677.
90. von Wolff, M., Ursel, S., Hahn, U., Steldinger, R., Strowitzki, T., J. Clin. Endocrin. Met. 2003, 88, 3885-3892.
91. Duchen, M. R., Diabetes 2004, 53, Suppl. 1, S96-S102.
92. Freemantle, S. J., Portland, H. B., Ewings, K., Dmitrovsky, F., DiPetrillo, K., Spinella, M. J., Dmitrovsky, E., Gene 2002, 291, 17-27.
93. Jonkers, J., Korswagen, H. C., Acton, D., Breuer, M., Berns, A., EMBO J. 1997, 16, 441-450.
94. Saitoh, T., Mine, T., Katoh, M., Int. J. Oncol. 2002, 20, 785-795
95. Hongisto, V., Smeds, N., Brecht, S., Herdegen, T., Courtney, M. J., Coffey, E. T., Mol. Cell. Biol. 2003, 23, 6027-6036.
96. Maldonado, V., Castilla, J. A., Martinez, L., Herruzo, A., Concha, A., Fontes, J., Mendoza, N., Garcia-Pena, M. L., Mendoza, J. L., Magan, R., Ortiz, A., Gonzalez, E., J. Assist. Reprod. Genet. 2003, 20, 474-481.
97. Richardson, M. R., Taylor, D. A., Casey, M. L., MacDonald, P. C., Stull, J. T. In Vitro Cell Dev. Biol. 1987, 23, 21-28.
98. Carson, D. D., Lagow, E., Thathiah, A., Al-Shami, R., Farach-Carson, M. C., Vemon, M., Yuan, L., Fritz, M. A., Lessey, B. Mol. Hum. Reprod. 2002, 8, 871-879.
99. Jokimaa, V., Oksjoki, S., Kujari, H., Vuorio, E., Anttila, L., Mol. Hum. Reprod. 2001, 7, 73-78.
100. Barnette, K. G., Sarkar, M. A., Glover, D. D., Li, P., Boyd, C., Lalka, D., Gynecol. Obstet. Invest. 1999, 47, 114-119.
101. Herrmann, U, Jr., Degiampietro, P., Peheim, E., Bachmann, C., Arch. Gynecol. 1987, 240, 233-240.
102. Meier, U., Beier-Hellwig, K., Klug, J., Linder, D., Beier, H. M., Hum. Reprod. 1995, 10, 1305-1310.
103. Berkova, N., Lemay, A., Dresser, D. W., Fontaine, J.-Y., Kerizit, J., Goupil, S., Mol. Hum. Reprod. 2001, 7, 747-754.
104. Li, C., Hong, Y., Tan, Y.-X., Zhou, H., Ai, J.-H., Li, S.-J., Zhang, L., Xia, Q.-C., Wu, J.-R., Wang, H.-Y., Zeng, R., Mol. Cell. Proteomics 2004, 3, 399-409.
105. Issaq, H. J., Conrads, T. P., Prieto, D. A., Tirumalai, R., Veenstra, T. D., Anal. Chem. 2003, 75, 149A-155A.
106. Petricoin III, E. F., Ornstein, D. K., Pawletz, C. P., Ardekani, A., Hackett, P. S., Hitt, B. A., Valassco, A., Trucco, C., Wiegand, L., Wood, K., Simone, C. V., Leving, P. J., Linehan, W. M., Emmert-Buck, M. R., Steinberg, S. M., Kohn, E. C., Liotta, L. A. J. Natl. Cancer Inst. 2002, 94, 1576-1578.
107. Kozak, K. R., Amneus, M. W., Pusey, S. M., Su, F., Luong, M. N., Luong, S. A., Reddy, S. T., Farias-Eisner, R., Proc. Natl. Acad. Sci. USA 2003, 100, 12343-12348.
108. Zhu, W., Wang, X., Ma, Y., Rao, M., Glimm, J., Kovach, J. S., Proc. Natl. Acad. Sci. USA 2003, 100, 14666-14671.
109. Diamandis, E. P., Lancet 2002, 360, 170.
110. Diamandis, E. P., J. Natl. Cancer Inst. 2003, 95, 489-490.
111. Diamandis, E. P., Clin. Chem. 2003, 49, 1271-1278.
112. Sorace, J. M., Zhan, B. BMC Bioinformatics 2003, 4, 24.
113. Cottingham, K., Anal. Chem. 2003, 75, 472A-476A.
114. Marshall, J., Kupchak, P., Zhu, W., Yantha, J., Vrees, T., Furesz, S., Jacks, K., Smith, C., Kireeva, I., Zhang, R., Takahashi, M., Stanton, E., Jackowski, G., J. Proteome Res. 2003, 2, 361-372.
115. Diamandis, E. P., Mol. Cell Proteomics 2004, 3, 367-378.
116. Baggerly, K. A., Morris, J. S., Coombes, K. R. Bioinformatics 2004, 20, 777-785.
117. Check, E., Nature 2004, 429, 496-497.
118. Marshall, J., Jankowski, A., Furesz, S., Kireeva, I., Barker, L., Dombrovsky, M., Zhu, W., Jacks, K., Ingratta, L., Bruin, J., Kristensen, E., Zhang, R., Stanton, E., Takahashi, M., Jackowski, G. J. Proteome Res. 2004, 3, 364-382.
119. Wagner, L., J. Natl. Cancer Inst. 2004, 96, 500-501.
120. He, Q. Y., Chen, J., Kung, H. F., Yuen, A. P., Chiu, J. F., Proteonics 2004, 4, 271-278.
121. Zhang, Z., Bast Jr., R. C., Fung, E. T., Yu, Y., Li, J., Rosenzweig, J. Proc. AACR Abstract No. 5739, 2003.
122. Ye, B., Cramer, D. W., Skates, S. J., Gygi, S. P., Pratomo, V., Fu, L., Hornick, N. K., Licklider, L. J., Schorge, J. O., Berkowitz, R. S., Mok, S. C., Clin. Cancer Res. 2003, 9, 2904-2911.
123. Chan, D. HUPO 2^{nd} Annual & IUBIMB XIX World Congress, Montreal, Quebec, Oct. 8-11, 2003.
124. Guo, J., Yang, E. C. C., DeSouza, L., Rodrigues, M. J., Romaschin, A. D., Colgan, T. J., Siu, K. W. M., Proc. 52^{nd} American Society for Mass Spectrometry Conference, Nashville, Tenn., May 23-27, 2004.
125. Creasman, W. T., Endometrial Carcinoma Available from: URL: http://www.emedicine.com/med/topic674.htm
126. National Cancer Institute of Canada: Canadian Cancer Statistics 2003, Toronto, Canada, 2003 [Serial online] April 2003; Available from: URL: http://www.bc.cancer.ca/vgn/images/portal/cit_{—}776/61/38/56158640niw_stats_en.pdf
127. American Cancer Society, Statistics Available from: URL: http://www.cancer.org/docroot/STT/stt_{—}0.asp
128. Bernstein, L., Deapen, D., Cerhan, J. R., Schwartz, S. M., J. Natl Cancer Inst. 1999, 91, 1654-1662.
129. Grady, G., Ernster V. L., Endometrial cancer In: Schottenfeld, D., Fraumeni Jr., J., Ed., Cancer epidemiology and prevention 2^{nd} ed. New York, Saunders, 1996. p. 1058-1089.
130. Endometrial cancer Available from: URL: http://health.allrefer.com/health/endometrial-cancer-info.html
131. Uterine Cancer (Endometrial carcinima) Available from: URL: http://alwaysyourchoice.com/ayc/adult/womens/uterine_cancer.php
132. Kurman, R. J., Ed., Blaustein's Pathology of the Female Genital Tract, 5^{th} ed. New York, Springer-Verlag, 2002.
133. ExPASy, URL: http://ca.expasy.org/
134. Odink, K., Cerletti, N., Bruggen, J., Clerc, G. R., Tarcsay, L., Zwadlo, G., Gerhards, G., Schlegel, R., Sorg, C., Nature 1987, 330, 80-82.
135. Stulik, J., Osterreicher, J., Koupilova, K., Knizek, J., Macela, A., Bures, J., Jandik, P., Langr, F., Dedic, K, Jungblut, P. R., Electrophoresis 1999, 20, 1047-1054.
136. Kerklioff, C., Eue, I., Sorg, C., Pathobiology 1999, 67, 230-232.
137. Kerkhoff, C., Klempt, M., Sorg, C., Biochim. Biophys. Acta. 1998, 1448, 200-211.
138. Schluesene, H. J., Kremsner, P. G., Meyermann, R., Acta Neuropathol. (Berl.) 1998, 96, 575-580.
139. Hunter, M. J., Chazin, W. J., J. Biol. Chem. 1998, 273, 12427-12435.
140. Bordmann, G., Burmeister, G., Saladin, S., Urassa, H., Mwankyusye, S., Weiss, N., Tanner, M., Clin. Diagn. Lab. Immunol. 1997, 4, 435-439.
141. Lugering, N., Stoll, R., Kucharzik, T., Burmeister, G., Sorg, C., Domschke, W., Clin. Exp. Immunol. 1995, 101, 249-253.
142. Fanjul, M., Renaud, W., Merten, M., Guy-Crotte, O., Hollande E., Figarella, C., Am. J. Physio. 1995, 268, C1241-C1251.
143. Schmid, K. W., Lugering, N., Stoll, R, Brinkbaumer, P., Winde, G., Domschke, W., Bocker, W., Sorg, C., Hum. Pathol. 1995, 26, 334-337.
144. Uchida, T., Fukawa, A., Uchida, M., Fujita, K., Saito, K., J. Proteome Res. 2002, 1, 495-499.
145. Lundy, F. T., Chalk, R., Lamey, P.-J., Shaw, C., Linden, G. J., J. Pathol. 2000, 1992, 540-544.
146. Bhardwaj, R. S., Zotz, C., Zwadlo-Kkarwasser, G., Roth, J., Geobeler, M., Mahnke, K., Falk, M., Meinardus-Hager, G., Sorg, C., Eur. J. Immunol. 1992, 22, 1891-1897.
147. Burkhardt, K., Radespiel-Troger, M., Rupprecht, H. D., Goppelt-Strube, M., Riess, R., Renders, L., Hauser, I. A., Kunzendorf, U., J. Am. Soc. Nephrol. 2001, 12, 1947-1957.
148. Roth, J., Teigelkamp, S., Weike, M., Gruri, L., Tummler, B., Sorg, C., Immunobiology 1992, 186, 304-314.
149. Munaron, L., Antoniotti, S., Pla, A. F., Lovisolo, D. Curr. Med. Chem. 2004, 11, 1533-1543.
150. Lin, J., Black, M., Tang, C., Zimmer, D., Rustandi, R. R., Weber, D. J., Carrier, F., J. Biol. Chem. 2001, 276, 35037-35041.
151. Lin, J., Yang, Q., Yan, Z., Markowitz, J., Wilder, P. T., Carrier, F., Weber, D. J. J. Biol. Chem. 2004, 279, 34071-34077.
152. Gebhardt, C., Breitenbach, U., Tuckermann, J. P., Dittrich, B. T., Richter, K. H., Angel, P. Oncogene 2002, 21, 4266-4276.
153. Kristinsson, J., Nygaard, K., Aadland, E., Barstad, S., Sauar, J., Hofstad, B., Stray, N., Stallemo, A., Haug, B., Ugstad, M., Ton, H., Fuglerud, P. Digestion 2001, 64, 104-110.
154. Tibble, J., Sigthorsson, G., Foster, R., Sherwood, R., Fagerhol, M., Bjarnason, I. Gut 2001, 49, 402-408.
155. Nacken, W., Roth, J., Sorg, C., Kerlchoff, C. Microsc. Res. Tech. 2003, 60, 569-580.
156. Donato, R. Int. J. Biochem. Cell Biol. 2001, 33, 637-668.

1. A method for detecting one or more endometrial markers or polynucleotides encoding the markers associated with an endometrial disease or an endometrium phase in a subject comprising:
(a) obtaining a sample from a subject;
(b) detecting in proteins extracted from the sample one or more endometrial markers or polynucleotides encoding the markers that are associated with the disease or phase; and
(c) comparing the detected amount with an amount detected for a standard.

2. A method of detecting an endometrial disease in a subject, the method comprising:
(a) detecting the levels of one or more endometrial markers associated with endometrial disease that in accordance with the method of claim 1; and
(b) comparing the levels in “(a)” with normal levels of expression of the endometrial markers in a control sample, wherein a significant difference in levels of endometrial markers, relative to the corresponding normal levels, is indicative of endometrial disease.

3. A method as claimed in claim 2 comprising:
(a) contacting a biological sample obtained from a subject with one or more binding agent that specifically binds to the endometrial markers or parts thereof; and
(b) detecting in the sample amounts of endometrial markers that bind to the binding agents, relative to a predetermined standard or cut-off value, and therefrom determining the presence or absence of the endometrial disease in the subject.

4. A method as claimed in claim 3 wherein the binding agent is an antibody.

5. A method for screening a subject for endometrial cancer comprising the method of claim 1 wherein in step “(b)” the one or more endometrial markers or polynucleotides encoding the markers detected are one or more endometrial cancer markers or polynucleotides encoding same and; comparing the amount of endometrial cancer markers detected to a predetermined standard, where detection of a level of endometrial cancer markers different than that of a standard or control is indicative of endometrial cancer.

6. A method of claim 5 wherein the level of endometrial cancer markers are significantly higher compared to the standard and are indicative of endometrial cancer.

7. A method of claim 5 wherein the level of endometrial cancer markers are significantly lower compared to the standard and are indicative of endometrial cancer.

8. A method as claimed in claim 5 wherein the sample is obtained from tissues, extracts, cell cultures, cell lysates, lavage fluid, or physiological fluids.

9. A method as claimed in claim 8 wherein the sample is obtained from a tumor tissue.

10. A method as claimed in claim 5 which further comprises detecting multiple cancer markers.

11. A method of claim 1 for determining the presence or absence of endometrial markers associated with an endometrial disease in a subject wherein one or more polynucleotide encoding an endometrial marker in a sample is detected from the subject and relating the detected amount to the presence of an endometrial disease.

12. A method as claimed in claim 11 wherein the polynucleotide detected is mRNA.

13. A method of claim 12 wherein the polynucleotide is detected by
(a) contacting the sample with oligonucleotides that hybridize to the polynucleotides; and
(b) detecting in the sample levels of nucleic acids that hybridize to the polynucleotides relative to a predetermined standard or cut-off value, and therefrom determining the presence or absence of an endometrial disease in the subject.

14. A method as claimed in claim 12 wherein the mRNA is detected using an amplification reaction.

15. A method as claimed in claim 14 wherein the amplification reaction is a polymerase chain reaction employing oligonucleotide primers that hybridize to the polynucleotides, or complements of such polynucleotides.

16. A method as claimed in claim 12 wherein the mRNA is detected using a hybridization technique employing oligonucleotide probes that hybridize to the polynucleotides or complements of such polynucleotides.

17. A method as claimed in claim 14 wherein the mRNA is detected by (a) isolating mRNA from the sample and combining the mRNA with reagents to convert it to cDNA; (b) treating the converted cDNA with amplification reaction reagents and primers that hybridize to the polynucleotides, to produce amplification products; (d) analyzing the amplification products to detect an amount of mRNA encoding one or more endometrial markers; and (e) comparing the amount of mRNA to an amount detected against a panel of expected values for normal tissue derived using similar primers.

18. (canceled)

19. A method for monitoring the progression of endometrial cancer in a subject, the method comprising: (a) detecting in a sample from the subject at a first time point, one or more endometrial cancer markers or polynucleotides encoding the markers using the method of claim 5; (b) repeating step (a) at a subsequent point in time; and (c) comparing levels detected in steps (a) and (b), and thereby monitoring the progression of endometrial cancer.

20. A method for determining in a subject whether endometrial cancer has metastasized or is likely to metastasize in the future, the method comprising comparing (a) levels of one or more endometrial cancer markers or polynucleotides encoding the markers, in a subject sample in accordance with the method of claim 1; and (b) normal levels or non-metastatic levels of the endometrial cancer markers or polynucleotides encoding the markers, in a control sample wherein a significant difference between the levels of expression in the subject sample and the normal levels or non-metastatic levels is an indication that the endometrial cancer has metastasized.

21. A method for assessing the aggressiveness or indolence of endometrial cancer comprising comparing: (a) levels of expression of one or more endometrial cancer markers or polynucleotides encoding the markers, in a subject sample obtained using the method of claim 1; and (b) normal levels of expression of the endometrial cancer markers or polynucleotides encoding the markers, in a control sample, wherein a significant difference between the levels in the subject sample and normal levels is an indication that the cancer is aggressive or indolent.

22. A diagnostic composition comprising an agent that binds to an endometrial cancer marker or hybridizes to a polynucleotide encoding such marker.

23. A method for assessing the potential efficacy of a test agent for inhibiting endometrial cancer in a subject, the method comprising: (i) detecting in accordance with the method of claim 1: (a) levels of one or more endometrial cancer markers, in a first sample obtained from a subject and exposed to the test agent, wherein the endometrial cancer markers, (b) levels of the endometrial cancer markers in a second sample obtained from the subject, wherein the sample is not exposed to the test agent, and (ii) comparing (a) and (b), wherein a significant difference in the levels of expression of the endometrial cancer markers in the first sample, relative to the second sample, is an indication that the test agent is potentially efficacious for inhibiting endometrial cancer in the subject.

24. A method of assessing the efficacy of a therapy for inhibiting endometrial cancer in a subject, the method comprising comparing: (i) detecting in accordance with the method of claim 1: (a) levels of one or more endometrial cancer markers in a first sample obtained from the subject; (b) levels of the endometrial cancer markers in a second sample obtained from the subject following therapy, and (ii) comparing (a) and (b), wherein a significant difference in the levels of expression of the endometrial cancer markers in the second sample, relative to the first sample, is an indication that the therapy is efficacious for inhibiting endometrial cancer in the subject.

25. A method of selecting an agent for inhibiting endometrial cancer in a subject the method comprising (a) obtaining a sample comprising cancer cells from the subject; (b) separately exposing aliquots of the sample in the presence of a plurality of test agents; (c) detecting levels of one or more endometrial cancer markers in each of the aliquots in accordance with the method of claim 1; and (d) selecting one of the test agents which alters the levels of endometrial cancer markers in the aliquot containing that test agent, relative to other test agents.

26. A method of inhibiting endometrial cancer in a subject, the method comprising (a) obtaining a sample comprising cancer cells from the subject; (b) separately maintaining aliquots of the sample in the presence of a plurality of test agents; (c) detecting levels of one or more endometrial cancer markers in each of the aliquots in accordance with the method of claim 1 and comparing them; and (d) administering to the subject at least one of the test agents which alters the levels of endometrial cancer markers in the aliquot containing that test agent, relative to other test agents.

27. A method of assessing the endometrial cancer cell carcinogenic potential of a test compound, the method comprising: (a) maintaining separate aliquots of endometrial cancer cells in the presence and absence of the test compound; and (b) detecting the expression of one or more endometrial cancer markers, in each of the aliquots in accordance with claim 1 and comparing them, wherein a significant difference in levels of endometrial cancer markers in the aliquot maintained in the presence of the test compound, relative to the aliquot maintained in the absence of the test compound, is an indication that the test compound possesses endometrial cancer cell carcinogenic potential.

28. An in vivo method for imaging an endometrial disease comprising:
(a) injecting a subject with one or more agent that binds to an endometrial marker, the agent carrying a label for imaging the endometrial marker;
(b) allowing the agent to incubate in vivo and bind to an endometrial marker; and
(c) detecting the presence of the label localized to diseased endometrial tissue.

29. A method as claimed in claim 28 wherein the agent is an antibody that specifically reacts with an endometrial marker.

30. Markers that distinguish an endometrium phase or endometrial disease identified by assaying for differential expression of polypeptides in endometrium samples in accordance with the method of claim 1.

31. Markers as claimed in claim 30 wherein differential expression is assayed using mass spectroscopy of polypeptides extracted from the samples.

32. Markers of claim 31 which are up-regulated in endometrial cancer.

33. Markers of claim 31 which are down-regulated in endometrial cancer.

34. A set of markers of claim 30 comprising a plurality of polypeptides comprising or consisting of at least 2, 3, 4, 5, 6, 7, 8, 9, 10, 11, 12, 13, 14, or 15 of the markers listed in Table 1, 4, 5, or 6.

35. A set of markers of claim 34 wherein the polypeptides are selected from the group consisting of polypeptides with the sequence of SEQ ID NOs. 1, 3, 6, 9, 11, 13, 15, 18, 21, 23, 30, 33, 36, 38, and 40.

36. A set of markers of claim 31 wherein the polypeptides are selected from the group consisting of polypeptides with the sequence of SEQ ID NOs. 1, 3, 6, 9, 11, 13, 15, 18, 21, 23, 26, 30, 33, 36, 38, 40, 42, 45, and 47.

37. A set of markers of claim 31 wherein the polypeptides are selected from the group consisting of polypeptides with the sequence of SEQ ID NOs. 26, 42, 45, and 47.

38. A method of claim 1 wherein the endometrial markers are one or more of the polypeptides listed in Table 1 or they have a sequence of SEQ ID NOs. 1, 3, 6, 9, 11, 13, 15, 18, 21, 23, 26, 30, 33, 36, 38, 40, 42, 45, and 47.

39. A method of claim 1 wherein the endometrial marker is chaperonin 10.

40. (canceled)

41. A method of determining uterine endometrial receptivity by first obtaining a serum, uterine fluid or endometrial biopsy sample from a subject and detecting the presence of an endometrial marker associated with a certain endometrium phase, wherein the presence or absence of an endometrial marker as compared to controls indicates uterine receptivity.

42. A method of claim 41 wherein the endometrium phase is the secretory or proliferative phase.

43. A method of monitoring the effects of ovarian hyperstimulation and/or ovulation induction treatments on uterine receptivity which comprises the method of claim 41 wherein: (a) the sample is obtained from a subject receiving the treatments; and (b) wherein the certain phase in which the presence of an endometrial marker is detected in the endometrium is at the time of fertilization, early embryogenesis, and implantation.

44. A method of determining a probability of successful implantation with an ovarian stimulation in vitro fertilization and embryo transfer procedure, comprising detecting the uterine endometrial receptivity in accordance with the method of claim 41:
(a) wherein the sample is obtained from a subject who has undergone an ovarian stimulation in vitro fertilization and embryo transfer procedure; and
(b) wherein determining a probability of successful implantation is based on the subject's determined endometrial marker level;
wherein a significantly different endometrial marker level relative to a standard level is associated with a decreased or increased probability of successful implantation.

45. A method of any of claim 41 wherein the endometrial marker is glutamate receptor subunit zeta 1, a tryptic fragment thereof, and/or macrophage migration inhibitory factor.

46. A method of contraception by interrupting the cyclic presence of an endometrial marker, in particular glutamate receptor subunit zeta 1, a tryptic fragment thereof, macrophage migration inhibitory factor, myosin light chain kinase 2, and/or tropomyosin 1 alpha chain.

47. (canceled)

48. A kit for determining the presence of an endometrial disease in a subject, comprising a known amount of one or more binding agent that specifically binds to an endometrial marker wherein the binding agent comprises a detectable substance, or it binds directly or indirectly to a detectable substance.

49. A kit for determining the presence of endometrial disease in a subject of claim 48 wherein the binding agent is oligonucleotide that hybridizes to a polynucleotide encoding an endometrial marker wherein the oligonucleotide is directly or indirectly labeled with a detectable substance.
